SEQUENCE LISTING (161 sequences)

SEQ ID NO: 1 - DNA, 21 bp, Artificial Sequence
  source: /note=“Description of Artificial Sequence: Synthetic oligonucleotide”
cccatcctcttccctattaag

SEQ ID NO: 2 - DNA, 21 bp, Artificial Sequence
  source: /note=“Description of Artificial Sequence: Synthetic oligonucleotide”
gtcctccacctaatgcctatg

SEQ ID NO: 3 - DNA, 21 bp, Artificial Sequence
  source: /note=“Description of Artificial Sequence: Synthetic oligonucleotide”
gaatgcaccaactacacacac

SEQ ID NO: 4 - DNA, 21 bp, Artificial Sequence
  source: /note=“Description of Artificial Sequence: Synthetic oligonucleotide”
gcgtttcaagagggagttcat

SEQ ID NO: 5 - DNA, 21 bp, Artificial Sequence
  source: /note=“Description of Artificial Sequence: Synthetic oligonucleotide”
ggctcaagccaccaatctatg

SEQ ID NO: 6 - DNA, 21 bp, Artificial Sequence
  source: /note=“Description of Artificial Sequence: Synthetic oligonucleotide”
cgctagagaactggcagtttg

SEQ ID NO: 7 - DNA, 21 bp, Artificial Sequence
  source: /note=“Description of Artificial Sequence: Synthetic oligonucleotide”
gtctgttctgctattcataac

SEQ ID NO: 8 - DNA, 21 bp, Artificial Sequence
  source: /note=“Description of Artificial Sequence: Synthetic oligonucleotide”
gccatcccaacagagatccta

SEQ ID NO: 9 - DNA, 21 bp, Artificial Sequence
  source: /note=“Description of Artificial Sequence: Synthetic oligonucleotide”
cgtggatgtggtggcacaact

SEQ ID NO: 10 - DNA, 21 bp, Artificial Sequence
  source: /note=“Description of Artificial Sequence: Synthetic oligonucleotide”
ccagaagaggagaaggatacc

SEQ ID NO: 11 - DNA, 21 bp, Artificial Sequence
  source: /note=“Description of Artificial Sequence: Synthetic oligonucleotide”
gcggataaagctgtatgctgt

SEQ ID NO: 12 - DNA, 21 bp, Artificial Sequence
  source: /note=“Description of Artificial Sequence: Synthetic oligonucleotide”
gacctcccatctaatcatgtc

SEQ ID NO: 13 - DNA, 21 bp, Artificial Sequence
  source: /note=“Description of Artificial Sequence: Synthetic oligonucleotide”
gaagaggagaaggataccaat

SEQ ID NO: 14 - DNA, 21 bp, Artificial Sequence
  source: /note=“Description of Artificial Sequence: Synthetic oligonucleotide”
ccagaggacctgagagatgat

SEQ ID NO: 15 - DNA, 21 bp, Artificial Sequence
  source: /note=“Description of Artificial Sequence: Synthetic oligonucleotide”
gccaagtgaccgtagtctcat

SEQ ID NO: 16 - DNA, 21 bp, Artificial Sequence
  source: /note=“Description of Artificial Sequence: Synthetic oligonucleotide”
agggactggaatacgctaatt

SEQ ID NO: 17 - DNA, 21 bp, Artificial Sequence
  source: /note=“Description of Artificial Sequence: Synthetic oligonucleotide”
cctggaatacttaagccagaa

SEQ ID NO: 18 - DNA, 21 bp, Artificial Sequence
  source: /note=“Description of Artificial Sequence: Synthetic oligonucleotide”
gactcactctcctgggatgtt

SEQ ID NO: 19 - DNA, 21 bp, Artificial Sequence
  source: /note=“Description of Artificial Sequence: Synthetic oligonucleotide”
ggcaccaaccaccactcagag

SEQ ID NO: 20 - DNA, 21 bp, Artificial Sequence
  source: /note=“Description of Artificial Sequence: Synthetic oligonucleotide”
cggctgcacaacttccaccag

SEQ ID NO: 21 - DNA, 21 bp, Artificial Sequence
  source: /note=“Description of Artificial Sequence: Synthetic oligonucleotide”
ccctgaggcccagtttgagat

SEQ ID NO: 22 - DNA, 21 bp, Artificial Sequence
  source: /note=“Description of Artificial Sequence: Synthetic oligonucleotide”
cgcactcagcctcaagaactc

SEQ ID NO: 23 - DNA, 21 bp, Artificial Sequence
  source: /note=“Description of Artificial Sequence: Synthetic oligonucleotide”
ctggccatcactcttccatgt

SEQ ID NO: 24 - DNA, 21 bp, Artificial Sequence
  source: /note=“Description of Artificial Sequence: Synthetic oligonucleotide”
caggccatctataaatgtctg

SEQ ID NO: 25 - DNA, 21 bp, Artificial Sequence
  source: /note=“Description of Artificial Sequence: Synthetic oligonucleotide”
cccgaaatagctgacacacta

SEQ ID NO: 26 - DNA, 21 bp, Artificial Sequence
  source: /note=“Description of Artificial Sequence: Synthetic oligonucleotide”
gcccataacggtacgtgaagg

SEQ ID NO: 27 - DNA, 21 bp, Artificial Sequence
  source: /note=“Description of Artificial Sequence: Synthetic oligonucleotide”
cgcgtttcaagagggagttca

SEQ ID NO: 28 - DNA, 21 bp, Artificial Sequence
  source: /note=“Description of Artificial Sequence: Synthetic oligonucleotide”
ccggcggataaagctgtatgc

SEQ ID NO: 29 - DNA, 21 bp, Artificial Sequence
  source: /note=“Description of Artificial Sequence: Synthetic oligonucleotide”
ggagcatttcaatctgctttc

SEQ ID NO: 30 - DNA, 21 bp, Artificial Sequence
  source: /note=“Description of Artificial Sequence: Synthetic oligonucleotide”
cctcaagaactccctggaata

SEQ ID NO: 31 - DNA, 21 bp, Artificial Sequence
  source: /note=“Description of Artificial Sequence: Synthetic oligonucleotide”
ctgacctcccatctaatcatg

SEQ ID NO: 32 - DNA, 21 bp, Artificial Sequence
  source: /note=“Description of Artificial Sequence: Synthetic oligonucleotide”
gagatcctatgattgacaaca

SEQ ID NO: 33 - DNA, 21 bp, Artificial Sequence
  source: /note=“Description of Artificial Sequence: Synthetic oligonucleotide”
gcccagtttgagatgccttat

SEQ ID NO: 34 - DNA, 21 bp, Artificial Sequence
  source: /note=“Description of Artificial Sequence: Synthetic oligonucleotide”
ctgctcctacctccaatacct

SEQ ID NO: 35 - DNA, 21 bp, Artificial Sequence
  source: /note=“Description of Artificial Sequence: Synthetic oligonucleotide”
cactggccatcactcttccat

SEQ ID NO: 36 - RNA, 18 nt, Artificial Sequence
  source: /note=“Description of Artificial Sequence: Synthetic oligonucleotide”
cuccuuaccauuaaacug

SEQ ID NO: 37 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
gagaaggaugacauucugutt

SEQ ID NO: 38 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
acagaaugucauccuucuctt

SEQ ID NO: 39 - protein, 21 aa, Artificial Sequence
  source: /note=“Description of Artificial Sequence: Synthetic peptide”
  VARIANT (6)..(6): /replace=“phospho-Tyr”
  VARIANT (13)..(13): /replace=“phospho-Tyr”
  VARIANT (16)..(16): /replace=“phospho-Tyr”
  misc_feature (1)..(21): /note=“Variant residues given in the sequence have no preference with respect to those in the annotations for variant positions”
CPPNAYELFAKGYEDYLQSPL

SEQ ID NO: 40 - protein, 20 aa, Artificial Sequence
  source: /note=“Description of Artificial Sequence: Synthetic peptide”
KNRPGPQTRSDLLLSGRDWN

SEQ ID NO: 41 - RNA, 21 nt, Artificial Sequence
  source: /note=“Description of Artificial Sequence: Synthetic oligonucleotide”
aagagggaguucauucaggaa

SEQ ID NO: 42 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
gggacuggaauacgcuaautt

SEQ ID NO: 43 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
auuagcguauuccaguccctt

SEQ ID NO: 44 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
ggaccugagagaugauauatt

SEQ ID NO: 45 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
uauaucaucucucaggucctt

SEQ ID NO: 46 - DNA, 21 bp, Artificial Sequence
  source: /note=“Description of Artificial Sequence: Synthetic oligonucleotide”
cctcaagaactccctggaata

SEQ ID NO: 47 - RNA, 25 nt, Artificial Sequence
  source: /note=“Description of Artificial Sequence: Synthetic oligonucleotide”
ggacaaucuggaaucucagacauau

SEQ ID NO: 48 - RNA, 25 nt, Artificial Sequence
  source: /note=“Description of Artificial Sequence: Synthetic oligonucleotide”
ggcuccagagaaagcagacaucauu

SEQ ID NO: 49 - RNA, 25 nt, Artificial Sequence
  source: /note=“Description of Artificial Sequence: Synthetic oligonucleotide”
gcggccauguuacaggagcugaauu

SEQ ID NO: 50 - DNA, 69 bp, Artificial Sequence
  source: /note=“Description of Artificial Sequence: Synthetic oligonucleotide”
gatcccgcccagtttgagatgccttatgtgtgctgtccataaggcatctcaaactgggctttttggaaa

SEQ ID NO: 51 - DNA, 69 bp, Artificial Sequence
  source: /note=“Description of Artificial Sequence: Synthetic oligonucleotide”
agcttttccaaaaagcccagtttgagatgccttatggacagcacacataaggcatctcaaactgggcgg

SEQ ID NO: 52 - DNA, 31 bp, Artificial Sequence
  source: /note=“Description of Artificial Sequence: Synthetic oligonucleotide”
aaaaacacttcatatgtctgagacctgtctc

SEQ ID NO: 53 - DNA, 31 bp, Artificial Sequence
  source: /note=“Description of Artificial Sequence: Synthetic oligonucleotide”
aatctcagacatatgaagtgtttcctgtctc

SEQ ID NO: 54 - RNA, 19 nt, Artificial Sequence
  source: /note=“Description of Artificial Sequence: Synthetic oligonucleotide”
ggccaucuauaaaugucug

SEQ ID NO: 55 - DNA, 19 bp, Artificial Sequence
  source: /note=“Description of Artificial Sequence: Synthetic oligonucleotide”
gagggagttcattcaggaa

SEQ ID NO: 56 - DNA, 19 bp, Artificial Sequence
  source: /note=“Description of Artificial Sequence: Synthetic oligonucleotide”
ggatgtggtggcataactt

SEQ ID NO: 57 - DNA, 19 bp, Artificial Sequence
  source: /note=“Description of Artificial Sequence: Synthetic oligonucleotide”
ggataaagctgtatgctgt

SEQ ID NO: 58 - DNA, 19 bp, Artificial Sequence
  source: /note=“Description of Artificial Sequence: Synthetic oligonucleotide”
ggataaagctgtatgctgt

SEQ ID NO: 59 - DNA, 20 bp, Artificial Sequence
  source: /note=“Description of Artificial Sequence: Synthetic oligonucleotide”
ctcttgtgaatgcgtctctt

SEQ ID NO: 60 - DNA, 20 bp, Artificial Sequence
  source: /note=“Description of Artificial Sequence: Synthetic oligonucleotide”
agctctgagttctcttccta

SEQ ID NO: 61 - RNA, 21 nt, Artificial Sequence
  source: /note=“Description of Artificial Sequence: Synthetic oligonucleotide”
gagggaguucauucaggaauu

SEQ ID NO: 62 - DNA, 19 bp, Artificial Sequence
  source: /note=“Description of Artificial Sequence: Synthetic oligonucleotide”
ggccatctataaatgtctg

SEQ ID NO: 63 - DNA, 19 bp, Artificial Sequence
  source: /note=“Description of Artificial Sequence: Synthetic oligonucleotide”
cagacatttatagatggcc

SEQ ID NO: 64 - RNA, 25 nt, Artificial Sequence
  source: /note=“Description of Artificial Sequence: Synthetic oligonucleotide”
gagcacagcacuuccugaaagauga

SEQ ID NO: 65 - RNA, 25 nt, Artificial Sequence
  source: /note=“Description of Artificial Sequence: Synthetic oligonucleotide”
agacgugguugugguggcauaacuu

SEQ ID NO: 66 - RNA, 25 nt, Artificial Sequence
  source: /note=“Description of Artificial Sequence: Synthetic oligonucleotide”
ccaucccaaccgagauccuaugauu

SEQ ID NO: 67 - RNA, 18 nt, Artificial Sequence
  source: /note=“Description of Artificial Sequence: Synthetic oligonucleotide”
ccgcuauugcaccuugaa

SEQ ID NO: 68 - RNA, 25 nt, Artificial Sequence
  source: /note=“Description of Artificial Sequence: Synthetic oligonucleotide”
cagccacugauggacaaucuggaau

SEQ ID NO: 69 - RNA, 25 nt, Artificial Sequence
  source: /note=“Description of Artificial Sequence: Synthetic oligonucleotide”
ccggcuacuuugagacugugcuuua

SEQ ID NO: 70 - RNA, 19 nt, Artificial Sequence
  source: /note=“Description of Artificial Sequence: Synthetic oligonucleotide”
ccgcuauugcaccuuggaa

SEQ ID NO: 71 - DNA, 20 bp, Artificial Sequence
  source: /note=“Description of Artificial Sequence: Synthetic oligonucleotide”
attgcgtccccgaaatagct

SEQ ID NO: 72 - DNA, 19 bp, Artificial Sequence
  source: /note=“Description of Artificial Sequence: Synthetic oligonucleotide”
gcggatggaagacaggcat

SEQ ID NO: 73 - DNA, 19 bp, Artificial Sequence
  source: /note=“Description of Artificial Sequence: Synthetic oligonucleotide”
accgctattgcaccttgga

SEQ ID NO: 74 - DNA, 19 bp, Artificial Sequence
  source: /note=“Description of Artificial Sequence: Synthetic oligonucleotide”
tccaaggtgcaatagcggt

SEQ ID NO: 75 - DNA, 19 bp, Artificial Sequence
  source: /note=“Description of Artificial Sequence: Synthetic oligonucleotide”
accgctattgcaccttgga

SEQ ID NO: 76 - DNA, 19 bp, Artificial Sequence
  source: /note=“Description of Artificial Sequence: Synthetic oligonucleotide”
tccaaggtgcaatagcggt

SEQ ID NO: 77 - DNA, 19 bp, Artificial Sequence
  source: /note=“Description of Artificial Sequence: Synthetic oligonucleotide”
ccgctattgcaccttggaa

SEQ ID NO: 78 - DNA, 25 bp, Artificial Sequence
  source: /note=“Description of Artificial Sequence: Synthetic oligonucleotide”
cagccactgatggacaatctggaat

SEQ ID NO: 79 - DNA, 25 bp, Artificial Sequence
  source: /note=“Description of Artificial Sequence: Synthetic oligonucleotide”
ccggctactttgagactgtgcttta

SEQ ID NO: 80 - DNA, 60 bp, Artificial Sequence
  source: /note=“Description of Artificial Sequence: Synthetic oligonucleotide”
gatccccggtttgatttcctctgcatttcaagagaatgcagaggaaatcaaaccttttta

SEQ ID NO: 81 - DNA, 60 bp, Artificial Sequence
  source: /note=“Description of Artificial Sequence: Synthetic oligonucleotide”
gatccccggactggaatacgctaattttcaagagaaattagcgtattccagtccttttta

SEQ ID NO: 82 - DNA, 60 bp, Artificial Sequence
  source: /note=“Description of Artificial Sequence: Synthetic oligonucleotide”
gatccccggtcttccagctttcctatttcaagagaataggaaagctggaagaccttttta

SEQ ID NO: 83 - DNA, 60 bp, Artificial Sequence
  source: /note=“Description of Artificial Sequence: Synthetic oligonucleotide”
gatccccgccaccactcttccatgttttcaagagaaacatggaagagtggtggcttttta

SEQ ID NO: 84 - DNA, 19 bp, Artificial Sequence
  source: /note=“Description of Artificial Sequence: Synthetic oligonucleotide”
ggtttgatttcctctgcat

SEQ ID NO: 85 - DNA, 19 bp, Artificial Sequence
  source: /note=“Description of Artificial Sequence: Synthetic oligonucleotide”
atgcagaggaaatcaaacc

SEQ ID NO: 86 - DNA, 19 bp, Artificial Sequence
  source: /note=“Description of Artificial Sequence: Synthetic oligonucleotide”
ggtcttccagctttcctat

SEQ ID NO: 87 - DNA, 19 bp, Artificial Sequence
  source: /note=“Description of Artificial Sequence: Synthetic oligonucleotide”
ataggaaagctggaagacc

SEQ ID NO: 88 - DNA, 19 bp, Artificial Sequence
  source: /note=“Description of Artificial Sequence: Synthetic oligonucleotide”
gccaccactcttccatgtt

SEQ ID NO: 89 - DNA, 19 bp, Artificial Sequence
  source: /note=“Description of Artificial Sequence: Synthetic oligonucleotide”
aacatggaagagtggtggc

SEQ ID NO: 90 - skipped

SEQ ID NO: 91 - DNA, 21 bp, Artificial Sequence
  source: /note=“Description of Artificial Sequence: Synthetic oligonucleotide”
ataaggcatctcaaactgggc

SEQ ID NO: 92 - RNA, 21 nt, Artificial Sequence
  source: /note=“Description of Artificial Sequence: Synthetic oligonucleotide”
ccgcuauugcaccuuggaauu

SEQ ID NO: 93 - RNA, 19 nt, Artificial Sequence
  source: /note=“Description of Artificial Sequence: Synthetic oligonucleotide”
acacuucauaugucugaga

SEQ ID NO: 94 - RNA, 19 nt, Artificial Sequence
  source: /note=“Description of Artificial Sequence: Synthetic oligonucleotide”
ucucagacauaugaagugu

SEQ ID NO: 95 - DNA, 19 bp, Artificial Sequence
  source: /note=“Description of Artificial Sequence: Synthetic oligonucleotide”
ggacctgagagatgatata

SEQ ID NO: 96 - DNA, 19 bp, Artificial Sequence
  source: /note=“Description of Artificial Sequence: Synthetic oligonucleotide”
gaggattgcagtggctctt

SEQ ID NO: 97 - protein, 283 aa, Homo sapiens
MASGTTTTAVKIGIIGGTGLDDPEILEGRTEKYVDTPFGKPSDALILGKIKNVDCVLLARHGRQHTIMPSKVNYQANIWALKEEGCTHVIVTTACGSLREEIQPGDIVIIDQFIDRTTMRPQSFYDGSHSCARGVCHIPMAEPFCPKTREVLIETAKKLGLRCHSKGTMVTIEGPRFSSRAESFMFRTWGADVINMTTVPEVVLAKEAGICYASIAMATDYDCWKEHEEAVSVDRVLKTLKENANKAKSLLLTTIPQIGSTEWSETLHNLKNMAQFSVLLPRH

SEQ ID NO: 98 - DNA, 4937 bp, Homo sapiens
ctccgcactgctcactcccgcgcagtgaggttggcacagccaccgctctgtggctcgcttggttcccttagtcccgagcgctcgcccactgcagattcctttcccgtgcagacatggcctctggcaccaccaccaccgccgtgaagattggaataattggtggaacaggcctggatgatccagaaattttagaaggaagaactgaaaaatatgtggatactccatttggcaagccatctgatgccttaattttggggaagataaaaaatgttgattgcgtcctccttgcaaggcatggaaggcagcacaccatcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacaggaccactatgagacctcagtccttctatgatggaagtcattcttgtgccagaggagtgtgccatattccaatggctgagccgttttgccccaaaacgagagaggttcttatagagactgctaagaagctaggactccggtgccactcaaaggggacaatggtcacaatcgagggacctcgttttagctcccgggcagaaagcttcatgttccgcacctggggggcggatgttatcaacatgaccacagttccagaggtggttcttgctaaggaggctggaatttgttacgcaagtatcgccatggcgacagattatgactgctggaaggagcacgaggaagcagtttcggtggaccgggtcttaaagaccctgaaagaaaacgctaataaagccaaaagcttactgctcactaccatacctcagatagggtccacagaatggtcagaaaccctccataacctgaagaatatggcccagttttctgttttattaccaagacattaaagtagcatggctgcccaggagaaaagaagacattctaattccagtcattttgggaattcctgcttaacttgaaaaaaatatgggaaagacatgcagctttcatgcccttgcctatcaaagagtatgttgtaagaaagacaagacattgtgtgtattagagactcctgaatgatttagacaacttcaaaatacagaagaaaagcaaatgactagtaaacatgtgggaaaaaatattacattttaagggggaaaaaaaaacccaccattctcttctccccctattaaatttgcaacaataaagggtggagggtaatctctactttcctatactgccaaagaatgtgaggaagaaatgggactctttggttatttattgatgcgactgtaaattggtacagtatttctggagggcaatttggtaaaatgcatcaaaagacttaaaaatacggacgtactttgtgctgggaactctacatctagcaatttctctttaaaaccatatcagagatgcatacaaagaattatatataaagaagggtgtttaataatgatagttataataataaataattgaaacaatctgaatcccttgcaattggaggtaaattatgtcttagttataattagattgtgaatcagccaactgaaaatcctttttgcatatttcaatgtcctaaaaagacacggttgctctatatatgaagtgaaaaaaggatatggtagcattttatagtactagttttgctttaaaatgctatgtaaatatacaaaaaaactagaaagaaatatatataaccttgttattgtatttgggggagggatactgggataatttttattttctttgaatctttctgtgtcttcacatttttctacagtgaatttaatcaaatagtaaagttgttgtaaaaataaaagtggatttagaaagatccagttcttgaaaacactgtttctggtaatgaagcagaatttaagttggtaatattaaggtgaatgtcatttaagggagttacatctttattctgctaaagaagaggatcattgatttctgtacagtcagaacagtacttgggtttgcaacagctttctgagaaaagctaggtgtttaatagtttaactgaaagtttaactatttaaaagactaaatgcacattttatggtatctgatattttaaaaagtaatgtttgattctcctttttatgagttaaattattttatacgagttggtaatttttgctttttaataaagtggaagcttgcttttttaactctttttttattgttattttatagaaatgctttttgttggccgggcacagttgctcatccatgtaatcccagcactgtgggaggccgagacgggtggatcacaaggtcaggagatcgagaccatcctggctaatgcgttgaaactccgtctctactaaaaatacaaaaaattagctgggcgtggtggtgggcacctgtagtcccagctactcaggaggctgaggcaggagaatggtgtgaacctgggaggtggagcttgcagtgagcagagcttgcagtgagacgagcttgtgccactgcactccagcctgggcaacagagtaagactcagtctcaaaaaaaaaaaaaagagtgaaatgctttttgtttgcttcagttttttatcatggggagatctttttcctcagaattgttttcttttcactgtaggctattacaggatacttcaggatcaagatacagaaccttttatttaaagagtttgtaaagtcaatgtgtttgtttgtgtctctgagattgacttcaagataataagctgctaattgtaaacaaaacagttaccctccagtattaatatgactcattagtgtgagccatttgggtcaagtatgattatgacccttggacttcctgatgtagtattaaatttcaactctggttatccattagcaatctgtagagaacttaatgaacctgaacccaggcttctctagctctggtaacgtgtgattgttttcactacaatatgatacatagatggtaccttacttttcctcattcttaataggtgtctaagaatgtcagggcaaaagtatgggcatttttcttgctatgttcagaaagtacagttctctccaacttgcagaggtacttttcttgattaaatagccttctctagcaacatcattttcagactaactaaatgaatgcagtatactcttttctttgttctcaatcattcactccttatgcaaagccaatataattttcctcataccttatgcttgaggatattgttgaagaacacttcctggaacacttctcacttgtgatgctgtactaattttttttttttaatttaagctagtatactaagtgaacaccatggtcagttgtgagcattttggtttccgcaaaggatggatggtgagcatcatgggaaagctgtagtttagtgacttagcccttagtgattaatagatttgcatgtacatagaagtctttgttggccttataatctgctgttatatttggcatggattttcatggttttgagaatgacatcctggccctgtggtccccgagggtcatggtccttgtgacctggcccctgttcactgcccccttcgctagcacgagttgctgtgcagggctggaggtagctaccatggcttgtttcaaggaaggaaactctggtacggtggcaccctcaggagtggaggacagtgaacttccttgaagagggagtgactaaggtgacctccaacctgccctgagccagctgccctgcaggtgccacgtgagcctgctctggcatccacaggatgctcctggagcctcttctctggctgctacctcagggcatggttgtggccccaccaacacctattttccaaataattattcattcttgtgacagtggcctgaacatgtttttaattttctcaacaagcatttagccagcacttatccagtgaaacaatttgataaggtttcaaggagtatctgatgggttaggaagtcacgaaatgaggagttcttgccacatttgcagagtccctccttgataaggtttggcggtgtccccacccaaatctcatgttgaattgtagttcccataatccccacatgttgtgggagggacccagtgggaggtaattaaatcatgggggtggttacccccacactgctgttctcatgatactgagttctcacaagtcctgtttgttttataaggggcttttcccccttttgctcaacacttcttcctgccatcatgtgaagaaggacgtgtttgtttccccttctgccacgattgtaagtttcctgaggccttcccagctatgtggaactgtgagttaattaaacctctttcctttataaattacccagtcatgggcagtcctttacagcagcatgagaatggactaatacactcctcaaatgttttgaagattgttgcaccttggaactaccagtgtgcacacaatctggctcaatgtatatattggcccagcaaggcaaagaactgaagttccaggatggaagaacctgtgttctcctcataatagtatagaataattcaagataggcaagaaggacagcagtaaatgaagaccatggaagaaaagaaggaatgccaaagatcgaggaaatctaccaagactagtagggtagtccagaagaagctgtttcagggcctgttgccagctatgcctttgagaacctcgggatcccaaagaatgaggggaatttcttcagaaagacaatctcggcatgcattatttctttgttttgaagattcactcatgttgcatgcatctgtagcttgtgccttttttattgcctagtagtattctgtcatatgcctatcttacaatttgattatctattcacctgttgatgaatgtttgaattttttccatttgaggaattttatgaataaagctgctataagcatgaaaaaaaaaaaaaaaaaa

SEQ ID NO: 99 - RNA, 19 nt, Artificial Sequence
  source: /note=“Description of Artificial Sequence: Synthetic oligonucleotide”
ccgcuauugcaccuuggaa

SEQ ID NO: 100 - RNA, 19 nt, Artificial Sequence
  source: /note=“Description of Artificial Sequence: Synthetic oligonucleotide”
caacagagauccuaugauu

SEQ ID NO: 101 - protein, 9 aa, Artificial Sequence
  source: /note=“Description of Artificial Sequence: Synthetic peptide”
MLQELNFGA

SEQ ID NO: 102 - protein, 9 aa, Artificial Sequence
  source: /note=“Description of Artificial Sequence: Synthetic peptide”
GMFSWFPIL

SEQ ID NO: 103 - protein, 9 aa, Artificial Sequence
  source: /note=“Description of Artificial Sequence: Synthetic peptide”
WMWWHNFRT

SEQ ID NO: 104 - protein, 9 aa, Artificial Sequence
  source: /note=“Description of Artificial Sequence: Synthetic peptide”
FEMPYVVRL

SEQ ID NO: 105 - protein, 9 aa, Artificial Sequence
  source: /note=“Description of Artificial Sequence: Synthetic peptide”
KKVWYEWAV

SEQ ID NO: 106 - protein, 9 aa, Artificial Sequence
  source: /note=“Description of Artificial Sequence: Synthetic peptide”
GLPAFLLPL

SEQ ID NO: 107 - protein, 9 aa, Artificial Sequence
  source: /note=“Description of Artificial Sequence: Synthetic peptide”
YAVEKNPNA

SEQ ID NO: 108 - protein, 9 aa, Artificial Sequence
  source: /note=“Description of Artificial Sequence: Synthetic peptide”
YLGLPAFLL

SEQ ID NO: 109 - protein, 9 aa, Artificial Sequence
  source: /note=“Description of Artificial Sequence: Synthetic peptide”
YLQSPLQPL

SEQ ID NO: 110 - protein, 9 aa, Artificial Sequence
  source: /note=“Description of Artificial Sequence: Synthetic peptide”
FLKDDGVSI

SEQ ID NO: 111 - protein, 9 aa, Artificial Sequence
  source: /note=“Description of Artificial Sequence: Synthetic peptide”
SMFWMRVPL

SEQ ID NO: 112 - protein, 9 aa, Artificial Sequence
  source: /note=“Description of Artificial Sequence: Synthetic peptide”
AILPTSIFL

SEQ ID NO: 113 - protein, 9 aa, Artificial Sequence
  source: /note=“Description of Artificial Sequence: Synthetic peptide”
YEWAVTAPV

SEQ ID NO: 114 - protein, 9 aa, Artificial Sequence
  source: /note=“Description of Artificial Sequence: Synthetic peptide”
AQFEMPYVV

SEQ ID NO: 115 - protein, 9 aa, Artificial Sequence
  source: /note=“Description of Artificial Sequence: Synthetic peptide”
SYLQYLEYL

SEQ ID NO: 116 - protein, 9 aa, Artificial Sequence
  source: /note=“Description of Artificial Sequence: Synthetic peptide”
SWFPILFPI

SEQ ID NO: 117 - protein, 9 aa, Artificial Sequence
  source: /note=“Description of Artificial Sequence: Synthetic peptide”
MFSWFPILF

SEQ ID NO: 118 - protein, 9 aa, Artificial Sequence
  source: /note=“Description of Artificial Sequence: Synthetic peptide”
MFWMRVPLV

SEQ ID NO: 119 - protein, 9 aa, Artificial Sequence
  source: /note=“Description of Artificial Sequence: Synthetic peptide”
NLESQTYEV

SEQ ID NO: 120 - protein, 9 aa, Artificial Sequence
  source: /note=“Description of Artificial Sequence: Synthetic peptide”
VRLHNFHQL

SEQ ID NO: 121 - protein, 9 aa, Artificial Sequence
  source: /note=“Description of Artificial Sequence: Synthetic peptide”
CLDGAQHFL

SEQ ID NO: 122 - protein, 9 aa, Artificial Sequence
  source: /note=“Description of Artificial Sequence: Synthetic peptide”
TLCDYSKRI

SEQ ID NO: 123 - protein, 9 aa, Artificial Sequence
  source: /note=“Description of Artificial Sequence: Synthetic peptide”
MWWHNFRTL

SEQ ID NO: 124 - protein, 9 aa, Artificial Sequence
  source: /note=“Description of Artificial Sequence: Synthetic peptide”
GFAGYFETV

SEQ ID NO: 125 - protein, 9 aa, Artificial Sequence
  source: /note=“Description of Artificial Sequence: Synthetic peptide”
IPGEYTSFL

SEQ ID NO: 126 - protein, 9 aa, Artificial Sequence
  source: /note=“Description of Artificial Sequence: Synthetic peptide”
CTLEFPVEV

SEQ ID NO: 127 - protein, 9 aa, Artificial Sequence
  source: /note=“Description of Artificial Sequence: Synthetic peptide”
VLHGFAGYF

SEQ ID NO: 128 - protein, 9 aa, Artificial Sequence
  source: /note=“Description of Artificial Sequence: Synthetic peptide”
GAYLGLPAF

SEQ ID NO: 129 - protein, 9 aa, Artificial Sequence
  source: /note=“Description of Artificial Sequence: Synthetic peptide”
LLKLEVQFI

SEQ ID NO: 130 - protein, 9 aa, Artificial Sequence
  source: /note=“Description of Artificial Sequence: Synthetic peptide”
KMHQRLIFR

SEQ ID NO: 131 - protein, 9 aa, Artificial Sequence
  source: /note=“Description of Artificial Sequence: Synthetic peptide”
TWMWWHNFR

SEQ ID NO: 132 - protein, 9 aa, Artificial Sequence
  source: /note=“Description of Artificial Sequence: Synthetic peptide”
LKLEVQFII

SEQ ID NO: 133 - protein, 9 aa, Artificial Sequence
  source: /note=“Description of Artificial Sequence: Synthetic peptide”
LYQDITLSI

SEQ ID NO: 134 - protein, 9 aa, Artificial Sequence
  source: /note=“Description of Artificial Sequence: Synthetic peptide”
AYLGLPAFL

SEQ ID NO: 135 - protein, 9 aa, Artificial Sequence
  source: /note=“Description of Artificial Sequence: Synthetic peptide”
KLYNEVRAC

SEQ ID NO: 136 - protein, 9 aa, Artificial Sequence
  source: /note=“Description of Artificial Sequence: Synthetic peptide”
KTWMWWHNF

SEQ ID NO: 137 - protein, 9 aa, Artificial Sequence
  source: /note=“Description of Artificial Sequence: Synthetic peptide”
TVLHGFAGY

SEQ ID NO: 138 - protein, 9 aa, Artificial Sequence
  source: /note=“Description of Artificial Sequence: Synthetic peptide”
QELNFGAYL

SEQ ID NO: 139 - protein, 9 aa, Artificial Sequence
  source: /note=“Description of Artificial Sequence: Synthetic peptide”
VWYEWAVTA

SEQ ID NO: 140 - protein, 9 aa, Artificial Sequence
  source: /note=“Description of Artificial Sequence: Synthetic peptide”
CMPVFHPRF

SEQ ID NO: 141 - protein, 9 aa, Artificial Sequence
  source: /note=“Description of Artificial Sequence: Synthetic peptide”
RLLKLEVQF

SEQ ID NO: 142 - protein, 9 aa, Artificial Sequence
  source: /note=“Description of Artificial Sequence: Synthetic peptide”
ILFPIKQPI

SEQ ID NO: 143 - protein, 9 aa, Artificial Sequence
  source: /note=“Description of Artificial Sequence: Synthetic peptide”
VTAPVCSAI

SEQ ID NO: 144 - protein, 9 aa, Artificial Sequence
  source: /note=“Description of Artificial Sequence: Synthetic peptide”
FDFLCMPVF

SEQ ID NO: 145 - protein, 9 aa, Artificial Sequence
  source: /note=“Description of Artificial Sequence: Synthetic peptide”
RYCTLEFPV

SEQ ID NO: 146 - protein, 9 aa, Artificial Sequence
  source: /note=“Description of Artificial Sequence: Synthetic peptide”
AAMLQELNF

SEQ ID NO: 147 - protein, 9 aa, Artificial Sequence
  source: /note=“Description of Artificial Sequence: Synthetic peptide”
ILPTSIFLT

SEQ ID NO: 148 - protein, 9 aa, Artificial Sequence
  source: /note=“Description of Artificial Sequence: Synthetic peptide”
FLAPISSSK

SEQ ID NO: 149 - protein, 9 aa, Artificial Sequence
  source: /note=“Description of Artificial Sequence: Synthetic peptide”
VALEIGADL

SEQ ID NO: 150 - protein, 9 aa, Artificial Sequence
  source: /note=“Description of Artificial Sequence: Synthetic peptide”
ADLPSNHVI

SEQ ID NO: 151 - protein, 9 aa, Artificial Sequence
  source: /note=“Description of Artificial Sequence: Synthetic peptide”
MHQRLIFRL

SEQ ID NO: 152 - protein, 9 aa, Artificial Sequence
  source: /note=“Description of Artificial Sequence: Synthetic peptide”
FLCMPVFHP

SEQ ID NO: 153 - protein, 9 aa, Artificial Sequence
  source: /note=“Description of Artificial Sequence: Synthetic peptide”
KNPNAVVTL

SEQ ID NO: 154 - protein, 9 aa, Artificial Sequence
  source: /note=“Description of Artificial Sequence: Synthetic peptide”
VLSKMHQRL

SEQ ID NO: 155 - protein, 9 aa, Artificial Sequence
  source: /note=“Description of Artificial Sequence: Synthetic peptide”
AGYFETVLY

SEQ ID NO: 156 - protein, 9 aa, Artificial Sequence
  source: /note=“Description of Artificial Sequence: Synthetic peptide”
FAGYFETVL

SEQ ID NO: 157 - protein, 9 aa, Artificial Sequence
  source: /note=“Description of Artificial Sequence: Synthetic peptide”
GRDWNTLIV

SEQ ID NO: 158 - protein, 9 aa, Artificial Sequence
  source: /note=“Description of Artificial Sequence: Synthetic peptide”
LLSGRDWNT

SEQ ID NO: 159 - DNA, 18 bp, Artificial Sequence
  source: /note=“Description of Artificial Sequence: Synthetic oligonucleotide”
ggactggaatacgctaat

SEQ ID NO: 160 - DNA, 19 bp, Artificial Sequence
  source: /note=“Description of Artificial Sequence: Synthetic oligonucleotide”
aattagcgtattccagtcc

SEQ ID NO: 161 - protein, 21 aa, Artificial Sequence
  source: /note=“Description of Artificial Sequence: Synthetic peptide”
  MOD_RES (3)..(3): Orn
SGXGKGGKGLGKGGAKRHRKV

(19) United States
(12) Patent Application Publication
(10) Pub. No.: US 2018/0010132 A1
(43) Pub. Date: Jan. 11, 2018

(54) INHIBITION OF PRMT5 TO TREAT MTAP-DEFICIENCY-RELATED DISEASES

(71) Applicant: NOVARTIS AG, Basel (CH)

(72) Inventors: Konstantinos John MAVRAKIS, Boston, MA (US); Earl Robert MCDONALD, III, Wayland, MA (US); Frank Peter STEGMEIER, Acton, MA (US)

(73) Assignee: Novartis AG, Basel (CH)

(21) Appl. No.: 15/510,542

(22) PCT Filed: Sep. 9, 2015
(86) PCT No.: PCT/IB2015/056902

§ 371 Date: Mar. 10, 2017

Related U.S. Application Data
(60) Provisional application No. 62/131,437, filed on Mar. 11, 2015.
(60) Provisional application No. 62/049,004, filed on Sep. 11, 2014.

(51) Int. Cl. C12N 15/113; A61K 45/06; A61K 31/7088; C07K 16/40; A61K 39/395; G01N 33/574; A61K 39/00

(52) U.S. Cl. CPC C12N 15/1137; A61K 39/3955; A61K 45/06; G01N 33/574; C12Y 201/01; A61K 31/7088; C07K 16/40; A61K 2039/505; C12N 2310/14; C12N 2320/31; C07K 2317/76

(57) ABSTRACT

The invention provides novel personalized therapies, kits, transmittable forms of information and methods for use in treating patients having cancer, wherein the cancer is MTAP-deficient and/or MTA-accumulating and thus amenable to therapeutic treatment with a PRMT5 inhibitor. Kits, methods of screening for candidate PRMT5 inhibitors, and associated methods of treatment are also provided.

### TECHNICAL FIELD

The present invention provides novel compositions, as well as diagnostic and treatment methods for diseases related to MTAP deficiency and/or MTA accumulation, including, but not limited to, types of cancer.

### BACKGROUND

Many types of cancer are associated with a poor prognosis.

Pancreatic cancer is associated with a poor long-term survival rate of only 10% to 15% after resection. Patients with positive microscopic resection margins have a worse survival. The median survival was 19.7 months with chemotherapy versus 14.0 months without. See, e.g., Neoptolemos et al. 2001 Ann. Surg. 234: 758-768.

Mesothelioma is a rare form of cancer that develops from cells of the mesothelium, the protective lining that covers many of the internal organs of the body. Mesothelioma is most commonly caused by exposure to asbestos. While mesothelioma is still relatively rare, rates have increased in the last twenty years. One study showed a survival rate of only 38% at 2 years and 15% at 5 years (median 19 months). See, e.g., Sugarbaker et al. 1999 J. Thorac. Card. Surg. 117: 54-65.

Glioblastoma is the most common and most aggressive malignant primary brain tumor in humans. It involves glial cells and accounts for half of all brain tumor cases and a fifth of all intracranial tumors. Treatment can involve surgery, radiation and chemotherapy. However, median survival with treatment is only 15 months.

An unmet medical need exists for new treatments for these and other types of cancer.

There is an increasing body of evidence that suggests a patient's genetic profile can be determinative to a patient's responsiveness to a therapeutic treatment. Given the numerous therapies available to an individual having cancer, a determination of the genetic factors that influence, for example, response to a particular drug, could be used to provide a patient with a personalized treatment regime. Such personalized treatment regimens offer the potential to maximize therapeutic benefit to the patient while minimizing related side effects that can be associated with alternative and less effective treatment regimens.

### SUMMARY OF THE INVENTION

According to a first aspect of the invention, methods for inhibiting the proliferation of cells that are MTAP-deficient and/or MTA-accumulating, including types of glioblastoma and other cancer cells, are provided. The methods comprise the step of administering, to a subject in need thereof, a PRMT5 inhibitor in an amount that is effective to inhibit the proliferation of the MTAP-deficient and/or MTA-accumulating cells, including cancer cells. In some embodiments, the MTAP-deficient and/or MTA-accumulating cells are also CDKN2A-deficient. Cells can be determined to be MTAP deficient by techniques known in the art, for example, immunohistochemistry utilizing an anti-MTAP antibody or derivative thereof, and/or genomic sequencing, and/or nucleic acid hybridization or amplification utilizing at least one probe or primer comprising a sequence of at least 12 contiguous nucleotides (nt) of the sequence of MTAP provided in SEQ ID NO: 98, wherein the primer is no longer than about 30 nt, about 50 nt, or about 100 nt in length. Cells are determined to be MTA overproducing or MTA accumulating by techniques known in the art; methods for detecting MTA include, as a non-limiting example, liquid chromatography-electrospray ionization-tandem mass spectrometry (LC-ESI-MS/MS).

In one embodiment, the invention provides use of a molecule that inhibits the cellular function of the PRMT5 protein for the treatment of a disease associated with MTAP deficiency and/or MTA accumulation, including, but not limited to, a cancer, including, for example, but not limited to: glioblastoma, bladder cancer, pancreatic cancer, mesothelioma, melanoma, lung squamous, lung adenocarcinoma, diffuse large B-cell lymphoma, leukemia, head and neck cancer, and cancers of the kidney, breast, endometrium, urinary tract, liver, soft tissue, pleura and large intestine.

Also provided is a use of a molecule that inhibits the cellular function of the PRMT5 protein for the manufacture of a medicament for treating a disease associated with MTAP deficiency and/or MTA accumulation, including, but not limited to, a cancer, including, for example, but not limited to: glioblastoma, bladder cancer, pancreatic cancer, mesothelioma, melanoma, lung squamous, lung adenocarcinoma, diffuse large B-cell lymphoma, leukemia, head and neck cancer, and cancers of the kidney, breast, endometrium, urinary tract, liver, soft tissue, pleura and large intestine.

The PRMT5 inhibitor may be selected from the group consisting of: a RNA inhibitor (e.g., a RNAi agent), a CRISPR, a TALEN, a zinc finger nuclease, an mRNA, an antibody or derivative thereof, a chimeric antigen receptor T cell (CART) or a low molecular weight (LMW) compound.

The PRMT5 inhibitor may be selected from the group consisting of: an antibody or derivative thereof, or a low molecular weight compound. In some embodiments, the antibody or a derivative thereof binds to a HLA-peptide complex comprising a peptide having the sequence of any of SEQ ID NOs: 101-158.

According to an embodiment, the method according to the first aspect comprises administering to a subject in need thereof, a PRMT5 inhibitor in combination with a second therapeutic agent.

In an embodiment, the second therapeutic agent is an anti-cancer agent, anti-allergic agent, anti-nausea agent (or anti-emetic), pain reliever, or cytoprotective agent.

According to one embodiment, the second therapeutic agent is an anti-cancer agent selected from the list consisting of: an HDAC inhibitor, fluorouracil (5-FU) and irinotecan, a HDM2 inhibitor, a purine analogue, 6-thioguanine, 6-mercaptopurine, and CDK4 inhibitors, including, but not limited to, LEE011, and inhibitors of HDM2i, PI3K/mTOR-I, MAPKi, RTKi (EGFRi, FGFRi, METi, IGFiRi, JAKi, and WNTi.

According to a second aspect of the invention, a method of determining if a subject afflicted with a cancer will respond to therapeutic treatment with a PRMT5 inhibitor is provided. The method comprises the steps: a) evaluating a test sample obtained from said subject for MTAP deficiency and/or MTA accumulation relative to a reference normal or non-cancerous control sample, wherein MTAP deficiency and/or MTA accumulation in the test sample indicates that the subject will respond to therapeutic treatment with a PRMT5 inhibitor; wherein the method comprises the following optional steps: b) determining the level of PRMT5 in the subject, wherein steps a) and b) can be performed in any order; c) administering a therapeutically effective amount of a PRMT5 inhibitor to the subject; and d) determining the level of PRMT5 in the subject following step c), wherein a decrease in the level of PRMT5 is correlated with the inhibition of the proliferation of the cancer, and wherein steps c) and d) are performed after steps a) and b). In some embodiments, the human cells are also CDKN2A-deficient. In some embodiments, the cells are determined to be MTAP deficient by any technique known in the art, for example, immunohistochemistry utilizing an anti-MTAP antibody or derivative thereof, and/or genomic sequencing, or nucleic acid hybridization or amplification utilizing at least one probe or primer comprising a sequence of at least 12 contiguous nucleotides (nt) of the sequence of MTAP provided in SEQ ID NO: 98, wherein the primer is no longer than about 30 nt. In some embodiments, cells are determined to be MTA-accumulating by any technique known in the art, for example, LC-ESI-MS/MS.

In an embodiment of the second aspect, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

The PRMT5 inhibitor may be selected from the group consisting of a RNA inhibitor (e.g., a RNAi agent), a CRISPR, a TALEN, a zinc finger nuclease, an mRNA, an antibody or derivative thereof, a chimeric antigen receptor T cell (CART) or a low molecular weight compound. In some embodiments, the antibody or a derivative thereof binds to a HLA-peptide complex comprising a peptide having the sequence of any of SEQ ID NOs: 101-158.

In some embodiments, the PRMT5 inhibitor is a short hairpin RNA (shRNA) or a short inhibitory RNA (siRNA) or other molecule capable of mediating RNA interference against PRMT5.

In some embodiments, the PRMT5 inhibitor is a molecule capable of mediating RNA interference against PRMT5 and comprising a sequence selected from the group consisting of SEQ ID NO: 1 to SEQ ID NO: 18, 41-49, 52-79, and 84-96.

According to a third aspect of the invention, a method of determining if a cancer cell is MTAP deficient and therefore sensitive to PRMT5 inhibition, is provided. The method comprises the steps of: a) measuring the level, activity, expression and/or presence of the MTAP gene or its protein product in the cancer cell; measuring the level, activity, expression and/or presence of the MTAP gene or its protein product in a non-cancerous or normal cell; wherein steps (a) and (b) can be performed in any order; and (c) comparing the level, activity, expression and/or presence of the MTAP gene or its protein product in the cancer cell and a non-cancerous or normal cell, wherein a lower level, activity, expression and/or presence of the MTAP gene or its protein product in the cancer cell indicates that this cell is MTAP deficient, wherein MTAP deficiency indicates the cell is sensitive to a PRMT5 inhibitor. In some embodiments, the cancer cell is also CDKN2A-deficient.

According to a fourth aspect of the invention, a method of determining the sensitivity of a cancer cell to a PRMT5 inhibitor is provided. The method comprises: comparing the level, activity, expression or presence of the MTAP gene or its protein product and/or MTA in said cancer cell with the level, activity, expression or presence of the MTAP gene or its protein product and/or MTA in a non-cancerous or a normal control cell to determine if the cancer cell is MTAP-deficient and/or MTA-accumulating, wherein MTAP deficiency and/or MTA accumulation or MTA accumulation in said cancer cell indicates that said cell is sensitive to a PRMT5 inhibitor. In some embodiments, the cancer cell is also CDKN2A-deficient.

In an embodiment, the cancer cell is a glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

In some embodiments, the PRMT5 inhibitor is a short hairpin RNA (shRNA) or a short inhibitory RNA (siRNA) or other molecule capable of mediating RNA interference against PRMT5.

In some embodiments, the PRMT5 inhibitor is molecule capable of mediating RNA interference against PRMT5 and comprising a sequence selected from the group consisting of SEQ ID NO: 1 to SEQ ID NO: 18, 41-49, 52-79, or 84-96.

The PRMT5 inhibitor may be a low molecular weight compound, a cyclic peptide, an aptamers or CRISPRs.

According to a fifth aspect of the invention, a method of screening for PRMT5 inhibitors is provided. The method comprises contacting a first sample containing one or more MTAP-deficient and/or MTA-accumulating cells with a candidate PRMT5 inhibitor and measuring the reduction in viability of said cells; contacting a second sample containing the same type of cells with a known PRMT5 inhibitor and measuring the reduction in viability of said cells; comparing the reduction in viability of the cells in the first sample with that of the second sample, to determine the potency of the candidate PRMT5 inhibitor. In some embodiments, the MTAP-deficient and/or MTA-accumulating cells are also CDKN2A-deficient.

According to a sixth aspect of the invention, a kit for predicting the sensitivity of a subject afflicted with a MTAP-deficiency-related cancer for treatment with a PRMT5 inhibitor is provided. The method comprises: i) reagents capable of detecting human MTAP-deficiency in cancer cells; and ii) instructions for how to use said kit. In some embodiments, the MTAP-deficient cells are also CDKN2A-deficient.

According to a seventh aspect of the invention, a composition comprising a PRMT5 inhibitor for use in treatment of cancer in a selected patient population is provided, wherein the patient population is selected on the basis of being afflicted with a MTAP-deficient and/or MTA-accumulating cancer.

In one embodiment, the cancer is selected from a group consisting of glioblastoma, bladder cancer, pancreatic cancer, mesothelioma, melanoma, lung squamous, lung adenocarcinoma, diffuse large B-cell lymphoma (DLBCL), leukemia, and head and neck cancer, and cancer of the kidney, breast, endometrium, urinary tract, liver, soft tissue, pleura and large intestine.

According to an eighth aspect of the invention, a therapeutic method of treating a subject afflicted with a cancer associated with MTAP deficiency and/or MTA accumulation is provided, comprising the steps of: contacting a test sample obtained from said subject with a reagent capable of detecting human MTAP-deficient and/or MTA-accumulating cancer cells; comparing the test sample with a reference sample taken from a non-cancerous or normal control subject, wherein MTAP deficiency and/or MTA accumulation in said test sample indicates said afflicted subject will respond to therapeutic treatment with a PRMT5 inhibitor; and administering a therapeutically effective amount of PRMT5 inhibitor to those subject identified in step b). In some embodiments, the cancer cells are also CDKN2A-deficient.

According to a ninth aspect of the invention, a therapeutic method of treating a subject afflicted with a cancer associated with MTAP deficiency and/or MTA accumulation is provided comprising the steps of: contacting a test sample obtained from said subject with a reagent capable of detecting human MTAP-deficient and/or MTA-accumulating cancer cells; comparing the test sample with a reference sample taken from a non-cancerous or normal control subject, wherein MTAP deficiency and/or MTA accumulation in said test sample indicates said afflicted subject will respond to therapeutic treatment with a PRMT5 inhibitor; and administering a therapeutically effective amount of the composition according to the seventh aspect of the invention. In some embodiments, the cancer cells are also CDKN2A-deficient.

According to a tenth aspect of the invention, a method of determining if a subject afflicted with a cancer associated with MTAP deficiency and/or MTA accumulation will respond to therapeutic treatment with a PRMT5 inhibitor is provided, comprising the steps of: contacting a test sample obtained from said subject with a reagent capable of detecting human cancer cells exhibiting MTAP deficiency and/or MTA accumulation; and comparing the test sample with a reference sample taken from a non-cancerous or normal control subject, wherein the detection of MTAP deficiency and/or MTA accumulation in said sample obtained from said afflicted subject indicates said afflicted subject will respond to therapeutic treatment with a PRMT5 inhibitor. In some embodiments, the method further comprises the step of determining the level of PRMT5 in the cancer cells. In many cancers, PRMT5 is over-expressed. The level of expression of PRMT5 can be taken into account when determining the therapeutically effective dosage of a PRMT5 inhibitor. In addition, during treatment, the levels of PRMT5 can be monitored to assess disease or treatment progression.

According to an eleventh aspect of the invention, a method of determining if a subject afflicted with a cancer associated with MTAP deficiency and/or MTA accumulation will respond to therapeutic treatment with a PRMT5 inhibitor is provided, comprising the steps of: contacting a test sample obtained from said subject with a reagent capable of detecting human cancer cells exhibiting MTAP deficiency and/or MTA accumulation; and comparing the test sample with a reference sample taken from a non-cancerous or normal control subject, wherein the detection of MTAP deficiency and/or MTA accumulation in said sample obtained from said afflicted subject indicates said afflicted subject will respond to therapeutic treatment with a PRMT5 inhibitor. In some embodiments, the method further comprises the step of determining the level of PRMT5 in the cancer cells. In many cancers, PRMT5 is over-expressed. The level of expression of PRMT5 can be taken into account when determining the therapeutically effective dosage of a PRMT5 inhibitor. In addition, during treatment, the levels of PRMT5 can be monitored to assess disease or treatment progression.

### BRIEF DESCRIPTION OF THE DRAWINGS

FIG. 1 shows a scatter plot representing the relative levels of MTAP expression for all cell line models screened. MTAP expression levels are plotted on the Y-axis whereas PRMT5 knockdown sensitive and insensitive models are categorically binned on the X-axis, each dot is representative of one cell line. The overwhelming majority of PRMT5 dependent cell lines do not express MTAP as determined by a value of <4.5.

FIG. 2 shows PRMT5 silencing in SNU449 cells. Stable cell lines expressing doxycycline-inducible shRNAs directed against PRMT5 (numbered accordingly) were generated and assessed for efficiency of knockdown after 5-days post doxycycline (dox) induction (+). Protein levels were compared to non-induced levels (−). Levels of histone H4R3me2 were also assessed upon PRMT5 knockdown and showed good correlation. The PRMT5 shRNAs with the most robust knockdown and modulation of the H4R3me2 mark are circled and were taken forward for further validation studies. GAPDH and total histone H3 levels were used as loading controls. Expression levels of the tetracycline repressor protein (TET^{R}) were used to confirm that comparable expression of the transactivators were present in each cell line.

FIG. 3 A-N show that MTA accumulation sensitizes MTAP-expressing cells to partial loss of PRMT5.

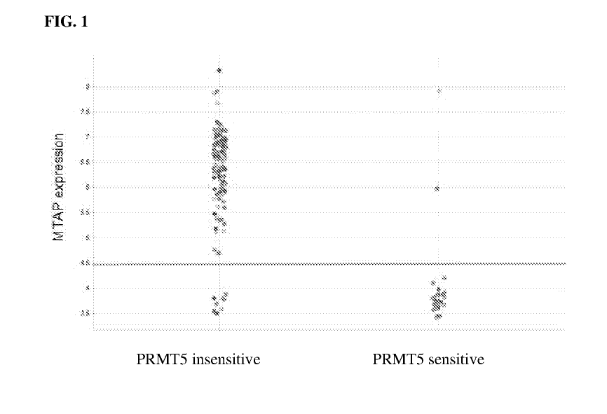

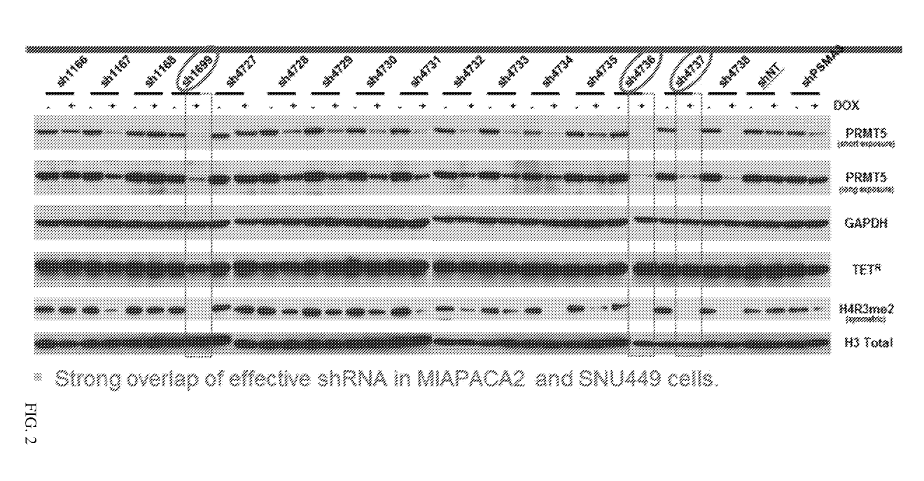

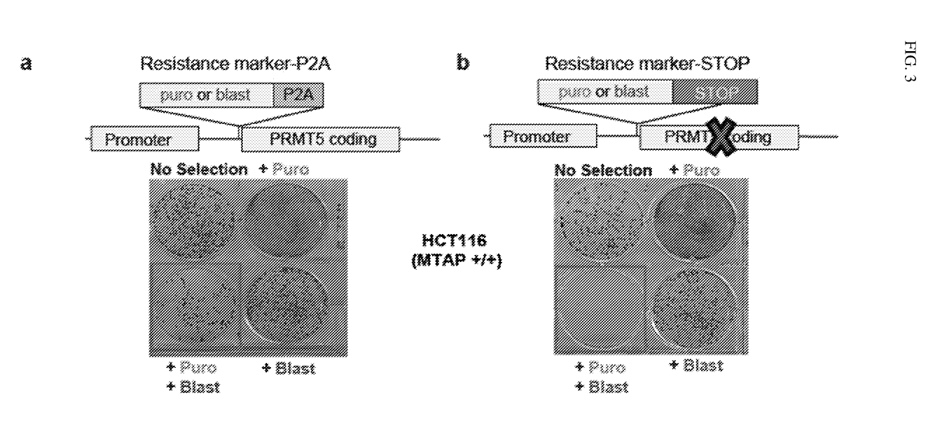

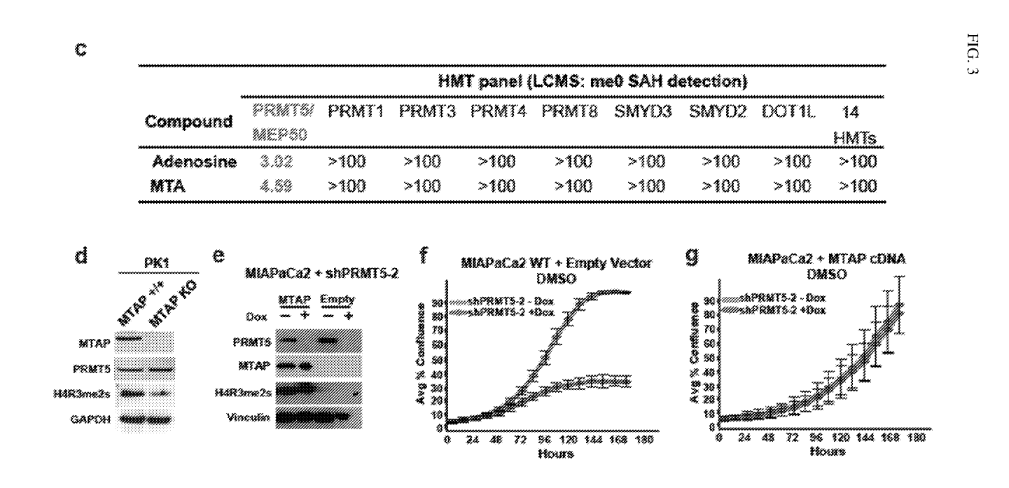

### DETAILED DESCRIPTION OF THE INVENTION

### Definitions

MTAP

By “MTAP” is meant methylthioadenosine phosphorylase, an enzyme in the methionine salvage pathway, also known as S-methyl-5′-thioadenosine phosphorylase; also known as BDMF; DMSFH; DMSMFH; LGMBF; MSAP; and c86fus. External IDs: OMIM: 156540 MGI: 1914152 HomoloGene:1838 chEMBL: 4941 GeneCards: MTAP Gene; Entrez 4507; RefSeq (mRNA): NM_002451; location: Chr 9: 21.8-21.93 Mb. By “wild-type” MTAP is meant that encoded by NM_002451, or having the same amino acid sequence thereof. Schmid et al. 2000 Oncogene 19: 5747-54.

The amino acid sequence of MTAP, as provided in NM_002451, is presented below (as SEQ ID NO: 97):

MASGTTTTAVKIGIIGGTGLDDPEILEGRTEKYVDTPFGKPSDAL
ILGKIKNVDCVLLARHGRQHTIMPSKVNYQANIWALKEEGCTHVI
VTTACGSLREEIQPGDIVIIDQFIDRTTMRPQSFYDGSHSCARGV
CHIPMAEPFCPKTREVLIETAKKLGLRCHSKGTMVTIEGPRFSSR
AESFMFRTMGADVINMITVPEVVLAKEAGICYASIAMATDYDCWK
EHEEAVSVDRVLKILKENANKAKSLLLTTIPQIGSTETNSETLHN
LKNMAQFSVLLPRH

(MTAP amino acid sequence, SEQ ID NO: 97)

The nucleotide (nt) sequence of MTAP, as provided in NM_002451, is presented below (as SEQ ID NO: 98):

| | |
|---|---|
| 1 | CTCCGCACTG CTCACTCCCG CGCAGTGAGG TTGGCACAGC CACCGCTCTG TGGCTCGCTT |
| 61 | GGTTCCCTTA GTCCCGAGCG CTCGCCCACT GCAGATTCCT TTCCCGTGCA GACATGGCCT |
| 121 | CTGGCACCAC CACCACCGCC GTGAAGATTG GAATAATTGG TGGAACAGGC CTGGATGATC |
| 181 | CAGAAATTTT AGAAGGAAGA ACTGAAAAAT ATGTGGATAC TCCATTTGGC AAGCCATCTG |
| 241 | ATGCCTTAAT TTTGGGGAAG ATAAAAAATG TTGATTGCGT CCTCCTTGCA AGGCATGGAA |
| 301 | GGCAGCACAC CATCATGCCT TCAAAGGTCA ACTACCAGGC GAACATCTGG GCTTTGAAGG |
| 361 | AAGAGGGCTG TACACATGTC ATAGTGACCA CAGCTTGTGG CTCCTTGAGG GAGGAGATTC |
| 421 | AGCCCGGCGA TATTGTCATT ATTGATCAGT TCATTGACAG GACCACTATG AGACCTCAGT |
| 481 | CCTTCTATGA TGGAAGTCAT TCTTGTGCCA GAGGAGTGTG CCATATTCCA ATGGCTGAGC |
| 541 | CGTTTTGCCC CAAAACGAGA GAGGTTCTTA TAGAGACTGC TAAGAAGCTA GGACTCCGGT |
| 601 | GCCACTCAAA GGGGACAATG GTCACAATCG AGGGACCTCG TTTTAGCTCC CGGGCAGAAA |
| 661 | GCTTCATGTT CCGCACCTGG GGGGCGGATG TTATCAACAT GACCACAGTT CCAGAGGTGG |
| 721 | TTCTTGCTAA GGAGGCTGGA ATTTGTTACG CAAGTATCGC CATGGCGACA GATTATGACT |
| 781 | GCTGGAAGGA GCACGAGGAA GCAGTTTCGG TGGACCGGGT CTTAAAGACC CTGAAAGAAA |
| 841 | ACGCTAATAA AGCCAAAAGC TTACTGCTCA CTACCATACC TCAGATAGGG TCCACAGAAT |
| 901 | GGTCAGAAAC CCTCCATAAC CTGAAGAATA TGGCCCAGTT TTCTGTTTTA TTACCAAGAC |
| 961 | ATTAAAGTAG CATGGCTGCC CAGGAGAAAA GAAGACATTC TAATTCCAGT CATTTTGGGA |
| 1021 | ATTCCTGCTT AACTTGAAAA AAATATGGGA AAGACATGCA GCTTTCATGC CCTTGCCTAT |
| 1081 | CAAAGAGTAT GTTGTAAGAA AGACAAGACA TTGTGTGTAT TAGAGACTCC TGAATGATTT |
| 1141 | AGACAACTTC AAAATACAGA AGAAAAGCAA ATGACTAGTA AACATGTGGG AAAAAATATT |
| 1201 | ACATTTTAAG GGGGAAAAAA AAACCCACCA TTCTCTTCTC CCCCTATTAA ATTTGCAACA |
| 1261 | ATAAAGGGTG GAGGGTAATC TCTACTTTCC TATACTGCCA AAGAATGTGA GGAAGAAATG |
| 1321 | GGACTCTTTG GTTATTTATT GATGCGACTG TAAATTGGTA CAGTATTTCT GGAGGGCAAT |
| 1381 | TTGGTAAAAT GCATCAAAAG ACTTAAAAAT ACGGACGTAC TTTGTGCTGG GAACTCTACA |
| 1441 | TCTAGCAATT TCTCTTTAAA ACCATATCAG AGATGCATAC AAAGAATTAT ATATAAAGAA |
| 1501 | GGGTGTTTAA TAATGATAGT TATAATAATA AATAATTGAA ACAATCTGAA TCCCTTGCAA |
| 1561 | TTGGAGGTAA ATTATGTCTT AGTTATAATT AGATTGTGAA TCAGCCAACT GAAAATCCTT |
| 1621 | TTTGCATATT TCAATGTCCT AAAAAGACAC GGTTGCTCTA TATATGAAGT GAAAAAAGGA |
| 1681 | TATGGTAGCA TTTTATAGTA CTAGTTTTGC TTTAAAATGC TATGTAAATA TACAAAAAAA |
| 1741 | CTAGAAAGAA ATATATATAA CCTTGTTATT GTATTTGGGG GAGGGATACT GGGATAATTT |
| 1801 | TTATTTTCTT TGAATCTTTC TGTGTCTTCA CATTTTTCTA CAGTGAATTT AATCAAATAG |
| 1861 | TAAAGTTGTT GTAAAAATAA AAGTGGATTT AGAAAGATCC AGTTCTTGAA AACACTGTTT |
| 1921 | CTGGTAATGA AGCAGAATTT AAGTTGGTAA TATTAAGGTG AATGTCATTT AAGGGAGTTA |
| 1981 | CATCTTTATT CTGCTAAAGA AGAGGATCAT TGATTTCTGT ACAGTCAGAA CAGTACTTGG |
| 2041 | GTTTGCAACA GCTTTCTGAG AAAAGCTAGG TGTTTAATAG TTTAACTGAA AGTTTAACTA |
| 2101 | TTTAAAAGAC TAAATGCACA TTTTATGGTA TCTGATATTT TAAAAAGTAA TGTTTGATTC |
| 2161 | TCCTTTTTAT GAGTTAAATT ATTTTATACG AGTTGGTAAT TTTTGCTTTT TAATAAAGTG |
| 2221 | GAAGCTTGCT TTTTTAACTC TTTTTTTATT GTTATTTTAT AGAAATGCTT TTTGTTGGCC |
| 2281 | GGGCACAGTT GCTCATCCAT GTAATCCCAG CACTGTGGGA GGCCGAGACG GGTGGATCAC |
| 2341 | AAGGTCAGGA GATCGAGACC ATCCTGGCTA ATGCGTTGAA ACTCCGTCTC TACTAAAAAT |
| 2401 | ACAAAAAATT AGCTGGGCGT GGTGGTGGGC ACCTGTAGTC CCAGCTACTC AGGAGGCTGA |
| 2461 | GGCAGGAGAA TGGTGTGAAC CTGGGAGGTG GAGCTTGCAG TGAGCAGAGC TTGCAGTGAG |
| 2521 | ACGAGCTTGT GCCACTGCAC TCCAGCCTGG GCAACAGAGT AAGACTCAGT CTCAAAAAAA |
| 2581 | AAAAAAAGAG TGAAATGCTT TTTGTTTGCT TCAGTTTTTT ATCATGGGGA GATCTTTTTC |
| 2641 | CTCAGAATTG TTTTCTTTTC ACTGTAGGCT ATTACAGGAT ACTTCAGGAT CAAGATACAG |
| 2701 | AACCTTTTAT TTAAAGAGTT TGTAAAGTCA ATGTGTTTGT TTGTGTCTCT GAGATTGACT |
| 2761 | TCAAGATAAT AAGCTGCTAA TTGTAAACAA AACAGTTACC CTCCAGTATT AATATGACTC |
| 2821 | ATTAGTGTGA GCCATTTGGG TCAAGTATGA TTATGACCCT TGGACTTCCT GATGTAGTAT |
| 2881 | TAAATTTCAA CTCTGGTTAT CCATTAGCAA TCTGTAGAGA ACTTAATGAA CCTGAACCCA |
| 2941 | GGCTTCTCTA GCTCTGGTAA CGTGTGATTG TTTTCACTAC AATATGATAC ATAGATGGTA |
| 3001 | CCTTACTTTT CCTCATTCTT AATAGGTGTC TAAGAATGTC AGGGCAAAAG TATGGGCATT |
| 3061 | TTTCTTGCTA TGTTCAGAAA GTACAGTTCT CTCCAACTTG CAGAGGTACT TTTCTTGATT |
| 3121 | AAATAGCCTT CTCTAGCAAC ATCATTTTCA GACTAACTAA ATGAATGCAG TATACTCTTT |
| 3181 | TCTTTGTTCT CAATCATTCA CTCCTTATGC AAAGCCAATA TAATTTTCCT CATACCTTAT |
| 3241 | GCTTGAGGAT ATTGTTGAAG AACACTTCCT GGAACACTTC TCACTTGTGA TGCTGTACTA |
| 3301 | ATTTTTTTTT TTTAATTTAA GCTAGTATAC TAAGTGAACA CCATGGTCAG TTGTGAGCAT |
| 3361 | TTTGGTTTCC GCAAAGGATG GATGGTGAGC ATCATGGGAA AGCTGTAGTT TAGTGACTTA |
| 3421 | GCCCTTAGTG ATTAATAGAT TTGCATGTAC ATAGAAGTCT TTGTTGGCCT TATAATCTGC |
| 3481 | TGTTATATTT GGCATGGATT TTCATGGTTT TGAGAATGAC ATCCTGGCCC TGTGGTCCCC |
| 3541 | GAGGGTCATG GTCCTTGTGA CCTGGCCCCT GTTCACTGCC CCCTTCGCTA GCACGAGTTG |
| 3601 | CTGTGCAGGG CTGGAGGTAG CTACCATGGC TTGTTTCAAG GAAGGAAACT CTGGTACGGT |
| 3661 | GGCACCCTCA GGAGTGGAGG ACAGTGAACT TCCTTGAAGA GGGAGTGACT AAGGTGACCT |
| 3721 | CCAACCTGCC CTGAGCCAGC TGCCCTGCAG GTGCCACGTG AGCCTGCTCT GGCATCCACA |
| 3781 | GGATGCTCCT GGAGCCTCTT CTCTGGCTGC TACCTCAGGG CATGGTTGTG GCCCCACCAA |
| 3841 | CACCTATTTT CCAAATAATT ATTCATTCTT GTGACAGTGG CCTGAACATG TTTTTAATTT |
| 3901 | TCTCAACAAG CATTTAGCCA GCACTTATCC AGTGAAACAA TTTGATAAGG TTTCAAGGAG |
| 3961 | TATCTGATGG GTTAGGAAGT CACGAAATGA GGAGTTCTTG CCACATTTGC AGAGTCCCTC |
| 4021 | CTTGATAAGG TTTGGCGGTG TCCCCACCCA AATCTCATGT TGAATTGTAG TTCCCATAAT |
| 4081 | CCCCACATGT TGTGGGAGGG ACCCAGTGGG AGGTAATTAA ATCATGGGGG TGGTTACCCC |
| 4141 | CACACTGCTG TTCTCATGAT ACTGAGTTCT CACAAGTCCT GTTTGTTTTA TAAGGGGCTT |
| 4201 | TTCCCCCTTT TGCTCAACAC TTCTTCCTGC CATCATGTGA AGAAGGACGT GTTTGTTTCC |
| 4261 | CCTTCTGCCA CGATTGTAAG TTTCCTGAGG CCTTCCCAGC TATGTGGAAC TGTGAGTTAA |
| 4321 | TTAAACCTCT TTCCTTTATA AATTACCCAG TCATGGGCAG TCCTTTACAG CAGCATGAGA |
| 4381 | ATGGACTAAT ACACTCCTCA AATGTTTTGA AGATTGTTGC ACCTTGGAAC TACCAGTGTG |
| 4441 | CACACAATCT GGCTCAATGT ATATATTGGC CCAGCAAGGC AAAGAACTGA AGTTCCAGGA |
| 4501 | TGGAAGAACC TGTGTTCTCC TCATAATAGT ATAGAATAAT TCAAGATAGG CAAGAAGGAC |
| 4561 | AGCAGTAAAT GAAGACCATG GAAGAAAAGA AGGAATGCCA AAGATCGAGG AAATCTACCA |
| 4621 | AGACTAGTAG GGTAGTCCAG AAGAAGCTGT TTCAGGGCCT GTTGCCAGCT ATGCCTTTGA |
| 4681 | GAACCTCGGG ATCCCAAAGA ATGAGGGGAA TTTCTTCAGA AAGACAATCT CGGCATGCAT |
| 4741 | TATTTCTTTG TTTTGAAGAT TCACTCATGT TGCATGCATC TGTAGCTTGT GCCTTTTTTA |
| 4801 | TTGCCTAGTA GTATTCTGTC ATATGCCTAT CTTACAATTT GATTATCTAT TCACCTGTTG |
| 4861 | ATGAATGTTT GAATTTTTTC CATTTGAGGA ATTTTATGAA TAAAGCTGCT ATAAGCATGA |
| 4921 | AAAAAAAAAA AAAAAAA |

(MTAP nt sequence, SEQ ID NO: 98)

The MTAP gene encodes an enzyme that plays a major role in polyamine metabolism and is important for the salvage of both adenine and methionine. The encoded enzyme is deficient in many cancers because this gene and the tumor suppressor p16 gene are co-deleted. Multiple alternatively spliced transcript variants have been described for this gene, but their full-length natures remain unknown.

As used herein, the term “MTAP-deficient”, “MTAP-deficiency”, “MTAP-null” and the like refer to cells (including, but not limited to, cancer cells, cell lines, tissues, tissue types, tumors, etc.) that have a significant reduction in post-translational modification, production, expression, level, stability and/or activity of MTAP relative to that in a control, e.g., reference or normal or non-cancerous cells. The reduction can be at least about 20%, 30%, 40%, 50%, 60%, 70%, 80% or 90%. In some embodiments, the reduction is at least 20%. In some embodiments, the reduction is at least 50%. The terms “MTAP-deficient and/or MTA accumulating”, “MTAP-deficient and/or MTA-accumulating”, “MTAP deficient and/or MTA overexpressing”, “MTAP deficient and/or MTA upregulated” and the like, regarding a cell or cells, etc., indicate that the cell or cells, etc., either are deficient in MTAP and/or overexpress, overproduce or accumulate MTA. MTAP-deficient cells include those wherein the MTAP gene has been mutated or deleted. As a non-limiting example, MTAP-deficient cells can have a homozygous deletion. MTAP knockdown is not lethal. In some embodiments, the MTAP-deficient cells are also CDKN2A-deficient. The MTAP deficiency can be detected using any reagent or technique known in the art, for example: immunohistochemistry utilizing an antibody to MTAP, and/or genomic sequencing, and/or nucleic acid hybridization and/or amplification utilizing at least one probe or primer comprising a sequence of at least 12 contiguous nucleotides (nt) of the sequence of MTAP provided in SEQ ID NO: 98, wherein the primer is no longer than about 30 nt.

A “MTAP-deficiency-related” disease (for example, a cancer) or a disease (for example, cancer) “associated with MTAP deficiency” and the like refer to an ailment (for example, cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, bladder cancer, pancreatic cancer, mesothelioma, melanoma, lung squamous, lung adenocarcinoma, diffuse large B-cell lymphoma (DLBCL), leukemia, and head and neck cancer, and cancer of the kidney, breast, endometrium, urinary tract, liver, soft tissue, pleura and large intestine. In a patient afflicted with a MTAP-deficiency-related disease, it is possible that some disease cells (e.g., cancer cells) can be MTAP-deficient while others are not. Similarly, some disease cells may be MTA-accumulating while others are not.

Table 1 shows the frequency of MTAP deficiency in various cancer types. For example, 49.40% of GBM are MTAP-deficient.

TABLE 1
Frequency of MTAP deficiency in various cancer types, as
determined in the present work

| | |
|---|---|
| GBM (Glioblastoma) | 49.40% |
| Bladder | 46.20% |
| Pancreatic | 21.40% |
| Melanoma | 19% |
| Lung squamous | 18.60% |
| Lung adenocarcinoma | 14.30% |
| DLBCL | 14.30% |
| Head and neck | 12.60% |

The cell lines sensitive to PRMT5 knockdown, as identified in this work, can be broken down by lineage. CNS (central nervous system), lung and pancreatic lineages are the top 3 lineages sensitive to PRMT5 loss.

The MTAP-deficient lines sensitive to PRMT5 knockdown, identified in this work, are as follows:
CNS, 22%
Lung, 18%
Pancreas, 15%
Skin, 9%
Kidney, 6%
Haematopoietic tissue, 6%
Breast, 6%
Endometrium, 3%
Urinary tract, 3%
Liver, 3%,
Soft tissue, 3%
Pleura, 3%
Large intestine, 3%

For example, 22% of the MTAP-deficient cell lines sensitive to PRMT5 inhibitor identified in this work were from CNS lines.

Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor.

Initial validation focused on pancreatic models due to the high unmet medical need. Miapaca2 is the second most sensitive pancreative line identified in this work, after SU8686. 16 shRNA were tested against Miapaca2 cells. A subset of PRMT5 shRNAs silences PRMT5 and decreased the H4R3me2 (sh1699, sh4732, sh4733, sh4736, sh4737, and sh4738). PRMT7 was not affected. sh4737 is pan-lethal; it kills all cells indiscriminately, and this is attributed to the fact that it is off-target (knocking down other targets beyond the intentional target it was designed for). PRMT5 silencing impaired colony formation of Miapaca2. PRMT5 silencing also leads to apoptosis (death) of Miapaca2 cells. PRMT5 silencing decreased H4R3me2 and proliferation of Miapaca2 cells. Expression of HA-PRMT5 rescued the knockdown phenotype in Miapaca2 cells. HA-PRMT5 is an overexpression construct expressing PRMT5 N-terminally tagged with HA to differentiate it from endogenous PRMT5. Knockdown of PRMT5 also reduced proliferation and foci formation of the MTAP-deficient cells lines SNU449 (liver cancer) and HCC-44 (lung cancer).

Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

MTA and MTA Accumulation

By “MTA” is meant the PRMT5 inhibitor also known as methyl-thioadenosine, S-methyl-5′-thioadenosine, [5′deoxy-5′-(methylthio)-fl-D-ribofuranosyl] adenine, 5′-methyl-thioadenosine, 5′-deoxy, 5′-methyl thioadenosine, and the like. MTA selectively inhibits PRMT5 methyltransferase activity. MTA is the sole catabolic substrate for MTAP. The terms “MTA accumulating”, “MTA overexpressing”, “MTA overproducing”, “MTA upregulated” and the like refer to cells (including, but not limited to, cancer cells, cell lines, tissues, tissue types, tumors, etc.) that have a significantly increased production, expression, level, stability and/or activity of MTA. MTA-accumulating cells include those wherein the cells comprise at least about 20%, 30%, 40%, 50%, 60%, 70%, 80%, 90%, 100%, or greater than 100%, higher production, expression, level, stability and/or activity of MTA than that in normal or non-cancerous cells. In some embodiments, MTA-accumulating cells include those wherein the cells comprise at least 20% higher production, expression, level, stability and/or activity of MTA than that in normal or non-cancerous cells. In some embodiments, MTA-accumulating cells include those wherein the cells comprise at least 50% higher production, expression, level, stability and/or activity of MTA than that in normal or non-cancerous cells. MTA levels in test samples (e.g., cells such as cancer cells being tested for MTA accumulation) and reference samples, and other cells, tissues, samples, etc., can be performed using any method known in the art. Such methods for detecting MTA include, as a non-limiting example, liquid chromatography-electrospray ionization-tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. Loss of MTAP is associated with accumulation of MTA, which can lead to a decrease in symmetric and asymmetric protein methylation by inhibition of PRMT function. Williams-Ashman et al. 1982 Biochem. Pharm. 31: 277-288; and Limm et al. 2013 Eur. J. Cancer 49, Issue 6. Lethality is specific to PRMT5 and not others.

As shown herein, addition or accumulation of MTA sensitizes MTAP-expressing cells to PRMT5 inhibition (see Example 5). We show here that MTA itself creates a synthetic sensitization to loss of PRMT5. PRMT5 is essential, but when PRMT5 inhibitor MTA is aberrantly raised in some cells (e.g., MTA accumulates), surviving cells will have a reduced but non-zero amount of PRMT5 activity. When a second PRMT5 inhibitor (or additional MTA) is systemically introduced, it will lower the PRMT5 activity in all cells receiving the inhibitor (or additional MTA). The normal cells, with a normal level of PRMT5 activity, will be able to survive a decrease in PRMT5. But aberrant cells, wherein PRMT5 activity is already reduced, will have PRMT5 activity further reduced, such that these cells cannot survive and/or proliferate. The therapeutic window of administration of a PRMT5 inhibitor, therefore, would be the dosage of PRMT5 inhibitor which does not kill normal cells (with a normal level of PRMT5 activity), but which kills cells (e.g., cancer cells), which already have a reduced PRMT5 activity (e.g., cells with MTAP deficiency or MTA accumulation).

As described further herein, a cancer cell, a cancer type, or a subject afflicted with a cancer, is “PRMT5 inhibitor sensitive,” “sensitive to treatment with PRMT5 inhibitors,” “sensitive to PRMT5 therapeutic inhibition,” or described in similar terms if it is amenable to treatment with a PRMT5 inhibitor, e.g., due to its MTAP deficiency and/or MTA accumulation character.

PRMT5

By “PRMT5” is meant the gene or protein Protein Arginine Methyltransferase 5, also known as HRMT1L5; IBP72; JBP1; SKB1; or SKB1Hs External IDs: OMIM: 604045, MGI: 1351645, HomoloGene: 4454, ChEMBL: 1795116, GeneCards: PRMT5 Gene; EC number 2.1.1.125. Ensembl ENSG00000100462; UniProt 014744; Entrez Gene ID: 10419; RefSeq (mRNA): NM_001039619. The mouse homolog is NM_013768.

Methyltransferases such as PRMT5 catalyse the transfer of one to three methyl groups from the co-factor S-adenosylmethionine (also known as SAM or AdoMet) to lysine or arginine residues of histone proteins. Arginine methylation is carried out by 9 different protein arginine methyltransferases (PRMT) in humans. Three types of methylarginine species exist: (1) Monomethylarginine (MMA); (2) Asymmetric dimethyl arginine (ADMA), which is produced by Type I methyl transferases (PRMT1, PRMT2, PRMT3, CARM1, PRMT6 and PRMT8); and (3) Symmetrical dimethylarginine (SDMA), which is produced by Type II methyl transferases (PRMT5 and PRMT7). PRMT1 and PRMT5 are the major asymmetric and symmetric arginine methyltransferases, respectively. Loss results in embryonic lethality. PRMT5 promotes symmetric dimethylation on histones at H3R8 and H4R3 (H4R3me2). Symmetric methylation of H4R3 is associated with transcriptional repression and can act as a binding site for DNMT3A. Loss of PRMT5 results in reduced DNMT3A binding and gene activation. Tumor suppressor gene ST7 and chemokines RNATES, IP10, CXCL11 are targeted and silenced by PRMT5. WO 2011/079236. Additional substrates include E2F1, p53, EGFR and CRAF. PRMT5 is part of a multi-protein complex comprising the co-regulatory factor WDR77 (also known as MEP50, a CDK4 substrate) during G1/S transition. Phosphorylation increases PRMT5/WDR77 activity. WDR77 is the non-catalytic component of the complex and mediates interactions with binding partners and substrates.

PRMT5 can also interact with pICIn or RioK1 adaptor proteins in a mutually exclusive fashion to modulate complex composition and substrate specificity.

PRMT5 has either a positive or negative effect on its substrates by arginine methylation when interacting with a number of complexes and is involved in a variety of cellular processes, including RNA processing, singal transduction, transcriptional regulation, and germ cell development. PRMT5 is a major pro-survival factor regulating eIF4E expression and p53 translation. PRMT5 triggers p53-dependent apoptosis and sensitized various cancer cells to Tumor necrosis factor (TNF)-related apoptosis-inducing ligand (TRAIL) without affecting TRAIL resistance in non-transformed cells.

PRMT5 mutations are embryonic lethal. PRMT5+/− mice are viable, but produce no viable homozygous PRMT5−/− offspring. Tee et al. 2010 Genes Dev. 24: 2772-7.

The term “PRMT5 inhibitor” refers to any compound capable of inhibiting the production, level, activity, expression or presence of PRMT5. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the PRMT5 protein, either by ATP-competitive inhibition of the active site, allosteric modulation of the protein structure, disruption of protein-protein interactions, or by inhibiting the transcription, translation, post-translational modification, or stability of PRMT5 protein.

A PRMT5 inhibitor can target any of the various domains of PRMT5. For example, PRMT5 is known to comprise a TIM barrel, a Rossman fold, a dimerization domain and a beta barrel. The catalytic domain consists of a SAM binding domain containing the nucleotide binding Rossman fold, followed by a beta-sandwich domain (involved in substrate binding) The TIM barrel is required for binding of adaptor proteins (RIOK1 and pICIn). A PRMT5 inhibitor can contact or attack any of these domains or any portion of PRMT5.

In some embodiments, a PRMT5 inhibitor competes with another compound, protein or other molecule which interacts with PRMT5 and is necessary for PRMT5 function.

As a non-limiting example, a PRMT5 inhibitor can compete with the co-factor S-adenosylmethionine (also known as SAM or AdoMet).

As another non-limiting example, a PRMT5 inhibitor can be a protein-protein interaction (PPI) inhibitor. For example, a PPI inhibitor may inhibit the ability of PRMT5 to properly interact with another protein.

Instead of interacting with PRMT5, a PRMT5 inhibitor can interact with a component necessary for PRMT5 function.

For example:

A PRMT5 inhibitor can act indirectly by interacting with and/or inhibiting WDR77. By “WDR77” is meant the gene or its product, also known as MEP-50; MEP50; Nbla10071; RP11-552M11.3; p44; p44/Mep50; or OMIM: 611734 MGI: 1917715 HomoloGene: 11466 GeneCards: WDR77 Gene. Friesen et al. 2002 J. Biol. Chem. 277:8243-7; Licciardo et al. 2003 Nucl. Acids Res. 31:999-1005; Furuno et al. 2006 Biochem. Biophys. Res. Comm. 345: 1051-8.

The PRMT5:WDR77 complex is required for PRMT5 methyltransferase activity. WDR77 comprises three WD40 domains. PRMT5 and WDR77 (also known as MEP50) form a hetero-octameric complex consisting of 4 monomers. WDR77 molecules bind to the outer surface by interacting solely with N-terminal TIM barrel domains of PRMT5.

The present work showed significant overlap between PRMT5 and WDR77 knockdown sensitive cell lines. The present disclosure thus encompasses methods of inhibiting the proliferation, growth and/or viability of MTAP-deficient and/or MTA-accumulating cells, comprising the step of administering an effective amount of a PRMT5 inhibitor, wherein the PRMT5 inhibitor inhibits WDR77. WDR77 knockdown has a modest effect compared to PRMT5 knockdown.

PRMT5 inhibitors include those compositions which inhibit WDR77 or inhibit the interaction (e.g., the protein-protein interaction) between WDR77 and PRMT5.

WDR77 inhibitors can include, without limitation: a RNA inhibitor (e.g., a RNAi agent), a CRISPR, a TALEN, a zinc finger nuclease, an mRNA, an antibody or derivative thereof, a chimeric antigen receptor T cell (CART) or a low molecular weight (LMW) compound.

WDR77 inhibitors include, but are not limited to, those known in the art.

For example, siRNAs to WDR77 are known in the art.

For example, Aggarwal et al. 2010 Cancer Cell 18: 329-340 shows a MEP50 (WDR77) siRNA with the sequence CUCCUUACCAUUAAACUG (SEQ ID NO: 36).

Additional RNAi agents to MEP50 (WDR77) are disclosed in:

Gu et al. 2013 Oncogene 31: 1888-1900; and

Ligr et al. 2011 PLoS One 6: 10.1371.

As another non-limiting example, a PRMT5 inhibitor can inhibit RIOK1. By “RIOK1” is meant RioK1, RIO Kinase 1, bA288G3.1, Serine/Threonine-Protein Kinase RIO1, EC 2.7.11.1; External Ids: HGNC: 18656; Entrez Gene: 83732; Ensembl: ENSG00000124784; UniProtKB: Q9BRS2.

In this work, the top correlating shRNA features to MTAP deficiency finds PRMT5 and RIOK1. This work also showed a significant overlap between PRMT5 and RIOK1 knockdown sensitive MTAP-deficient cancer cell lines. Many MTAP-deficient cancer cell lines were sensitive to RIOK1 knockdown. A subset of lines sensitive to PRMT5 knockdown are sensitive to RIOK1 loss, but PRMT5 shows a more robust phenotype. A subset of lines sensitive to PRMT5 knockdown are insensitive to RIOK1 loss. The present disclosure thus encompasses methods of inhibiting the proliferation, growth and/or viability of MTAP-deficient and/or MTA-accumulating cells, comprising the step of administering an effective amount of a PRMT5 inhibitor, wherein the PRMT5 inhibitor inhibits RIOK1.

RIOK1 inhibitors can include, without limitation: a RNA inhibitor (e.g., a RNAi agent), a CRISPR, a TALEN, a zinc finger nuclease, an mRNA, an antibody or derivative thereof, a chimeric antigen receptor T cell (CART) or a low molecular weight (LMW) compound.

RIOK1 inhibitors include, but are not limited to, those known in the art.

For example, siRNAs to RioK1 are known in the art. For example, Guderian et al. 2011 J. Biol. Chem. 286: 1976-1986 shows RioK1 siRNAs with the sequences GAGAAGGAUGACAUUCUGUTT (SEQ ID NO: 37) and ACAGAAUGUCAUCCUUCUCTT (SEQ ID NO: 38).

Additional RIOK1 RNAi agents are disclosed in: Read et al. 2013 PLoS Genetics 10.1371.

As another non-limiting example, a PRMT5 inhibitor can act indirectly by inhibiting pICIN.

pICln is an essential, highly conserved 26-kDa protein whose functions include binding to Sm proteins in the cytoplasm of human cells and mediating the ordered and regulated assembly of the cell's RNA-splicing machinery by the survival motor neurons complex. pICln also interacts with PRMT5, the enzyme responsible for generating symmetric dimethylarginine modifications on the carboxyl-terminal regions of three of the canonical Sm proteins. Pesiridis et al. 2009. J. Biol. Chem. 284: 21347-21359. The present disclosure thus encompasses methods of inhibiting the proliferation, growth and/or viability of MTAP-deficient and/or MTA-accumulating cells, comprising the step of administering an effective amount of a PRMT5 inhibitor, wherein the PRMT5 inhibitor inhibits pICln.

This work showed significant overlap between PRMT5 and pICIN knockdown by shRNAs in efficacy against MTAP-deficient cancer cell lines.

pICIN inhibitors can include, without limitation: a RNA inhibitor (e.g., a RNAi agent), a CRISPR, a TALEN, a zinc finger nuclease, an mRNA, an antibody or derivative thereof, a chimeric antigen receptor T cell (CART) or a low molecular weight (LMW) compound.

The present disclosure also notes that PRMT5 is normally found in both the nucleus and cytoplasm. A PRMT5 inhibitor may inhibit PRMT5 function by reducing the post-translational modification, production, expression, level, stability and/or activity of PRMT5 in the nucleus, in the cytoplasm, or both the nucleus and cytoplasm. An inhibitor could, for example, reduce PRMT5 in the cytoplasm, but not the nucleus, or vice versa.

According to the present invention, an PRMT5 inhibitor includes, as non-limiting examples: an antibody or derivative thereof, a RNA inhibitor (e.g., a RNAi agent), a therapeutic modality, including but not limited to, a low molecular weight compound, a CRISPR, a TALEN, a zinc finger nuclease, an mRNA, or a chimeric antigen receptor T cell (CART).

In any method described herein, the PRMT5 inhibitor can inhibit PRMT5 indirectly by inhibiting WDR77, RIOK1, and/or pICIN.

Antibody

The term “antibody” (e.g., an “antibody to PRMT5”) and the like as used herein refers to whole antibodies that interact with (e.g., by binding, steric hindrance, stabilizing/destabilizing, spatial distribution) an antigen or epitope (e.g., a PRMT5 epitope or antigen). A naturally occurring IgG “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “epitope binding domain” or “EBD” refers to portions of a binding molecule (e.g., an antibody or epitope-binding fragment or derivative thereof), that specifically interacts with (e.g., by binding, steric hindrance, stabilizing/destabilizing, spatial distribution) a binding site on a target epitope. EBD also refers to one or more fragments of an antibody that retain the ability to specifically interact with (e.g., by binding, steric hindrance, stabilizing/destabilizing, spatial distribution) a PRMT5 epitope and inhibit signal transduction. Examples of antibody fragments include, but are not limited to, an scFv, a Fab fragment, a monovalent fragment consisting of the VL, VH, CL and CH1 domains; a F(ab).sub.2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).

Such single chain antibodies are also intended to be encompassed within the terms “fragment”, “epitope-binding fragment” or “antibody fragment”. These fragments are obtained using conventional techniques known to those of skill in the art, and the fragments are screened for utility in the same manner as are intact antibodies.

Antibody fragments can be incorporated into single chain molecules comprising a pair of tandem Fv segments (VH—CH1-VH—CH1) which, together with complementary light chain polypeptides, form a pair of antigen binding regions (Zapata et al., (1995) Protein Eng. 8:1057-1062; and U.S. Pat. No. 5,641,870), and also include Fab fragments, F(ab′) fragments, and anti-idiotypic (anti-Id) antibodies (including, e.g., anti-Id antibodies to antibodies of the invention), and epitope-binding fragments of any of the above.

EBDs also include single domain antibodies, maxibodies, unibodies, minibodies, triabodies, tetrabodies, v-NAR and bis-scFv, as is known in the art (see, e.g., Hollinger and Hudson, (2005) Nature Biotechnology 23: 1126-1136), bispecific single chain diabodies, or single chain diabodies designed to bind two distinct epitopes. EBDs also include antibody-like molecules or antibody mimetics, which include, but not limited to minibodies, maxybodies, Fn3 based protein scaffolds, Ankrin repeats (also known as DARpins), VASP polypeptides, Avian pancreatic polypeptide (aPP), Tetranectin, Affililin, Knottins, SH3 domains, PDZ domains, Tendamistat, Neocarzinostatin, Protein A domains, Lipocalins, Transferrin, and Kunitz domains that specifically bind epitopes, which are within the scope of the invention. Antibody fragments can be grafted into scaffolds based on polypeptides such as Fibronectin type III (Fn3) (see U.S. Pat. No. 6,703,199, which describes fibronectin polypeptide monobodies).

The present invention also encompasses an antibody to PRMT5, which is an isolated antibody, monovalent antibody, bivalent antibody, multivalent antibody, bivalent antibody, biparatopic antibody, bispecific antibody, monoclonal antibody, human antibody, recombinant human antibody, or any other type of antibody or epitope-binding fragment or derivative thereof.

The phrase “isolated antibody”, as used herein, refers to antibody that is substantially free of other antibodies having different antigenic specificities (e.g., an isolated antibody that specifically binds PRMT5 is substantially free of antibodies that specifically bind antigens other tha PRMT5). An isolated antibody that specifically binds PRMT5 may, however, have cross-reactivity to other antigens, such as PRMT5 molecules from other species. Moreover, an isolated antibody may be substantially free of other cellular material and/or chemicals.

The term “monovalent antibody” as used herein, refers to an antibody that binds to a single epitope on a target molecule such as PRMT5.

The term “bivalent antibody” as used herein, refers to an antibody that binds to two epitopes on at least two identical PRMT5 target molecules. The bivalent antibody may also crosslink the target PRMT5 molecules to one another. A “bivalent antibody” also refers to an antibody that binds to two different epitopes on at least two identical PRMT5 target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for the PRMT5, the mutivalent antibody (e.g., a PRMT5 biparatopic antibody) has a binding moiety for two domains of PRMT5, respectively.

The multivalent antibody mediates biological effect or which modulates a disease or disorder in a subject (e.g., by mediating or promoting cell killing, or by modulating the amount of a substance which is bioavailable.

The term “multivalent antibody” also refers to a single binding molecule that has more than one antigen-binding moieties for two separate WRM target molecules. For example, an antibody that binds to both a PRMT5 target molecule and a second target molecule that is not PRMT5. In one embodiment, a multivalent antibody is a tetravalent antibody that has four epitope binding domains. A tetravalent molecule may be bispecific and bivalent for each binding site on that target molecule.

The term “biparatopic antibody” as used herein, refers to an antibody that binds to two different epitopes on a single PRMT5 target. The term also includes an antibody, which binds to two domains of at least two PRMT5 targets, e.g., a tetravalent biparatopic antibody.

The term “bispecific antibody” as used herein, refers to an antibody that binds to two or more different epitopes on at least two different targets (e.g., a PRMT5 and a target that is not PRMT5).

The phrases “monoclonal antibody” or “monoclonal antibody composition” as used herein refers to polypeptides, including antibodies, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The phrase “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; A1 Lazikani et al., (1997) J. Mol. Bio. 273:927 948); Kabat et al., (1991) Sequences of Proteins of Immunological Interest, 5th edit, NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding site” as used herein comprises an area on a PRMT5 target molecule to which an antibody or antigen binding fragment selectively binds.

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics.

Generally, antibodies specific for a particular target antigen will bind to an epitope on the target antigen in a complex mixture of proteins and/or macromolecules.

As used herein, the term “Affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a K_{D} of 10⁻8 M or less, 10⁻⁹ M or less, or 10⁻¹⁰ M, or 10⁻¹¹ M or less, or 10⁻¹² M or less, or 10⁻¹³ M or less for a target antigen. However, high affinity binding can 10 vary for other antibody isotypes. For example, high affinity binding for an IgM isotype refers to an antibody having a K_{D} of 10⁻⁷ M or less, or 10⁻⁸ M or less.

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

A PRMT5 inhibitor which is an antibody can be prepared; alternatively, many PRMT5 antibodies are known in the art.

Any inhibitory anti-PRMT5 antibody or fragment thereof can be used with any method disclosed herein.

RNAi Agent

As used herein, the term “RNAi agent,” (e.g., a “RNAi agent to PRMT5”, “siRNA to PRMT5”, or “PRMT5 siRNA”) and the like refer to an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target mRNA (e.g., the PRMT5 mRNA) and which mediates the targeted cleavage of the RNA transcript via an RNA-induced silencing complex (RISC) pathway. In one embodiment, the RNAi agent is an oligonucleotide composition that activates the RISC complex/pathway. In another embodiment, the RNAi agent comprises an antisense strand sequence (antisense oligonucleotide). In one embodiment, the RNAi comprises a single strand. This single-stranded RNAi agent oligonucleotide or polynucleotide can comprise the sense or antisense strand, as described by Sioud 2005 J. Mol. Bioi. 348:1079-1090, and references therein. Thus the disclosure encompasses RNAi agents with a single strand comprising either the sense or antisense strand of an RNAi agent described herein. The use of the RNAi agent to PRMT5 results in a decrease of PRMT5 production, expression, level, and/or activity, e.g., a “knock-down” or “knock-out” of the PRMT5 target gene or protein product thereof. In some embodiments, the PRMT5 inhibitor is molecule capable of mediating RNA interference against PRMT5 and comprising a sequence selected from the group consisting of SEQ ID NO: 1 to SEQ ID NO: 18, 41-49, 52-79, or 84-96.

RNA interference is a post-transcriptional, targeted gene-silencing technique that, usually, uses double-stranded RNA (dsRNA) to degrade messenger RNA (mRNA) containing the same sequence as the dsRNA. The process of RNAi occurs naturally when ribonuclease III (Dicer) cleaves longer dsRNA into shorter fragments called siRNAs. Naturally-occurring siRNAs (small interfering RNAs) are typically about 21 to 23 nucleotides long and comprise about 19 base pair duplexes. The smaller RNA segments then mediate the degradation of the target mRNA. Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex.

“RNAi” (RNA interference) has been studied in a variety of systems. Early work in Drosophila embryonic lysates (Elbashir et al. 2001 EMBO J. 20: 6877 and Tuschl et al. International PCT Publication No. WO 01/75164) revealed certain parameters for siRNA length, structure, chemical composition, and sequence that are beneficial to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of an siRNA duplex is usually required for siRNA activity. Later work showed that a 3′-terminal dinucleotide overhang can be replaced by a 3′ end cap, provided that the 3′ end cap still allows the molecule to mediate RNA interference; the 3′ end cap also reduces sensitivity of the molecule to nucleases. See, for example, U.S. Pat. Nos. 8,097,716; 8,084,600; 8,404,831; 8,404,832; and 8,344,128. Additional later work on artificial RNAi agents showed that the strand length could be shortened, or a single-stranded nick could be introduced into the sense strand. Additional formats of siRNAs are shown in, for example, PCT/US14/58703 and PCT/US14/59301. In addition, mismatches can be introduced between the sense and anti-sense strands and a variety of modifications can be used. Any of the these and various other formats for RNAi agents known in the art can be used to produce RNAi agents to PRMT5.

In some embodiments, the RNAi agent to PRMT5 is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

Kits for RNAi synthesis are commercially available, e.g., from New England Biolabs and Ambion.

A suitable RNAi agent can be selected by any process known in the art or conceivable by one of ordinary skill in the art. For example, the selection criteria can include one or more of the following steps: initial analysis of the PRMT5 gene sequence and design of RNAi agents; this design can take into consideration sequence similarity across species (human, cynomolgus, mouse, etc.) and dissimilarity to other (non-PRMT5) genes; screening of RNAi agents in vitro (e.g., at 10 nM in cells); determination of EC50 in HeLa cells; determination of viability of various cells treated with RNAi agents, wherein it is desired that the RNAi agent to PRMT5 not inhibit the viability of these cells; testing with human PBMC (peripheral blood mononuclear cells), e.g., to test levels of TNF-alpha to estimate immunogenicity, wherein immunostimulatory sequences are less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein lmmunostimulatory sequences are less desired; determination of gene knockdown in vivo using subcutaneous tumors in test animals; PRMT5 target gene modulation analysis, e.g., using a pharmacodynamic (PD) marker, and optimization of specific modifications of the RNAi agents.

Specific RNAi agents include: the shRNAs to PRMT5 disclosed herein (particularly those having a target sequence of any of SEQ ID NOs: 1 to 18, 41-49, 52-79, or 84-96, or a target sequence comprising 15 contiguous nt of a PRMT5 target sequence thereof). Additional RNAi agents to PRMT5 can be prepared, or are known in the art. It is noted that in the present disclosure a RNAi agent to PRMT5 may be recited to target a particular PRMT5 sequence, indicating that the recited sequence may be comprised in the sequence of the sense or anti-sense strand of the RNAi agent; or, in some cases, a sequence of at least 15 contiguous nt of this sequence may be comprised in the sequence of the sense or anti-sense strand. It is also understood that some of the target sequences are presented as DNA, but the RNAi agents targeting these sequences can be RNA, or any nucleotide, modified nucleotide or substitute disclosed herein.

### Additional Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The terms “biomarker” or “marker” are used interchangeably herein. A biomarker is a nucleic acid or polypeptide and the presence or absence of a mutation or differential expression of the polypeptide is used to determine sensitivity to any PRMT5 inhibitor. For example, MTAP is a biomarker in a cancer cell when it is deficient, mutated, deleted, or decreased in post-translational modification, production, expression, level, stability and/or activity, as compared to MTAP in normal cell or control cell.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Example vectors for libraries include bacteriophage (also known as “phage”), viruses that infect bacteria, for example, lambda phage. The library can then be probed for the specific cDNA (and thus mRNA) of interest.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

“Gene expression” or alternatively a “gene product” refers to the nucleic acids or amino acids (e.g., peptide or polypeptide) generated when a gene is transcribed and translated.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non cancerous cell or tissue) is undetectable.

A high expression level of the gene can occur because of over expression of the gene or an increase in gene copy number. The gene can also be translated into increased protein levels because of deregulation or absence of a negative regulator. Lastly, high expression of the gene can occur due to increased stabilization or reduced degradation of the protein, resulting in accumulation of the protein.

A “gene expression profile” or “gene signature” refers to a pattern of expression of at least one biomarker that recurs in multiple samples and reflects a property shared by those samples, such as mutation, response to a particular treatment, or activation of a particular biological process or pathway in the cells. A gene expression profile differentiates between samples that share that common property and those that do not with better accuracy than would likely be achieved by assigning the samples to the two groups at random. A gene expression profile may be used to predict whether samples of unknown status share that common property or not. Some variation between the biomarker(s) and the typical profile is to be expected, but the overall similarity of biomarker(s) to the typical profile is such that it is statistically unlikely that the similarity would be observed by chance in samples not sharing the common property that the biomarker(s) reflects.

As used herein, the term “inhibit”, “inhibiting”, or “inhibit the proliferation” of a cancer cell refers to slowing, interrupting, arresting or stopping the growth of the cancer cell, and does not necessarily indicate a total elimination of the cancer cell growth. The terms “inhibit” and “inhibiting”, or the like, denote quantitative differences between two states, refer to at least statistically significant differences between the two states. For example, “an amount effective to inhibit growth of cancer cells” means that the rate of growth of the cells will be at least statistically significantly different from the untreated cells. Such terms are applied herein to, for example, rates of cell proliferation.

This disclosure shows that deficiency of the gene MTAP or its protein product predicts response of cancer cells to PRMT5 inhibition.

A “wild-type,” “normal,” or “non-mutant” human PRMT5 refers to sequence of PRMT5 of Entrez Gene ID: 10419. A “wild-type,” “normal,” or “non-mutant” human MTAP has the amino acid sequence of SEQ ID NO: 97 or NM 002451. The terms “normal”, “non-cancerous”, “reference”, “control” and the like, in reference to a cell, tissue, sample, etc., indicate that that cell, tissue, sample, etc., is normal with reference to a particular measured quality, such as production, level, activity and/or expression of PRMT5, MTAP, MTA, etc.

A “mutant,” or “mutation” is any change in DNA or protein sequence that deviates from wild type gene or protein product sequence. This includes without limitation; single base nucleic acid changes or single amino acid changes, insertions, deletions and truncations of the wild type MTAP gene and its corresponding protein.

The term “isolated” means separated from constituents, cellular and otherwise, in which the polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A “metastatic cell or tissue” means that the cell can invade and destroy neighboring body structures.

The term “PBMC” refers to peripheral blood mononuclear cells and includes “PBL”—peripheral blood lymphocytes.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, siRNAs, shRNAs, RNAi agents, and primers. A polynucleotide can be modified or substituted at one or more base, sugar and/or phosphate, with any of various modifications or substitutions described herein or known in the art. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in PCR: A Practical Approach, M. MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (Sambrook et al., Molecular Cloning: A Laboratory Manual, 2nd edition (1989)).

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

A cell is “sensitive,” displays “sensitivity” for inhibition, or is “amenable to treatment” with a PRMT5 inhibitor when the cell viability is reduced and/or the rate of cell proliferation is reduced upon treatment with a PRMT5 inhibitor when compared to an untreated control.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories), polyHIPE(R)™ resin (obtained from Aminotech, Canada), polyamide resin (obtained from Peninsula Laboratories), polystyrene resin grafted with polyethylene glycol (TentaGelR™, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087 and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity can be assessed by measuring levels of messenger RNA using a gene chip such as the Affymetrix® HG-U133-Plus-2 GeneChips (Affymetrix, Santa Clara, Calif.). High-throughput, real-time quanititation of RNA of a large number of genes of interest thus becomes possible in a reproducible system.

The terms “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68° C. or 0.015M sodium chloride, 0.0015M sodium citrate, and 50% formamide at 42° C. An example of a “moderately stringent” hybridization is the conditions of: 0.015M sodium chloride, 0.0015M sodium citrate at 50-65° C. or 0.015M sodium chloride, 0.0015M sodium citrate, and 20% formamide at 37-50° C. The moderately stringent conditions are used when a moderate amount of nucleic acid mismatch is desired. One of skill in the art will appreciate that washing is part of the hybridization conditions. For example, washing conditions can include 02.X-0.1×SSC/0.1% SDS and temperatures from 42-68° C., wherein increasing temperature increases the stringency of the wash conditions.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Suppressing” or “suppression” of tumor growth indicates a reduction in tumor cell growth when contacted with a PRMT5 inhibitor compared to tumor growth without contact with a PRMT5 inhibitor compound. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage. A “subject,” “individual” or “patient” is used interchangeably herein, which refers to a vertebrate, preferably a mammal, more preferably a human. Mammals include, but are not limited to, mice, simians, humans, farm animals, sport animals, and pets.

The terms “synthetic lethality,” and “synthetic lethal” are used to refer to a combination of mutations in two or more genes leads to reduced cell viability and/or a reduced rate of cell proliferation, whereas a mutation in only one of these genes does not. As a non-limiting example, a reduction of the production, level, activity, expression or presence of PRMT5 via use of a PRMT5 inhibitor is an example of a synthetic lethality in cells which are MTAP-deficient and/or MTA-accumulating.

A “reference” or “control,” “normal”, “wild-type” tissue, cell or sample, or the like, refers to a tissue, cell or sample used, as a non-limiting example, as a reference as a tissue, cell or sample which is not MTAP-deficient and/or MTA-accumulating, for comparison with a test tissue, cell or sample from a subject, in order to determine if the test tissue, cell or sample is MTAP-deficient and/or MTA-accumulating or not. In various embodiments, the control is a non-cancerous cell.

### DETAILED DESCRIPTION

The present invention provides novel diagnostic and treatment methods for a subject with a MTAP-deficiency-related disease, such as a cancer, by targeting the PRMT5 expression or function. The present invention also provides novel diagnostic and treatment methods for a subject with a disease related to MTA accumulation, such as a cancer, by targeting the PRMT5 expression or function. The present invention is based, in part, on the discovery that MTAP-deficient and/or MTA-accumulating cancer lines are sentitive to inhibition of the PRMT5 gene. These types of cancer include, but are not limited to, glioblastoma, bladder cancer, pancreatic cancer, mesothelioma, melanoma, lung squamous, lung adenocarcinoma, diffuse large B-cell lymphoma (DLBCL), leukemia, and head and neck cancer, and cancer of the kidney, breast, endometrium, urinary tract, liver, soft tissue, pleura and large intestine, which are MTAP-deficient. In this work, in almost all cases, inhibition of PRMT5 did not seem to alter the proliferation or viability of cell lines expressing MTAP.

In some embodiments, the MTAP-deficient cells are also CDKN2A-deficient. However, deficiency of CDKN2A and MTAP are distinct in their response to the loss of PRMT5. Loss of CDKN2A is not sufficient; but loss of MTAP is necessary for sensitivity to PRMT5 knockdown.

PRMT5 emerged from an EpiCellecta screen as a potential synthetic lethal with CDKN2A loss. In other words, many cell lines with loss of CDKN2A were sensitive to the knockdown of PRMT5. However, statistical robustness of the finding was weak, as many CDKN2A mutants were not sensitive to knockdown of PRMT5. A subsequent pooled shRNA screen was performed of 277 cell lines of diverse cancer types from the Novartis/Broad Cancer Cell Line Encyclopedia (CCLE), as described in Nature. 2012 Mar. 28; 483(7391):603-7. PRMT5 correlation with CDKN2A loss was much more robust in these new data, but several CDKN2A deleted cell lines were still insensitive to PRMT5 knockdown. Partitioning of the PRMT5-sensitive versus PRMT5-insensitive cell lines revealed MTAP deletion or low expression as the top stratifier. MTAP is a gene located on the same chromosome as CDKN2A and the two are often, but not always, both deleted.

MTAP loss thus predicts response to PRMT5 knockdown. Knockdown of the gene PRMT5 very specifically inhibits the proliferation of MTAP-deficient and/or MTA-accumulating cancers.

None of the other members of the PRMT family were synthetic lethal in MTAP-deficient cells. Loss of PRMT7, for example, did not have the same negative impact on proliferation of MTAP-deficient cells as PRMT5.

MTAP is an enzyme in the methionine salvage pathway. Without being bound by any particular theory, this disclosure suggests that the methionine salvage pathway maintains methionine levels in vivo through a degradation pathway that leads from S-adenosylmethionine (SAM, AdoMet) through methylthioadenosine (MTA). Loss of MTAP is associated with accumulation of MTA, which can lead to a decrease in symmetric and asymmetric protein methylation by inhibition of PRMT function. Williams-Ashman et al. 1982 Biochem. Pharm. 31: 277-288; and Limm et al. 2013 Eur. J. Cancer 49, Issue 6. Lethality is specific to PRMT5 and not others.

PRMT5 inhibition represents a possibly therapeutically useful node to inhibit the proliferation of MTAP deficiency and/or MTA accumulation-related cancers, while potentially sparing many of the side-effects and toxicities of cytotoxic chemotherapy

In various aspects, the present disclosure provides a method for inhibiting proliferation of cancer cells in a subject, the method comprising the step of administering a PRMT5 inhibitor to a subject in need thereof, in an amount that is effective to inhibit proliferation of the MTAP-deficient and/or MTA-accumulating cells. In some embodiments, the MTAP-deficient and/or MTA-accumulating cells are cancer cells. In some embodiments, the MTAP-deficiency-related cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine. According to the present invention, a PRMT5 inhibitor includes, but is not limited to, a low molecular weight compound, a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART).

The present disclosure further provides use of a PRMT5 inhibitor, such as low molecular weight compound, a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART), for the treatment of a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. Also provided is a use of a PRMT5 inhibitor, including, but not limited to, low molecular weight compound, a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART), for the manufacture of a medicament for treating a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a method of treating MTAP-deficient and/or MTA-accumulating cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine, by administering to a subject in need thereof a therapeutically effective amount of a pharmaceutical composition comprising a molecule that inhibits PRMT5 expression, wherein said molecule is a low molecular weight compound.

The present disclosure further provides use of a low molecular weight compound for the treatment of a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. Also provided is a use of a low molecular weight compound for the manufacture of a medicament for treating a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In another embodiment, the present invention provides a method of treating MTAP-deficient and/or MTA-accumulating cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine, by administering to a subject in need thereof a therapeutically effective amount of a pharmaceutical composition comprising a molecule that inhibits the cellular function of the PRMT5 protein.

The present disclosure further provides use of a molecule that inhibits the cellular function of the PRMT5 protein for the treatment of a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. Also provided is a use of a molecule that inhibits the cellular function of the PRMT5 protein for the manufacture of a medicament for treating a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In another embodiments, the present invention provides a method of treating MTAP-deficient and/or MTA-accumulating cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine, by administering to a subject in need thereof a therapeutically effective amount of a pharmaceutical composition comprising a molecule inhibits PRMT5 expression, wherein said molecule is a RNA inhibitor, including, but not limited to, a low molecular weight compound, a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART). Examples of such RNA inhibitor are described herein.

In another embodiments, the present invention provides a method of treating MTAP-deficient and/or MTA-accumulating cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine, by administering to a subject in need thereof a therapeutically effective amount of a pharmaceutical composition comprising an inhibitor that inhibits PRMT5 expression, wherein the inhibitor includes, but not limited to, a low molecular weight compound, a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART). Examples of such antibodies or antibody derivatives are described herein.

The present disclosure further provides use of a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART) for the treatment of a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. Also provided is a use of a a RNA inhibitor (e.g., a RNAi agent), a CRISPR, a TALEN, a zinc finger nuclease, an mRNA, an antibody or derivative thereof, an antibody-drug conjugate, or a chimeric antigen receptor T cell (CART) for the manufacture of a medicament for treating a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a method of determining if a subject afflicted with a cancer will respond to therapeutic treatment with a PRMT5 inhibitor, comprising the steps of: a) contacting a test sample obtained from said subject with a reagent capable of detecting human cancer cells have MTAP deficiency and/or MTA accumulation; and b) comparing the test sample with a reference sample taken from a non-cancerous or normal control subject, wherein the presence of MTAP deficiency and/or MTA accumulation in said sample obtained from said afflicted subject indicates said afflicted subject will respond to therapeutic treatment with a PRMT5 inhibitor. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. In some embodiments, the method further comprises the step of determining the level of PRMT5 in the cancer cells. In many cancers, PRMT5 is over-expressed. Chung et al. 2013 J. Biol. Chem. 288: 35534-47. The level of expression of PRMT5 can be taken into account when determining the therapeutically effective dosage of a PRMT5 inhibitor. In addition, during treatment, the levels of PRMT5 can be monitored to assess disease or treatment progression.

In one embodiment, the present invention provides a method of determining the sensitivity of a cancer cell associated with the loss of PRMT5 function through PRMT5 inhibitor, comprising the steps of: a) assaying for MTAP-deficiency, in said cancer cell; and b) comparing the production, level, activity, expression or presence of MTAP in a non-cancerous or normal control cell, wherein MTAP deficiency in said cancer cell indicates said cell is sensitive to a PRMT5 inhibitor. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a method of determining the sensitivity of a cancer cell to a PRMT5 inhibitor, comprising the steps of: a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similiarity in PRMT5 expression, and the presence of said MTAP deficiency in said cancer cell, indicates said cell is sensitive to a PRMT5 inhibitor. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a method of screening for PRMT5 inhibitors, said method comprising the steps of: a) contacting a test sample containing one or more MTAP-deficient and/or MTA-accumulating cells with a candidate PRMT5 inhibitor; b) measuring the reduction in proliferation and/or viability of said cells in said sample; c) contacting a reference sample containing the same type of MTAP-deficient and/or MTA-accumulating cells with a known PRMT5 inhibitor; d) measuring the reduction in proliferation and/or viability of said cells in said test sample; e) comparing the reduction in proliferation and/or viability of said test sample with proliferation and/or viability of said reference sample, wherein a reduction in proliferation and/or viability of said test sample relative to the reference sample indicates said candidate is a PRMT5 inhibitor. In some embodiments, the test sample comprises MTAP-deficient and/or MTA-accumulating cancer cells. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a kit for predicting the sensitivity of a subject afflicted with a MTAP-deficiency-related cancer for treatment with a PRMT5 inhibitor, comprising: i) reagents capable of detecting human MTAP-deficient cancer cells; and ii) instructions for how to use said kit. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a composition comprising a PRMT5 inhibitor for use in treatment of cancer in a selected patient population, wherein the patient population is selected on the basis of being afflicted with a MTAP-deficient and/or MTA-accumulating cancer. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In one embodiment, the present invention provides a therapeutic method of treating a subject afflicted with a cancer associated with MTAP deficiency and/or MTA accumulation is provided comprising the steps of: a) contacting a test sample obtained from said subject with a reagent capable of detecting human MTAP-deficient and/or MTA-accumulating cancer cells; b) comparing the test sample with a reference sample taken from a non-cancerous or normal control subject, wherein MTAP deficiency and/or MTA accumulation in said test sample indicates said afflicted subject will respond to therapeutic treatment with a PRMT5 inhibitor; and c) administering a therapeutically effective amount of PRMT5 inhibitor to those subject identified in step b). In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. In some embodiments, the method further comprises the step of determining the level of PRMT5 in the cancer cells. In many cancers, PRMT5 is over-expressed. Chung et al. 2013 J. Biol. Chem. 288: 35534-47. The level of expression of PRMT5 can be taken into account when determining the therapeutically effective dosage of a PRMT5 inhibitor. In addition, during treatment, the levels of PRMT5 can be monitored to assess disease or treatment progression.

In one embodiment, the present invention provides a therapeutic method of treating a subject afflicted with a cancer associated with MTAP deficiency and/or MTA accumulation comprising the steps of: a) contacting a test sample obtained from said subject with a reagent capable of detecting human MTAP-deficient and/or MTA-accumulating cancer cells; b) comparing the test sample with a reference sample taken from a non-cancerous or normal control subject, wherein MTAP deficiency and/or MTA accumulation in said test sample indicates said afflicted subject will respond to therapeutic treatment with a PRMT5 inhibitor; and c) administering a therapeutically effective amount of the composition according to some embodiments. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. In some embodiments, the method further comprises the step of determining the level of PRMT5 in the cancer cells. In many cancers, PRMT5 is over-expressed. The level of expression of PRMT5 can be taken into account when determining the therapeutically effective dosage of a PRMT5 inhibitor. In addition, during treatment, the levels of PRMT5 can be monitored to assess disease or treatment progression.

In one embodiment, the present invention provides a method of determining if a subject afflicted with a cancer associated with MTAP deficiency and/or MTA accumulation will respond to therapeutic treatment with a PRMT5 inhibitor, comprising the steps of: a) contacting a test sample obtained from said subject with a reagent capable of detecting human cancer cells exhibiting MTAP deficiency and/or MTA accumulation; and b) comparing the test sample with a reference sample taken from a non-cancerous or normal control subject, wherein the detection of MTAP deficiency and/or MTA accumulation in said sample obtained from said afflicted subject indicates said afflicted subject will respond to therapeutic treatment with a PRMT5 inhibitor. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. In some embodiments, the method of determining if a subject has a cancer comprising MTAP-deficient and/or MTA-accumulating cancer cells further comprises the step of determining the level of PRMT5 in the cancer cells. In many cancers, PRMT5 is over-expressed. The level of expression of PRMT5 can be taken into account when determining the therapeutically effective dosage of a PRMT5 inhibitor. In addition, during treatment, the levels of PRMT5 can be monitored to assess disease or treatment progression.

In one embodiment, the present invention provides a method of determining if a subject afflicted with a cancer associated with MTAP deficiency and/or MTA accumulation will respond to therapeutic treatment with a PRMT5 inhibitor, comprising the steps of: a) contacting a test sample obtained from said subject with a reagent capable of detecting human cancer cells exhibiting MTAP deficiency and/or MTA accumulation; and b) comparing the test sample with a reference sample taken from a non-cancerous or normal control subject, wherein the detection of MTAP deficiency and/or MTA accumulation in said sample obtained from said afflicted subject indicates said afflicted subject will respond to therapeutic treatment with a PRMT5 inhibitor. In some embodiments, the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine. In some embodiments, the method further comprises the step of determining the level of PRMT5 in the cancer cells. In many cancers, PRMT5 is over-expressed. The level of expression of PRMT5 can be taken into account when determining the therapeutically effective dosage of a PRMT5 inhibitor. In addition, during treatment, the levels of PRMT5 can be monitored to assess disease or treatment progression.

Identification of a Role of PRMT5 in Cancer

To systematically search for epigenetic synthetic lethal interactions, a pooled-shRNA screen was performed across a large collection of cancer cell lines using a library targeting a diverse set of epigenetic regulators.

While RNAi has proven to be a very powerful forward genetic approach, the robustness and reproducibility of RNAi screens has been challenged by the prevalence of off-target effects and inability to predict high-potency shRNAs with great confidence (Sigoillot, F. D., and King, R. W., 2011 ACS Chem Biol 6(1): 47-60). In an effort to overcome these limitations, a library of approximately 20 shRNAs per gene against 7500 human genes was generated. This library was packaged as a lentiviral pool and infected onto approximately 300 human cancer cell lines. After passaging the infected cell lines for two weeks, the cell lines were harvested and the representation of the library was quantified in the starting and ending populations by deep sequencing (hiSeq 2500). Comparing the frequency of shRNAs over time in the infected cancer lines allows the ranking of relative viability effects of the shRNAs and discovery of genes that are required for the proliferation of specific lines. This genetic screening has revealed that a subset of cancer cell lines are particularly sensitive to depletion of the PRMT5 protein.

This subset of lines comprises those which are MTAP-deficient. A variety of patient stratification strategies could be employed to find patients likely to be sensitive to PRMT5 depletion, including but not limited to, testing for MTAP deficiency and/or MTA accumulation.

As shown in the examples and herein, knockdown of the gene PRMT5 very specifically inhibits the growth of MTAP-deficient and/or MTA-accumulating cancers.

PRMT5 inhibition represents an attractive therapeutic target for MTAP-deficient and/or MTA-accumulating cancers.

In some embodiments, the present invention provides compositions and methods wherein the PRMT5 inhibitor is an antibody or derivative thereof, an antibody-drug conjugate, a RNA inhibitor (e.g., a RNAi agent), a CRISPR, a TALEN, a zinc finger nuclease, an mRNA, or a chimeric antigen receptor T cell (CART), or a low molecular weight compound.

Antibodies to PRMT5

In some embodiments, the present invention provides a PRMT5 inhibitor which is an antibody or epitope-binding fragment or derivative thereof, and methods of using the same. Various types of antibodies and epitope-binding fragments and derivatives thereof are known in the art, as are methods of producing these. Any of these, including but not limited to those described herein, can be used to produce a PRMT5 inhibitor, which can be used in various methods of inhibiting PRMT5 and treating a PRMT5-related disease, including, but not limited to, a disease associated with MTAP deficiency and/or MTA accumulation, including, but not limited to, a cancer, including, but not limited to, glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

In certain embodiments of the invention, the antibody to PRMT5 is an intrabody.

Single chain antibodies expressed within the cell (e.g. cytoplasm or nucleus) are called intrabodies. Due to the reducing environment within the cell, disulfide bridges, believed to be critical for antibody stability, are not formed. Thus, it was initially believed that applications of intrabodies are not suitable. But several cases are described showing the feasibility of intrabodies (Beerli et al., 1994 J Biol Chem, 269, 23931-6; Biocca et al., 1994 Bio/Technology, 12, 396-9; Duan et al., 1994 Proceedings of the National Academy of Sciences of the United States of America, 91, 5075-9; Gargano and Cattaneo, 1997 FEBS Lett, 414, 537-40; Greenman et al., 1996 J Immunol Methods, 194, 169-80; Martineau et al., 1998 Journal of Molecular Biology, 280, 117-27; Mhashilkar et al., 1995 EMBO Journal, 14, 1542-51; Tavladoraki et al., 1993 Nature, 366, 469-72). In these cases, intrabodies work by, e.g., blocking the cytoplasmic antigen and therefore inhibiting its biological activity.

Such intracellular antibodies are also referred to as intrabodies and may comprise a Fab fragment, or preferably comprise a scFv fragment (see, e.g., Lecerf et al., Proc. Natl. Acad. Sci. USA 98:4764-49 (2001). The framework regions flanking the CDR regions can be modified to improve expression levels and solubility of an intrabody in an intracellular reducing environment (see, e.g., Worn et al., J. Biol. Chem. 275:2795-803 (2000). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell Biol. 15:1182-91 (1995); Lener et al., Eur. J. Biochem. 267:1196-205 (2000)). An intrabody may be introduced into a cell by a variety of techniques available to the skilled artisan including via a gene therapy vector, or a lipid mixture (e.g., Provectin™ manufactured by Imgenex Corporation, San Diego, Calif.), or according to photochemical internalization methods.

Intrabodies can be derived from monoclonal antibodies which were first selected with classical techniques (e.g., phage display) and subsequently tested for their biological activity as intrabodies within the cell (Visintin et al., 1999 Proceedings of the National Academy of Sciences of the United States of America, 96, 11723-11728). For additional information, see: Cattaneo, 1998 Bratisl Lek Listy, 99, 413-8; Cattaneo and Biocca, 1999 Trends In Biotechnology, 17, 115-21. The solubility of an intrabody can be modified by either changes in the framework (Knappik and Pluckthun, 1995 Protein Engineering, 8, 81-9) or the CDRs (Kipriyanov et al., 1997; Ulrich et al., 1995 Protein Engineering, 10, 445-53). Additional methods for producing intrabodies are described in the art, e.g., U.S. Pat. Nos. 7,258,985 and 7,258,986.

In one embodiment, antigen-binding proteins, including, but not limited to, antibodies, that are able to target cytosolic/intracellular proteins, for example, the PRMT5 protein. The disclosed antibodies target a peptide/MHC complex as it would typically appear on the surface of a cell following antigen processing of PRMT5 protein and presentation by the cell. HLA class I binds to peptides approximately 9 amino acids in length and presents them on the surface of the cell to cytotoxic T lymphocytes. The presentation of these peptides is the product of cytoplasmic cleavage by enzymes and active transport by transporter proteins. Further, the binding of particular peptides after processing and localization is heavily influenced by the amino acid sequence of the particular HLA protein. Most of these steps are amenable to in vitro characterization, allowing one to predict the likelihood that a particular amino acid sequence, derived from a larger peptide or protein of interest, will be successfully processed, transported, bound by MHC class I, and presented to cytotoxic T lymphocytes. In that regard, the antibodies mimic T-cell receptors in that the antibodies have the ability to specifically recognize and bind to a peptide in an MHC-restricted fashion, that is, when the peptide is bound to an MHC antigen. The peptide/MHC complex recapitulates the antigen as it would typically appear on the surface of a cell following antigen processing and presentation of the PRMT5 protein to a T-cell.

The accurate prediction for a particular step in this process is dependent upon models informed by experimental data. The cleavage specificity of the proteasome, producing peptides often <30 amino acids in length, can be determined by in vitro assays. The affinity for the transporter complex can similarly be determined by relatively straight-forward in vitro binding assays. The MHC class I protein's affinity is highly variable, depending on the MHC allele, and generally must be determined on an allele-by-allele basis. One approach is to elute the peptides presented by the MHC protein on the cell surface to generate a consensus motif. An alternative approach entails generating cells deficient in a peptide processing step such that most or all of the MHC proteins on the cell surface are not loaded with a peptide. Many different peptides can be washed over the cells in parallel and monitored for binding. The set of peptides that do and do not bind can be used to train a classifier (including, but not limited to, an artificial neural network or support vector machine) to discriminate between the two peptide sets. This trained classifier can then be applied to novel peptides to predict their binding to the MHC allele. Alternatively, the affinity for each peptide can be used to train a regression model, which can then be used to make quantitative predictions regarding the MHC protein's affinity for an untested peptide. The collection of such datasets is laborious, so methods exist to combine data collected for one HLA allele with the knowledge of the amino acid differences between that particular allele and another unstudied MHC allele to predict its peptide binding specificity.

Additional methods for constructing antibodies to cytosolic peptides including, but not limited to, PRMT5 are described in, for example, WO 2012/135854. This document describes production of antibodies which recognize and bind to epitopes of a peptide/MHC complex, including, but not limited to, a peptide/HLA-A2 or peptide/HLA-A0201 complex. In some embodiments of the invention, the peptide is portion of PRMT5.

HLA class I binds to peptides approximately 9 amino acids in length and presents them on the surface of the cell to cytotoxic T lymphocytes. The presentation of these peptides is the product of cytoplasmic cleavage by enzymes and active transport by transporter proteins. Further, the binding of particular peptides after processing and localization is heavily influenced by the amino acid sequence of the particular HLA protein. Most of these steps are amenable to in vitro characterization, allowing one to predict the likelihood that a particular amino acid sequence, derived from a larger peptide or protein of interest, will be successfully processed, transported, bound by MHC class I, and presented to cytotoxic T lymphocytes.

The accurate prediction for a particular step in this process is dependent upon models informed by experimental data. The cleavage specificity of the proteasome, producing peptides often <30 amino acids in length, can be determined by in vitro assays. The affinity for the transporter complex can similarly be determined by relatively straight-forward in vitro binding assays. The MHC class I protein's affinity is highly variable, depending on the MHC allele, and generally must be determined on an allele-by-allele basis. One approach is to elute the peptides presented by the MHC protein on the cell surface to generate a consensus motif. An alternative approach entails generating cells deficient in a peptide processing step such that most or all of the MHC proteins on the cell surface are not loaded with a peptide. Many different peptides can be washed over the cells in parallel and monitored for binding. The set of peptides that do and do not bind can be used to train a classifier (including, but not limited to, an artificial neural network or support vector machine) to discriminate between the two peptide sets. This trained classifier can then be applied to novel peptides to predict their binding to the MHC allele. Alternatively, the affinity for each peptide can be used to train a regression model, which can then be used to make quantitative predictions regarding the MHC protein's affinity for an untested peptide. The collection of such datasets is laborious, so methods exist to combine data collected for one HLA allele with the knowledge of the amino acid differences between that particular allele and another unstudied MHC allele to predict its peptide binding specificity.

One such machine learning approach that combines prediction of likely proteasomal cleavage, transporter affinity, and MHC affinity is SMM (Stabilized Matrix Method, Tenzer S et al, 2005. PMID 15868101), which we used to scan the PRMT5 wildtype protein sequence, and generated a number of peptides predicted to be well-processed and high-affinity MHC binders (see Example 4).

This approach can be extended to mutations specific to an indication: a mutation leading to an amino acid change alters the peptide sequence and can lead to a peptide that produces a different score than the wildtype sequence. By focusing on such mutations and selecting those mutant peptide sequences that score highly, one can generate peptides that are presented solely in a diseased state because the sequence simply does not exist in a non-diseased individual. Cross-reactivity can be further minimized by also evaluating the wildtype sequence and selecting for downstream analyses only those peptides whose non-mutant sequence is not predicted to be processed and presented by MHC efficiently.

Once appropriate peptides have been identified, peptide synthesis may be done in accordance with protocols well known to those of skill in the art. Peptides may be directly synthesized in solution or on a solid support in accordance with conventional techniques (See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520 1998.). Peptides may then be purified by high-pressure liquid chromatography and the quality assessed by high-performance liquid chromatography analysis. Purified peptides may be dissolved in DMSO diluted in PBS (pH7.4) or saline and stored at −80 C. The expected molecular weight may be confirmed using matrix-assisted laser desorption mass spectrometry.

Subsequent to peptide selection, binding of the peptide to HLA-A may be tested. In one method, binding activity is tested using the antigen-processing deficient T2 cell line, which stabilizes expression of HLA-A on its cell surface when a peptide is loaded exogenously in the antigen-presenting groove by incubating the cells with peptide for a sufficient amount of time. This stabilized expression is read out as an increase in HLA-A expression by flow cytometry using HLA-A2 specific monoclonal antibodies (for example, BB7.2) compared to control treated cells. In another method, presence of the peptide in the HLA-A2 antigen-presenting groove of T2 cells may be detected using targeted mass spectrometry. The peptides are enriched using a MHC-specific monoclonal Ab (W6/32) and then specific MRM assays monitor the peptides predicted to be presented (See for example, Kasuga, Kie. (2013) Comprehensive Analysis of MHC Ligands in Clinical material by Immunoaffinity-Mass Spectrometry, Helena Backvall and Janne Lethio, The Low Molecular Weight Proteome: Methods and Protocols (203-218), New York, N.Y.: Springer Sciences+Business Media and Kowalewski D and Stevanovic S. (2013) Biochemical Large-Scale Identification of MHC Class I Ligands, Peter van Endert, Antigen Processing: Methods and Protocols, Methods in Molecular Biology, Vol 960 (145-158), New York, N.Y.: Springer Sciences+Business Media). This strategy differs slightly than the normally applied tandem mass spectrometry based peptide sequencing. Heavy labeled internal standards are used for identification which results in a more sensitive and quantitative approach.

Once a suitable peptide has been identified the next step would be identification of specific antibodies to the peptide/HLA-A complex, the “target antigen”, utilizing conventional antibody generation techniques including, but not limited to, phage display or hybridoma technology in accordance with protocols well known to those skilled in the art. The target antigen (for example, the peptide/HLA-A02-01 complex) is prepared by bringing the peptide and the HLA-A molecule together in solution to form the complex. Next, selection of Fab or scFv presenting phage that bind to the target antigen are selected by iterative binding of the phage to the target antigen, which is either in solution or bound to a solid support (for example, beads or mammalian cells), followed by removal of non-bound phage by washing and elution of specifically bound phage. The targeted antigen may be first biotinylated for immobilization, for example, to streptavidin-conjugated (for example, Dynabeads M-280).

Positive Fab or scFv clones may be then tested for binding to peptide/HLA-A2 complexes on peptide-pulsed T2 cells by flow cytometry. T2 cells pulsed with the specific peptide or a control irrelevant peptide may be incubated with phage clones. The cells are washed and bound phage are detected by binding an antibody specific for the coat protein (for example, M13 coat protein antibody) followed by a fluorescent labelled secondary antibody to detect the coat protein antibody (for example, anti-mouse Ig). Binding of the antibody clones to human tumor cells expressing both HLA-A2 and the target (for example, PRMT5) can also be assessed by incubating the tumor cells with phage as described or purified Fab or scFv flow cytometry and appropriate secondary antibody detection.

An alternative method to isolating antibodies specific to the peptide/HLA-A2 complex may be achieved through conventional hybridoma approaches in accordance with protocols well known to those of skill in the art. In this method, the target antigen is injected into mice or rabbits to elicit an immune response and monoclonal antibody producing clones are generated. In one embodiment, the host mouse may be one of the available human HLA-A2 transgenic animals which may serve to reduce the abundance of non-specific antibodies generated to HLA-A2 alone. Clones may then be screened for specific binding to the target antigen using standard ELISA methods (for example, incubating supernatant from the clonal antibody producing cells with biotinylated peptide/MHC complex captured on streptavidin coated ELISA plates and detected with anti-mouse antibodies). The positive clones can also be identified by incubating supernatant from the antibody producing clones with peptide pulsed T2 cells by flow cytometry and detection with specific secondary antibodies (for example, fluorescent labelled anti-mouse IgG antibodies). Binding of the antibody clones to human tumor cells expressing both HLA-A2 and the target (for example, PRMT5) can also be assessed by incubating the tumor cells with supernatant or purified antibody from the hybridoma clones by flow cytometry and appropriate secondary antibody detection.

Accordingly, the present invention provides an antibody or a fragment thereof that binds to a HLA-peptide complex comprising a peptide having the sequence of any of SEQ ID NOs: 101 to 158, as described in Example 4.

Immunotherapy

Adoptive cell transfer has been shown to be a promising treatment for various types of cancer. Adoptive cell transfer in cancer therapy involves the transfer of autologous or allogeneic immune effector cells (including, but not limited to, T cells) to enhance immune response against the tumor in a patient having cancer. Recent methods of adoptive cell transfer that have shown promise in cancer therapy include the genetic modification of cells prior to delivery to the patient to express molecules that target antigens expressed on cancer cells and improve the anti-cancer immune response. Examples of such molecules include T cell receptors (TCRs) and chimeric antigen receptors (CARs), which are described in further detail below.

TCR is a disulfide-linked membrane-anchored heterodimer present on T cell lymphocytes, and normally consisting of an alpha (α) chain and a beta (β) chain. Each chain comprises a variable (V) and a constant (C) domain, wherein the variable domain recognizes an antigen, or an MHC-presented peptide. Signaling is mediated through interaction between the antigen-bound αβ heterodimer to CD3 chain molecules, e.g., CD3zeta (ζ). Upon binding of a TCR to its antigen, a signal transduction cascade is initiated that can result in T cell activation, T cell expansion, and antitumor effect, e.g., increased cytolytic activity against tumor cells.

In TCR gene therapy, naturally occurring or modified TCRα and TCRβ chains with a known specificity and avidity for tumor antigens are introduced and expressed in a T cell. Briefly, a tumor antigen-specific T cell clone, e.g., with high affinity to the target antigen, is isolated from a donor or patient sample, e.g., a blood or PBMC sample. The tumor antigen-specific TCR α and β chains are isolated using standard molecular cloning techniques known in the art, and a recombinant expression vector for delivery into a host PBMC or T cell population, or subpopulation thereof, is generated. The host cell population is transduced, and the TCR-engineered cells are expanded and/or activated ex vivo prior to administration to the patient. T cells redirected with TCRs that target tumor antigens, including, but not limited to, glycoprotein-100 (gp100) and MART-1, have shown success in recent studies. TCR-redirected T cells recognizing any antigens that are uniquely or preferentially expressed on tumor cells can be used in the present invention.

The TCR chains can be modified to improve various TCR characteristics for enhancing therapeutic efficacy. Modifications can be made to the TCR to improve TCR surface expression by any of the following: utilizing promoters that drive high level of gene expression in T cells, e.g., retroviral long terminal repeats (LTRs), CMV, MSCV, SV40 promoters (Cooper et al., J. Virol., 2004; Jones et al., Hum. Gene Ther., 2009); introducing other regulatory elements that can enhance transgene expression, e.g., woodchuck hepatitis virus posttranscriptional regulatory element which increases RNA stability (Zufferey et al., J. Virol., 1999); codon optimization (Gustafsson et al., Trends Biotechnol., 2004); or eliminating mRNA instability motifs or cryptic splice sites (Scholten et al., Clin. Immunol., 2006); or a combination thereof. To reduce TCR chain mispairing between the introduced and endogenous TCR chains, and promote the preferential pairings of the introduced TCR chains with each other, any one of the following: introducing foreign constant domains, e.g., from another organism, to the TCRα and TCRβ chains, e.g., murine constant domains (Cα and Cβ) for human TCR chains; increasing interchain affinity by engineering a second disulfide bond in the introduced TCR, e.g., introducing additional cysteine residues in the Cα and Cβ domains (Kuball et al., Blood, 2007); or introducing mutations, e.g., point mutations, that increase the “knob in hole” interface between the TCRα and TCRβ chain (Voss et al., J. Immunol., 2008); or fusing signaling domains, e.g., CD3z domains, directly to the variable domains of the TCRα and TCRβ (Sebestyen et al., 2008); or any combination thereof. The different TCR modifications described above merely represent example modifications, and do not represent an exhaustive or comprehensive list of modifications. Other modifications that increase specificity, avidity, or function of the TCRs or the engineered T cells expressing the TCRs can be readily envisioned by the ordinarily skilled artisan. Methods for introducing the TCRs into host cells and administration of the TCR-engineered cells are further described below.

Single-chain TCRs has been described in, e.g., Willemsen R A et al, Gene Therapy 2000; 7: 1369-1377; Zhang T et al, Cancer Gene Ther 2004; 11: 487-496; Aggen et al, Gene Ther. 2012 April; 19(4):365-74.

Chimeric antigen receptors (CARs) are based upon TCRs, and generally comprise 1) an extracellular antigen binding domain; 2) a transmembrane domain; and 3) an intracellular domain comprising one or more intracellular signaling domains. Similar to TCR gene therapy, CAR gene therapy generally comprises isolating a host cell population from a donor or patient, e.g., PBMCs, T cells, or a subpopulation thereof, and introducing the CAR molecule to the host cells such that the host cells express the CAR. The CAR-redirected T cells are then expanded and activated ex vivo using methods known in the art, including, but not limited to, stimulation by anti-CD3 and anti-CD28 antibodies prior to delivery to the patient.

The antigen binding domain of a CAR refers to a molecule that has affinity for an antigen that is expressed on a target cell, e.g., a cancer cell. The antigen binding domain can be a ligand, a counterligand, or an antibody or antigen-binding fragment thereof, e.g., an Fab, Fab′, F(ab′)₂, or Fv fragment, an scFv antibody fragment, a linear antibody, single domain antibody including, but not limited to, an sdAb (either VL or VH), a camelid VHH domain, a nanobody, and multi-specific antibodies formed from antibody fragments. The antibody or fragment thereof can be humanized. Any antibodies or fragments thereof that recognize and bind to tumor antigens known in the art can be utilized in a CAR.

Accordingly, the present invention provides a CAR comprising an antibody or antibody fragment (e.g., scFv) that recognize a HLA-peptide complex, wherein the complex comprising a peptide having the sequence of any of SEQ ID NOs 101 to 158.

The transmembrane domain of a CAR refers to a polypeptide that spans the plasma membrane, linking the extracellular antigen binding domain to the intracellular domain. A transmembrane domain can include one or more additional amino acids adjacent to the transmembrane region, e.g., one or more amino acid associated with the extracellular or intracellular region of the protein from which the transmembrane was derived (e.g., 1, 2, 3, 4, 5, 6, 7, 8, 9, 10 up to 15 amino acids of the extracellular or intracellular region). Examples of transmembrane domains can be derived from any one or more of the following: the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp. Additional sequences, e.g., hinge or spacer sequence, can be disposed between a transmembrane domain and another sequence or domain to which it is fused.

The intracellular domain of a CAR includes at least one primary signaling domain and, optionally, one or more co-stimulatory signaling domains, which are responsible for activation of at least one of the normal effector functions of the immune cell in which the CAR has been introduced. Examples of primary signaling domains include TCR zeta, FcR gamma, FcR beta, CD3 gamma, CD3 delta, CD3 epsilon, CD5, CD22, CD32, CD79a, CD79b, CD66d, DAP10, and DAP12. Examples of costimulatory signaling domains include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, and PAG/Cbp. The intracellular signaling sequences may be linked to each other in random or specified order, and may be separated by a short oligo or polypeptide linker.

Introduction of the TCR and CAR molecules described above to a host cell can be accomplished using any methods known in the art. The host cells are isolated from a patient, or optionally, a donor, and can be immune effector cells, preferably T cells. In some embodiments, specific subpopulations of the immune effector cells may be preferred, for example, tumor infiltrating lymphocytes (TIL), CD4⁺ T cells, CD8⁺ T cells, helper T cells (Th cells), or NK cells. Subpopulations of immune effector cells can be identified or isolated from a patient or a donor by the expression of surface markers, e.g., CD4, CD8. The host cells can be modified by transduction or transfection of an expression vector, e.g., a lentiviral vector, a retroviral vector, or a gamma-retroviral vector, encoding the TCR or CAR molecule for sustained or stable expression of the TCR or CAR molecule. With regard to TCR, the a and p chain may be in different expression vectors, or in a single expression vector. In other embodiments, the host cells are modified by in vitro transcribed RNA encoding the TCR or CAR molecule, to transiently express the TCR or CAR. The RNA encoding the TCR or CAR molecule can be introduced to the host cell by transfection, lipofection, or electroporation. The TCR or CAR-modified host cells are cultured under conditions sufficient for expression of the TCR or CAR molecules. In some aspects, the engineered cells are expanded and/or activated using methods known in the art, including, but not limited to, culturing in the presence of specific cytokines or factors that stimulate proliferation and activation known in the art. Examples include culturing in the presence of IL-2, and/or anti-CD3/CD28 antibodies.

The patient can receive one or more doses of a therapeutic amount of TCR or CAR-engineered cells. The therapeutic amount of TCR or CAR-engineered cells in each dosage can be determined by a physician with consideration of individual differences in age, weight, tumor size, extent of infection or metastasis, and condition of the patient. It can generally be stated that a pharmaceutical composition comprising the immune TCR or CAR-engineeered cells described herein may be administered at a dosage of 10⁴ to 10⁹ cells/kg body weight, in some instances 10⁵ to 10⁶ cells/kg body weight, including all integer values within those ranges. The pharmaceutical compositions may also be administered multiple times at these dosages. The cells can be administered by using infusion techniques that are commonly known in immunotherapy (see, e.g., Rosenberg et al., New Eng. J. of Med. 319:1676, 1988), e.g., intravenous injection, or direct delivery to the site of the tumor.

Cancer vaccines generally involve inoculating a patient with a reagent designed to induce an antigen specific immune response. Preventative cancer vaccines are typically administered prior to diagnosis or development of a cancer to reduce the incidence of cancer. Preventative cancer vaccines are designed to target infectious agents, e.g., oncogenic viruses, by stimulating the immune system to recognize the infectious agents for protecting the body against future exposure. Therapeutic cancer vaccines aim to treat cancer after diagnosis by delaying or inhibiting cancer cell growth, causing tumor regression, preventing cancer relapse, or eliminating cancer cells that are not killed by other forms of treatment.

Cancer vaccines may comprise peptides or proteins, antibodies, glycoproteins, recombinant vectors or other recombinant microorganisms, killed tumor cells, protein- or peptide-activated dendritic cells. The composition of the cancer vaccine depends upon multiple factors, including, but not limited to, the particular tumor antigen that is targeted, the disease and disease stage, and whether the vaccine is administered in combination with another mode of cancer therapy. Adjuvants known in the art that modify or boost the immune response can be added to the cancer vaccine composition.

Antibody cancer vaccines have been developed, including anti-idiotype vaccines which comprise antibodies that recognize the antigenic determinants of tumor antigen-specific antibodies, called idiotopes. Thus, these anti-idiotype antibodies mimic distinct tumor antigens and act as surrogate antigens for triggering humoral and/or cellular immune response in the patient against the tumor cells. The anti-idiotype antibodies can also be fragments thereof that recognize idiotopes, e.g., single chain antibodies, scFv fragments, and sdAbs. Anti-idiotype cancer vaccines have had some success in clinical trials for treating melanoma, lung cancer, colorectal carcinoma, breast cancer, and ovarian carcinomas (Ladjemi et al., Front Oncol., 2012).

Other therapies that can be used in the context of the present invention include passive immunotherapy through delivery of antibodies that target a tumor antigen to a patient. The most common form of passive immunotherapy is monoclonal antibody therapy, in which monoclonal antibodies target the tumor cell resulting in tumor cell death through antibody-dependent cell-mediated cytotoxicity (ADCC) or complement-dependent cytotoxicity.

Various anti-PRMT5 antibodies include, but are not limited to, those known in the art.

A PRMT5 inhibitor which is an antibody can be prepared; alternatively, many PRMT5 antibodies are known in the art.

For example, Meister et al. demonstrated an inhibitory anti-PRMT5 antibody which reduced methylation by a complex of PRMT5, pICIN, and other proteins. Meister et al. 2001 Curr. Biol. 11: 1990-1994.

Additional anti-PRMT5 antibodies are known, and have been published in:
Ancelin et al. 2006. Nat. Cell. Biol. 8: 623-630;
Liu et al. 2011 Cancer Cell 19: 283-294 (which shows a PRMT5 antibody generated using the PRMT5 fragment CPPNA(pY/Y)ELFAKG(pY/Y)ED(pY/Y)LQSPL, SEQ ID NO: 39, wherein Y is tyrosine, and pY is phosphorylated tyrosine);
Sif et al. 1998 Genes Dev. 12: 2842-2851;
Sif et al. 2001 Genes Dev. 15: 603-618;
Pal et al. 2003 Mol. Cell. Biol. 23: 7475 (using a polyclonal anti-PRMT5 antibody, to GST-PRMT5, aa 4-637);
Pal et al. 2004 Mol. Cell. Biol. 24: 9630-9645;
Pal et al. 2007 EMBO J. 26: 3558-3569;
Wang et al. 2008 Mol. Cell. Biol. 28: 6262;
Boisvert et al. 2002 J. Cell Biol. 159: 957-969 (using the PRMT5 fragment KNRPGPQTRSDLLLSGRDWN, SEQ ID NO: 40, as an antigenic epitope);
Boisvert et al. 2005 Genes Dev. 19: 671-676;
Guderian et al. 2011 J. Biol. Chem. 286: 1976-1986;
Ostareck-Lederer et al. 2006 J. Biol. Chem. 281: 11115-11125

Anti-PRMT5 antibodies are also available commercially. These are available from, for example:
Abcam (3766, as used in Lacroix et al. 2008 EMBO J. 9: 452-458);
BD Biosciences (611538, as used in Dacwag et al. 2007 Mol. Cell. Biol. 27: 384)
Cell Signaling Technology, Boston, Mass. (polyclonal antibody, as used in Maloney et al. 2007 Cancer Res. 67: 3239-3253);
Chemicon, Temecula (as used in Eckert et al. 2008 BMC Dev. Biol. 8);
Santa Cruz Biotechnology, Santa Cruz, Calif. (as used in Lu et al. 2012 Oncogen. 1, e29);
Sigma-Aldrich (as used in Teng et al. 2007 Cancer Res. 67: 10491-10500);
Transduction Laboratories (as used in Fabbrizio et al. 2002 EMBO J. 3: 641-645; and Amente et al. 2005 FEBS Lett. 579: 683-689); and
Upstate Biotechnology (polyclonal antibody, as used in Zhou et al. 2010 Cell Res. 20: 1023-1033; and Gonsalvez et al. 2006 Curr. Biol. 16: 1077-1089; and Cesaro et al. 2009 J. Biol. Chem. 284: 32321-32330; 07405, as used in Lacroix et al. 2008 EMBO J. 9: 452-458; and 12-303, Le Guezennec et al. 2006 Mol. Cell. Biol. 26: 843).

All references to PRMT5 antibodies cited immediately above are hereby incorporated by reference in their entirety.

Any inhibitory anti-PRMT5 antibody or fragment thereof can be used with any method disclosed herein.

All the documents listed herein describing a PRMT5 inhibitor, including, but not limited to, an antibody, a RNAi agent, a low molecular weight compound, or any other PRMT5 inhibitor, are hereby incorporated in their entirety by reference.

Any anti-PRMT5 antibody described herein or known in the art can be used in the methods described herein. For example, any of the anti-PRMT5 antibodies described herein can be used in a method of inhibiting proliferation of MTAP-deficient cells in a subject in need thereof, the method comprising the step of administering to the subject, a PRMT5 inhibitor in an amount that is effective to inhibit proliferation of the MTAP-deficient cells.

PRMT5 RNAi Agents and Therapies

In some embodiments, the present invention provides a RNAi agent to PRMT5, and methods of using a RNAi agent to PRMT5. RNAi agents to PRMT5 include those compositions capable of mediating RNA interference, including, inter alia, shRNAs and siRNAs. In some embodiments, the RNAi agent comprises an antisense strand and a sense strand.

An embodiment of the invention provides a composition comprising an RNAi agent comprising a first (sense) or second (antisense) strand, wherein the sense and/or antisense strand comprises at least 15 contiguous nucleotides differing by 0, 1, 2, or 3 nucleotides from the sequence of an RNAi agent to PRMT5 selected from any sequence provided herein (e.g., in SEQ ID NOs: 1-35 or 1-18, 41-49, 52-79, or 84-96, or RNAi agent comprising a sequence comprising 15 contiguous nt of the PRMT5 target sequence of any of these sequences capable of mediating RNA interference against PRMT5). In another embodiment, the present invention provides a composition comprising an RNAi agent comprising a sense strand and an antisense strand, wherein the antisense strand comprises at least 15 contiguous nucleotides differing by 0, 1, 2, or 3 nucleotides from the antisense strand of an RNAi agent to PRMT5 from any sequence provided herein.

In another embodiment, the present invention provides a composition comprising an RNAi agent comprising a sense strand and an antisense strand, wherein the sense strand comprises at least 15 contiguous nucleotides differing by 0, 1, 2, or 3 nucleotides from the sense strand and the antisense strand comprises at least 15 contiguous nucleotides differing by 0, 1, 2, or 3 nucleotides from the antisense strand of an RNAi agent to PRMT5 listed immediately above.

In one embodiment, the present invention provides particular compositions comprising an RNAi agent comprising an antisense strand, wherein the antisense strand comprises at least 15 contiguous nucleotides from the antisense strand of an RNAi agent to PRMT5 selected from any one or more of the provided herein (e.g., in SEQ ID NOs: 1-35 or 1-18, 41-49, 52-79, or 84-96). In another embodiment, the present invention provides a composition comprising an RNAi agent comprising a sense strand and an antisense strand, wherein the sequence of the antisense strand is the sequence of the strand of an RNAi agent to PRMT5 sequence provided herein (e.g., in SEQ ID NOs: 1-35 or 1-18, 41-49, 52-79, or 84-96). In another embodiment, the present invention provides a composition comprising an RNAi agent comprising a sense strand and an antisense strand, wherein the sequence of the antisense strand comprises the sequence of the antisense strand of an RNAi agent to PRMT5 selected from any one or more of the sequences in Table 3.

Additional RNAi agents to PRMT5 are known in the art.

Specific RNAi agents include: The shRNAs to PRMT5 disclosed herein (particularly those having a target sequence of any of SEQ ID NOs: 1 to 18).

Additional RNAi agents to PRMT5 can be prepared, or are known in the art. Various PRMT5 RNAi agents disclosed in the art include:
Bandyopadhyay et al. 2012 Mol. Cell. Biol. 32: 1202-1213 (which shows a PRMT5 siRNA which targets the PRMT5 sequence AAGAGGGAGUUCAUUCAGGAA, SEQ ID NO: 41);
Bao et al. 2013 J. Hist. Cyt. 61: 206 (which discloses PRMT5 RNAi agents which target the PRMT5 sequences GGGACUGGAAUACGCUAAUTT, SEQ ID NO: 42, and AUUAGCGUAUUCCAGUCCCTT, SEQ ID NO: 43; and GGACCUGAGAGAUGAUAUATT, SEQ ID NO: 44, and UAUAUCAUCUCUCAGGUCCTT, SEQ ID NO: 45);
Bezzi et al. 2013 Genes Dev. 27: 1903-1916 (which shows a PRMT5 RNAi agent which targets the PRMT5 sequence CCTCAAGAACTCCCTGGAATA, SEQ ID NO: 46);
Cesaro et al. 2009 J. Biol. Chem. 284: 32321-32330 [which describes PRMT5 siRNAs which target the PRMT5 sequences GGACAAUCUGGAAUCUCAGACAUAU, SEQ ID NO: 47 (nt 1039-1064); GGCUCCAGAGAAAGCAGACAUCAUU, SEQ ID NO: 48 (nt 1363-1388); and GCGGCCAUGUUACAGGAGCUGAAUU, SEQ ID NO: 49 (nt 404-429)];
Chung et al. 2013 J. Biol. Chem. 288: 35534-35547 (wherein PRMT5 shRNA plasmids were constructed using sense GATCCCGCCCAGTTTGAGATGCCTTATGTGTGCTGTCCATAAGGCATCTCA AACTGGGCTTTTTGGAAA, SEQ ID NO: 50, and antisense AGCTTTTCCAAAAAGCCCAGTTTGAGATGCCTTATGGACAGCACACATAA GGCATCTCAAACTGGGCGG, SEQ ID NO: 51, primers; or sense AAAAACACTTCATATGTCTGAGACCTGTCTC, SEQ ID NO: 52, and antisense AATCTCAGACAT-ATGAAGTGTTTCCTGTCTC, SEQ ID NO: 53, primers);
Gonsalvez et al. 2007 J. Biol. Chem. 178: 733-740 (which describes a PRMT5 RNAi agent which targets PRMT5 sequence GGCCAUCUAUAAAUGUCUG, SEQ ID NO: 54);
Girardot et al. 2014 Nucl. Acids Res. 42: 235-248 (which shows PRMT5 shRNAs which target PRMT5 sequences GAGGGAGTTCATTCAGGAA, SEQ ID NO: 55, and GGATGTGGTGGCATAACTT, SEQ ID NO: 56);
Gkountela et al. 2014 Stem Cell Rev. Rep. 10: 230-239;
Gu et al. 2012 Biochem. J. 446: 235-241 (which used a PRMT5 shRNA targeting PRMT5 sequence GGATAAAGCTGTATGCTGT, SEQ ID NO: 57);
Gu et al. 2012 PLoS ONE 7: e44033 (which shows a PRMT5 shRNA which targets PRMT5 sequence GGATAAAGCTGTATGCTGT, SEQ ID NO: 58);
Han et al. 2013 Stem Cells 31: 953-965 (which shows a PRMT5 shRNA which targets PRMT5 sequences CTCTTGTGAATGCGTCTCTT, SEQ ID NO: 59, and AGCTCTGAGTTCTCTTCCTA, SEQ ID NO: 60);
Harris et al. J. Biol. Chem. 289: 15328-15339 (which discloses a PRMT5 siRNA which targets the sequence GAGGGAGUUCAUUCAGGAAUU, SEQ ID NO: 61);
He et al. 2011 Nucl. Acids Res. 39: 4719-4727 (which shows two shRNAs to PRMT5 which target PRMT5 sequence nt 1016-1034, GGCCATCTATAAATGTCTG, SEQ ID NO: 62, or CAGACATTTATAGATGGCC, SEQ ID NO: 63);
Huang et al. 2011 J. Biol. Chem. 286: 44424-44432 (which describes the use of a pool of PRMT5 RNAi agents which target PRMT5 sequences GAGCACAGCACUUCCUGAAAGAUGA, SEQ ID NO: 64, AGACGUGGUUGUGGUGGCAUAACUU, SEQ ID NO: 65, and CCAUCCCAACCGAGAUCCUAUGAUU, SEQ ID NO: 66);
Jansson et al. 2008 Nat. Cell. Biol. 10: 1431-1439 (which discloses a PRMT5 siRNA which targets PRMT5 sequence CCGCUAUUGCACCUUGAA, SEQ ID NO: 67);
Kanade et al. 2012 J. Biol. Chem. 287: 7313-7323 (which discloses several PRMT5 RNAi agents, including those that target PRMT5 sequences CAGCCACUGAUGGACAAUCUGGAAU, SEQ ID NO: 68, and CCGGCUACUUUGAGACUGUGCUUUA, SEQ ID NO: 69);
La Thangue, WO 2011/077133 and U.S. Patent Application Pub. No. 20130011497 (application. Ser. No. 13/518,200), which disclose PRMT5 RNAi agents which target the PRMT5 sequences 5′ CCGCUAUUGCACCUUGGAA (SEQ ID NO: 99), and CAACAGAGAUCCUAUGAUU (SEQ ID NO:100);
Liu et al. 2011 Cancer Cell 19: 283-294;
Nicholas et al. 2013 PLoS ONE (which discloses a PRMT5 RNAi agent which targets PRMT5 sequence CCGCUAUUGCACCUUGGAA, SEQ ID NO: 70);
Paul et al. 2012 Cell Death and Diff. 19: 900-908 (which shows PRMT5 shRNAs with sequences ATTGCGTCCCCGAAATAGCT, SEQ ID NO: 71, and GCGGATGGAAGACAGGCAT, SEQ ID NO: 72);
Richard et al. 2005 Biochem. J. 388: 379-386 (which used a PRMT5 siRNA which targeted the sequence of accession no. XM 033433, nt 1598-1620);
Scoumanne et al. 2009 Nucl. Acids Res. 1-12 (which discloses PRMT5 shRNAs which target PRMT5 sequences ACCGCTATTGCACCTTGGA, SEQ ID NO: 73; TCCAAGGTGCAATAGCGGT, SEQ ID NO: 74; ACCGCTATTGCACCTTGGA, SEQ ID NO: 75; and TCCAAGGTGCAATAGCGGT, SEQ ID NO: 76);
Tabata et al. 2009 Genes to Cells 14: 17-28 (which shows a PRMT5 siRNA which targets PRMT5 sequence CCGCTATTGCACCTTGGAA, SEQ ID NO: 77);
Tanaka et al. 2009 Mol. Cancer Res. 7: 557 (which shows PRMT5 siRNAs to PRMT5 se quences of nt 973-961, CAGCCACTGATGGACAATCTGGAAT, SEQ ID NO: 78, and nt 1655-1679, CCGGCTACTTTGAGACTGTGCTTTA, SEQ ID NO: 79);
Tee et al. 2010 Genes Dev. 24: 2772-2777 (which discloses PRMT5 shRNA sequences of GATCCCCGGTTTGATTTCCTCTGCATTTCAAGAGAATGCAGAGGAAATCA AACCTTTTTA, SEQ ID NO: 80, and GATCCCCGGACTGGAATACGCTAATTTTCAAGAGAAATTAGCGTATTCCA GTCCTTTTTA, SEQ ID NO: 81, and GATCCCCGGTCTTCCAGCTTTCCTATTTCAAGAGAATAGGAAAGCTGGAA GACCTTTTTA, SEQ ID NO: 82, and GATCCCCGCCACCACTCTTCCATGTTTTCAAGAGAAACATGGAAGAGTGG TGGCTTTTTA, SEQ ID NO: 83, wherein the PRMT5 target sequences are GGTTTGATTTCCTCTGCAT, SEQ ID NO: 84; ATGCAGAGGAAATCAAACC, SEQ ID NO: 85; GGACTGGAATACGCTAAT; AATTAGCGTATTCCAGTCC; GGTCTTCCAGCTTTCCTAT, SEQ ID NO: 86; ATAGGAAAGCTGGAAGACC, SEQ ID NO: 87; GCCACCACTCTTCCATGTT, SEQ ID NO: 88; and AACATGGAAGAGTGGTGGC, SEQ ID NO: 89);
Wei et al. 2012 Cancer Sci 103: 1640-1650 (which presents an anti-PRMT5 shRNA which targets the PRMT5 sequence ATAAGGCATCT-CAAACTGGGC, SEQ ID NO: 91);
Yan et al. 2014 Cancer Res. 74: 1752 (which discloses PRMT5 siRNAs which target the PRMT5 sequences CCGCUAUUGCACCUUGGAAUU, SEQ ID NO: 92, ACACUUCAUAUGUCUGAGA, SEQ ID NO: 93, and UCUCAGACAUAUGAAGUGU, SEQ ID NO: 94); and
Zhao et al. 2009 Nature Struct. Mol. Biol. 16: 304 (which used PRMT5 shRNAs targeting sequences GGACCTGAGAGATGATATA, SEQ ID NO: 95, and GAGGATTGCAGTGGCTCTT, SEQ ID NO: 96); and
WO 2011/077133.

All references to PRMT5 RNAi agents cited immediately above are hereby incorporated by reference in their entirety.

It is noted that in the present disclosure a RNAi agent to PRMT5 may be recited to target a particular PRMT5 sequence, indicating that the recited sequence may be comprised in the sequence of the sense or anti-sense strand of the RNAi agent; or, in some cases, a sequence of at least 15 contiguous nt of this sequence may be comprised in the sequence of the sense or anti-sense strand. It is also understood that some of the target sequences are presented as DNA, but the RNAi agents targeting these sequences can be RNA, or any nucleotide, modified nucleotide or substitute disclosed herein, provided that the molecule can still mediate RNA interference.

All the documents listed herein describing a PRMT5 inhibitor, including, but not limited to, a RNAi agent, a low molecular weight compound, an antibody, or any other PRMT5 inhibitor, are hereby incorporated in their entirety by reference.

The invention contemplates any PRMT5 inhibitor described herein for used in any method described herein.

Any anti-PRMT5 RNAi agent described herein or known in the art can be used in the methods described herein. For example, any of the anti-PRMT5 RNAi agents described herein (or a RNAi agent comprising 15 contiguous nt of a PRMT5 target sequence disclosed herein capable of mediating RNA interference against PRMT5) can be used in a method of inhibiting proliferation of MTAP-deficient and/or MTA-accumulating cells in a subject in need thereof, the method comprising the step of administering to the subject, a PRMT5 inhibitor in an amount that is effective to inhibit proliferation of the MTAP-deficient and/or MTA-accumulating cells.

In some embodiments, the antisense and sense strand can be two physically separated strands, or can be components of a single strand or molecule, e.g., they are linked a loop of nucleotides or other linker. A non-limiting example of the former is a siRNA; a non-limiting example of the latter is a shRNA. The can also, optionally, exist single-stranded nicks in the sense strand, or one or more mismatches between the antisense and sense strands.

The disclosure also provides combination of paired antisense and sense strands from any two sequences provided herein (e.g., in SEQ ID NOs: 1-35 or 1-18, 41-49, 52-79, or 84-96). Additional modified sequences (e.g., sequences comprising one or more modified base) of each of the compositions above are also contemplated as part of the disclosure.

In various embodiments, the RNAi agent can comprise nucleotides, modified nucleotides and/or nucleotide substitutes. A nucleotide consists of a sugar, a base and a phosphate. Any of these (the sugar, base and/or phosphate) can be modified to make a modified nucleotide.

In one embodiment, the antisense strand is about 30 or fewer nucleotides in length.

In one embodiment, the antisense strand forms a duplex region with a sense strand, wherein the duplex region is about 15 to 30 nucleotide pairs in length.

In one embodiment, the antisense strand is about 15 to about 30 nucleotides in length, including about 19 to about 23 nucleotides in length. In one embodiment, the antisense strand has at least the length selected from about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides and 30 nucleotides. RNAi agents comprising nucleotides, modified nucleotides and/or nucleotide substitutes can be of any of these lengths.

In one embodiment, the RNAi agent comprises a modification that causes the RNAi agent to have increased stability in a biological sample or environment.

In one embodiment, the RNAi agent comprises at least one sugar backbone modification (e.g., phosphorothioate linkage) or at least one 2′-modified nucleotide.

In one embodiment, the RNAi agent comprises: at least one 5′-uridine-adenine-3′ (5′-ua-3′) dinucleotide, wherein the uridine is a 2′-modified nucleotide; at least one 5′-uridine-5 guanine-3′ (5′-ug-3′) dinucleotide, wherein the 5′-uridine is a 2′-modified nucleotide; at least one 5′-cytidine-adenine-3′ (5′-ca-3′) dinucleotide, wherein the 5′-cytidine is a 2′-modified nucleotide; or at least one 5′-uridine-uridine-3′ (5′-uu-3 ‘) dinucleotide, wherein the 5’-uridine is a 2′-modified nucleotide. These dinucleotide motifs are particularly prone to serum nuclease degradation (e.g. RNase A). Chemical modification at the 2′-position of the first pyrimidine nucleotide in the motif prevents or slows down such cleavage. This modification recipe is also known under the term ‘endo light’.

In one embodiment, the RNAi agent comprises a 2′-modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In one embodiment, all pyrimidines (uridine and cytidine) are 2′-O-methyl-modified nucleosides. In some embodiments, one or more nucleotides can be modified, or RNA can be substituted with DNA, or a nucleotide substitute such as: a peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), and unlocked nucleic acid (UNA).

In some embodiments, the sense and/or antisense strand can terminate at the 3′ end with a phosphate or modified internucleoside linker, and further comprise, in 5′ to 3′ order: a spacer, a second phosphate or modified internucleoside linker, and a 3′ end cap. In some embodiments, modified internucleoside linker is selected from phosphorothioate, phosphorodithioate, phosphoramidate, boranophosphonoate, an amide linker, and a compound of formula (I):

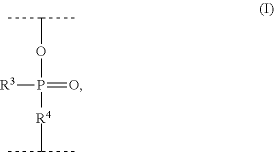

where R³ is selected from O—, S—, NH₂, BH₃, CH₃, C₁₋₆ alkyl, C₆₋₁₀ aryl, C₁₋₆ alkoxy and C₆₋₁₀ aryl-oxy, wherein C₁₋₆ alkyl and C₆₋₁₀ aryl are unsubstituted or optionally independently substituted with 1 to 3 groups independently selected from halo, hydroxyl and NH₂; and R⁴ is selected from O, S, NH, and CH₂. In some embodiments, the spacer can be a sugar, alkyl, cycloakyl, ribitol or other type of abasic nucleotide, 2′-deoxy-ribitol, diribitol, 2′-methoxyethoxy-ribitol (ribitol with 2′-MOE), C₃₋₆ alkyl, or 4-methoxybutane-1,3-diol (5300). In some embodiments, the 3′ end cap can be selected from any of various 3′ end caps described herein or known in the art. In some embodiments, one or more phosphates can be replaced by a modified internucleoside linker.

In one embodiment, the RNAi agent comprises at least one blunt end.

In one embodiment, the RNAi agent comprises an overhang having 1 nt to 4 nt.

In one embodiment, the RNAi agent comprises an overhang at the 3′-end of the antisense strand of the RNAi agent.

In one embodiment, the RNAi agent is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

In one embodiment, the composition further comprises a second RNAi agent to PRMT5.

RNAi agents of the present invention can be delivered or introduced (e.g., to a cell in vitro or to a patient) by any means known in the art.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art including, but not limited to, electroporation and lipofection. Further approaches are described below or known in the art.

Delivery of RNAi agent to tissue is a problem both because the material must reach the target organ and must also enter the cytoplasm of target cells. RNA cannot penetrate cellular membranes, so systemic delivery of naked RNAi agent is unlikely to be successful. RNA is quickly degraded by RNAse activity in serum. For these reasons, other mechanisms to deliver RNAi agent to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PE1), bacterial delivery (tkRNAi), and also chemical modification (LNA) of siRNA to improve stability. Xia et al. 2002 Nat. Biotechnol. 20 and Devroe et al. 2002. BMC Biotechnol. 21: 15, disclose incorporation of siRNA into a viral vector. Other systems for delivery of RNAi agents are contemplated, and the RNAi agents of the present invention can be delivered by various methods yet to be found and/or approved by the FDA or other regulatory authorities.

Liposomes have been used previously for drug delivery (e.g., delivery of a chemotherapeutic). Liposomes (e.g., cationic liposomes) are described in PCT publications W002/100435A1, W003/015757A1, and W004029213A2; U.S. Pat. Nos. 5,962,016; 5,030,453; and 6,680,068; and U.S. Patent Application 2004/0208921. A process of making liposomes is also described in W004/002453A1. Furthermore, neutral lipids have been incorporated into cationic liposomes (e.g., Farhood et al. 1995). Cationic liposomes have been used to deliver RNAi agent to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Publ. 2003/0012812.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

Chemical transfection using lipid-based, amine-based and polymer-based techniques, is disclosed in products from Ambion Inc., Austin, Tex.; and Novagen, EMD Biosciences, Inc, an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete 1 0, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammal's circulatory system.

A variety of molecules have been used for cell-specific RNAi agent delivery. For example, the nucleic acid-condensing property of protamine has been combined with specific antibodies to deliver siRNAs. Song et al. 2005 Nat Biotch. 23: 709-717. The self-assembly PEGylated polycation polyethylenimine has also been used to condense and protect siRNAs. Schiffelers et al. 2004 Nucl. Acids Res. 32: 49, 141-110.

The siRNA-containing nanoparticles were then successfully delivered to integrin overexpressing tumor neovasculature. Hu-Lieskovan et al. 2005 Cancer Res. 65: 8984-8992.

The RNAi agents of the present invention can be delivered via, for example, Lipid nanoparticles (LNP); neutral liposomes (NL); polymer nanoparticles; double-stranded RNA binding motifs (dsRBMs); or via modification of the RNAi agent (e.g., covalent attachment to the dsRNA).

Lipid nanoparticles (LNP) are self-assembling cationic lipid based systems. These can comprise, for example, a neutral lipid (the liposome base); a cationic lipid (for siRNA loading); cholesterol (for stabilizing the liposomes); and PEG-lipid (for stabilizing the formulation, charge shielding and extended circulation in the bloodstream). The cationic lipid can comprise, for example, a headgroup, a linker, a tail and a cholesterol tail. The LNP can have, for example, good tumor delivery, extended circulation in the blood, small particles (e.g., less than 100 nm), and stability in the tumor microenvironment (which has low pH and is hypoxic).

Neutral liposomes (NL) are non-cationic lipid based particles.

Polymer nanoparticles are self-assembling polymer-based particles.

Double-stranded RNA binding motifs (dsRBMs) are self-assembling RNA binding proteins, which will need modifications.

Several other molecules may be suitable to inhibit PRMT5, including, but not limited to, low molecular weight compounds, RNAi agents, CRISPRs, TALENs, ZFNs, and antibodies.

Additional PRMT5 Inhibitors

In one embodiment, the disclosure comprises a low molecular weight compound inhibiting PRMT5 gene expression. that inhibits PRMT5 expression.

In another embodiment, the present invention provides a molecule that inhibits the cellular function of the PRMT5 protein, such as a part of a methylation pathway.

The PRMT5 inhibitor of the present disclosure can also be, inter alia, derived from a CRISPR/Cas system, TALEN, or ZFN.

CRISPR to Inhibit PRMT5

By “CRISPR” (e.g., a “CRISPR to PRMT5” or “CRISPR to inhibit PRMT5”) and the like is meant a set of clustered regularly interspaced short palindromic repeats, or a system comprising such a set of repeats designed for a particular target (e.g., PRMT5). By “Cas” is meant a CRISPR-associated protein. By “CRISPR/Cas” system is meant a system derived from CRISPR and Cas which can be used to silence, enhance or mutate the PRMT5 gene.

Naturally-occurring CRISPR/Cas systems are found in approximately 40% of sequenced eubacteria genomes and 90% of sequenced archaea. Grissa et al. 2007. BMC Bioinformatics 8: 172. This system is a type of prokaryotic immune system that confers resistance to foreign genetic elements such as plasmids and phages and provides a form of acquired immunity. Barrangou et al. 2007. Science 315: 1709-1712; Marragini et al. 2008 Science 322: 1843-1845.

The CRISPR/Cas system has been modified for use in gene editing (silencing, enhancing or changing specific genes) in eukaryotes such as mice or primates. Wiedenheft et al. 2012. Nature 482: 331-8. This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the PRMT5 CRISPR/Cas system, the spacers are derived from the PRMT5 gene sequence. The repeats generally show some dyad symmetry, implying the formation of a secondary structure such as a hairpin, but they are not truly palindromic.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. 2010. Science 327: 167-170; Makarova et al. 2006 Biology Direct 1: 7. The spacers thus serve as templates for RNA molecules, analogously to siRNAs. Pennisi 2013. Science 341: 833-836.

As these naturally occur in many different types of bacteria, the exact arrangements of the CRISPR and structure, function and number of Cas genes and their product differ somewhat from species to species. Haft et al. 2005 PLoS Comput. Biol. 1: e60; Kunin et al. 2007. Genome Biol. 8: R61; Mojica et al. 2005. J. Mol. Evol. 60: 174-182; Bolotin et al. 2005. Microbiol. 151: 2551-2561; Pourcel et al. 2005. Microbiol. 151: 653-663; and Stern et al. 2010. Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. 2008. Science 321: 960-964. In other prokaryotes, Cas6 processes the CRISPR transcript. The CRISPR-based phage inactivation in E. coli requires Cascade and Cas3, but not Cm′ or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi 2013. Science 341: 833-836.

The CRISPR/Cas system can thus be used to edit the PRMT5 gene (adding or deleting a basepair), e.g., repairing a damaged PRMT5 gene (e.g., if the damage to PRMT5 results in high post-translational modification, production, expression, level, stability or activity of PRMT5), or introducing a premature stop which thus decreases expression of an over-expressed PRMT5. The CRISPR/Cas system can alternatively be used like RNA interference, turning off the PRMT5 gene in a reversible fashion. In a mammalian cell, for example, the RNA can guide the Cas protein to the PRMT5 promoter, sterically blocking RNA polymerases.

Artificial CRISPR/Cas systems can be generated which inhibit PRMT5, using technology known in the art, e.g., that described in U.S. patent application Ser. No. 13/842,859.

TALEN to Inhibit PRMT5

By “TALEN” (e.g., a “TALEN to PRMT5” or “TALEN to inhibit PRMT5”) and the like is meant a transcription activator-like effector nuclease, an artificial nuclease which can be used to edit a gene (e.g., the PRMT5 gene).

TALENs are produced artificially by fusing a TAL effector DNA binding domain to a DNA cleavage domain. Transcription activator-like effects (TALEs) can be engineered to bind any desired DNA sequence, including a portion of the PRMT5 gene. By combining an engineered TALE with a DNA cleavage domain, a restriction enzyme can be produced which is specific to any desired DNA sequence, including a PRMT5 sequence. These can then be introduced into a cell, wherein they can be used for genome editing. Boch 2011 Nature Biotech. 29: 135-6; and Boch et al. 2009 Science 326: 1509-12; Moscou et al. 2009 Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is a wild-type or mutated FokI endonuclease. Several mutations to FokI have been made for its use in TALENs; these, for example, improve cleavage specificity or activity. Cermak et al. 2011 Nucl. Acids Res. 39: e82; Miller et al. 2011 Nature Biotech. 29: 143-8; Hockemeyer et al. 2011 Nature Biotech. 29: 731-734; Wood et al. 2011 Science 333: 307; Doyon et al. 2010 Nature Methods 8: 74-79; Szczepek et al. 2007 Nature Biotech. 25: 786-793; and Guo et al. 2010 J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. 2011 Nature Biotech. 29: 143-8.

A PRMT5 TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the PRMT5 gene or introduce such a defect into a wt PRMT5 gene, thus decreasing expression of PRMT5.

TALENs specific to sequences in PRMT5 can be constructed using any method known in the art, including various schemes using modular components. Zhang et al. 2011 Nature Biotech. 29: 149-53; Geibler et al. 2011 PLoS ONE 6: el9509.

Zinc Finger Nuclease to Inhibit PRMT5

By “ZFN” or “Zinc Finger Nuclease” (e.g., a “ZFN to PRMT5” or “ZFN to inhibit PRMT5”) and the like is meant a zinc finger nuclease, an artificial nuclease which can be used to edit a target gene (e.g., the PRMT5 gene).

Like a TALEN, a ZFN comprises a FokI nuclease domain (or derivative thereof) fused to a DNA-binding domain. In the case of a ZFN, the DNA-binding domain comprises one or more zinc fingers. Carroll et al. 2011. Genetics Society of America 188: 773-782; and Kim et al. Proc. Natl. Acad. Sci. USA 93: 1156-1160.

A zinc finger is a small protein structural motif stabilized by one or more zinc ions. A zinc finger can comprise, for example, Cys₂His₂, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. 1998 Proc. Natl. Acad. Sci. USA 95: 10570-5.

Also like a TALEN, a ZFN can create a double-stranded break in the DNA, which can create a frame-shift mutation if improperly repaired, leading to a decrease in the expression and level of PRMT5 in a cell. ZFNs can also be used with homologous recombination to mutate, or repair defects, in the PRMT5 gene.

ZFNs specific to sequences in PRMT5 can be constructed using any method known in the art. Cathomen et al. Mol. Ther. 16: 1200-7; and Guo et al. 2010. J. Mol. Biol. 400: 96.

Low Molecular Weight Compounds to Inhibit PRMT5

Many small molecules have been found which have inhibitory properties against PRMT5.

Examples of inhibitors to PRMT5 activity include, but are not limited to, those known in the art. Example PRMT5 inhibitors include, as non-limiting examples:

PRMT inhibitors disclosed by Cheng, et al in a publication J. Biol. Chem., 2004, 279, 23, 23892-23899;

Sinefungin (5′-Deoxy-5′-(1,4-diamino-4-carboxybutyl)adenosine) inhibits PRMT5 activity, methylating the substate E2-F-1, as disclosed in the Declaration of La Thangue, dated Apr. 23, 2014, in U.S. Patent Application Publ. No. 20130011497 (U.S. patent application Ser. No. 13/518,200), and a publication by Antonysamy et al. 2012 Proc. Natl. Acad. Sci. U.S.A. 109: 17960-17965 and having the molecular structure

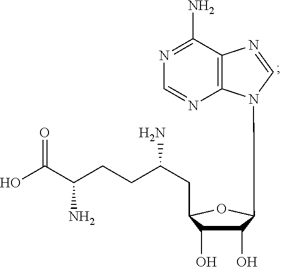

PRMT5 inhibitors CMP5, HLCL7 and CMP12, as disclosed in a publication by Roach et al. 2013 Blood 122 (21);

PRMT5 inhibitors BLL-1 and BLL-3, as discosed in publications by Parekh et al., 2011 Sem. Cancer Biol. 21: 335-346, and Yan et al. 2013 Cancer Res. 73 (8), Supp. 1;

PRMT5 inhibitors selected from: compound CMP5 (BLL1) and various derivatives thereof, including BLL2-BLL8 and BLL36, as disclosed in U.S. Pat. Appl. Publ. No. US20130059892 and International Pat. Publ. No. WO 2011/079236 to Baiocchi et al.;

PRMT5 inhibitors CMP5 and BLL54, as disclosed in a publication by Gordon, 2012, Targeting Protein Arginine Methytransferase 5 (PRMT5) Overexpression by Use of Small Molecule PRMT5 Inhibitors in Glioblastoma Multiforme (GBM), Honors Research Thesis, Ohio State University;

a cell line study disclosing that inhibition of PRMT5 induces lymphoma cell death in different non-Hodgkin lymphoma cell lines through reactivation of the retinoblastoma tumor pathway and polycomb repressor complex 2 (PRC2) silencing in a publication by Chung et al. 2013 J. Biol. Chem. 288: 35534-47;

Lysine and arginine protein methyltransferase inhibitors of Formulas I, II and III:

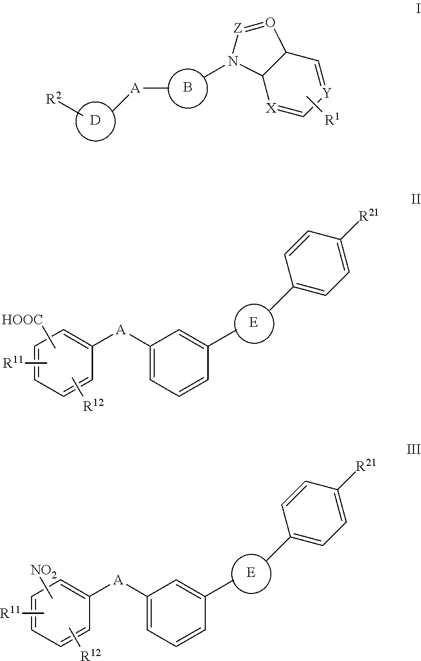

### Wherein:

Q is chosen from —CH— and —N—;
X is chosen from —CH— and —N—;
Y is chosen from —CR¹— and —N—;
Z is chosen from —CH— and —N—;
R¹ is chosen from (C₁-C₄)alkyl, halogen and optionally substituted aryl;
B is chosen from
(a) aryl optionally substituted with from one to three substituents chosen independently from halogen, OH, —NR⁵R⁹, (C₁-C₄)alkyl, (C₁-C₄)alkoxy, —COOR⁵, —NH(C═O)R⁵, —NH(C═O)NR⁵R⁹, —NH(C═O)OR⁷, —O(C═O)NR⁵R⁹ and —NHSO₂R⁷; (b) heteroaryl, optionally substituted with from one to three substituents chosen independently from halogen, OH, —NR⁵R⁹, (C₁-C₄)alkyl, (C₁-C₄)alkoxy, —COOR⁵, NH(C═O)R⁵, —NH(C═O)NR⁵R⁹, —NH(C═O)OR⁷, —O(C═O)NR⁵R⁹ and —NHSO₂R⁷; and (c) non-aromatic heterocyclyl;
A is (C₂-C₇)-alkylene in which one or more —CH₂— may be replaced by a radical chosen from —CH(OH)—, —CH(NH₂)—, CHF, CF₂, —C(═O)—, —CH(O-loweralkyl)-, —CH(NH-loweralkyl)-, —O—, —S—, —SO—, —SO₂—, —NH— and —N[(C₁-C₄)alkyl]-; or two adjacent —CH₂— may be replaced by —CH═CH—;
D is chosen from a (C₄-C₁₂)carbocycle, a 4- to 7-membered monocyclic heterocycle and a 7- to 12-membered bicyclic heterocycle;
R² represents from one to three substituents each independently chosen from hydrogen, COOH, OH, SO₂NH-Het, SO₂(C₁-C₄)alkyl, acylsulfonamide, NO₂, halogen, (C₁-C₄)alkyl, (C₁-C₄)alkoxy, halo(C₁-C₄)alkyl, halo(C₁-C₄)alkoxy, cyano, phenyl, substituted phenyl, heterocyclyl, —CHO, —CH(R⁵)NR⁵R⁹ and —NR⁵R⁹, with the proviso that at least one instance of R² must be other than hydrogen;
Het is an optionally substituted heteroaryl;
R⁵ is chosen independently in each occurrence from hydrogen, (C₁-C₄)alkyl, aryl and heteroaryl;
R⁷ is chosen independently in each occurrence from (C₁-C₄)alkyl and aryl; and
R⁹ is chosen from hydrogen, (C₁-C₄)alkyl, aryl and heteroaryl, or, R⁵ and R⁹ taken together with the nitrogen to which they are attached, form a 5-8-membered nitrogen heterocycle; E is chosen from
(a) aryl, optionally substituted with from one to three substituents chosen independently from halogen, OH, —NR⁵R⁹, (C₁-C₄)alkyl, (C₁-C₄)alkoxy, halo(C₁-C₄)alkyl, halo(C₁-C₄)alkoxy;
(b) heteroaryl, optionally substituted with from one to three substituents chosen independently from halogen, OH, —NR⁵R⁹, (C₁-C₄)alkyl, (C₁-C₄)alkoxy, halo(C₁-C₄)alkyl, halo(C₁-C₄)alkoxy;
(c) non-aromatic heterocyclyl, optionally substituted with from one to three substituents chosen independently from halogen, OH, —NR⁵R⁹, (C₁-C₄)alkyl, (C₁-C₄)alkoxy, halo(C₁-C₄)alkyl, and halo(C₁-C₄)alkoxy;
R¹ is one or two substituents chosen from H, (C₁-C₄)alkyl and halo(C₁-C₄)alkyl;
R⁵ is chosen independently in each occurrence from hydrogen, (C₁-C₄)alkyl, aryl and heteroaryl;
R⁷ is chosen from (C₁-C₄)alkyl and aryl; and
R⁹ is chosen from hydrogen, (C₁-C₄)alkyl, aryl and heteroaryl, or, R⁵ and R⁹ taken together with the nitrogen to which they are attached, form a 5-8-membered nitrogen heterocycle;
R¹¹ and R¹² are chosen independently from H, CH₃, OH, CF₃, halogen and (C₁-C₄)alkoxy; and
R²¹ is one or two substituents chosen from hydrogen, (C₁-C₄)alkyl, halo(C₁-C₄)alkyl, cyano, NO₂, halogen, (C₁-C₄)acyl and (C₁-C₄)alkoxycarbonyl, as disclosed in WO 2011/082098;

PRMT inhibitors of Formulas IV, V and VI:

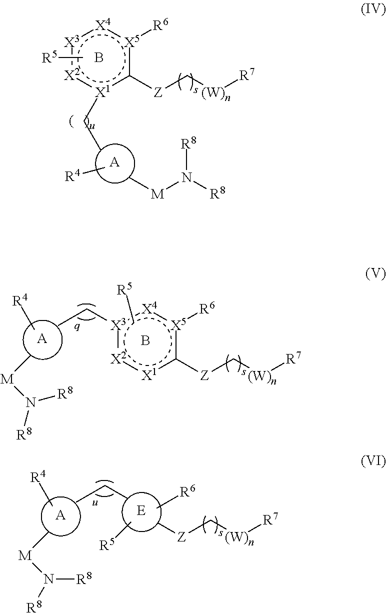

and N-oxides, hydrates, solvates, pharmaceutically acceptable salts, prodrugs and complexes thereof and racemic mixtures, diastereomers, enantiomers and tautomers thereof, wherein A is a cycloalkyl ring, a heterocyclic ring, a heteroaryl ring, or an aryl ring; B is selected from the group consisting of phenyl, and a 5- or 6-membered heteroaryl, wherein when B is a 5-membered heteroaryl, X⁴ is a bond, and X¹, X², X³ and X⁵ are each independently selected from the group consisting of C, N, O and S, provided that at least one of X¹, X², X³ and X⁵ is N, O or S, and provided that for Formula (IV), X¹ is not O or S, and for Formula (V), X³ is not O or S; and when B is a 6-membered heteroaryl, each of X¹, X², X³, X⁴ and X⁵ are independently C or N, provided that at least one of X¹, X², X³, X⁴ and X⁵ are N; E is a 5 to 10-membered heterocycle, preferably a 9-membered heterocycle; M is selected from the group consisting of

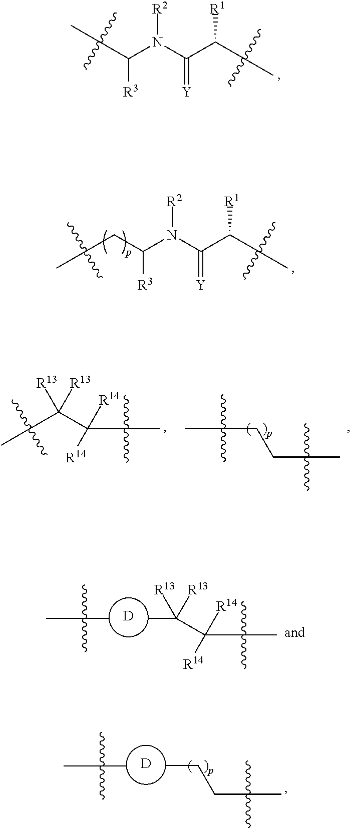

or M is selected from the group consisting of

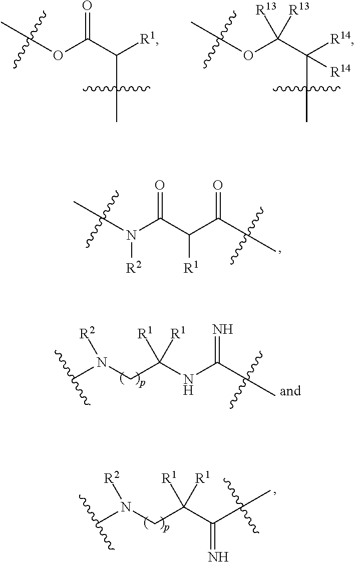

or M is selected from the group consisting of

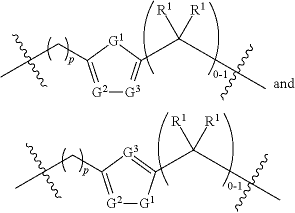

or M is selected from the group consisting of

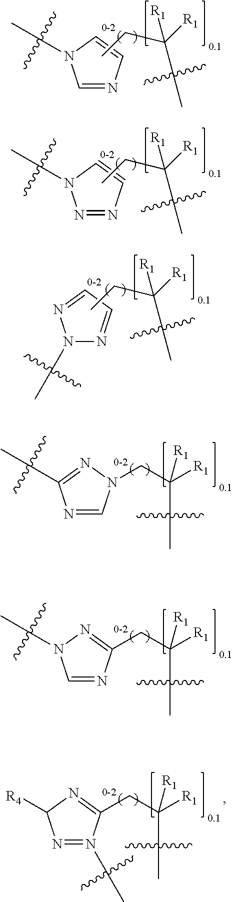

wherein p is 1, 2 or 3; each R¹³ is independently selected from the group consisting of H and C₁-C₄alkyl; each R¹⁴ is independently selected from the group consisting of H and C₁-C₄alkyl; or alternatively, R⁸ and R¹⁴ may join to form a 4, 5- or 6-membered saturated ring containing one N atom; and ring D is a heterocycle, preferably selected from the group consisting of

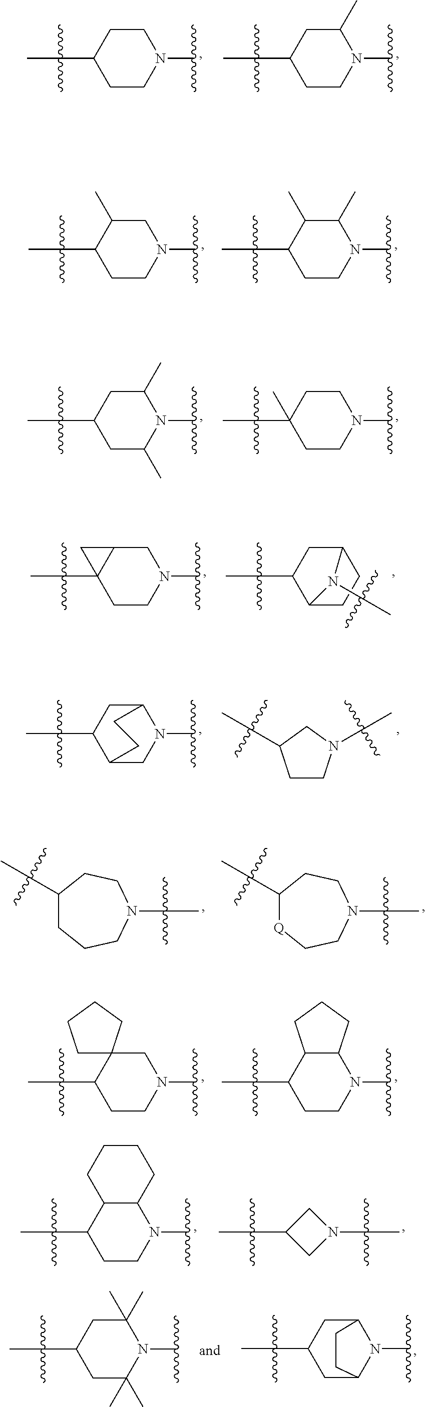

wherein the left side of ring D as shown is attached to ring A; and wherein Q is selected from the group consisting of —N(R¹⁵)—, O and S; and R¹⁵ is C₁-C₆alkyl; and each R¹ is independently selected from the group consisting of H, —OH, —CF₃, —CHF₂, —CH₂F, halo, —CN, alkyl, alkenyl, alkynyl, aryl, heteroaryl, alkoxy, cycloalkyl, heterocyclyl, —O-alkyl, —S(O)₀₋₁-alkyl, —O-cycloalkyl, —S(O)₀₋₁-cycloalkyl, —O-heterocyclyl, —S(O)₀₋₁-heterocyclyl, —O-aryl, —S(O)₀₋₁aryl, —O-heteroaryl, —S(O)₀₋₁-heteroaryl, -alkyl-cycloalkyl, -alkyl-heterocyclyl, -alkyl-aryl, -alkyl-heteroaryl and ═O (R¹ is preferably H, Me, Et, propyl, iso-propyl, —CF₃, CH₂Ph, OH or OPh; R² is selected from the group consisting of H, alkyl, aryl, heteroaryl, cycloalkyl, heterocyclyl, -alkyl-aryl, -alkyl-heteroaryl, -alkyl-cycloalkyl and -alkyl-heterocycle, each of which is optionally substituted (preferably R² is H, Me or Et); or R¹ and R² together form a 5-, 6- or 7-membered heterocycle, each of which is optionally substituted; or R² optionally bonds with Ring A to form a 5 or 6 membered heterocycle fused to ring A; R³ is selected from the group consisting of H, —OH, —CF₃, —CHF₂, —CH₂F, halo, —CN, alkyl, alkenyl, alkynyl, aryl, heteroaryl, alkoxy, cycloalkyl, heterocyclyl, —O-alkyl, —S(O)₀₋₁-alkyl, —O-cycloalkyl, —S(O)₀₋₁-cycloalkyl, —O-heterocyclyl, —S(O)₀₋₁-heterocyclyl, —O-aryl, —S(O)₀₋₁-aryl, —O-heteroaryl, —S(O)₀₋₁-heteroaryl, -alkyl-cycloalkyl, -alkyl-heterocyclyl, -alkyl-aryl, -alkyl-heteroaryl and ═O (preferably R³ is H or C₁-C₄ alky); or R² together with R³ optionally form a 4-, 5-, 6- or 7-membered heterocycle, each of which is optionally substituted; R⁴ is selected from the group consisting of H, —OH, halo, —CN, alkyl, alkenyl, alkynyl, aryl, heteroaryl, alkoxy, cycloalkyl, heterocyclyl, —O-alkyl, —S(O)₀₋₁-alkyl, —O-cycloalkyl, —S(O)₀₋₁-cycloalkyl, —O-heterocyclyl, —S(O)₀₋₁-heterocyclyl, —O-aryl, —S(O)₀₋₁aryl, —O-heteroaryl, —S(O)₀₋₁-heteroaryl, -alkyl-cycloalkyl, -alkyl-heterocyclyl, -alkyl-aryl, -alkyl-heteroaryl and ═O, each of which is optionally substituted, (preferably R⁴ is selected from the group consisting of H, halogen, CN, alkyl, substituted alkyl, —O—(C₁-C₄alkyl), —S—(C₁-C₁alkyl) and —S(O)₂—(C₁-C₄alkyl)); R⁵ is selected from the group consisting of H, —NO₂, halo, —CN, —CF₃, —CH₂F, —OH, —SH, C₂-C₆alkenyl, C₂-C₆alkynyl, alkoxy, cycloalkyl, heterocyclyl, aryl, heteroaryl, —O-alkyl, —S(O)₀₋₁-alkyl, —O-cycloalkyl, —S(O)₀₋₁-cycloalkyl, —O-heterocyclyl, —S(O)₀₋₁-heterocyclyl, ═O, —O-aryl, —S(O)₀₋₁-aryl, —O-heteroaryl, —S(O)₀₋₁-heteroaryl, —O—C(O)—N(R²)₂, —C(O)—NH2, —C(O)—N(R²)₂, (preferably R⁵ is selected from the group consisting of H, Me, Et, propyl, iso-propyl, OMe, OEt, SMe, SO₂Me, CF₃ and OCF₃); R⁶ is selected from the group consisting of H, —CN, alkyl, alkenyl, alkynyl, halo, —OH, —SH, ═O, —CF₃, alkoxy, aryl, heteroaryl, cycloalkyl, heterocyclyl, —O— alkyl, —S(O)₀₋₁-alkyl, —O-cycloalkyl, —S(O)₀₋₁-cycloalkyl, —O-heterocyclyl, —S(O)₀₋₁-heterocyclyl, —O-aryl, —S(O)₀₋₁-aryl, —O-heteroaryl and —S(O)₀₋₁-heteroaryl, (preferably R⁶ is selected from the group consisting of H, Me, Et, —NH₂, —CF₃ and —NO₂); R⁷ is selected from the group consisting of cycloalkyl, substituted cycloalkyl, heterocycle, substituted heterocycle, aryl, substituted aryl, heteroaryl and substituted heteroaryl, alkyl, optionally substituted alkyl; each R⁸ is independently selected from the group consisting of H and C₁-C₄alkyl; Y is nil (i.e., ═Y is —H), 0, S or —N(R⁸); G¹ is 0, S or NR^{B}; G² is N or CH; and G³ is N or CH; and Z is a moiety selected from the group consisting of a bond, —O—, —N(R⁹)—, —C(O)—, optionally substituted aryl, optionally substituted heteroaryl, optionally substituted -aryl-N(R²)—, optionally substituted -heteroaryl-N(R²)—, —C(═O)N(R¹⁰)—, —N(R¹⁰)C(═O)—, —N(R¹⁰)C(═O)— N(R¹⁰)—, —N(R¹⁰)C(═O)O—, —C(═S)N(R¹⁰)—, —N(R¹⁰)C(═S)—, —N(R¹⁰)C(═S)— N(R¹⁰)—, —N(R¹⁰)C(═S)O—, —N(R¹⁰)—S(O)₂—, —S(O)₂—N(R¹⁰)—, up.10)- and —N(R¹⁰)—C(O)—O—; wherein R¹⁰ is selected from the group consisting of H, alkyl, aryl, heteroaryl, cycloalkyl, heterocyclyl, -alkyl-aryl, -alkyl-heteroaryl, -alkyl-cycloalkyl and -alkyl-heterocycle, each of which is optionally substituted (preferably R¹⁰ is H, or Me); W is selected from the group consisting of a bond, an optionally substituted C₁-C₄alkyl, —S(O)₀₋₂—, —N(R¹⁰)—C(O)—O—, —O—C(S)—N(R¹⁰)—, —N(R¹⁰)—S(O)₂—, —S(O)₂—N(R¹⁰)—, —C(O)—, —C(S)—, —O—C(O)— and —C(O)—O—; or R⁶ together with W optionally form a 5- or 6-membered heterocycle; or W together with R⁷ optionally form a 5- or 6-membered heterocycle, wherein the heterocycle is optionally substituted; or R⁶ together with Z form an optionally substituted heteroaryl; u is 0 or 1; s is 0, 1, 2 or 3; and n is 0 or 1; or —Z—(CH₂)ₛ—(W)ₙ—R⁷ is an optionally substituted —C(O)-heterocycle or an optionally substituted 5- to 10-membered heteroaryl, preferably selected from the group consisting of

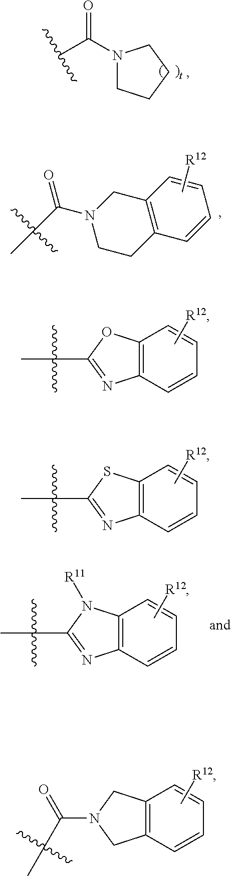

wherein t is 1, 3 or 4; and R′² is selected from the group consisting of hydrogen, halogen, haloalkyl, cyano, nitro, alkyl, cycloalkyl, alkenyl, cycloalkenyl, alkynyl, heterocycle, aryl, heteroaryl, —OR, —SR, —S(═O)R, —S(═O)₂R, —P(═O)₂R, —S(═O)₂OR, —P(═O)₂OR, —N(R)(R), —N(R)S(═O)₂R, —S(═O)₂N(R)(R), —N(R)P(═O)₂R, —P(═O)₂N(R)(R), —C(═O)OR, —C(═O)R, —C(═O)N(R)(R), —C(═S)N(R)(R), —OC(═O)R, —OC(═O)N(R)(R), —OC(═S)N(R)(R), —N(R)C(═O)OR, —N(R)C(═S)OR, —N(R)C(═O)N(R)(R), —N(R)C(═S)N(R)(R), —N(R)S(═O)₂N(R)(R), —N(R)P(═O)₂N(R)(R), —N(R)C(═O)R, —N(R)C(═S)R and —N(R)P(═O)₂R, wherein each R is independently selected from the group consisting of hydrogen, alkyl, cycloalkyl, alkenyl, cycloalkenyl, alkynyl, heterocycle, aryl and heteroaryl; provided that —Z—(CH₂)ₛ—(W)ₙ— is not —O—O— or —O—CH₂—O—; and provided that Formula (IV) excludes those compounds wherein (1) M is

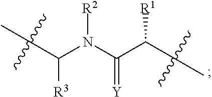

R⁸ are both H; Y is O; R³ is H or C₁-C₄alkyl; A is phenyl; u is O; Z is a moiety selected from the group consisting of

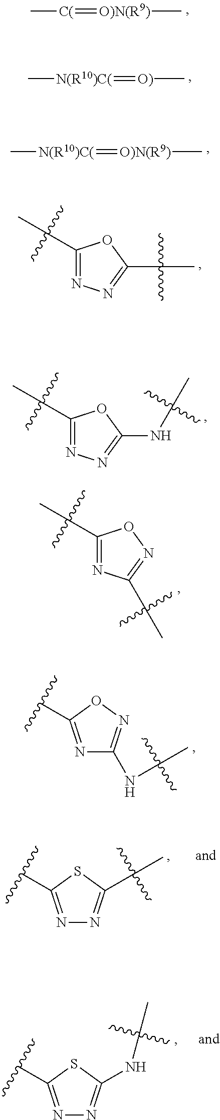

### W is O; or (2) M is

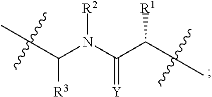

R⁸ are both H; Y is O; R³ is H or C₁-C₄alkyl; A is phenyl; u is 0; and —Z—(CH₂)ₘ—(W)ₙ—R⁷ is selected from the group consisting of

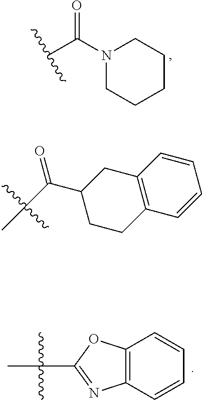 [SMILES: *C(=O)N1CCCCC1 ; *C(=O)C1CCc2ccccc2C1 ; *c1nc2ccccc2o1]

as disclosed in U.S. Pat. No. 8,338,437 and WO 2008/104077;

PRMT5 inhibitors SAM, MTA, AMI-1, -6, -9 and compounds 1-5 disclosed by Bonham et al, in a publication FEBS, 2010, 277, 2096-2108;

inhibitors of protein arginine methyl transferases of Formula VII and VIld:

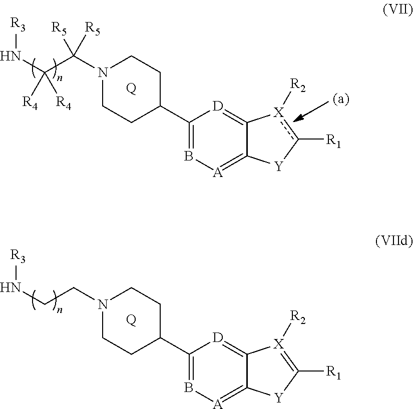

wherein:

Ring Q is

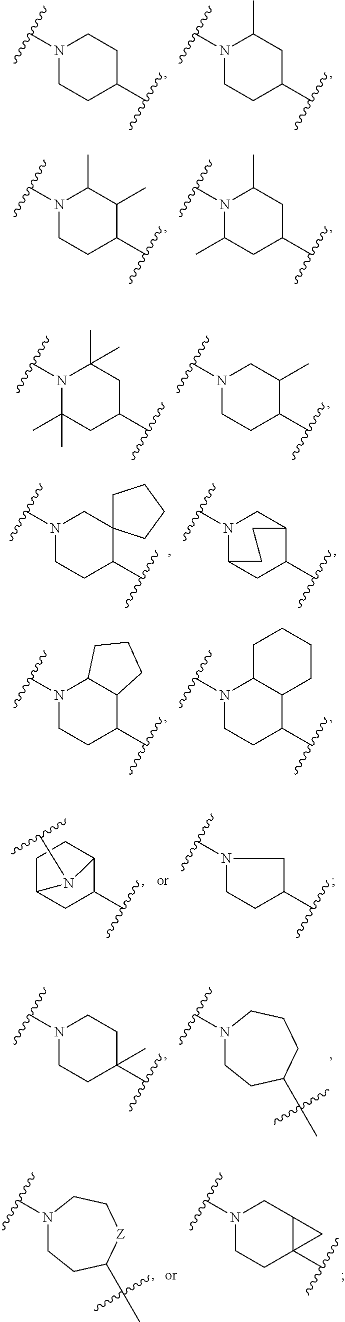

bond (a) is an optional double or single bond;
X is C (i.e., carbon) or N (i.e., nitrogen);

### Y is NH, N-Me, or CH;

Z is N—R₆, O, or S, where R₆ is C₁-C₆ alkyl;
wherein when bond (a) is a single bond, X is —CR—, R is independently H or C₁₋₄ alkyl and CR₂ is H or C₁₋₄ alkyl; alternatively, R₂ and R may join to form a

3-6 membered cycloalkyl ring;

A, B and D are each independently N or C, in which C may be optionally substituted with H, Me, Et, halogen, CN, NO₂, OMe, OEt, SMe, SO₂Me, CF₃, or OCF₃;

R₁ is aryl, substituted aryl, heterocycle, or substituted heterocycle;

R₂ is H, Me, Et, halogen, CN, NO₂, OMe, OEt, SMe, SO₂Me, CF₃, or OCF₃, provided that when X is N, R₂ is nil;

R₃ is H or C₁-C₄ alkyl; and

R₄ is independently H or C₁₋₄ alkyl;

R₅ is independently H, C₁₋₄ alkyl; alternatively, R5 and R3 may join to form a 4, 5, or 6 membered saturated ring containing one N; and

n is 1, 2, or 3, as disclosed in WO 2006/113458;

PRMT5 inhibitors of formula (I)

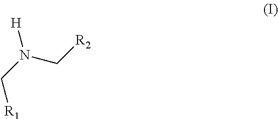

wherein

### R is

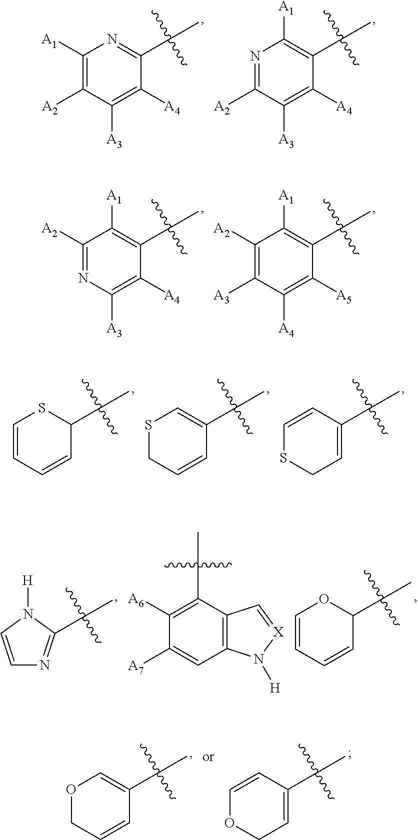

### R is

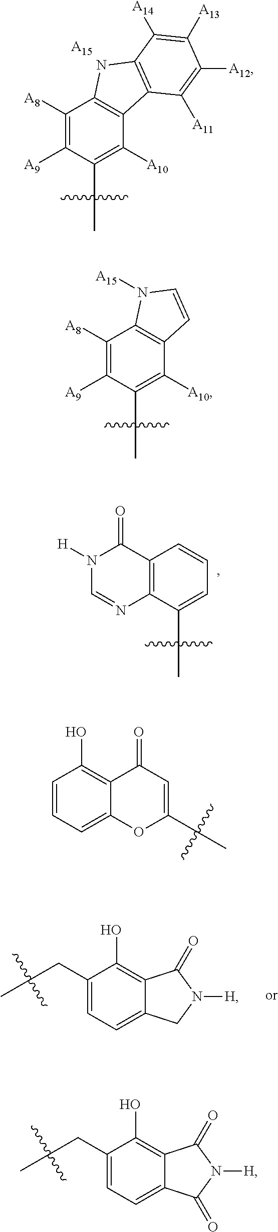

Ai, A₂, A₃, A₄, and A₅ are each individually hydrogen, halo, alkyl, alkoxyl, acetoxyl, alkylacetoxyl, —OH, trihalomethyl, —NH₂ or —NO₂;
A₆ and A₇ are each individually hydrogen, OH or NH₂;
A₈, A₉, Aio, An, A₁₂, A₁₃ and A₁₄ are each individually hydrogen, halo, alkyl, alkoxyl, acetoxyl, alkylacetoxyl, —OH, trihalomethyl, —NH₂ or —NO₂; and
Ai5 is alkyl (1-6 carbons in length); or
a salt thereof;
PRMT5 inhibitors of formula:

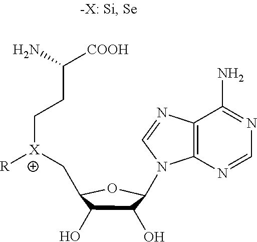

As disclosed in a publication by Bothwell, et al in a publication Org. Lett., 2014, 16, 3056-3059;
PRMT5 inhibitors disclosed by Mai et al in a publication J. Med. Chem., 2008, 51, 2279-2290;
PRMT5 inhibitors disclosed in U.S. Pat. Appl. Publ. No. 2010/0151506;
PRMT5 inhibitors disclosed by Bothwell, et al in a publication Org. Lett., 2014, S1-S46;

PRMT5 inhibitors of Formula VIII:

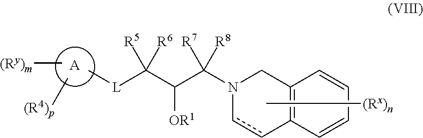

wherein:

[symbol] represents a single or double bond;

R¹ is hydrogen, R^{z}, or —C(O)R^{z}, wherein R^{z} is optionally substituted C₁₋₆ alkyl;
L is —O—, —N(R)—, —C(R²)(R³)—, —O—CR²R³, —N(R)—CR²R³—, —O—CR²R³—O—, —N(R)—CR²R³—O, —N(R)—CR²R³—N(R)—, —O—CR²R³—N(R)—, —CR²R³—O—, —CR²R³—N(R)—, —O—CR²R³—CR⁹R¹⁰—, —N(R)—CR²R³—CR⁹R¹⁰—, —CR²R³—CR⁹R¹⁰—O—, —CR²R³—CR⁹R¹⁰—N(R)—, or —CR²R³—CR⁹R¹⁰—;
each R is independently hydrogen or optionally substituted C₁₋₆ aliphatic;
R² and R³ are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, -OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR B SO₂R A, and —SO₂N(R B)₂; or R 2 and R 3 are taken together with their intervening atoms to form an optionally substituted carbocyclic or heterocyclic ring;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R groups are taken together with their intervening atoms to form an optionally substituted heterocyclic ring;
Ring A is a monocyclic or bicyclic, saturated, partially unsaturated, or aromatic ring having 0-4 heteroatoms independently selected from nitrogen, oxygen, and sulfur;

### R⁴ is —Li-Cy;

L₁ is a bond, —O—, —S—, —N(R)—, —C(O)—, —C(O)N(R)—, —N(R)C(O)N(R)—, —N(R)C(O)—, —N(R)C(O)O—, —OC(O)N(R)—, —SO₂—SO₂N(R)—, —N(R)SO₂—OC(O)—, —C(O)O—, or an optionally substituted, straight or branched, C1-6 aliphatic chain wherein one, two, or three methylene units of hi are optionally and independently replaced by —O—, —S—, —N(R)—, —C(O)—, —C(O)N(R)—, —N(R)C(O)N(R)—, —N(R)C(O)—, —N(R)C(O)O—OC(O)N(R)—, —SO₂—, —SO₂N(R)—, —N(R)SO₂—OC(O)—, or —C(O)O—;
Cy is an optionally substituted, monocyclic, bicyclic or tricyclic, saturated, partially unsaturated, or aromatic ring having 0-4 heteroatoms independently selected from nitrogen, oxygen, and sulfur;
R⁵, R⁶, R⁷, and R⁸ are independently hydrogen, halo, or optionally substituted aliphatic;
R⁹ and R¹⁰ are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, and —SO₂N(R^{B})₂; or R⁹ and R^{m} are taken together with their intervening atoms to form an optionally substituted carbocyclic or heterocyclic ring;
each R^{y} is independently selected from the group consisting of halo, —CN, —NO₂, optionally substituted aliphatic, optionally substituted carbocyclyl; optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, and —SO₂N(R^{B})₂;
each R^{x} is independently selected from the group consisting of halo, —CN, optionally substituted aliphatic, —OR′, and —N(R″)₂;
R′ is hydrogen or optionally substituted aliphatic; each R″ is independently hydrogen or optionally substituted aliphatic, or two R″ are taken together with their intervening atoms to form a heterocyclic ring;
n is 0, 1, 2, 3, 4, 5, 6, 7, 8, 9, or 10, as valency permits;
m is 0, 1, 2, 3, 4, 5, 6, 7, or 8, as valency permits; and
p is 0 or 1;
wherein, and unless otherwise specified,
heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur;
carbocyclyl or carbocyclic refers to a radical of a non-aromatic cyclic hydrocarbon group having from 3 to 10 ring carbon atoms and zero heteroatoms in the non-aromatic ring system;
aryl refers to a radical of a monocyclic or polycyclic aromatic ring system having 6-14 ring carbon atoms and zero heteroatoms provided in the aromatic ring system; and
heteroaryl refers to a radical of a 5-10 membered monocyclic or bicyclic aromatic ring system having ring carbon atoms and 1-4 ring heteroatoms provided in the aromatic ring system, wherein each heteroatom is independently selected from nitrogen, oxygen and sulfur;
provided that when L is —O— and Ring A is phenyl, p is 1; and
provided that the compound is not one of the following:

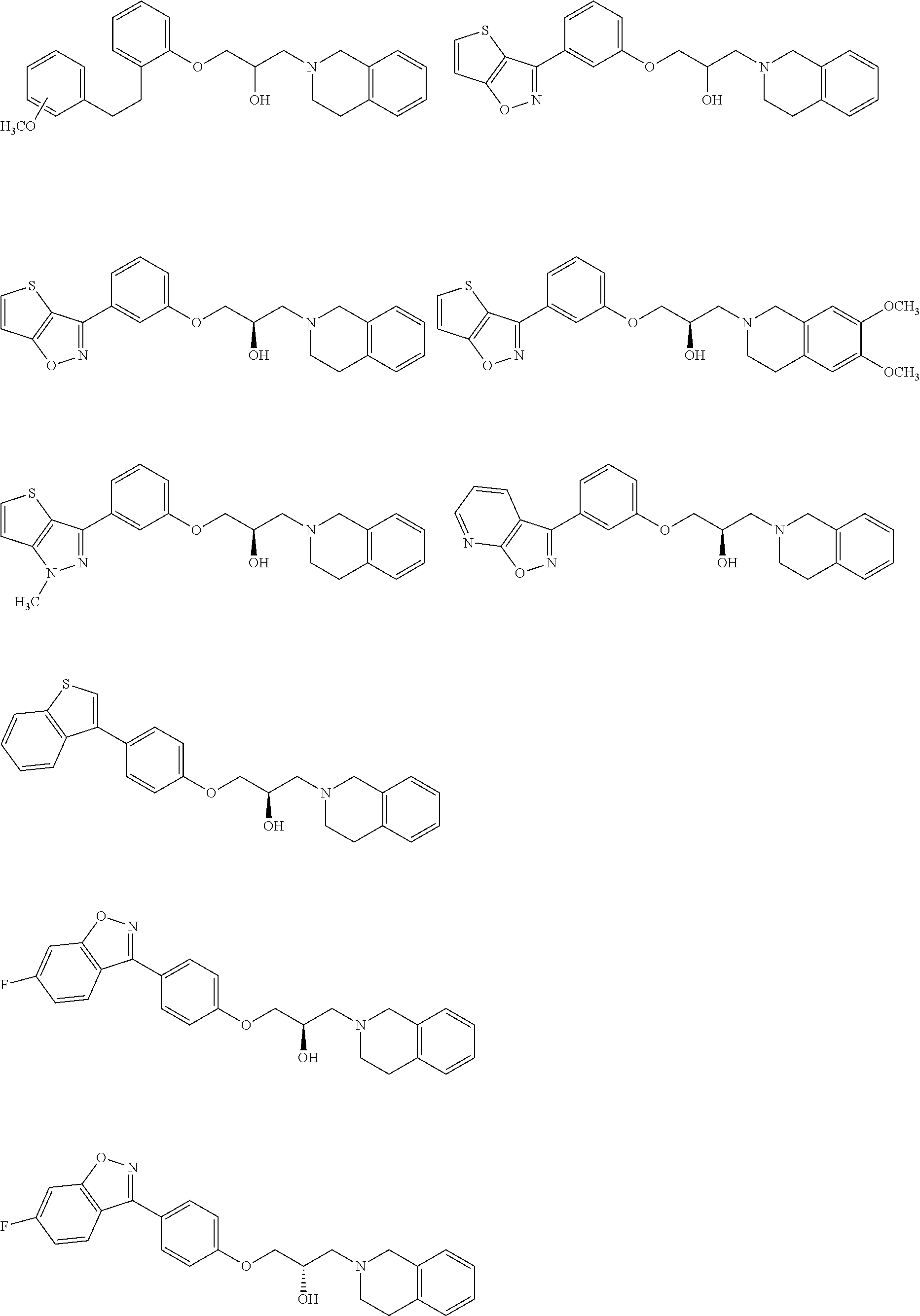

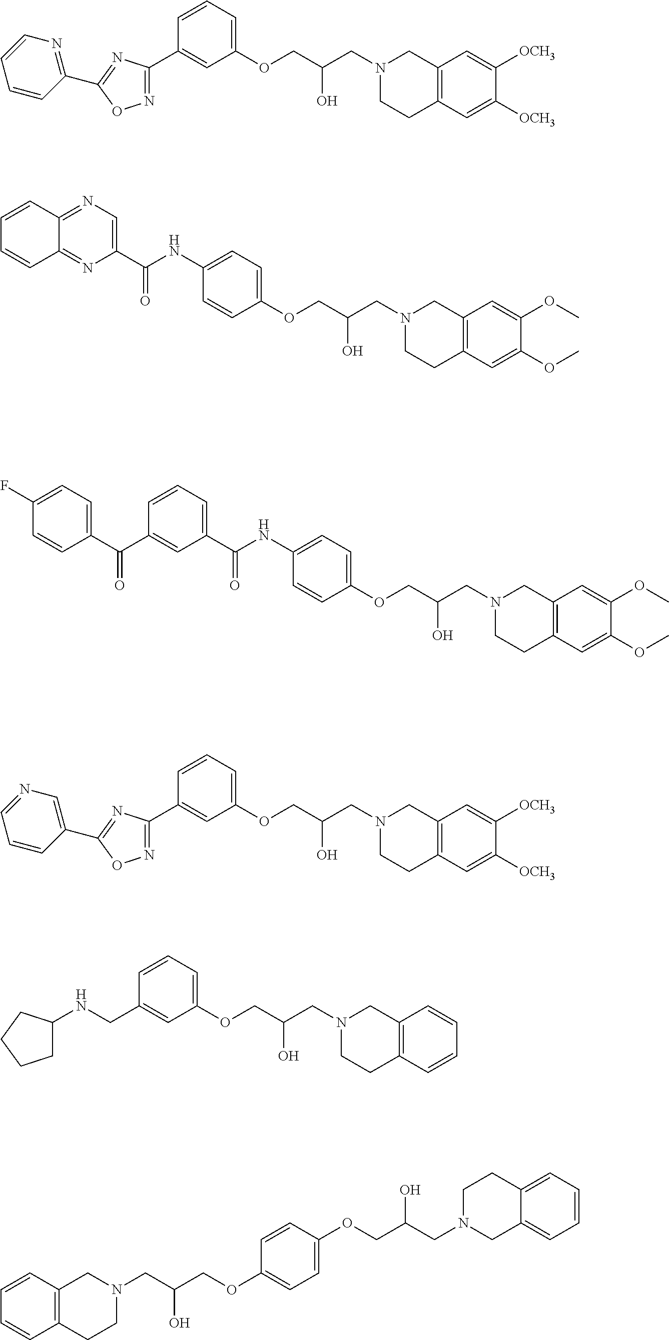 [SMILES: COc1cc2c(cc1OC)CN(CC(O)COc1cccc(-c3noc(-c4ccccn4)n3)c1)CC2 ; COc1cc2c(cc1OC)CN(CC(O)COc1ccc(NC(=O)c3cnc4ccccc4n3)cc1)CC2 ; COc1cc2c(cc1OC)CN(CC(O)COc1ccc(NC(=O)c3cccc(C(=O)c4ccc(F)cc4)c3)cc1)CC2 ; COc1cc2c(cc1OC)CN(CC(O)COc1cccc(-c3noc(-c4cccnc4)n3)c1)CC2 ; OC(COc1cccc(CNC2CCCC2)c1)CN1CCc2ccccc2C1 ; OC(COc1ccc(OCC(O)CN2CCc3ccccc3C2)cc1)CN1CCc2ccccc2C1]

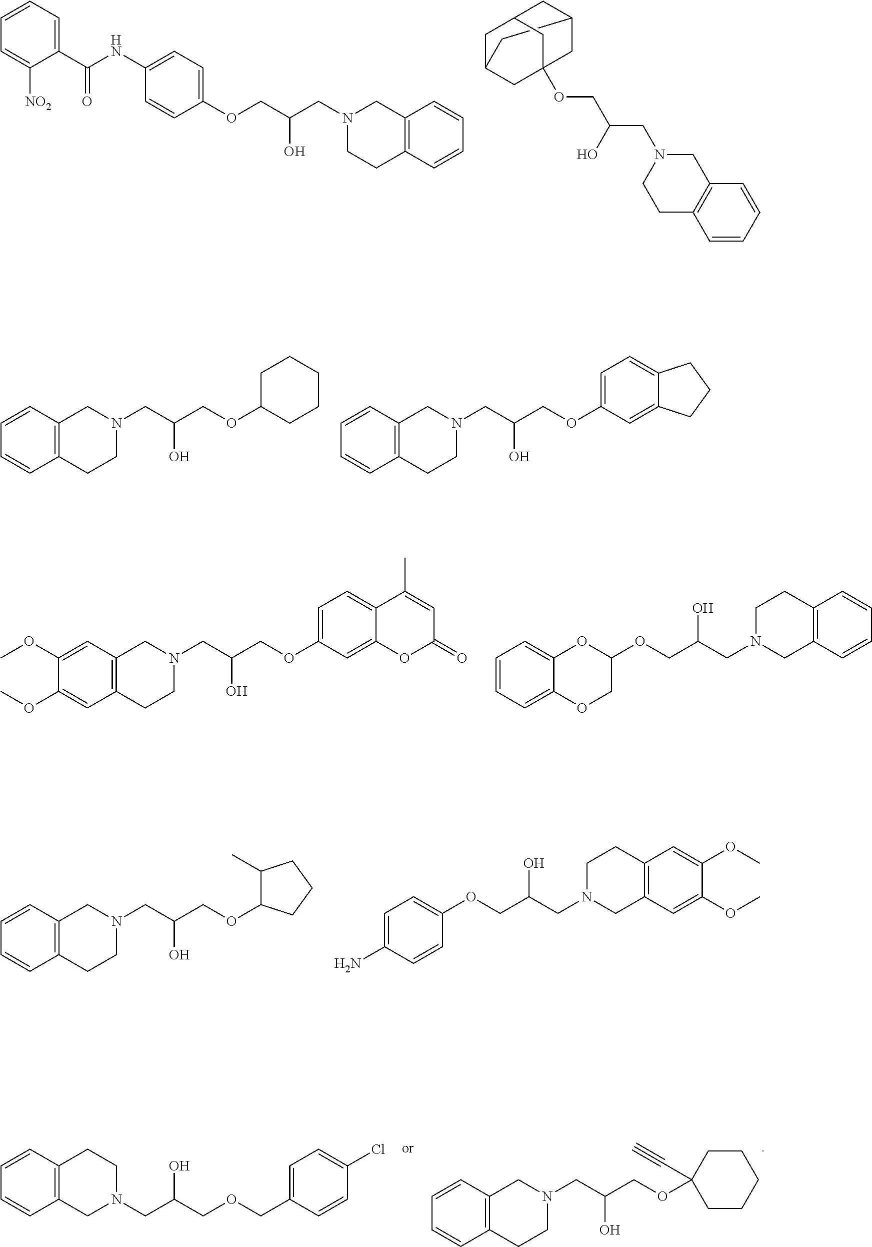

as disclosed in WO 2014/100695, WO 2014/100716, WO 2014/100719, WO 2014/100730, WO 2014/100734, and WO 2014/100764;

inhibitors of PRMT5 of Formula (A):

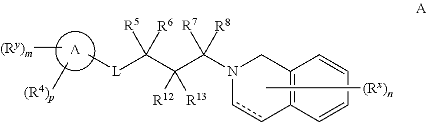

or a pharmaceutically acceptable salt thereof,
wherein
represents a single or double bond;
R 12 is hydrogen, halogen, or optionally substituted C₁₋₃ alkyl;
R¹³ is hydrogen, halogen, optionally substituted C₁₋₃alkyl, —NR^{A1}R^{A2}, or —OR¹;
R^{A1} and R^{A2} are each independently hydrogen, optionally substituted C₁₋₃ alkyl, a nitrogen protecting group, or R^{A1} and R^{A2} are taken together with the intervening nitrogen atom to form an optionally substituted 3-6 membered heterocyclic ring;
R¹ is hydrogen, R^{z}, or —C(0)R^{z}, wherein R^{z} is optionally substituted C₁₋₆ alkyl;
L is —O—, —N(R)—, —C(R²)(R³)—, —O—CR²R³, —N(R)—CR²R³—, —O—CR²R³—O—, —N(R)—CR²R³-0, —N(R)—CR²R³—N(R)—, —O—CR²R³—N(R)—, —CR²R³—O—, —CR²R³—N(R)—, —O—CR²R³—CR⁹R¹⁰—, —N(R)—CR²R³—CR⁹R¹⁰—, —CR²R³—CR⁹R¹⁰—O—, —CR²R³—CR⁹R¹⁰—N(R)—, or —CR²R³—CR⁹R¹⁰—;
each R is independently hydrogen or optionally substituted C₁₋₆ aliphatic;
R² and R³ are independently selected from the group consisting of hydrogen, halo, —CN, —NO₂, optionally substituted aliphatic, optionally substituted carbocyclyl; optionally substituted phenyl, optionally substituted heteroczclyl, optionally substituted heteroaryl, —OR^{A}, —N(R^{B})₂, —SR^{A}, —C(=0)R^{A}, —C(0)OR^{A}, —C(0)SR^{A}, —C(0)N(R^{B})₂, —C(0)N(R^{B})N(R^{B})₂, —OC(0)R^{A}, —OC(0)N(R^{B})₂, —NR^{B}C(0)R^{A}, —NR^{B}C(0)N(R^{B})₂, —NR^{B}C(0)N(R^{B})N(R^{B})₂, —NR^{B}C(0)OR^{A}, —SC(0)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(0)R^{A}, —OS(0)₂R^{A}, —S0₂R^{A}, —NR B S0₂R A, and —S0₂N(R B)₂; or R² and R³ are taken together with their intervening atoms to form an optionally substituted carbocyclic or heterocyclic ring; or R² and R³ are taken together with their intervening atoms to form an optionally substituted carbocyclic or heterocyclic ring;
each R is independently selected from the group consisting of hydrogen, optionally substituted aliphatic, optionally substituted carbocyclyl, optionally substituted heterocyclyl, optionally substituted aryl, and optionally substituted heteroaryl;
each R is independently selected from the group consisting of hydrogen, optionally substituted aliphatic, optionally substituted carbocyclyl, optionally substituted heterocyclyl, optionally substituted aryl, and optionally substituted heteroaryl, or two R groups are taken together with their intervening atoms to form an optionally substituted heterocyclic ring;
Ring A is a monocyclic or bicyclic, saturated, partially unsaturated, or aromatic ring having 0-4 heteroatoms independently selected from nitrogen, oxygen, and sulfur;

### R⁴ is -L Cy;

U is a bond, -0-, —S—, —N(R)—, —C(O)—, —C(0)N(R)—, —N(R)C(0)N(R)—, —N(R)C(0)-, —N(R)C(0)0-OC(0)N(R)—, —S0₂—S0₂N(R)—, —N(R)S0₂—OC(O)—, —C(0)0-, or an optionally substituted, straight or branched, Ci_6 aliphatic chain wherein one, two, or three methylene units of hi are optionally and independently replaced by -0-, —S—, —N(R)—, —C(O)—, —C(0)N(R)—, —N(R)C(0)N(R)—, —N(R)C(0)-, —N(R)C(0)0-OC(0)N(R)—, —S0₂—S0₂N(R)—, —N(R)SO₂—OC(O)—, or —C(0)0-;
Cy is an optionally substituted, monocyclic, bicyclic or tricyclic, saturated, partially unsaturated, or aromatic ring having 0-4 heteroatoms independently selected from nitrogen, oxygen, and sulfur;
R⁵, R⁶, R⁷, and R⁸ are each independently hydrogen, halo, or optionally substituted aliphatic;
R⁹ and R¹⁰ are each independently selected from the group consisting of hydrogen, halo, —CN, —N0₂, optionally substituted aliphatic, optionally substituted carbocyclyl;
optionally substituted phenyl, optionally substituted heterocyclyl, optionally substituted heteroaryl, —OR^{A}, —N(R^{B})₂, —SR^{A}, —C(=0)R^{A}, —C(0)OR^{A}, —C(0)SR^{A}, —C(0)N(R^{B})₂, —C(0)N(R^{B})N(R^{B})₂, —OC(0)R^{A}, —OC(0)N(R^{B})₂, —NR^{B}C(0)R^{A}, —NR^{B}C(0)N(R^{B})₂, —NR^{B}C(0)N(R^{B})N(R^{B})₂, —NR^{B}C(0)OR^{A}, —SC(0)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(0)R^{A}, —OS(0)₂R^{A}, —S0₂R^{A}, —NR^{B}S0₂R^{A}, and —S0₂N(R^{B})₂; or R⁹ and R¹⁰ are taken together with their intervening atoms to form an optionally substituted carbocyclic or heterocyclic ring;
each R^{y} is independently selected from the group consisting of halo, —CN, —N0₂, optionally substituted aliphatic, optionally substituted carbocyclyl; optionally substituted phenyl, optionally substituted heterocyclyl, optionally substituted heteroaryl, —OR, —N(R)₂, —SR, —C(=0)R^{A}, —C(0)OR^{A}, —C(0)SR^{A}, —C(0)N(R^{B})₂, —C(0)N(R^{B})N(R^{B})₂, —OC(0)R^{A}, —OC(0)N(R^{B})₂, —NR^{B}C(0)R^{A}, —NR^{B}C(0)N(R^{B})₂, —NR^{B}C(0)N(R^{B})N(R^{B})₂, —NR^{B}C(0)OR^{A}, —SC(0)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(0)R^{A}, —OS(0)₂R^{A}, —S0₂R^{A}, —NR^{B}S0₂R^{A}, and —S0₂N(R^{B})₂;
each R^{x} is independently selected from the group consisting of halo, —CN, optionally substituted aliphatic, —OR′, and —N(R″)₂;
R′ is hydrogen or optionally substituted aliphatic;
each R″ is independently hydrogen or optionally substituted aliphatic, or two R″ are taken together with their intervening atoms to form a heterocyclic ring;
n is 0, 1, 2, 3, 4, 5, 6, 7, 8, 9, or 10, as valency permits;
m is 0, 1, 2, 3, 4, 5, 6, 7, or 8, as valency permits; and
p is 0 or 1, as disclosed in WO 2014/14100695;

inhibitors of PRMT5 of Formula I:

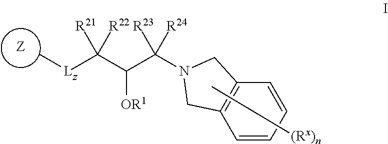

or a pharmaceutically acceptable salt thereof,
wherein
R¹ is hydrogen, R^{z} or —C(0)R^{z}, is optionally substituted C₁₋₆ alkyl;
L_{z} is a linker:
Ring Z is an optionally substituted, monocyclic or bicyclic, saturated, partially unsaturated, or aromatic ring having 0-4 heteroatoms independently selected from nitrogen, oxygen, and sulfur;
R 21, R 22, R 23, and R 2̂4 are independently hydrogen, halo, or optionally substituted aliphatic;
each R^{x} is independently selected from the group consisting of halo, —CN, optionally substituted aliphatic, and —OR;
R′ is hydrogen or optionally substituted aliphatic; and
n is 0, 1, 2, 3, 4, 5, 6, 7, or 8;
wherein, and unless otherwise specified,
heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur;
carhocyclyl car carbocyclic refers to a radical of a non-aromatic cyclic hydrocarbon group having from 3 to 10 ring carbon atoms and zero heteroatoms in the non-aromatic ring system;
aryl refers to a radical of a monocyclic or polycyclic aromatic ring system having 6-14 ring carbon atoms and zero heteroatoms provided in the aromatic ring system; and heteroaryl refers to a radical of a 5-10 membered monocyclic or bicyclic aromatic ring system having ring carbon atoms and 1-4 ring heteroatoms provided in the aromatic ring system, wherein each heteroatom is independently selected from nitrogen, oxygen and sulfur, as disclosed in WO 2014/100734,

inhibitors of PRMT5 of Formula I:

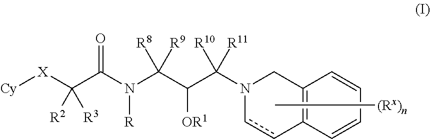

or a pharmaceutically acceptable salt thereof,
wherein
represents a single or double bond;
R¹ is hydrogen, R^{z}, or —C(0)R^{z}, wherein R^{z} is optionally substituted C₁₋₆ alkyl;
X is a bond, -0-, —N(R)—, —CR⁴R⁵—, -0-CR⁴R⁵, —N(R)—CR⁴R⁵—, -0-CR⁴R⁵-0-, —N(R)— CR⁴R⁵-0, —N(R)—CR⁴R⁵—N(R)—, -0-CR⁴R⁵—N(R)—, —CR⁴R⁵-0-, —CR⁴R⁵—N(R)—, -0-CR⁴R⁵—CR⁶R⁷—, —N(R)—CR⁴R⁵—CR⁶R⁷—, —CR⁶R⁷—CR⁴R⁵-0-, —CR⁶R⁷—CR⁴R⁵—N(R)—, or —CR⁶R⁷—C⁴R⁵— each R is independently hydrogen or optionally substituted C₁₋₆ aliphatic;
R² and R³ are independently selected from the group consisting of hydrogen, halo, —CN, —N0₂, optionally substituted aliphatic, optionally substituted carbocyclyl, optionally substituted phenyl, optionally substituted heterocyclyl, optionally substituted heteroaryl, —OR_{A}, —N(R^{B})₂, —SR^{A}, —C(=0)R^{A}, —C(0)OR^{A}, —C(0)SR^{A}, —C(0)N(R¹)₂, —C(0)N(R^{B})N(R^{B})₂, —OC(0)R^{A}, —OC(0)N(R^{B})₂, —NR^{B}C(0)R^{A}, —NR^{B}C(0)N(R^{B})₂, —NR^{B}C(0)N(R^{B})N(R_{B})₂, NR^{B}C(0)OR^{A}, —SC(0)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}—C(═S)N(R^{B})₂, —NR^{B}C(═S, R^{A}, —S(0)R^{A}—OS(0)₂R^{A}, —S0₂R^{A}, —NR^{B}S0₂R A, and —S0₂N(R^{B})₂; or R² and R³ are taken together with their intervening atoms to form an optionally substituted carbocyclic or heterocyclic ring;
R⁴ and R⁵ are independently selected from the group consisting of hydrogen, halo, —CN, —N0₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(=0)R^{A}, —C(0)OR^{A}, —C(0)SR^{A}, —C(0)N(R^{B})₂, —C(0)N(R^{B})N(R^{B})₂, —OC(O)R^{A}, —OC(0)N(R^{B})₂, —NR^{B}C(0)R^{A}, —NR^{B}C(0)N(R^{B})₂, —NR^{B}C(0)N(R^{B})N(R^{B})₂, NR^{B}C(0)OR^{A}, SC(0)R^{A}, —C(═NR^{B})R^{A}, —C(═NNR^{B})R^{A}, —C(═NOR^{A})R^{A}, —C(═NR^{B})(R³)₂, —NR^{B}C(═NR^{B})R^{B}, —C(═S)R^{A}, —C(═S)N(R^{B})₂, —NR^{B}C(═S)R^{A}—S(O)R^{A}—OS(0)₂R^{A}, —S0₂R^{A}, —NR^{B}S0₂R^{A}, and —S0₂N(R^{B})₂; or R⁴ and R⁵ are taken together with their intervening atoms to form an optionally substituted carbocyclic or heterocyclic ring; R⁶ and R⁷ are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(=0)R^{A}, —C(0)OR^{A}, —C(0)SR^{A}, —C(0)N(R^{B})₂, —C(0)N(R^{B})N(R^{B})₂, —OC(0)R^{A}, —OC(0)N(R^{B)}₂, —NR^{B}C(0)R^{A}, —NR^{B}C(0)N(R^{B})₂, —NR^{B}C(0)N(R^{B})N(R^{B})₂, —NR^{B}C(0)OR^{A}, —SC(0)R^{A}, —C(═NR^{B})R^{A}, —C(═NNR^{B}) R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(0)R^{A}, —OS(O)₂R^{A}, —S0₂R^{A}, —NR^{B}S0₂R^{A} and —S0₂N(R^{B})₂, or R⁶ and R⁷ are taken together with their intervening atoms to form an optionally substituted carbocyclic or heterocyclic ring;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R is independently selected from the group consisting of hydrogen, optionally substituted aliphatic, optionally substituted carbocyclyl, optionally substituted heterocyclyl, optionally substituted aryl, and optionally substituted heteroaryl, or two R groups are taken together with their intervening atoms to form an optionally substituted heterocyclic ring;
R⁸, R⁹, R¹⁰, and R¹¹ are independently hydrogen, halo, or optionally substituted aliphatic;
Cy is a monocyclic or bicyclic, saturated, partially unsaturated, or aromatic ring having 0-4 heteroatoms independently selected from nitrogen, oxygen, and sulfur, wherein Cy is substituted with 0, 1, 2, 3, or 4 R^{y} groups;
each R^{y} is independently selected from the group consisting of halo, —CN, —N0₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(±0)R^{A}, —C(0)OR^{A}, —C(0)SR^{A}, —C(0)N(R^{B})₂, —C(0)N(R^{B})N(R^{B})₂, —OC(0)R^{A}, —OC(0)N(R^{B})₂, —NR^{B}C(0)R^{A}, —NR^{B}C(0)N(R^{B})₂, —NR^{B}C(0)N(R^{B})N(R^{B})₂, —NR^{B}C(0)OR^{A}, SC(0)R^{A}, —C(═NR^{B})R^{A}, —C(═NNR^{B})R^{A}, —C(═NOR^{A})R^{A}, —C(═NR^{B})N(R^{B3})₂, —NR^{B}C(═NR^{B})R^{B}, —C(═S)R^{A}, —C(═S)N(R^{B})₂, —NR^{B}C(═S)R^{A}, —S(0)R^{A}, —OS(0)₂R^{A}, —S0₂R^{A}, —NR^{B}S0₂R^{A}, and —S0₂N(R^{B})₂; or an R^{y} group may be optionally taken together with R² or R³ to form an optionally substituted 5- to 6-membered carbocyclic or heterocyclic ring fused to Cy;
each R^{x} is independently selected from the group consisting of halo, —CN, optionally substituted aliphatic; —OR′, and —N(R″)₂;
R′ is hydrogen or optionally substituted aliphatic; each R″ is independently hydrogen or optionally substituted aliphatic, or two R″ are taken together with their intervening atoms to form an optionally substituted heterocyclic ring having 1-2 heteroatoms independently selected from nitrogen, oxygen, and sulfur; and
n is 0, 1, 2, 3, 4, 5, 6, 7, 8, 9, or 10, as valency permits;
wherein, and unless otherwise specified,
heterocyclyl or heterocyclic refers to a radical of a 3-10 membered non-aromatic ring system having ring carbon atoms and 1-4 ring heteroatoms, wherein each heteroatom is independently selected from nitrogen, oxygen; and sulfur;
carbocyclyl or carbocyclic refers to a radical of a non-aromatic cyclic hydrocarbon group having from 3 to 10 ring carbon atoms and zero heteroatoms in the non-aromatic ring system;
aryl refers to a radical of a monocyclic or polycyclic aromatic ring system having 6-14 ring carbon atoms and zero heteroatoms provided in the aromatic ring system; and
heteroaryl refers to a radical of a 5-10 membered monocyclic or bicyclic aromatic ring system having ring carbon atoms and 1-4 ring heteroatoms provided in the aromatic ring system, wherein each heteroatom is independently selected from nitrogen, oxygen and sulfur, as disclosed in WO 2014/100730;

inhibitors of PRMT5 of Formula (I):

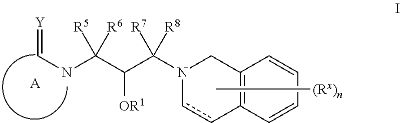

or a pharmaceutically acceptable salt thereof,
wherein
represents a single or double bond;
Ring A is an optionally substituted, 5- to 12-membered, monocyclic or bicyclic, heterocyclyl heteroaryl having 1-4 heteroatoms independently selected from nitrogen, oxygen, and sulfur;
R¹ is hydrogen, R^{z}, or —C(0)R^{z}, wherein R^{z} is optionally substituted C₁₋₆ alkyl;

### Y is O or S;

R⁵, R⁶, R⁷, and R⁸ are independently hydrogen, halo, or optionally substituted aliphatic;
each R^{x} is independently selected from the group consisting of halo, —CN, optionally substituted aliphatic, —OR′, and —N(R″)₂;
R′ is hydrogen or optionally substituted aliphatic;
each R″ is independently hydrogen or optionally substituted aliphatic, or two R″ are taken together with their intervening atoms to form a heterocyclic ring; and
n is 0, 1, 2, 3, 4, 5, 6, 7, 8, 9, or 10, as valency permits;
wherein, and unless otherwise specified,
heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur;
carbocyclyl or carbocyclic refers to a radical of a non-aromatic cyclic hydrocarbon group having from 3 to 10 ring carbon atoms and zero heteroatoms in the non-aromatic ring system;
aryl refers to a radical of a monocyclic or polycyclic aromatic ring system having 6-14 ring carbon atoms and zero heteroatoms provided in the aromatic ring system; and heteroaryl refers to a radical of a 5-10 membered monocyclic or bicyclic aromatic ring system having ring carbon atoms and 1-4 ring heteroatoms provided in the aromatic ring system, wherein each heteroatom is independently selected from nitrogen, oxygen and sulfur, as disclosed in WO 2014/100716;

inhibitors of PRMT5 inhibitors of Formula (I):

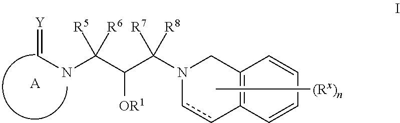

or a pharmaceutically acceptable salt thereof,
wherein
represents a single or double bond;
Ring A is an optionally substituted, 5- to 12-membered, monocyclic or bicyclic, heterocyclyl or heteroaryl having 1-4 heteroatoms independently selected from nitrogen, oxygen, and sulfur;
R¹ is hydrogen, —C(O)R^{z}, wherein R^{z} is optionally substituted C₁₋₆ alkyl;

### Y is O or S;

R⁵, R⁶, R⁷, and R⁸ are independently hydrogen, halo, or optionally substituted aliphatic; each R^{x} is independently selected from the group consisting of halo, —CN, optionally substituted aliphatic, —OR′, and —N(R″)₂;
R′ is hydrogen or optionally substituted aliphatic;
each R″ is independently hydrogen or optionally substituted aliphatic, or two R″ are taken together with their intervening atoms to form a heterocyclic ring; and
n is 0, 1, 2, 3, 4, 5, 6, 7, 8, 9, or 10, as valency permits;
wherein, and unless otherwise specified;
heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur;
carbocyclyl or carbocyclic refers to a radical of a non-aromatic cyclic hydrocarbon group having from 3 to 10 ring carbon atoms and zero heteroatoms in the non-aromatic ring system; and
aryl refers to a radical of a monocyclic or polycyclic aromatic ring system having 6-14 ring carbon atoms and zero heteroatoms provided in the aromatic ring system; and heteroaryl refers to a radical of a 0.5-10 membered monocyclic or bicyclic aromatic ring system having ring carbon atoms and 1-4 ring heteroatoms provided in the aromatic ring system, wherein each heteroatom is independently selected from nitrogen, oxygen and sulfur, as disclosed in WO 2014/100764.

In some embodiments, the PRMT5 inhibitor is sinefungin, HLCL7, CMP12, BLL-1, BLL-3, any of BLL2-BLL8, BLL36, CMP5 (BLL1), CMP5 derivatives, BLL54, any of the compounds designated herein as Formulas I-VIII (including VIId); any of these can use used in any of the methods disclosed herein, wherein in the case of a discrepancy between the document incorporated by reference and this disclosure in regards to chemical structures, the document incorporated by reference controls in regards to chemical structures.

In other embodiments, the PRMT5 inhibitor is selected from:

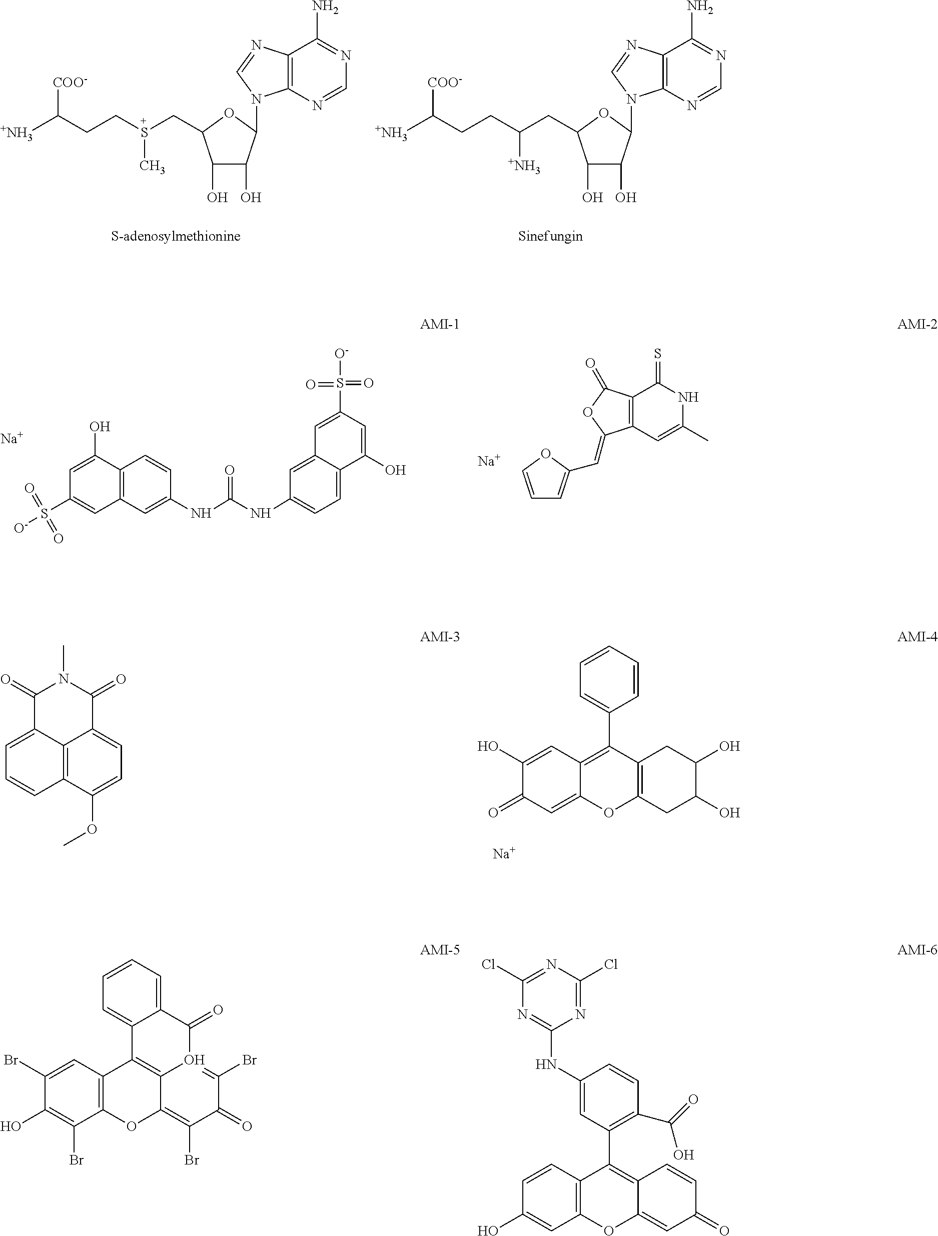

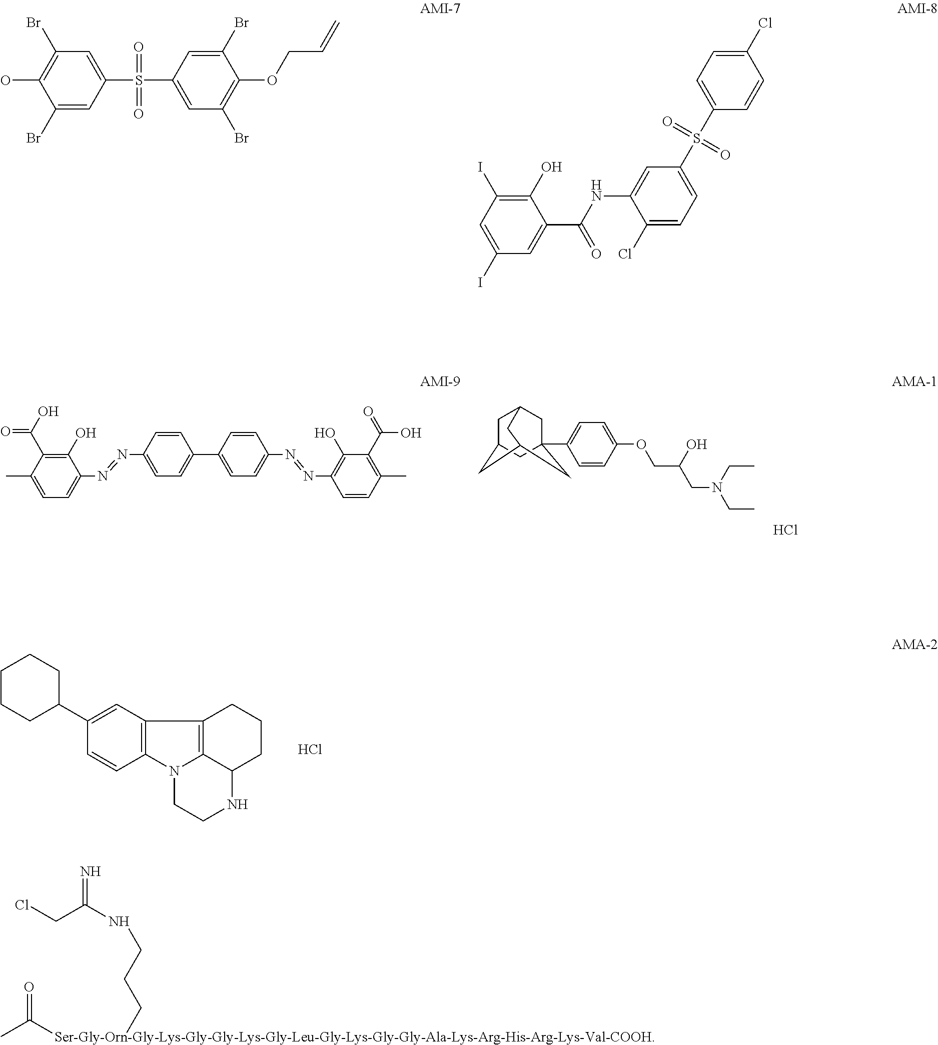

Eosin (AMI-5), curcumin, resveratrol, GW5074,

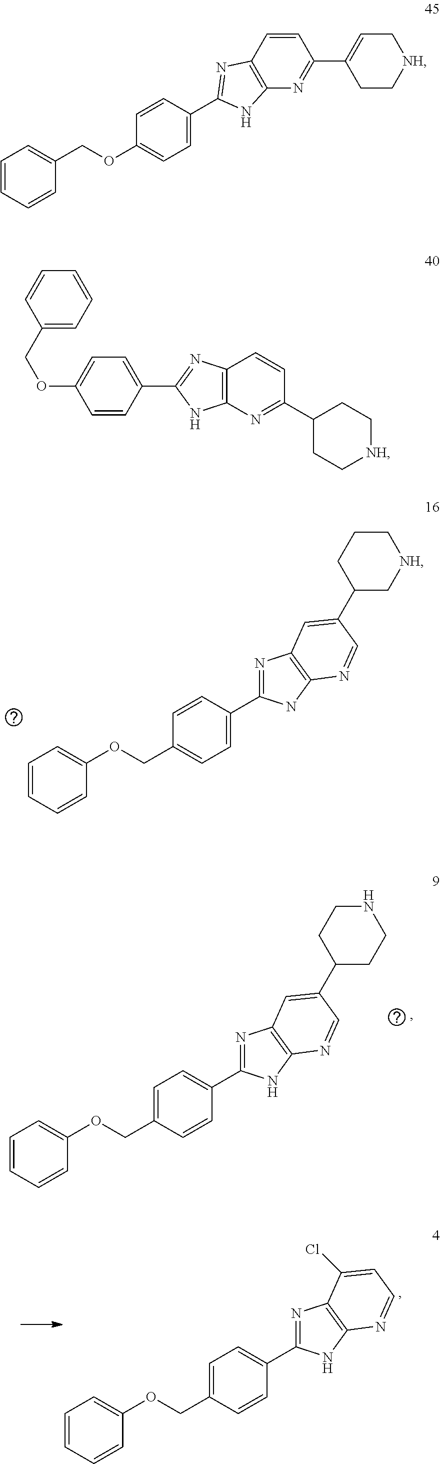 [SMILES: C1=C(c2ccc3nc(-c4ccc(OCc5ccccc5)cc4)[nH]c3n2)CCNC1 ; c1ccc(COc2ccc(-c3nc4ccc(C5CCNCC5)nc4[nH]3)cc2)cc1 ; c1ccc(OCc2ccc(-c3nc4cc(C5CCCNC5)cnc4[nH]3)cc2)cc1 ; c1ccc(OCc2ccc(-c3nc4cc(C5CCNCC5)cnc4[nH]3)cc2)cc1 ; Clc1ccnc2[nH]c(-c3ccc(COc4ccccc4)cc3)nc12]

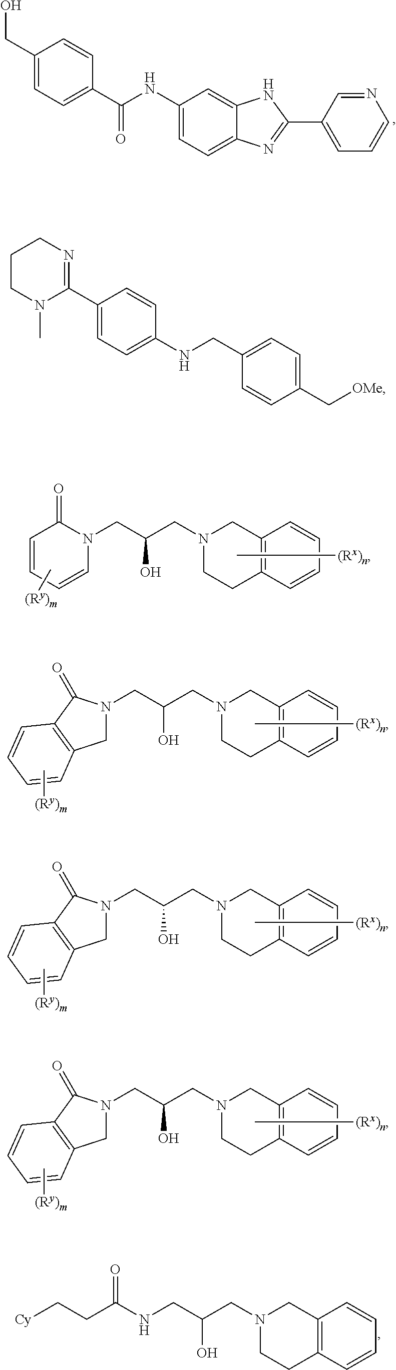

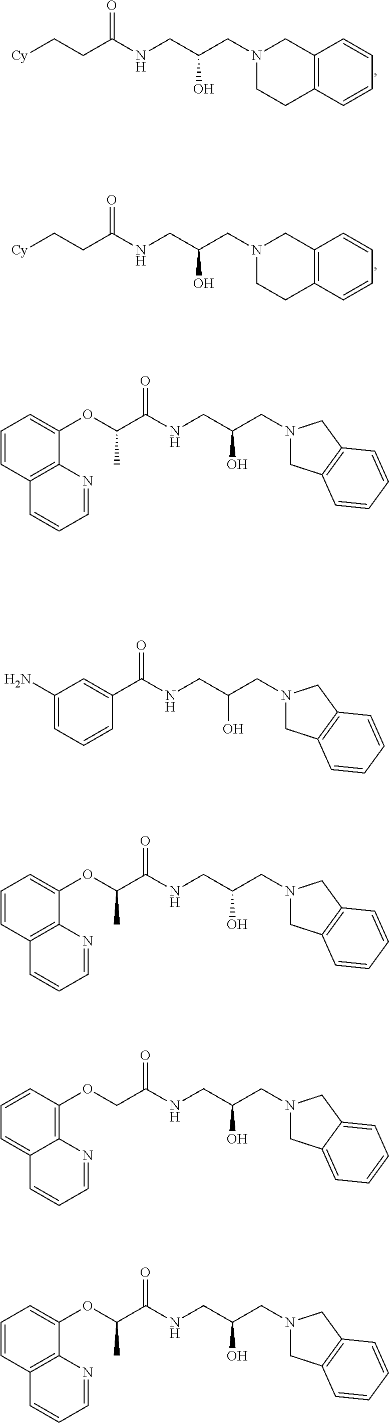

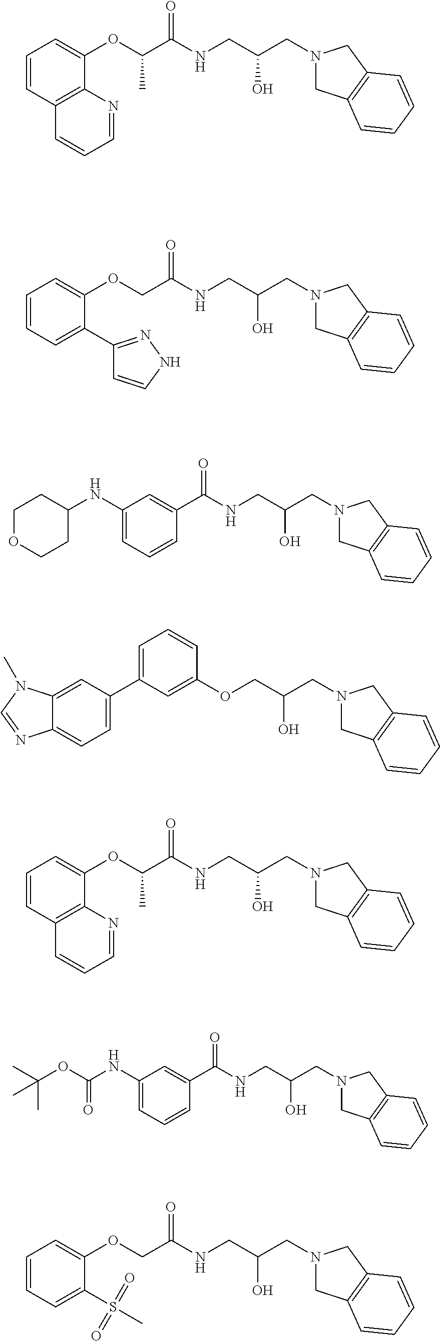

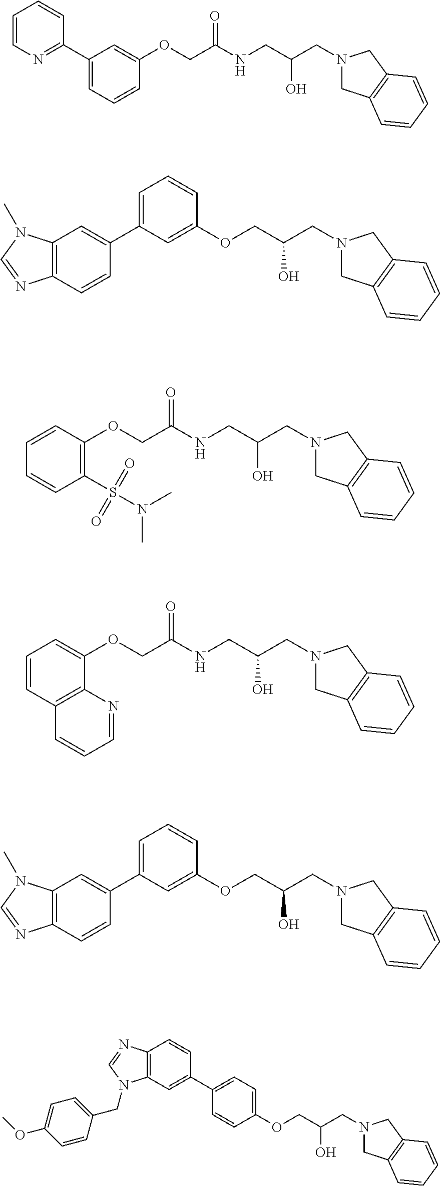 [SMILES: O=C(COc1cccc(-c2ccccn2)c1)NCC(O)CN1Cc2ccccc2C1 ; Cn1cnc2ccc(-c3cccc(OC[C@@H](O)CN4Cc5ccccc5C4)c3)cc21 ; CN(C)S(=O)(=O)c1ccccc1OCC(=O)NCC(O)CN1Cc2ccccc2C1 ; O=C(COc1cccc2cccnc12)NC[C@@H](O)CN1Cc2ccccc2C1 ; Cn1cnc2ccc(-c3cccc(OC[C@H](O)CN4Cc5ccccc5C4)c3)cc21 ; COc1ccc(Cn2cnc3ccc(-c4ccc(OCC(O)CN5Cc6ccccc6C5)cc4)cc32)cc1]

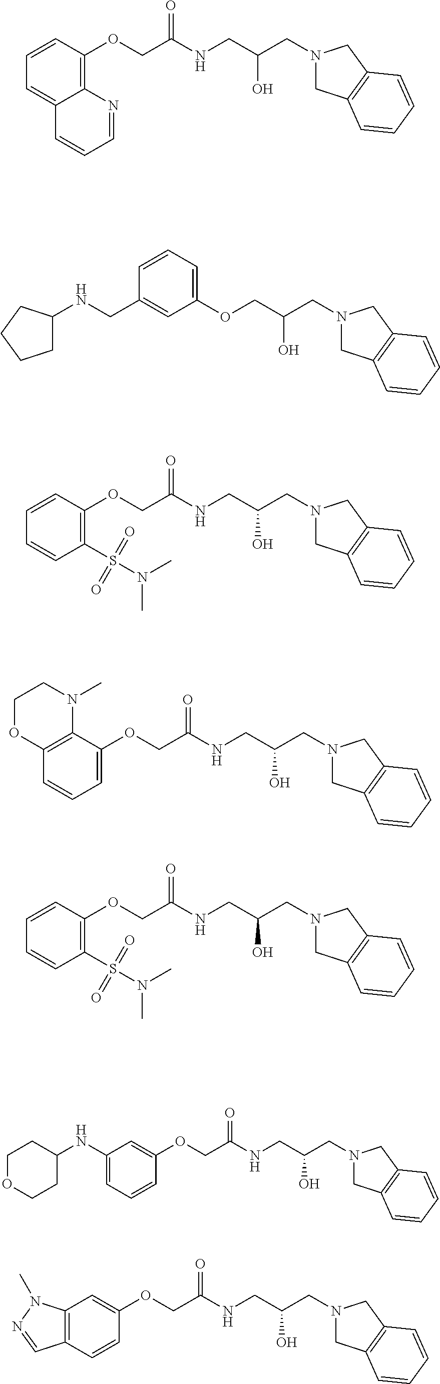

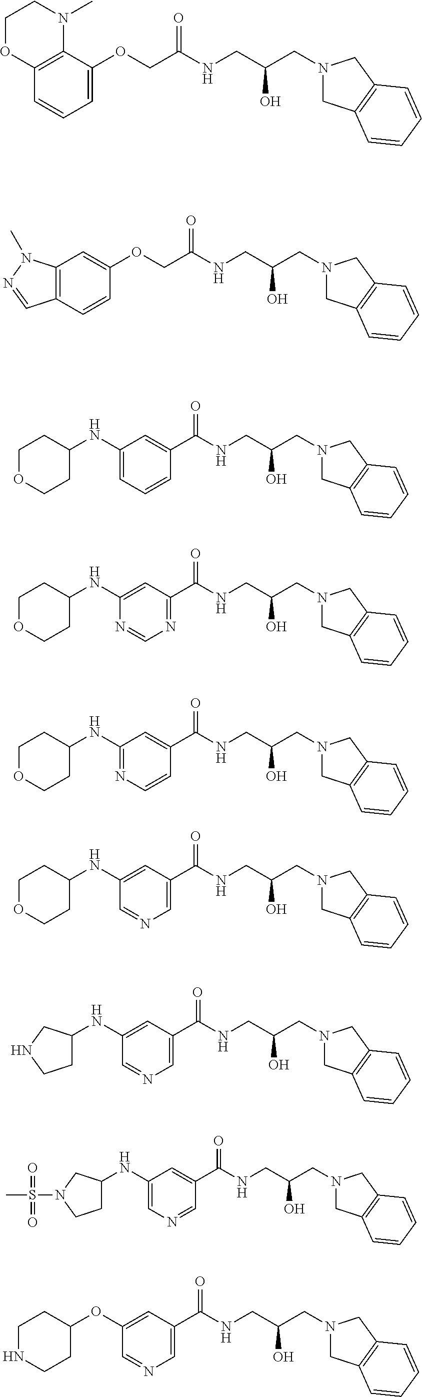

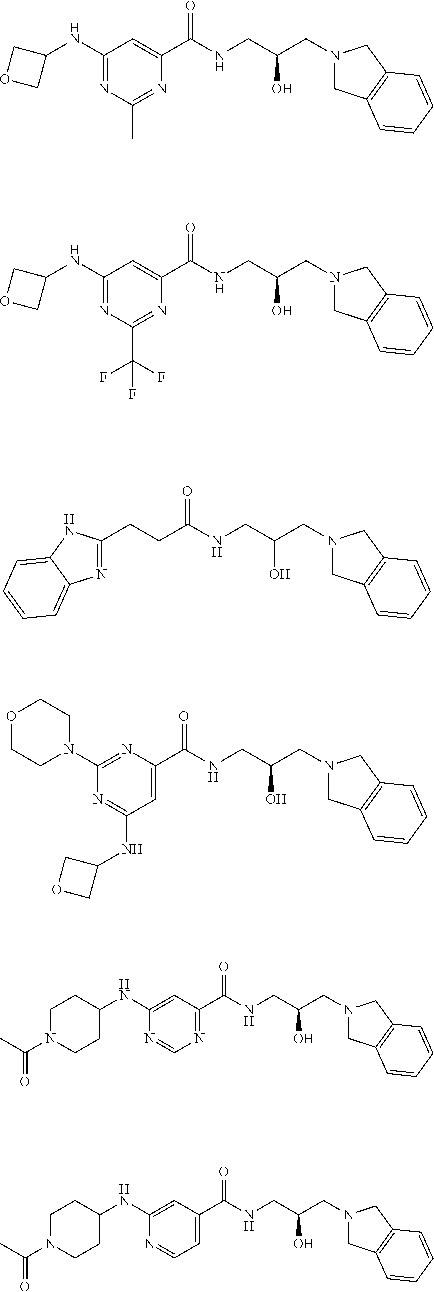 [SMILES: Cc1nc(NC2COC2)cc(C(=O)NC[C@H](O)CN2Cc3ccccc3C2)n1 ; O=C(NC[C@H](O)CN1Cc2ccccc2C1)c1cc(NC2COC2)nc(C(F)(F)F)n1 ; O=C(CCc1nc2ccccc2[nH]1)NCC(O)CN1Cc2ccccc2C1 ; O=C(NC[C@H](O)CN1Cc2ccccc2C1)c1cc(NC2COC2)nc(N2CCOCC2)n1 ; CC(=O)N1CCC(Nc2cc(C(=O)NC[C@H](O)CN3Cc4ccccc4C3)ncn2)CC1 ; CC(=O)N1CCC(Nc2cc(C(=O)NC[C@H](O)CN3Cc4ccccc4C3)ccn2)CC1]

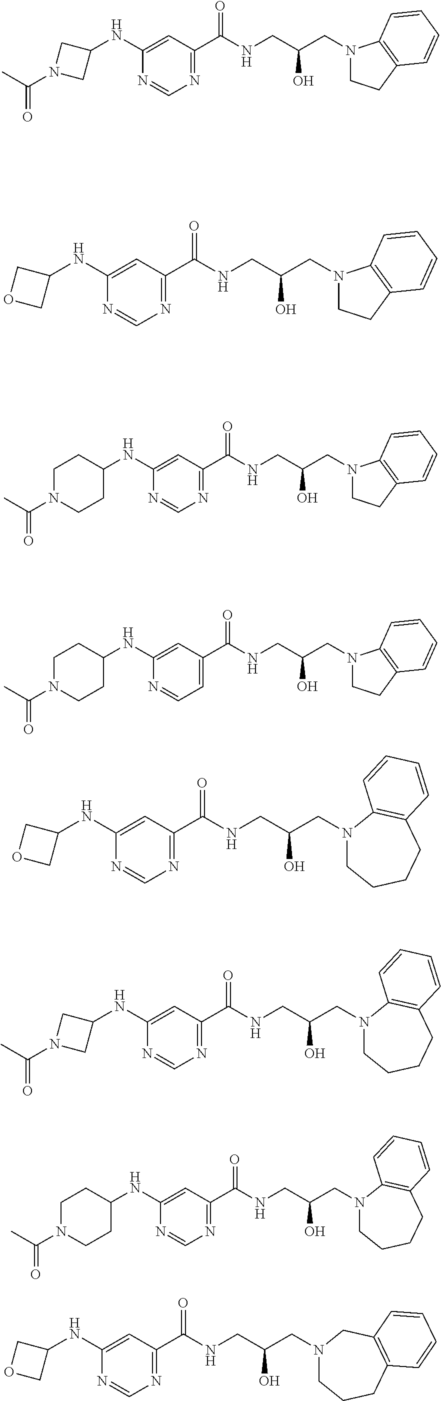

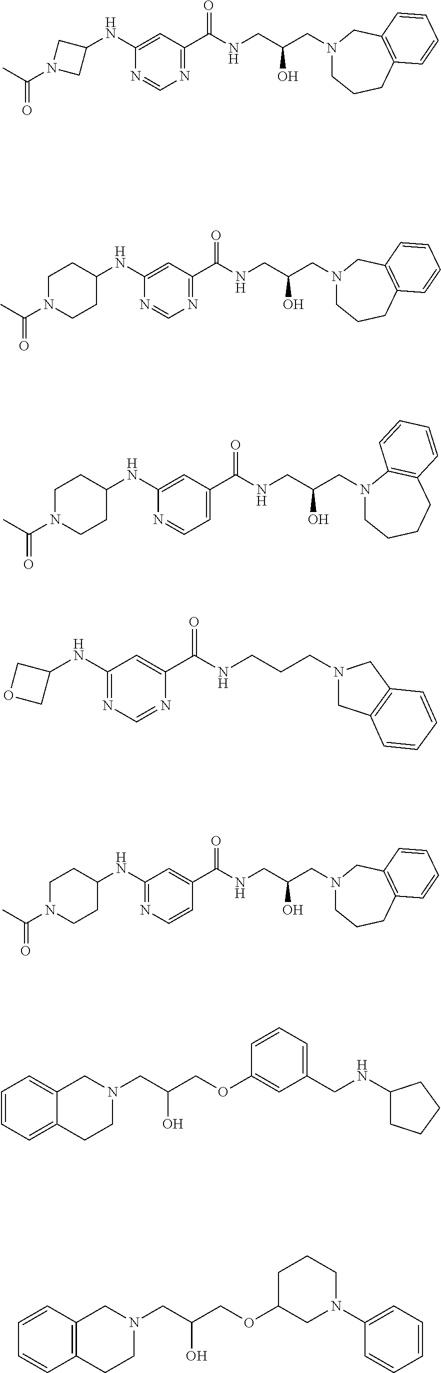

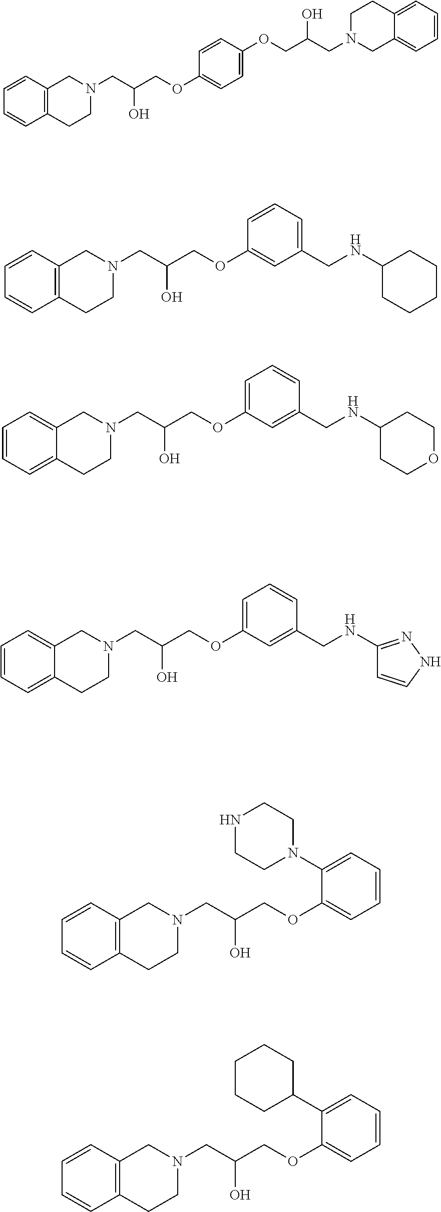 [SMILES: OC(COc1ccc(OCC(O)CN2CCc3ccccc3C2)cc1)CN1CCc2ccccc2C1 ; OC(COc1cccc(CNC2CCCCC2)c1)CN1CCc2ccccc2C1 ; OC(COc1cccc(CNC2CCOCC2)c1)CN1CCc2ccccc2C1 ; OC(COc1cccc(CNc2cc[nH]n2)c1)CN1CCc2ccccc2C1 ; OC(COc1ccccc1N1CCNCC1)CN1CCc2ccccc2C1 ; OC(COc1ccccc1C1CCCCC1)CN1CCc2ccccc2C1]

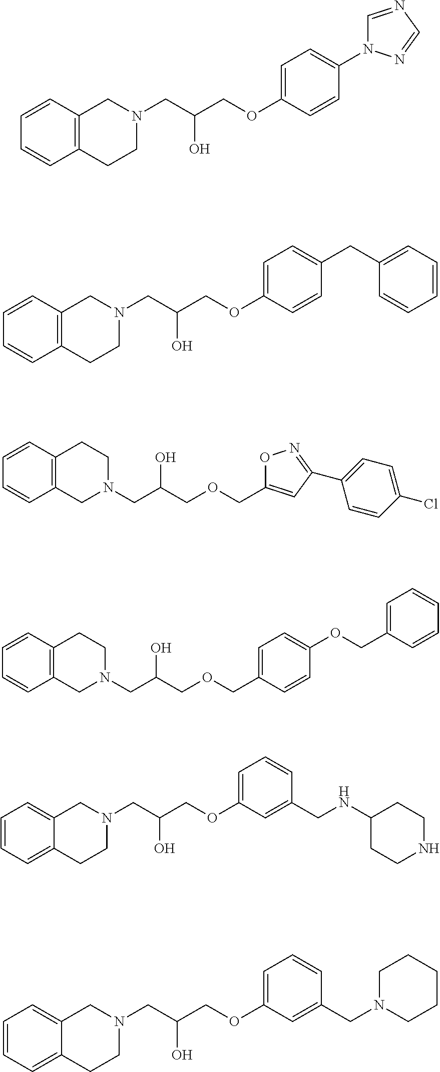 [SMILES: OC(COc1ccc(-n2cncn2)cc1)CN1CCc2ccccc2C1 ; OC(COc1ccc(Cc2ccccc2)cc1)CN1CCc2ccccc2C1 ; OC(COCc1cc(-c2ccc(Cl)cc2)no1)CN1CCc2ccccc2C1 ; OC(COCc1ccc(OCc2ccccc2)cc1)CN1CCc2ccccc2C1 ; OC(COc1cccc(CNC2CCNCC2)c1)CN1CCc2ccccc2C1 ; OC(COc1cccc(CN2CCCCC2)c1)CN1CCc2ccccc2C1]

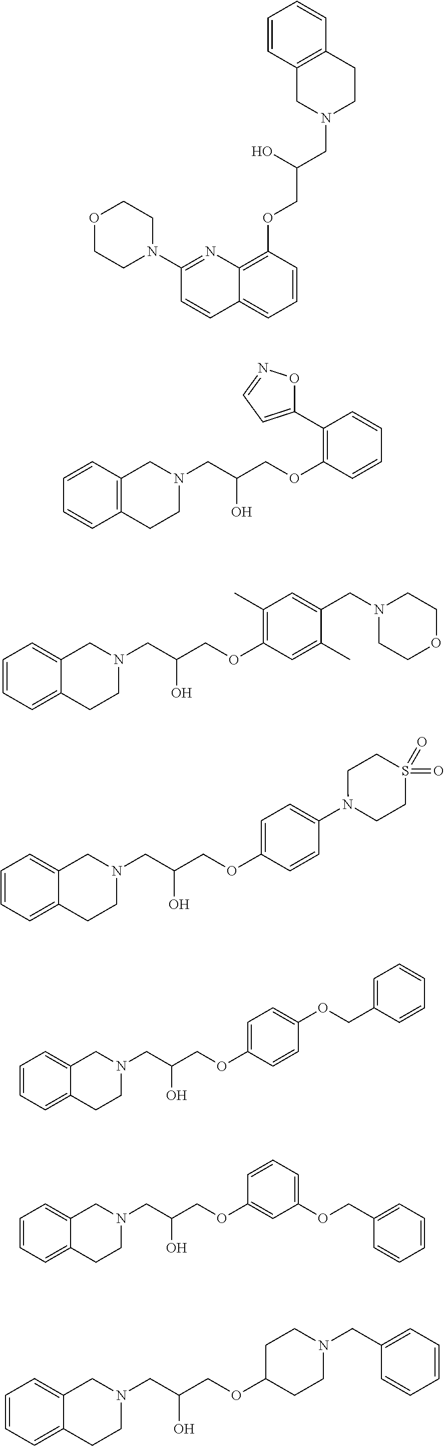

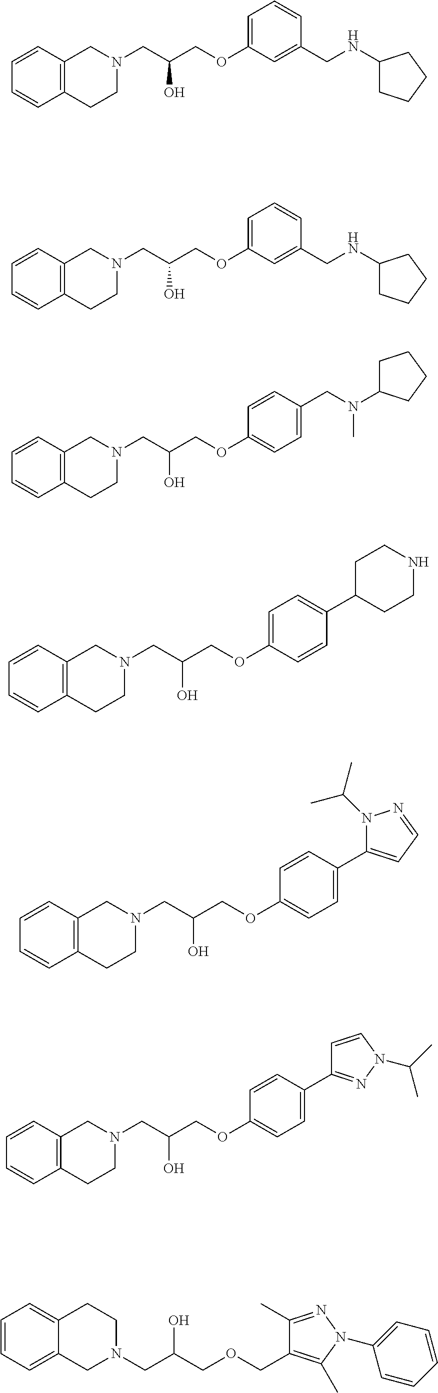

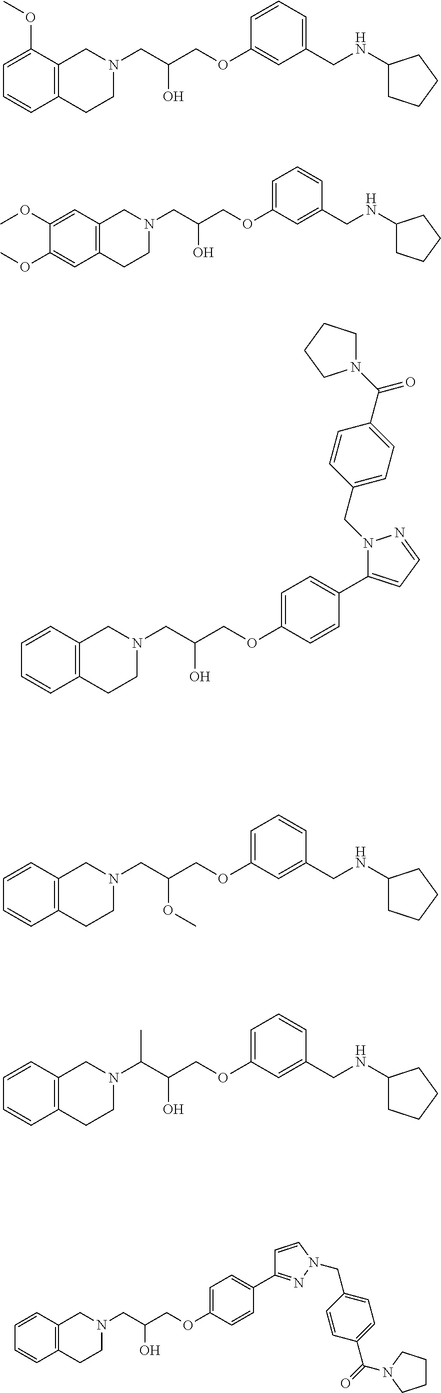 [SMILES: COc1cccc2c1CN(CC(O)COc1cccc(CNC3CCCC3)c1)CC2 ; COc1cc2c(cc1OC)CN(CC(O)COc1cccc(CNC3CCCC3)c1)CC2 ; O=C(c1ccc(Cn2nccc2-c2ccc(OCC(O)CN3CCc4ccccc4C3)cc2)cc1)N1CCCC1 ; COC(COc1cccc(CNC2CCCC2)c1)CN1CCc2ccccc2C1 ; CC(C(O)COc1cccc(CNC2CCCC2)c1)N1CCc2ccccc2C1 ; O=C(c1ccc(Cn2ccc(-c3ccc(OCC(O)CN4CCc5ccccc5C4)cc3)n2)cc1)N1CCCC1]

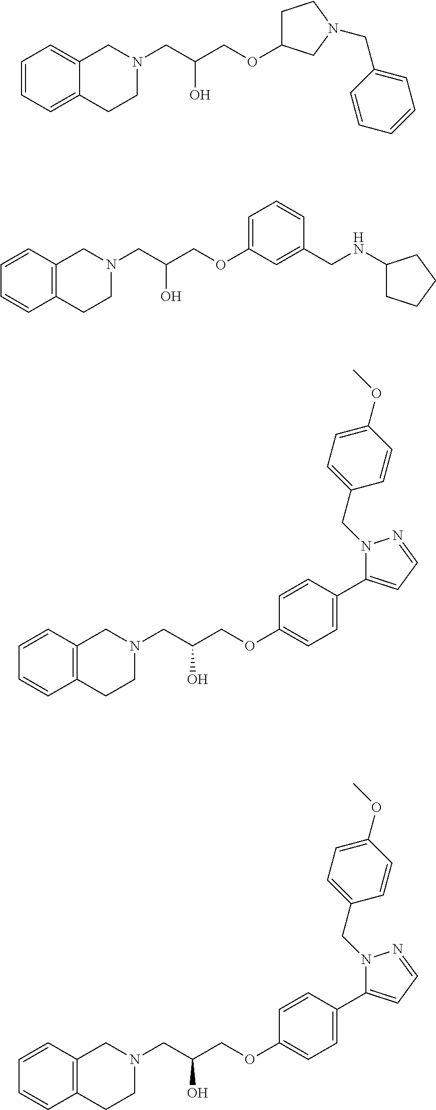 [SMILES: OC(COC1CCN(Cc2ccccc2)C1)CN1CCc2ccccc2C1 ; OC(COc1cccc(CNC2CCCC2)c1)CN1CCc2ccccc2C1 ; COc1ccc(Cn2nccc2-c2ccc(OC[C@H](O)CN3CCc4ccccc4C3)cc2)cc1 ; COc1ccc(Cn2nccc2-c2ccc(OC[C@@H](O)CN3CCc4ccccc4C3)cc2)cc1]

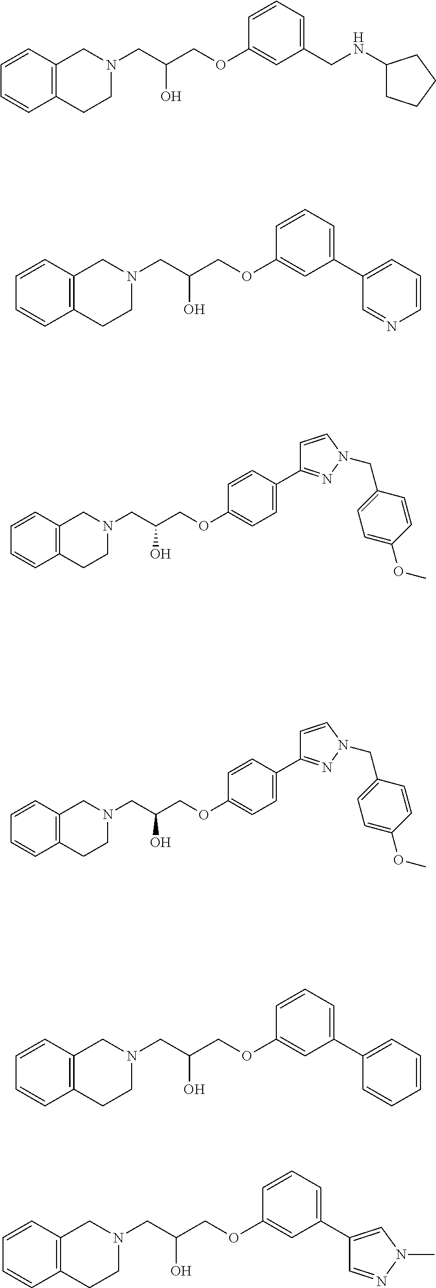 [SMILES: OC(COc1cccc(CNC2CCCC2)c1)CN1CCc2ccccc2C1 ; OC(COc1cccc(-c2cccnc2)c1)CN1CCc2ccccc2C1 ; COc1ccc(Cn2ccc(-c3ccc(OC[C@H](O)CN4CCc5ccccc5C4)cc3)n2)cc1 ; COc1ccc(Cn2ccc(-c3ccc(OC[C@@H](O)CN4CCc5ccccc5C4)cc3)n2)cc1 ; OC(COc1cccc(-c2ccccc2)c1)CN1CCc2ccccc2C1 ; Cn1cc(-c2cccc(OCC(O)CN3CCc4ccccc4C3)c2)cn1]

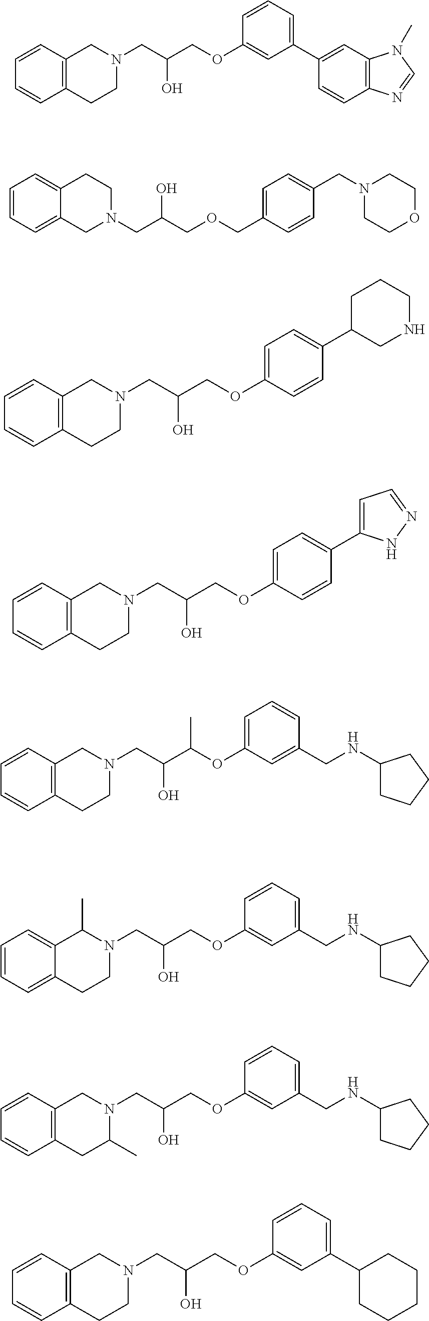

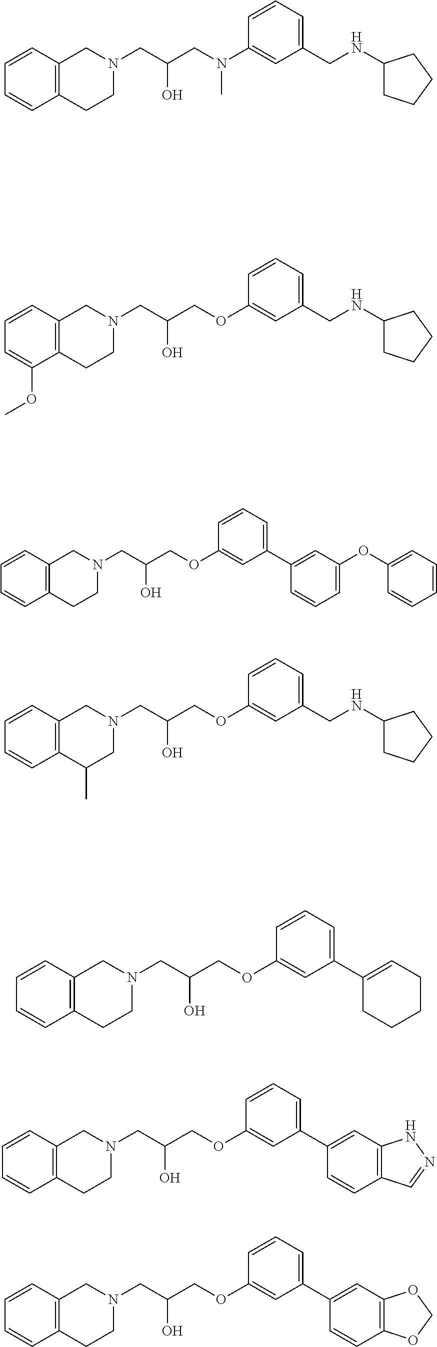

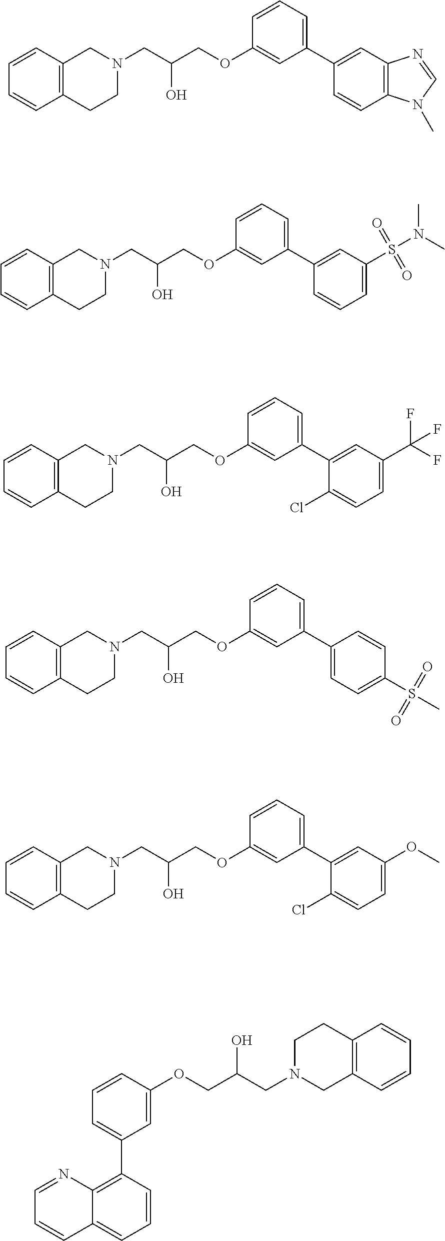 [SMILES: Cn1cnc2cc(-c3cccc(OCC(O)CN4CCc5ccccc5C4)c3)ccc21 ; CN(C)S(=O)(=O)c1cccc(-c2cccc(OCC(O)CN3CCc4ccccc4C3)c2)c1 ; OC(COc1cccc(-c2cc(C(F)(F)F)ccc2Cl)c1)CN1CCc2ccccc2C1 ; CS(=O)(=O)c1ccc(-c2cccc(OCC(O)CN3CCc4ccccc4C3)c2)cc1 ; COc1ccc(Cl)c(-c2cccc(OCC(O)CN3CCc4ccccc4C3)c2)c1 ; OC(COc1cccc(-c2cccc3cccnc23)c1)CN1CCc2ccccc2C1]

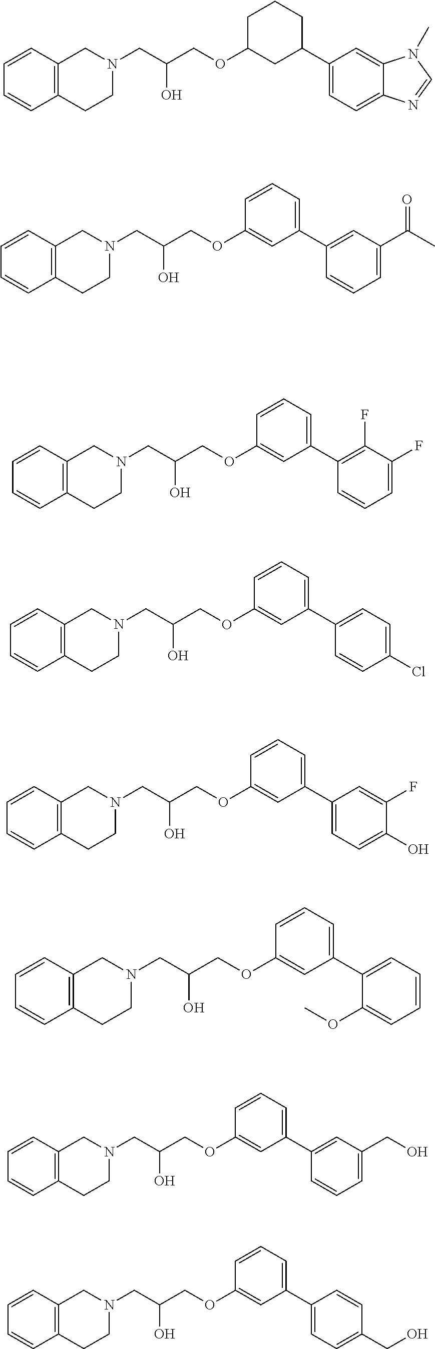

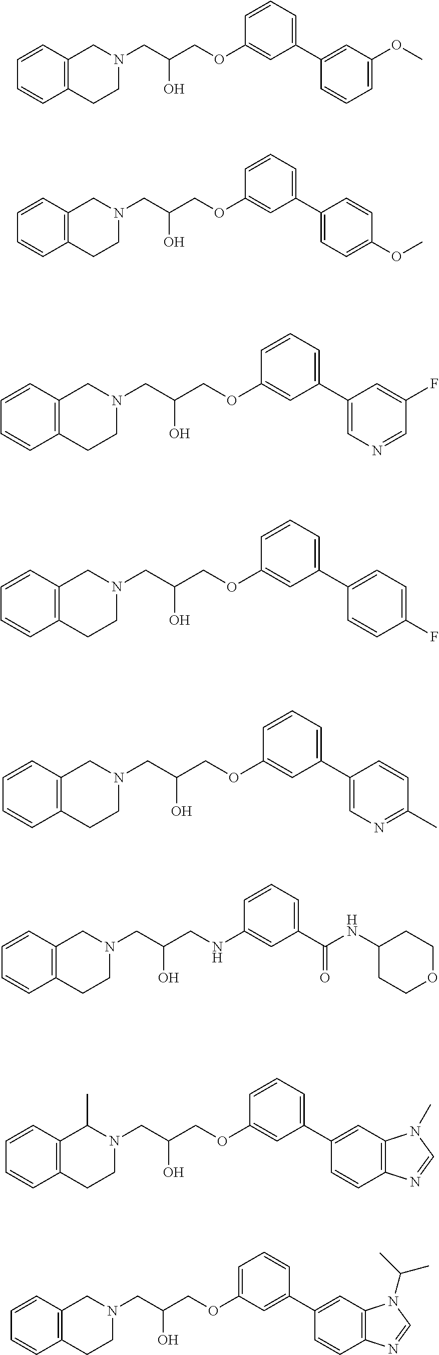

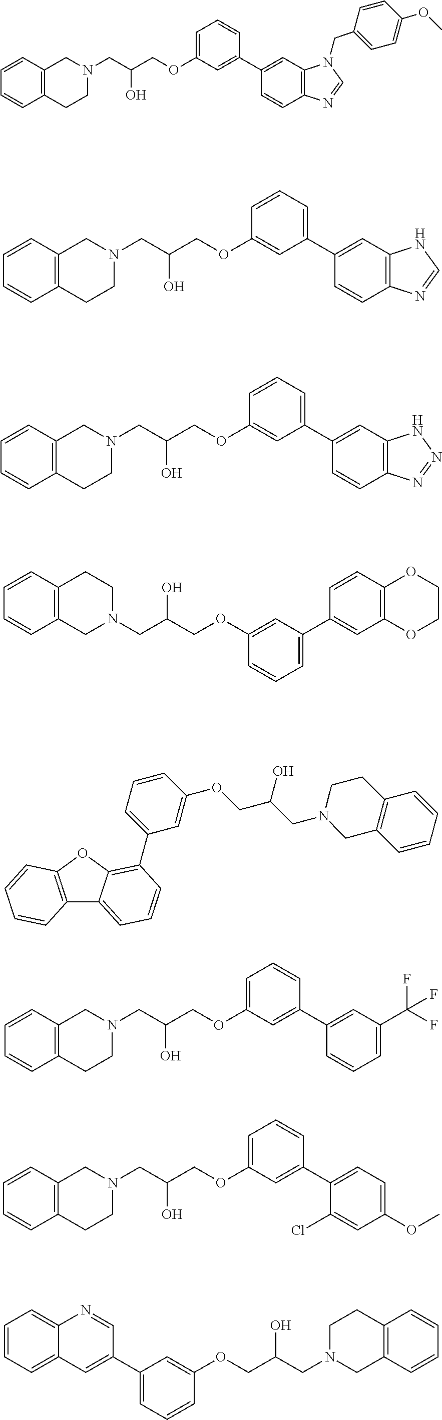

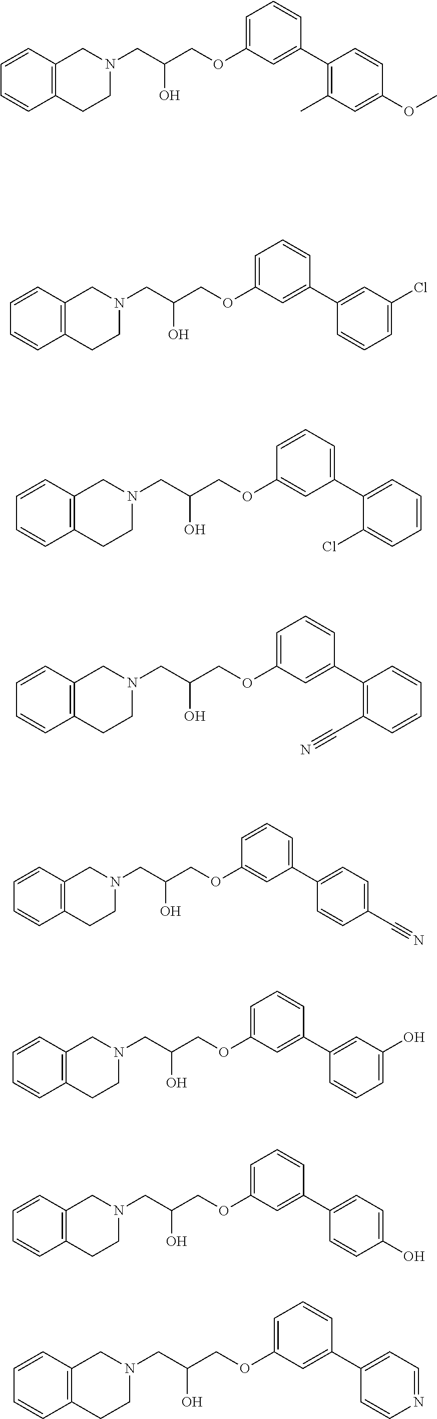

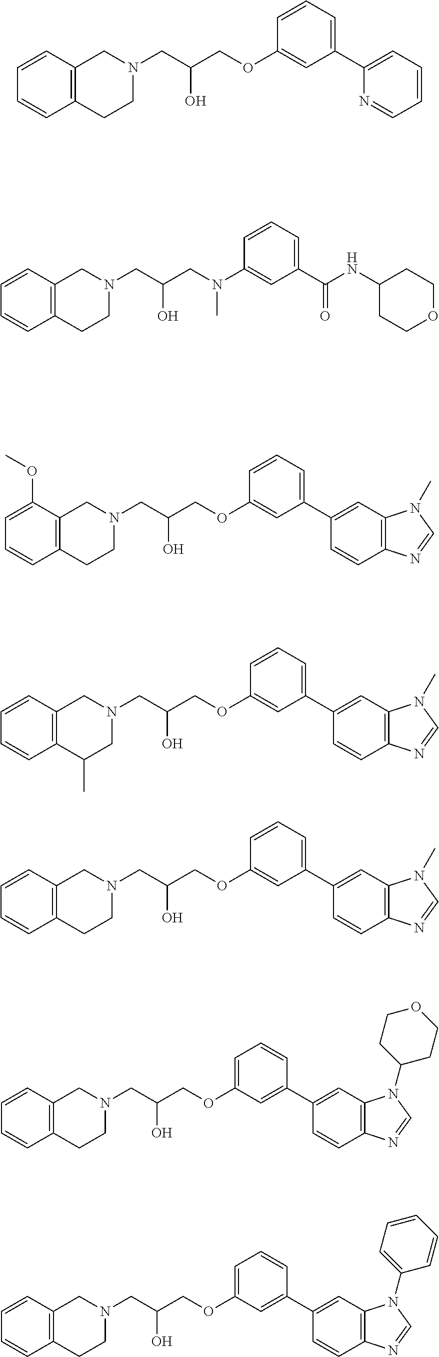

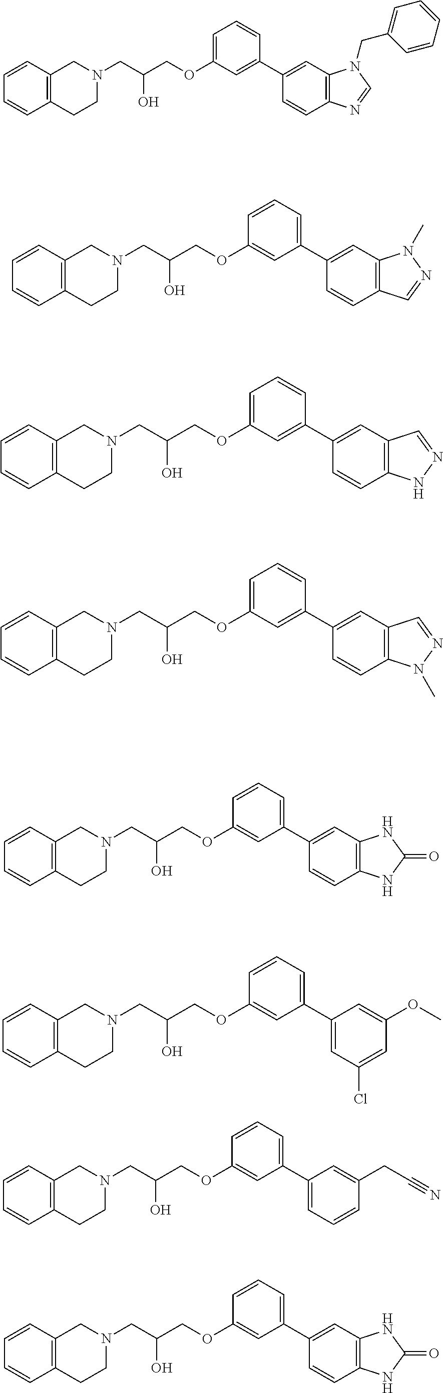

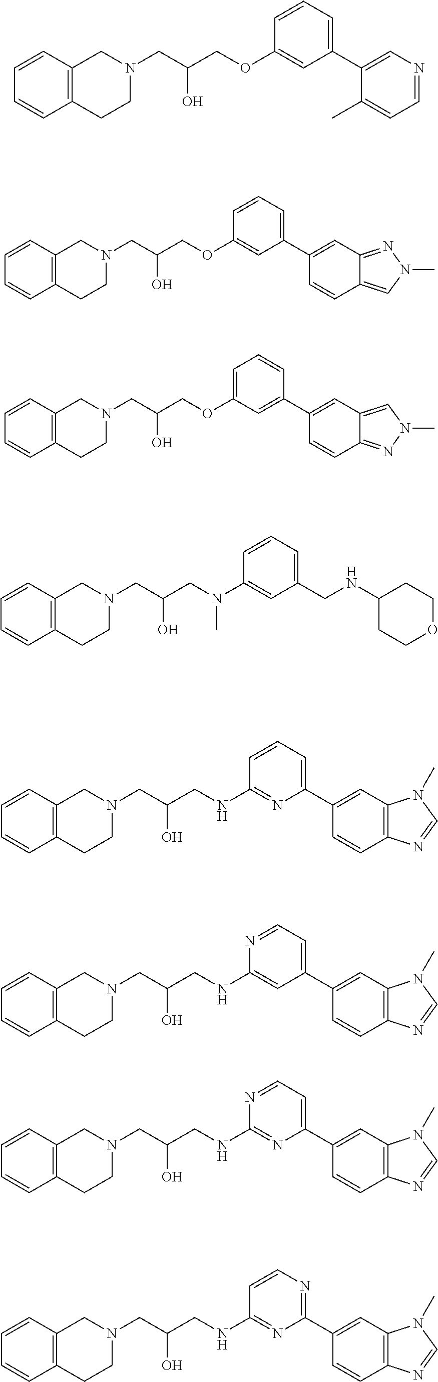

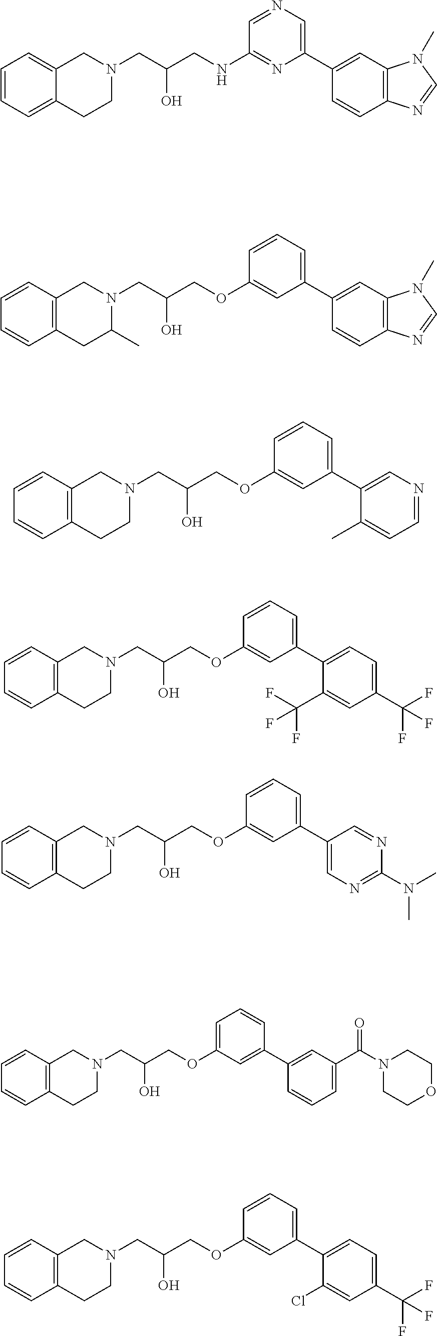

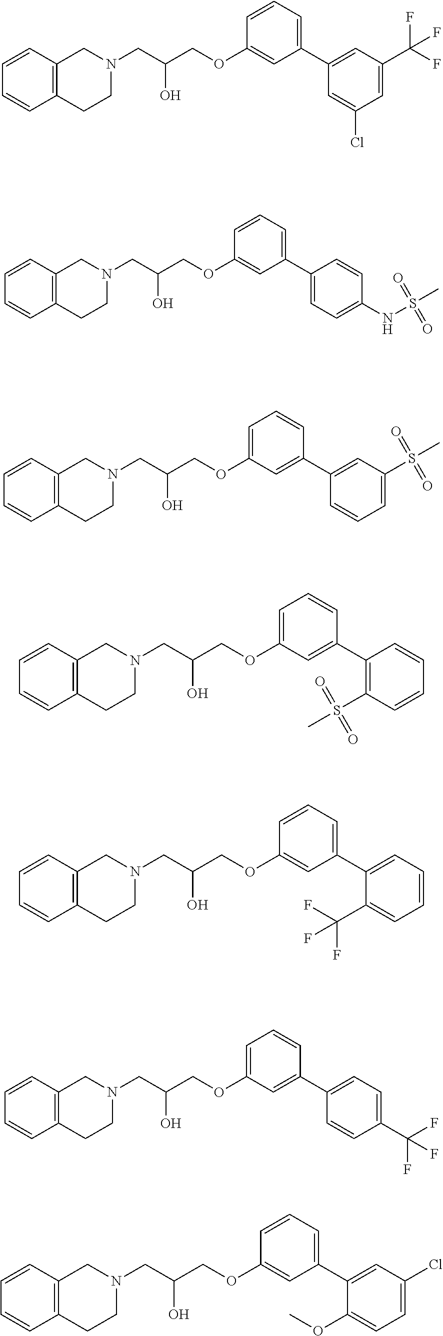

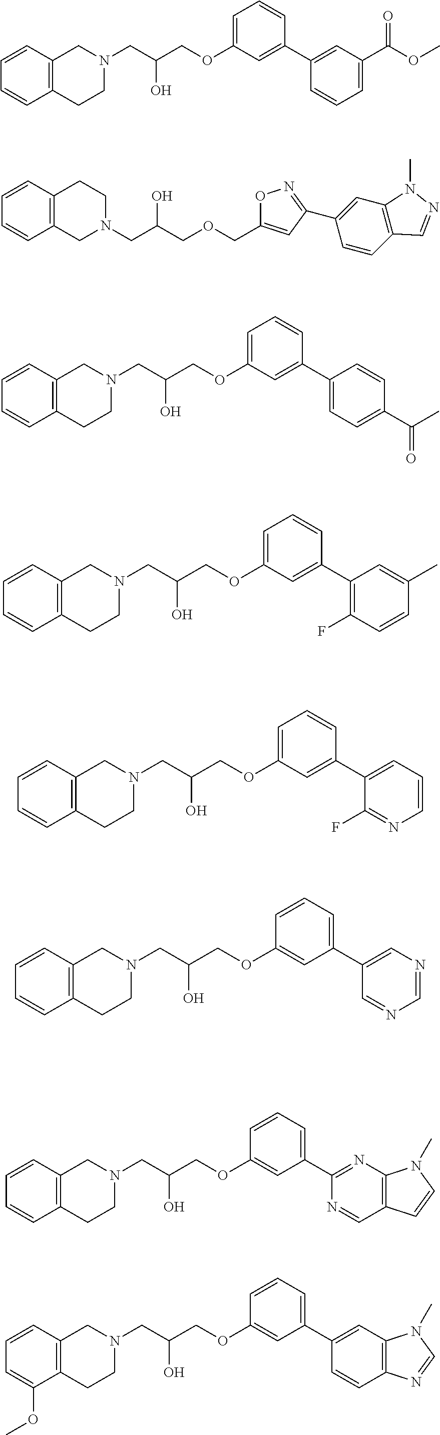

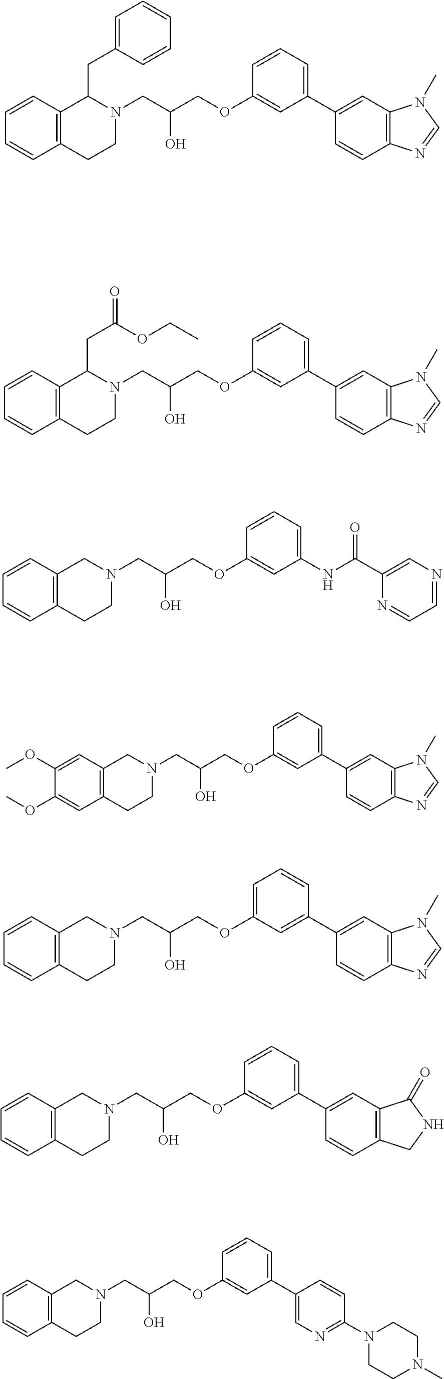

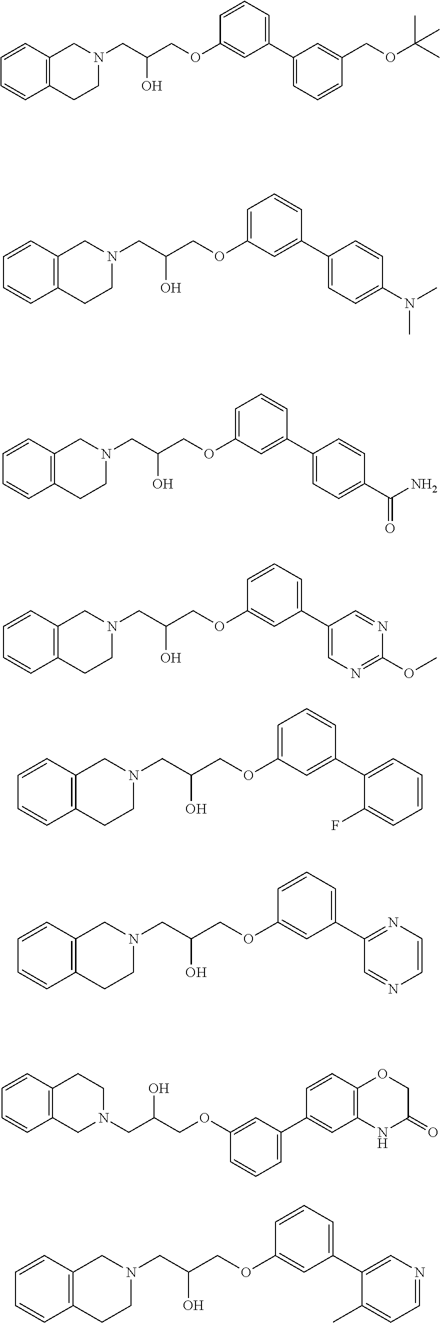

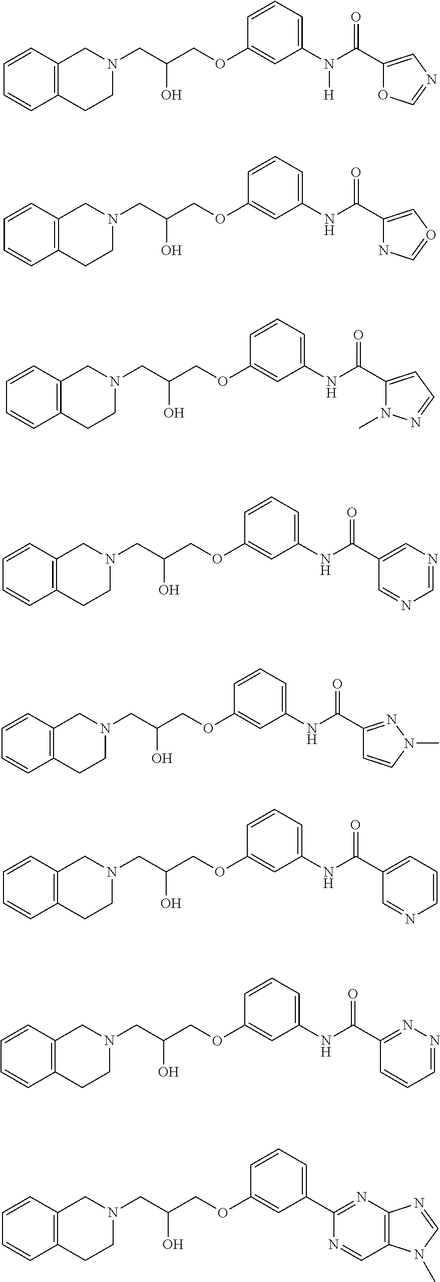

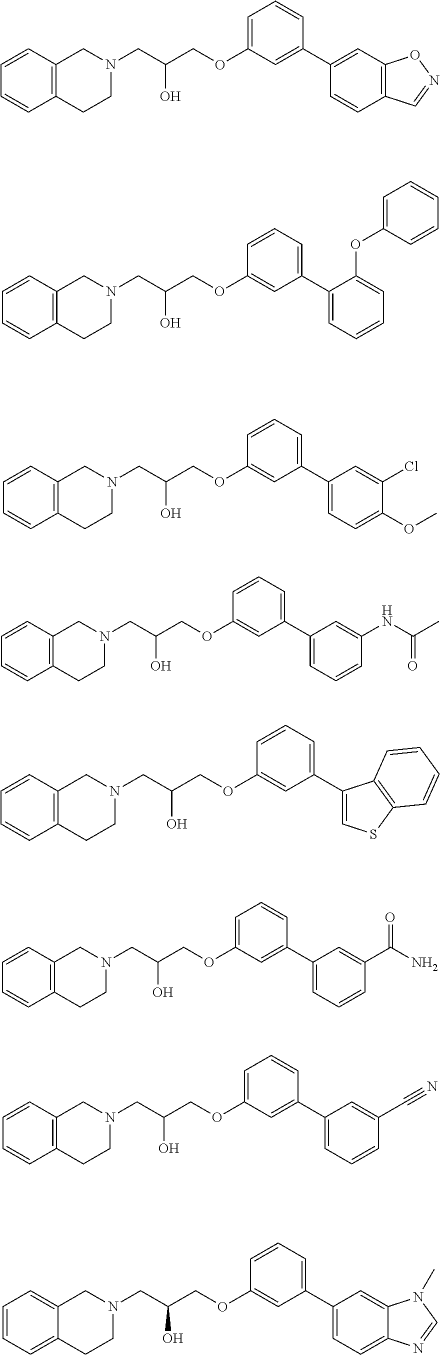

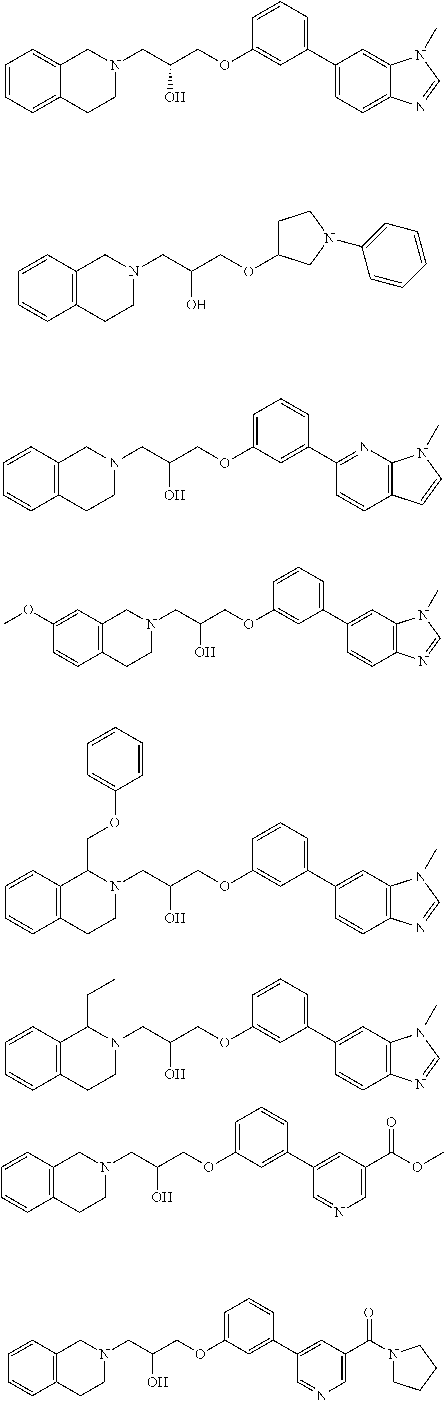

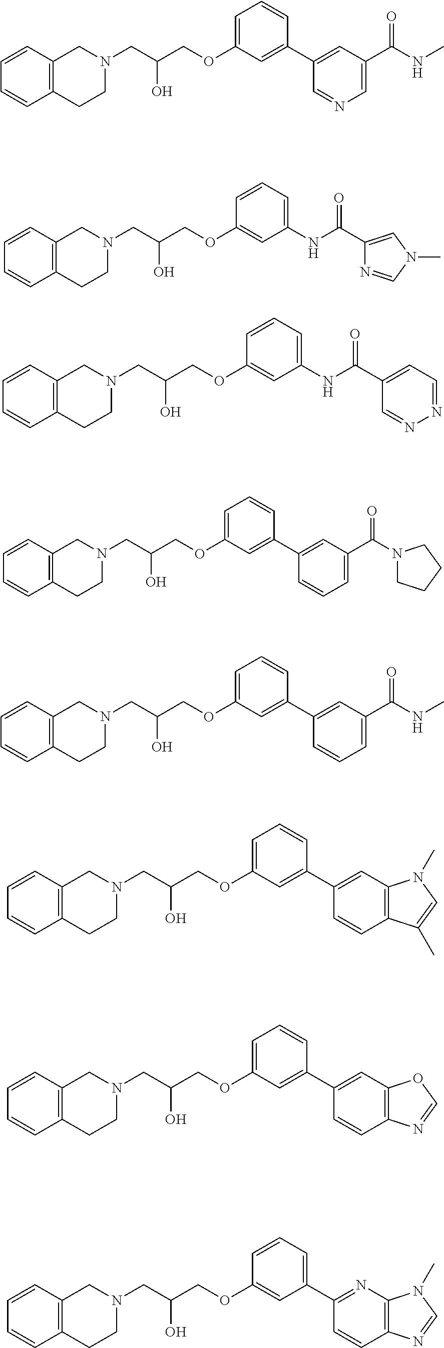

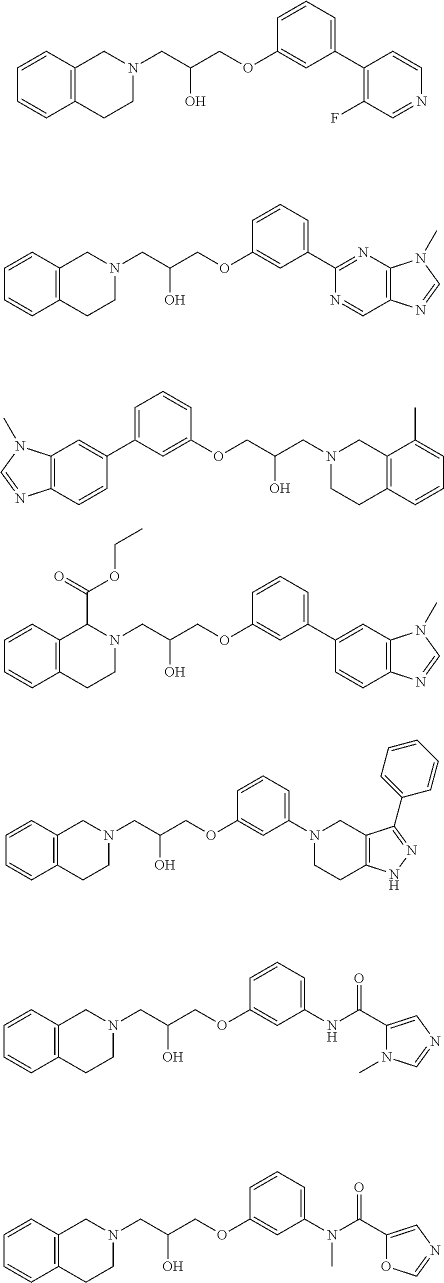

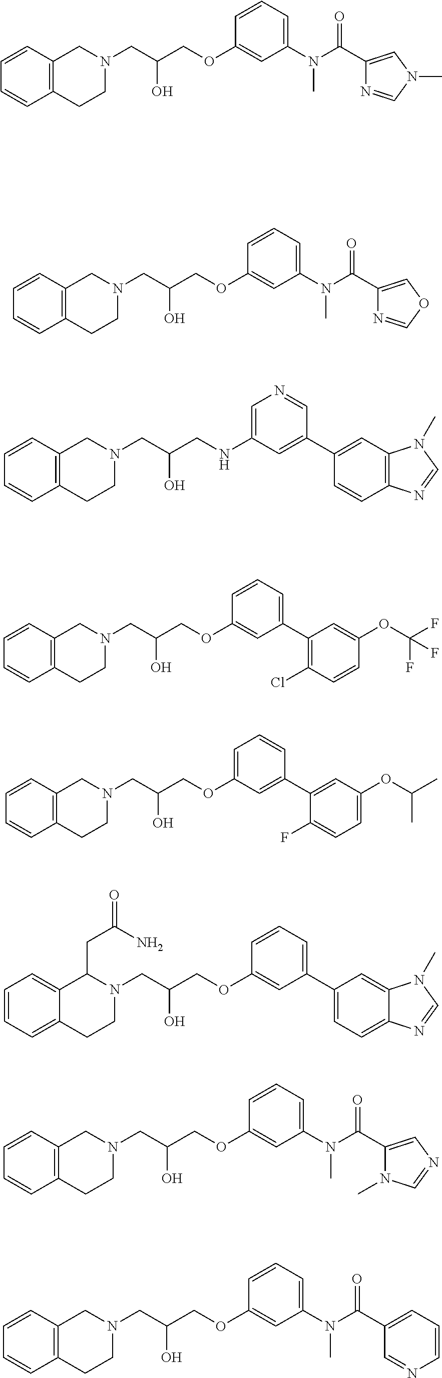

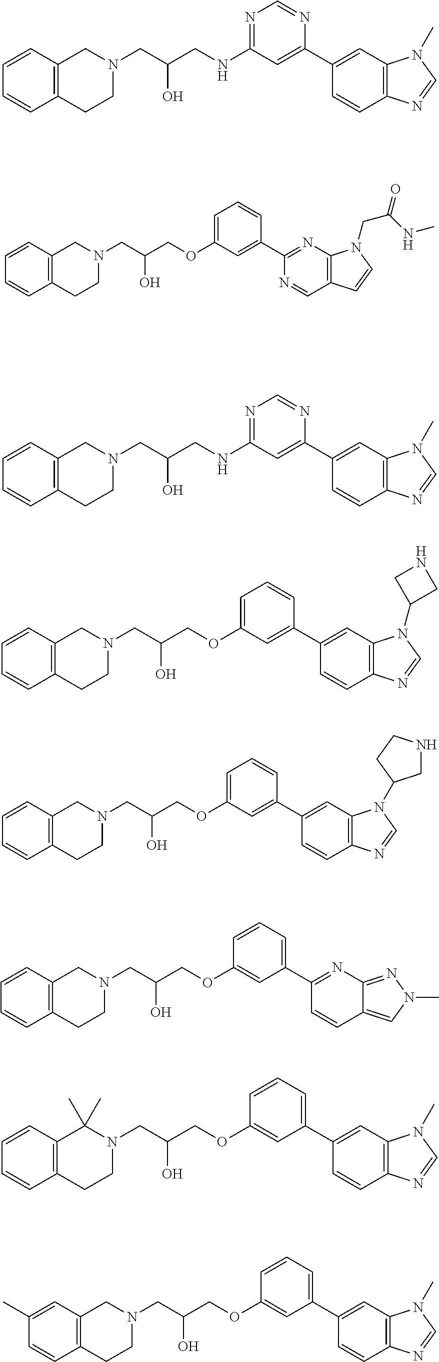

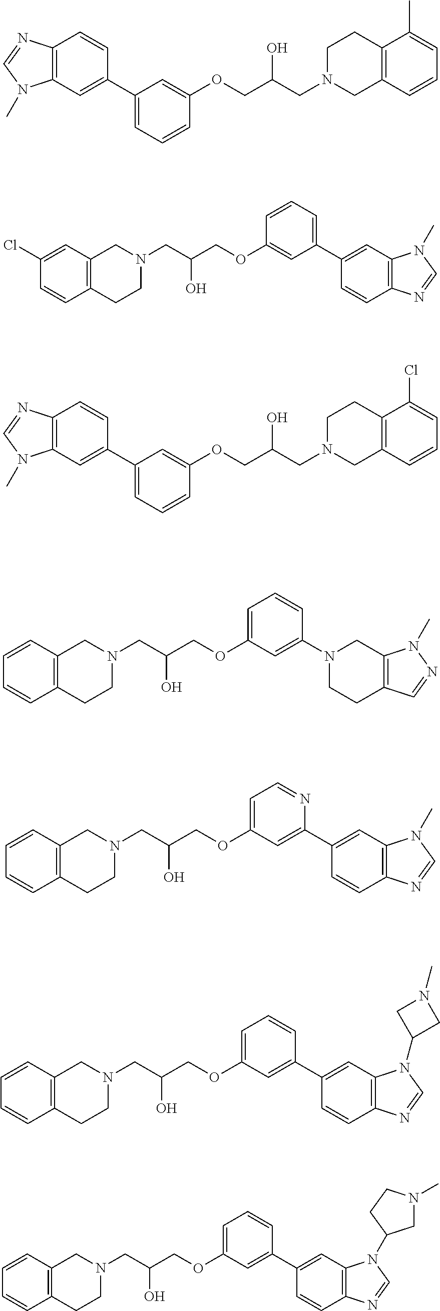

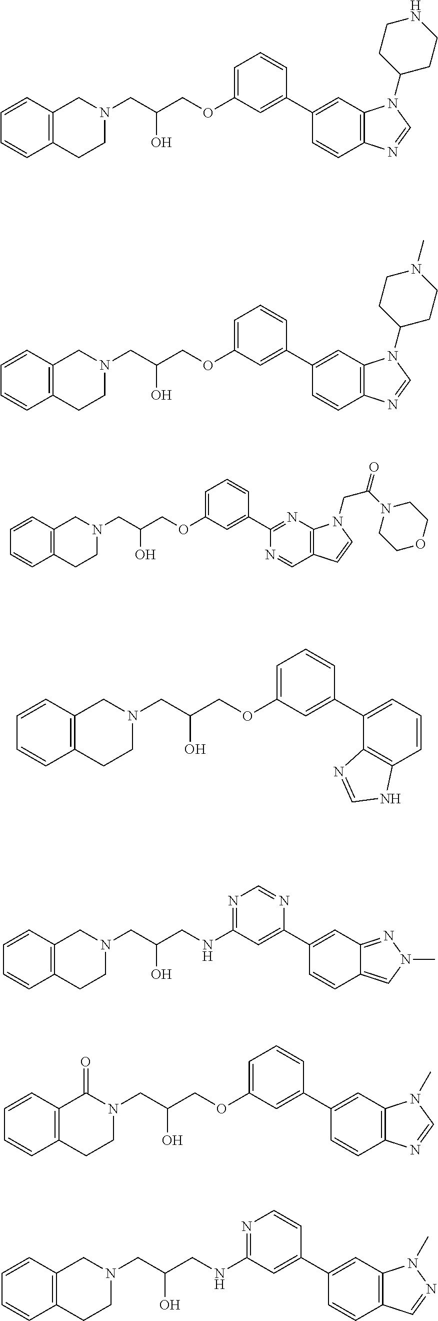

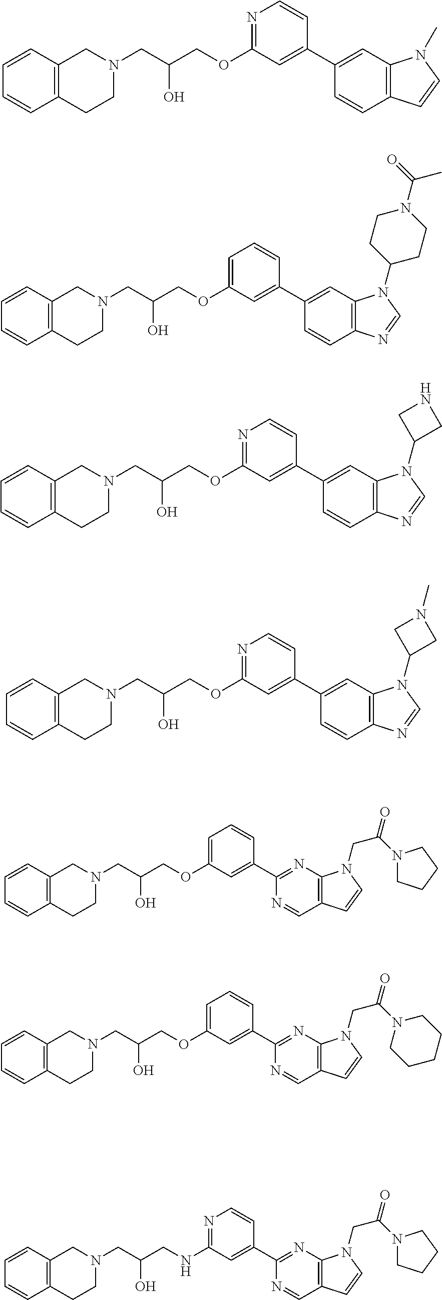

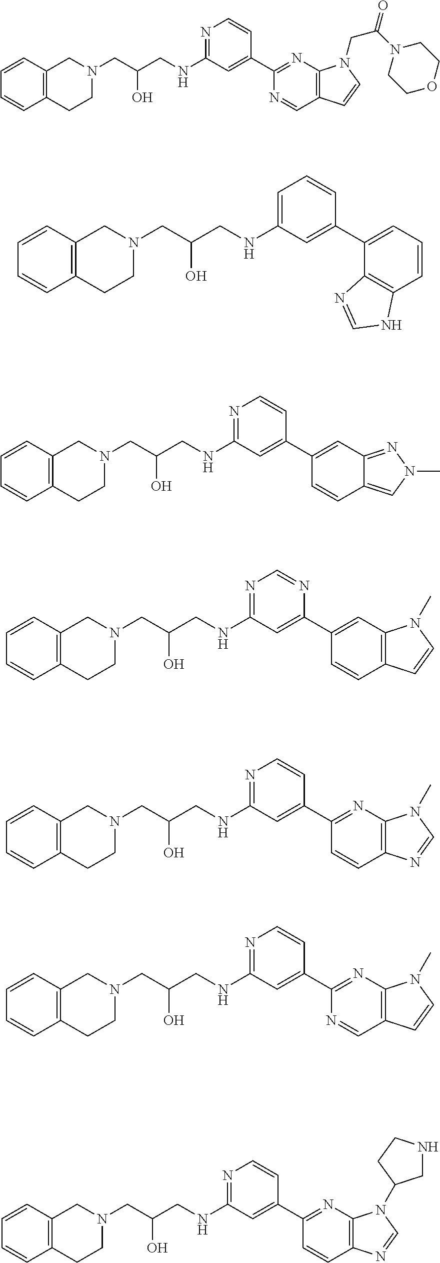

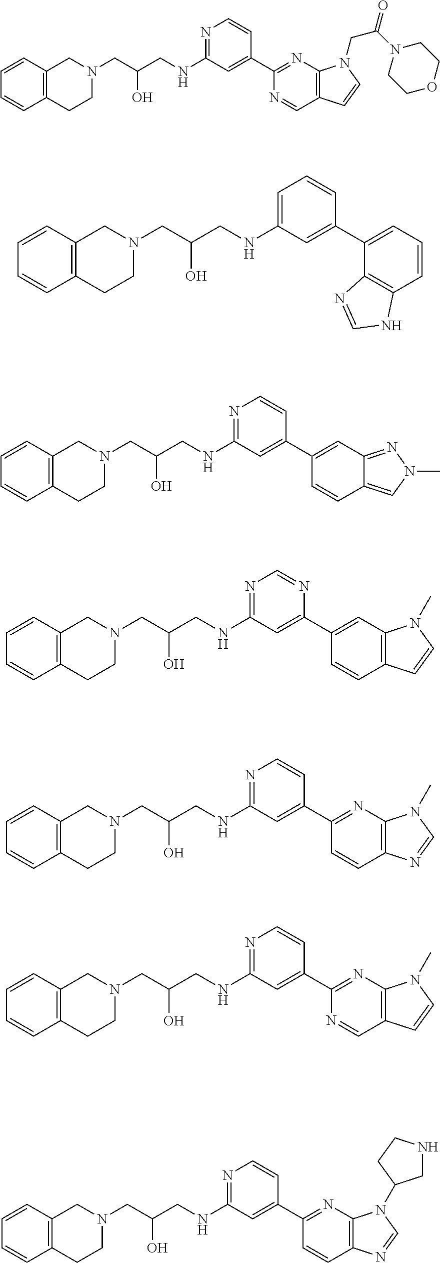

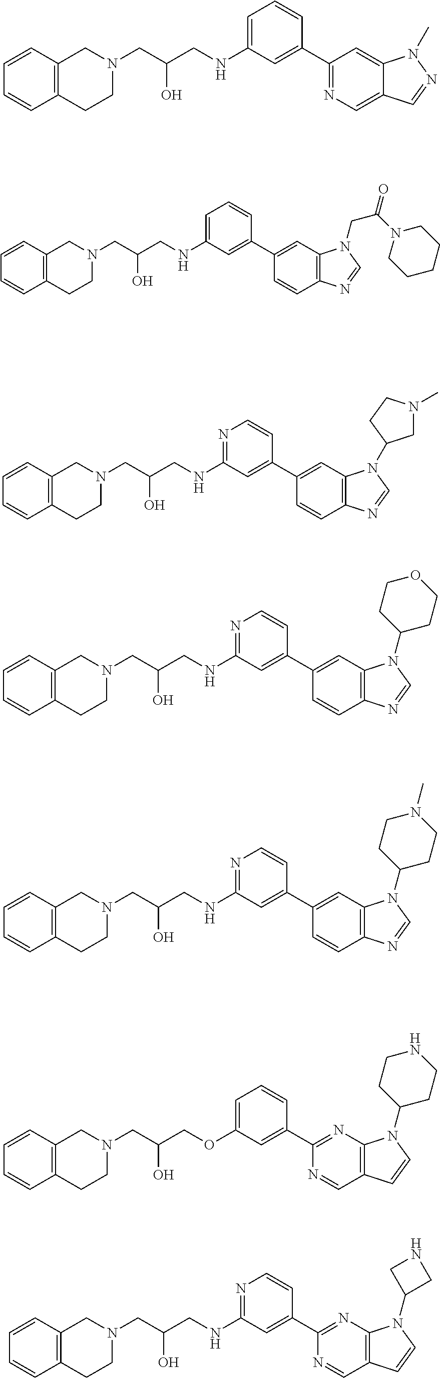

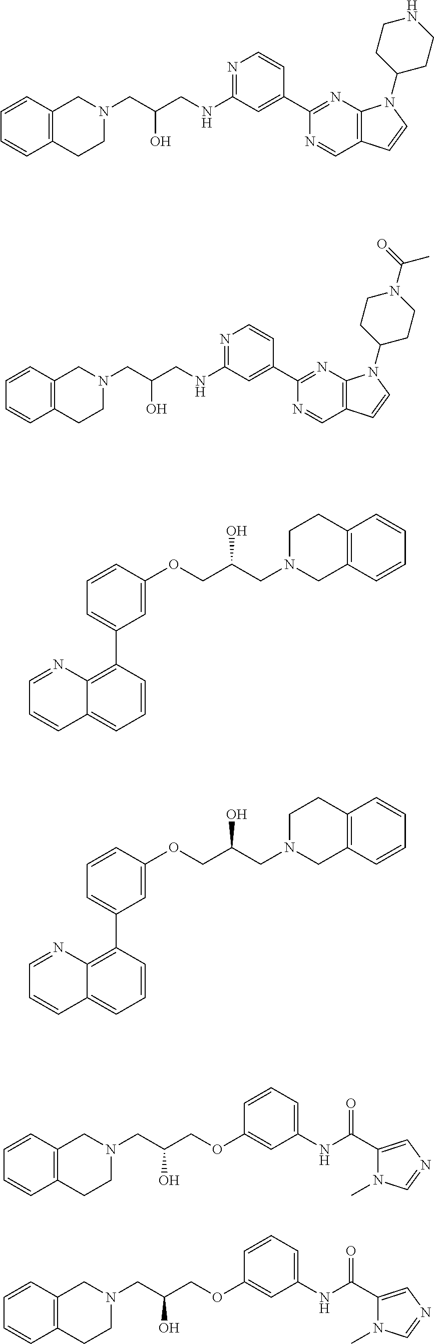 [SMILES: OC(CNc1cc(-c2ncc3ccn(C4CCNCC4)c3n2)ccn1)CN1CCc2ccccc2C1 ; CC(=O)N1CCC(n2ccc3cnc(-c4ccnc(NCC(O)CN5CCc6ccccc6C5)c4)nc32)CC1 ; O[C@@H](COc1cccc(-c2cccc3cccnc23)c1)CN1CCc2ccccc2C1 ; O[C@H](COc1cccc(-c2cccc3cccnc23)c1)CN1CCc2ccccc2C1 ; Cn1cncc1C(=O)Nc1cccc(OC[C@H](O)CN2CCc3ccccc3C2)c1 ; Cn1cncc1C(=O)Nc1cccc(OC[C@@H](O)CN2CCc3ccccc3C2)c1]

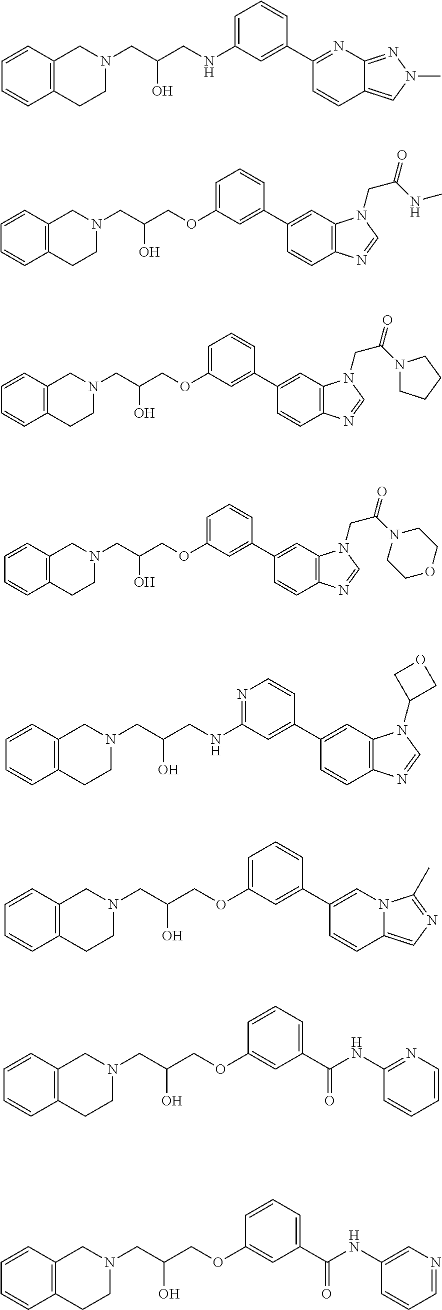

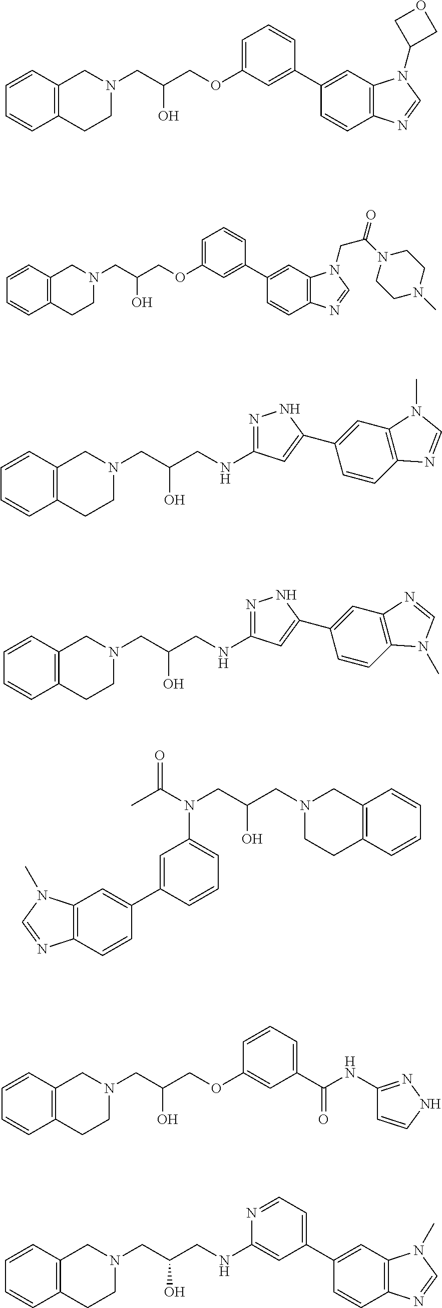

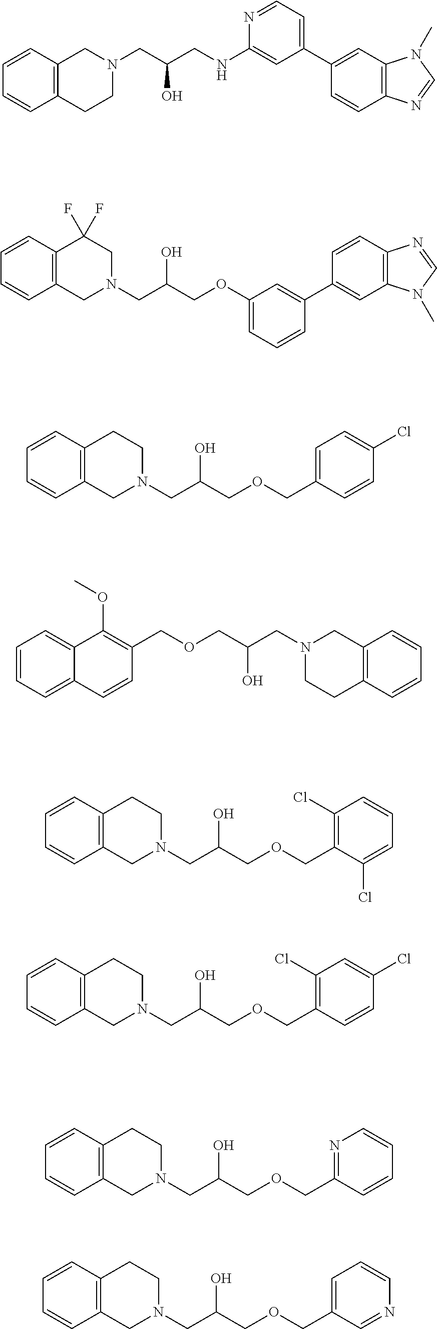

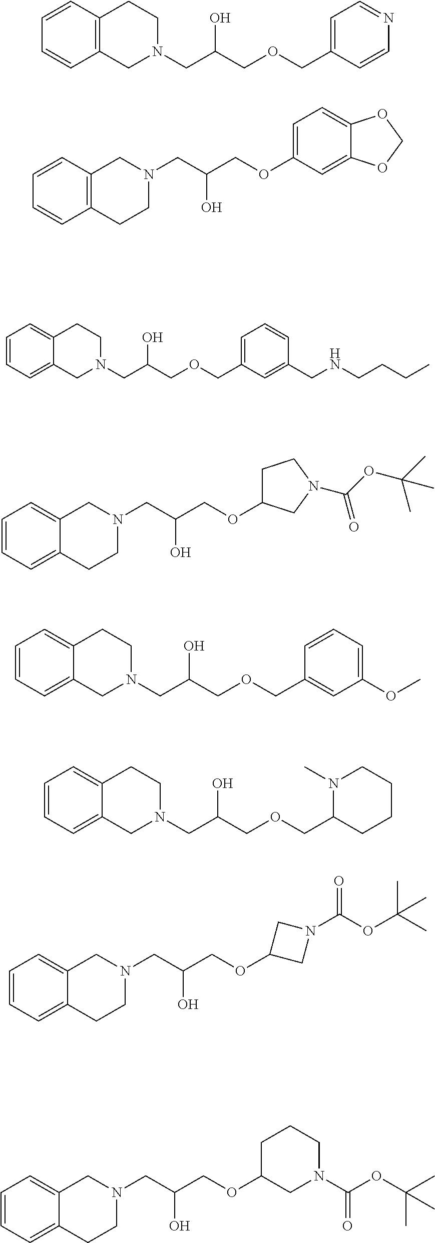

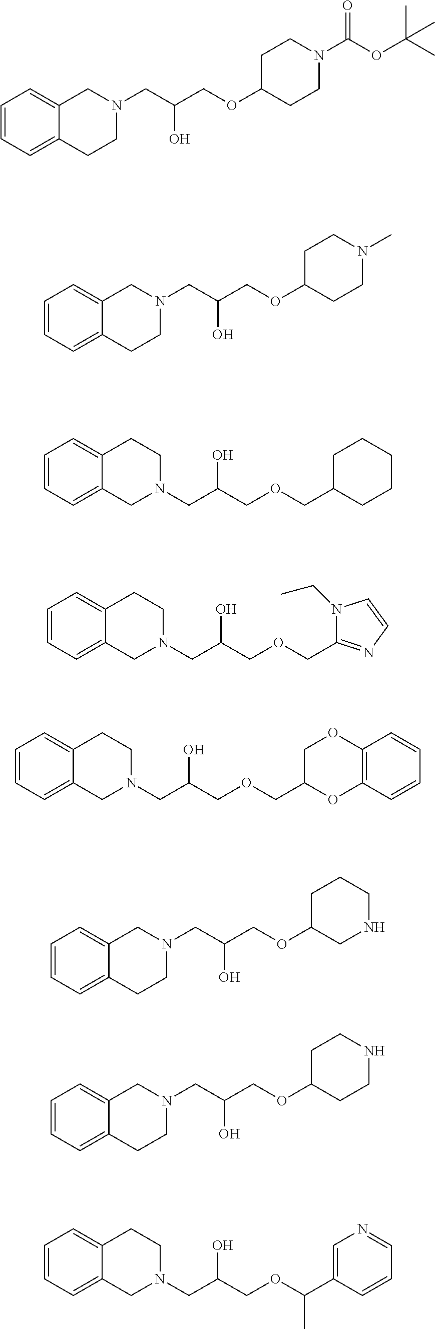

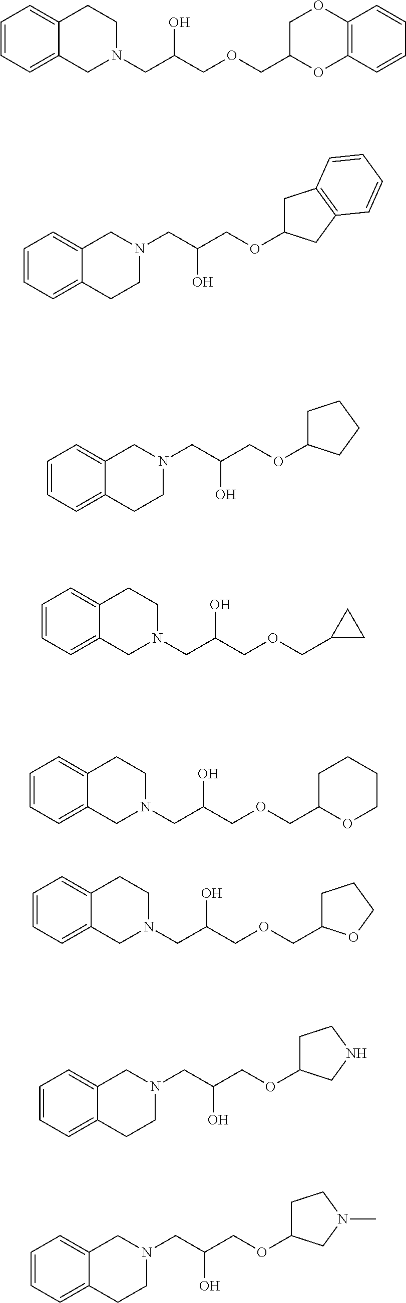

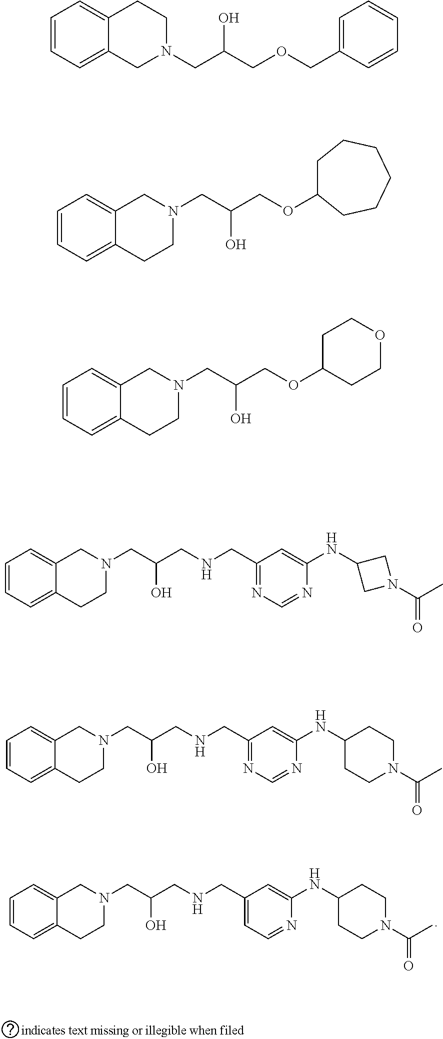 [SMILES: OC(COCc1ccccc1)CN1CCc2ccccc2C1 ; OC(COC1CCCCCC1)CN1CCc2ccccc2C1 ; OC(COC1CCOCC1)CN1CCc2ccccc2C1 ; CC(=O)N1CC(Nc2cc(CNCC(O)CN3CCc4ccccc4C3)ncn2)C1 ; CC(=O)N1CCC(Nc2cc(CNCC(O)CN3CCc4ccccc4C3)ncn2)CC1 ; CC(=O)N1CCC(Nc2cc(CNCC(O)CN3CCc4ccccc4C3)ccn2)CC1]

Any of the PRMT5 inhibitors described herein or known in the art can be used in the methods described herein. For example, the PRMT5 inhibitors described herein can be used in a method of inhibiting proliferation of MTAP-deficient and/or MTA-accumulating cells in a subject in need thereof, the method comprising the step of: administering to the subject, a PRMT5 inhibitor in an amount that is effective to inhibit proliferation of the MTAP-deficient and/or MTA-accumulating cells.

The PRMT5 inhibitors disclosed herein and in the art can be used in the methods of the present disclosure, wherein the proliferation and/or viability of a MTAP-deficient and/or MTA-accumulating cell, including, but not limited to, a cancer cell, can be decreased by administration of aPRMT5 inhibitor or a combination of PRMT5 inhibitors or a PRMT5 inhibitor and an anti-cancer agent selected from a HDAC inhibitor, a mTor inhibitor, and a PI3K inhibitor.

In addition, this disclosure notes that some of the documents describing PRMT5 inhibitors noted above involved the use of Z138, HARA and/or TE1 cells, which data produced in the present work have shown (via Western blots, RNA expression analysis, gene copy number analyses, scatter plots, and/or other methods) to have an intact MTAP locus and/or express MTAP.

Combination Therapies

Many potential combination partners exist for treatment with PRMT5 inhibition. The treatment could be partnered with current standards of care in the cancer types to be treated, as well as potential future drugs that might be approved.

PRMT5 inhibitors of the instant disclosure can be used as part of a combination with other therapies. The term “Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

By “combination”, there is meant either a fixed combination in one dosage unit form, or a combined administration where a compound of the present invention and a combination partner may be administered independently at the same time or separately within time intervals that especially allow that the combination partners show a cooperative, e.g. synergistic effect. The single components may be packaged together in a kit or separately. One or both of the components (e.g., powders or liquids) may be reconstituted or diluted to a desired dose prior to administration.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

In certain instances, compounds of the present invention are combined with other therapeutic agents, including, but not limited to, other anti-cancer agents, anti-allergic agents, anti-nausea agents (or anti-emetics), pain relievers, cytoprotective agents, and combinations thereof.

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the compounds of the present invention include:

Some patients may experience allergic reactions to the compounds of the present invention and/or other anti-cancer agent(s) during or after administration; therefore, anti-allergic agents are often administered to minimize the risk of an allergic reaction. Suitable anti-allergic agents include corticosteroids, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present invention and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs including, but not limited to, hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present invention, can be prepared and administered as described in the art, including, but not limited to, in the documents cited above.

In one embodiment, the present invention provides pharmaceutical compositions comprising at least one compound of the present invention (e.g., a compound of the present invention) or a pharmaceutically acceptable salt thereof together with a pharmaceutically acceptable carrier suitable for administration to a human or animal subject, either alone or together with other anti-cancer agents.

In one embodiment, the present invention provides methods of treating human or animal subjects suffering from a cellular proliferative disease, including, but not limited to, cancer. The present invention provides methods of treating a human or animal subject in need of such treatment, comprising administering to the subject a therapeutically effective amount of a compound of the present invention (e.g., a compound of the present invention) or a pharmaceutically acceptable salt thereof, either alone or in combination with other anti-cancer agents.

In particular, compositions will either be formulated together as a combination therapeutic or administered separately.

In combination therapy, the compound of the present invention and other anti-cancer agent(s) may be administered either simultaneously, concurrently or sequentially with no specific time limits, wherein such administration provides therapeutically effective levels of the two compounds in the body of the patient.

In a preferred embodiment, the compound of the present invention and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

A compound of the present invention may also be used to advantage in combination with known therapeutic processes, for example, the administration of hormones or especially radiation. A compound of the present invention may in particular be used as a radiosensitizer, especially for the treatment of tumors which exhibit poor sensitivity to radiotherapy.

In certain instances, compounds of the present invention are combined with other therapeutic agents, including, but not limited to, other anti-cancer agents, anti-allergic agents, anti-nausea agents (or anti-emetics), pain relievers, cytoprotective agents, and combinations thereof.

Specific compounds and classes of compounds acting via a specific mechanism have been identified to be particularly effective in conjunction with PRMT5 inhibitors. For example, PRMT5 is known to associate with SWI/SNF chromatin remodeling complexes along with other co-repressor molecules like HDAC2. PRMT5 activity on target H4R3 and H3R8 is enhanced when lysine residues become deacetylated by HDAC enzmes. Thus, HDAC inhibitors have been tested and found to be effective when used in conjunction with PRMT5 inhibitors. The combination of a PRMT5 inhibitor, a HDAC inhibitor and a DNA methyltransferase inhibitor was synergistic. WO 011/079236.

A PRMT5 inhibitor can also be administered or co-administered in any order with an inhibitor of a protein which interacts with or is required for PRMT5 function, including, but not limited to, pICIN, WDR77 or RIOK1.

Thus, PRMT5 inhibitors of the present disclosure can be used in combination with other compounds, for example: HDAC inhibitor or DNA methyltransferase inhibitor. In some embodiments, the HDAC inhibitor is Trichostatin A. In some embodiments, the DNA methyltransferase inhibitor is 5-azacytidine. Any of the compounds can be used in combination with any PRMT5 inhibitor described herein or known in the art, in any method described herein.

A PRMT5 inhibitor can be administered in combination with a HDM2 inhibitor and/or with 5-FU. The loss has been observed of wild-type p53 as a consequence of HDM2 activation resulting from CDKN2A deletion. This relates to the inability of MTAP deleted cells to salvage ATP and methionine from endogenous methyl-thioadenosine (MTA). As a consequence tumor cells become differentially sensitive towards 5-FU and other purine analogues (e.g., 6-thioguanine, 6-mercaptopurine). Given that CDKN2A/MTAP loss also leads to deregulation of p16/CDK4/6 pathway, another combination is with a CDK4 inhibitor, including, but not limited to, LEE011. Thus, a PRMT5 inhibitor can be administered or co-administered in any order with any one or more of the following: a HDM2 inhibitor, 5-FU, a purine analogue, 6-thioguanine, 6-mercaptopurine, CDK4 inhibitor, or LEE011, or inhibitors of HDM2i, PI3K/mTOR-I, MAPKi, RTKi (EGFRi, FGFRi, METi, IGFiRi, JAKi, or WNTi.

Additional combination therapies are provided below.

Given the high frequency at which MTAP-loss is found across target indications/tumors this disclosure presents the following additional combination options:

(A) Combination of a PRMT5 inhibitor with drugs towards which MTAP-loss tumors in general and irrespective of dignity can be expected to be highly sensitive, even more so when combined with a PRMT5 inhibitor, e.g., 5-FU and analogues thereof; and purine analogues (e.g. 6-thioguanine, mercaptopurine and others). There exists the option of using MTA (methylthioadenosine) as a co-medication as this can leverage the tolerability and/or alleviate toxicity in normal tissues.

(B) Combination of a PRMT5 inhibitor with targeted treatments contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

(C) Combination of a PRMT5 inhibitor with immunotherapy

(D) Combination of a PRMT5 inhibitor with disease-specific huMABs (e.g., an anti-HER3 huMAB)

(E) Combination of a PRMT5 inhibitor with ADCs/ADCCs contingent on the expression of relevant surface targets on target tumors of interest

(F) Combination of a PRMT5 inhibitor with disease-specific and established 1st/2nd line Gold-Standard treatments.

A PRMT5 inhibitor can be administered or co-administered in any order with any known chemotherapeutic or therapeutic agent in a combination therapy.

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the compounds of the present invention include fluorouracil (5-FU) and irinotecan.

Further compounds of particular interest for combinations with the compounds of the present invention include: EGFR-inhibitors, such as cetuximab, panitumimab, erlotinib, gefitinib and EGFRi NOS; MAPK-pathway inhibitors, such as BRAFi, panRAFi, MEKi, ERKi; PI3K-mTOR pathway inhibitors, such as alpha-specific PI3Ki, pan-class I PI3Ki, mTOR/PI3Ki), particularly also evirolimus and analogues thereof.

Some patients may experience allergic reactions to the compounds of the present invention and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present invention and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytrilt), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present invention, can be prepared and administered as described in the art, such as in the documents cited above.

In one embodiment, the present invention provides pharmaceutical compositions comprising at least one compound of the present invention (e.g., a compound of the present invention) or a pharmaceutically acceptable salt thereof together with a pharmaceutically acceptable carrier suitable for administration to a human or animal subject, either alone or together with other anti-cancer agents.

In one embodiment, the present invention provides methods of treating human or animal subjects suffering from a cellular proliferative disease, such as cancer. The present invention provides methods of treating a human or animal subject in need of such treatment, comprising administering to the subject a therapeutically effective amount of a compound of the present invention (e.g., a compound of the present invention) or a pharmaceutically acceptable salt thereof, either alone or in combination with other anti-cancer agents.

In particular, compositions will either be formulated together as a combination therapeutic or administered separately.

In combination therapy, the compound of the present invention and other anti-cancer agent(s) may be administered either simultaneously, concurrently or sequentially with no specific time limits, wherein such administration provides therapeutically effective levels of the two compounds in the body of the patient.

In a preferred embodiment, the compound of the present invention and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

A compound of the present invention may also be used to advantage in combination with known therapeutic processes, for example, the administration of hormones or especially radiation. A compound of the present invention may in particular be used as a radiosensitizer, especially for the treatment of tumors which exhibit poor sensitivity to radiotherapy.

Any of the PRMT5 inhibitors described herein or known in the art can be used in a method of inhibiting proliferation of MTAP-deficient cells in a subject in need thereof, the method comprising the step of administering to the subject, a PRMT5 inhibitor in an amount that is effective to inhibit proliferation of the MTAP-deficient cells. Any of the PRMT5 inhibitors described herein or known in the art can be used in a method of inhibiting proliferation of MTA-accumulating cells in a subject in need thereof, the method comprising the step of administering to the subject, a PRMT5 inhibitor in an amount that is effective to inhibit proliferation of the MTA-accumulating cells. Any of the PRMT5 inhibitors described herein or known in the art can be used in a method of inhibiting proliferation of MTAP-deficient and/or MTA-accumulating cells in a subject in need thereof, the method comprising the step of administering to the subject, a PRMT5 inhibitor in an amount that is effective to inhibit proliferation of the MTAP-deficient and/or MTA-accumulating cells. The disclosure also encompasses method of detecting MTAP-deficiency in cells, including but not limited to cancer cells, and methods of preparing samples (e.g., of cells, tissues, tumors, etc.) for evaluating the samples for MTAP deficiency.

Sample Preparation

The invention provides, among other things, an assay for the detection of MTAP deficiency and/or MTA accumulation.

The method can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascite, serous fluid, sputum, lacrimal fluid, stool, and urine, or in a tissue such as a tumor tissue. The tumor tissue can be fresh tissue or paraffin-embedded tissue.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

Generally, a solid tumor sample can be a test sample of cells or tissue that are obtained from a subject with cancer by biopsy or surgical resection. A sample of cells or tissue can be removed by needle aspiration biopsy. For this, a fine needle attached to a syringe is inserted through the skin and into the tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A sample of cells or tissue can also be removed by incisional or core biopsy. For this, a cone, a cylinder, or a tiny bit of tissue is removed from the region of interest. CT imaging, ultrasound, or an endoscope is generally used to guide this type of biopsy. More particularly, the entire cancerous lesion may be removed by excisional biopsy or surgical resection. In the present invention, the test sample is typically a sample of cells removed as part of surgical resection.

The test sample of, for example tissue, may also be stored in, e.g., RNAlater (Ambion; Austin Tex.) or flash frozen and stored at −80° C. for later use. The biopsied tissue sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a fixed or wax-embedded tissue sample.

Cancers amenable for treatment according to the present invention include glioblastoma, bladder cancer, pancreatic cancer, mesothelioma, melanoma, lung squamous, lung adenocarcinoma, diffuse large B-cell lymphoma (DLBCL), leukemia, and head and neck cancer, and cancer of the kidney, breast, endometrium, urinary tract, liver, soft tissue, pleura and large intestine. This disclosure notes that a subset of PRMT5 inhibitors may be neurotoxic. Potential PRMT5 inhibitors thus should be evaluated for this and other toxicities. Neurotoxic PRMT5 inhibitors can be modified to prevent transit across the blood-brain barrier, thus increasing their usefulness for treating non-CNS (central nervous system) MTAP-deficient and/or MTA-accumulating cancers.

Detection of PRMT5 Sensibility

Samples, once prepared, can be tested for MTAP deficiency and/or MTA accumulation, either or both of which indicates that the sample (or, more usefully, similar cells from the patient) are sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization or amplification utilizing at least one probe or primer comprising a sequence of at least 12 contiguous nucleotides (nt) of the sequence of MTAP provided in SEQ ID NO: 98, wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487 and in U.S. Pat. No. 4,683,195 and U.S. Pat. No. 4,683,203.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one aspect, gene expression is detected and quantitated by hybridization to a probe that specifically hybridizes to the appropriate probe for that biomarker. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

Detection of Polypeptides

Expression level of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject afflicted with a cancer will respond to therapeutic treatment with a PRMT5 inhibitor, comprising the steps of: a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor; wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method comprises the following optional steps:

b) determining the level of PRMT5 in the subject, wherein steps a) and b) can be performed in any order;

c) administering a therapeutically effective amount of a PRMT5 inhibitor to the subject; and

d) determining the level of PRMT5 in the subject following step c), wherein a decrease in the level of PRMT5 is correlated with the inhibition of the proliferation of the cancer, and wherein steps c) and d) are performed after steps a) and b).

Assaying for Biomarkers and PRMT5 Inhibitor Treatment

A number of patient stratification strategies could be employed to find patients likely to be sensitive to PRMT5 depletion, including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

Survival of MTAP-deficient and/or MTA-accumulating cancer cells or tumors can be assayed for after PRMT5 inhibitor administration in order to determine if the patient remains sensitive to the PRMT5 inhibitor treatment. In addition, survival can be assayed for in multiple timepoints after a single administration of a PRMT5 inhibitor. For example, after an initial bolus of an PRMT5 inhibitor is administered, survival can be assayed for at 1 hour, 2 hours, 3 hours, 4 hours, 8 hours, 16 hours, 24 hours, 48 hours, 3 days, 1 week or 1 month or several months after the first treatment.

Survival can be assayed for after each PRMT5 inhibitor administration, so if there are multiple PRMT5 inhibitor administrations, then assaying for survival for after each administration can determine continued patient sensitivity. The patient could undergo multiple PRMT5 inhibitor administrations and then assayed for survival at different timepoints. For example, a course of treatment may require administration of an initial dose of PRMT5 inhibitor, a second dose a specified time period later, and still a third dose hours after the second dose. Survival can be assayed for at 1 hour, 2 hours, 3 hours, 4 hours, 8 hours, 16 hours, 24 hours, 48 hours, 3 days, 1 week or 1 month or several months after administration of each dose of a PRMT5 inhibitor.

Finally, different PRMT5 inhibitors can be administered and followed by assaying for survival of MTAP deficiency and/or MTA accumulation-related cells or tumors. In this embodiment, more than one PRMT5 inhibitor is chosen and administered to the patient. Survival can then be assayed for after administration of each different PRMT5 inhibitor. This assay can also be done at multiple timepoints after administration of the different WNR inhibitor. For example, a first PRMT5 inhibitor could be administered to the patient and survival assayed for at 1 hour, 2 hours, 3 hours, 4 hours, 8 hours, 16 hours, 24 hours, 48 hours, 3 days, 1 week or 1 month or several months after administration. A second PRMT5 inhibitor could then be administered and survival can be assayed for again at 1 hour, 2 hours, 3 hours, 4 hours, 8 hours, 16 hours, 24 hours, 48 hours, 3 days, 1 week or 1 month or several months after administration of the second PRMT5 inhibitor.

Kits for assessing the activity of any PRMT5 inhibitor can be made. For example, a kit comprising nucleic acid primers for PCR or for microarray hybridization can be used for assessing PRMT5 inhibitor sensitivity (i.e., amenability to treatment with one or more PRMT5 inhibitors).

It is well known in the art that cancers can become resistant to chemotherapeutic treatment, especially when that treatment is prolonged. Assaying for MTAP deficiency and/or MTA accumulation can be done after prolonged treatment with any chemotherapeutic to determine if the cancer would be sensitive to the PRMT5 inhibitor. If the patient has been previously treated with another chemotherapeutic or another PRMT5 inhibitor, it is useful to assay for MTAP deficiency and/or MTA accumulation to determine if the tumor is sensitive to a PRMT5 inhibitor. This assay can be especially beneficial to the patient if the cancer goes into remission and then re-grows or has metastasized to a different site.

Kits

In some embodiments kits related to methods of the invention are provided.

In one embodiment, a for predicting the sensitivity of a subject afflicted with a MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

One skilled in the art will recognize many methods and materials similar or equivalent to those described herein, which could be used in the practice of the present invention. Indeed, the present invention is in no way limited to the methods and materials described.

### EXAMPLES

### Example 1

Materials and Methods

Library Design and Construction.

A custom 55,000 element shRNA library focused on enzymes with small molecule ligandable domains was constructed using chip based oligonucleotide synthesis and cloned as a pool into the Bbsl restriction sites of the pRSI16 lentiviral plasmid (Cellecta). The shRNA library targeted 2702 genes with an average of 20 unique shRNAs/gene. The shRNA includes 2 G/U mismatches in the passenger strand, a 7 nucleotide loop, and a 21 nucleotide targeting sequence. Targeting sequences were designed using a proprietary algorithm (Cellecta). The oligo corresponding to each shRNA was synthesized with a unique 22 nucleotide barcode for measuring representation by NGS (Next Generation Sequencing). Sequencing of the plasmid pool showed excellent normalization with >90% clones present at a representation of +/−5-fold from the median counts in the pool.

Viral Packaging. 2.1×10⁸ 293 T cells per plate were plated on multiple 5-layer CellStack flasks (Corning) 24 hrs prior to transfection. Cells were transfected according to the manufactures recommended protocol. For each flask, cells were transfected using 510.3 uL of TransIT reagent diluted in 18390 uL of OPTI-MEM that was combined with 75.6 ug of the plasmid pool and 94.5 ug of the Cellecta packaging mix (containing the psPAX2 and pMD2 plasmids that encode Gag/Pol and VSV-G respectively). Virus was harvested at 48 hrs post transfection, aliquotted, and frozen at −80 C for later use. Viral titer was measured by infecting HCT116 cells with a 10-point viral dose response curve and measuring the percentage infected cells by monitoring expression of the RFP expression cassette that is part of the viral construct by FACS. Typical viral titers were in the range of 1-5×10⁶ TU/mL using this procedure.

Viral Transduction and Pooled shRNA Screening:

Screening of the shRNA library was carried out across over 200 cell lines. For each cell line the optimal puromycin dose required to achieve >95% cell killing in 72 hrs was determined by measuring cell viability with a Cell TiterGlo assay for a 6-point dose response ranging from 0 to 5 ug puromycin. The volume of virus required to give an MOI of 0.3 was determined using a 10 point dose response ranging from 0 to 400 uL of viral supernatant in the presence of 8 ug/mL polybrene. Infectivity was determined as the % RFP positive cells as measured by FACS.

For large-scale infections, 60-million cells were plated 24 hrs prior to infection in S-layer cellstack flasks. On the day of infection, the culture media was replaced with fresh media containing 8 ug/mL polybrene and sufficient virus was added to give an MOI of 0.5 was added. 24 hrs after infection, the culture media was replaced with fresh media containing puromycin. 72 hrs following puromycin addition, cells were trypsinized, and 60 million cells were plated into new flasks. An aliquot of cells was used to measure transduction efficiency determined by measuring the % RFP positive cells and was typically >90%. Cells were maintained in culture and split as needed to ensure they did not exceed 90% confluence during the course of the screen. At each split, 60 million cells were passaged into new flasks, ensuring a representation of >1000 cells/shRNA in the library and the % RFP positive cells was measured to ensure stability of the transduced population over time. When the cells reached 5-population doublings, 100 million cells were harvested by centrifugation and stored at −20° C. for genomic DNA purification.

Purification of Genomic DNA & PCR for Library Production.

100 million cells were resuspended in in 10 ml PBS according to the QIAmp DNA Blood Maxi Kit (Qiagen). This resuspension is then treated with ProteinaseK, RNaseA and Buffer AL and are incubated for lysis, and processed for gDNA isolation as directed. The final DNA concentration is assayed using Picogreen reagent giving a typical yield of 2.5 ug gDNA per million cells.

For NGS library generation, the barcodes are amplified in 24×100 uL PCR reactions using 4 ug of gDNA per reaction with Titanium Taq and Primers #3323 (PEFwdGEX), #3324 (PECellectaA), #3197-3223 (one of 24 indexing oligos) for 28 cycles. The product was analyzed by agarose gel electrophoresis to check for the expected ˜120 bp product and purified using the Agencourt. AMPure XP PCR cleanup kit (Beckman Coulter) and the amount of purified product quantified gel electrophoresis and an Advanced Analytical Fragment Analyzer. Barcode representation was measured by Next Generation Sequencing on an Illumina HiSeq 2500. A plasmid control was run on each sequencing flow cell to control for sequencing effects on barcode representation.

Data Analysis.

Counts from each sample were normalized to 50 million reads. The number of reads observed for each barcode at day 14 post infection was divided by the number of reads for the corresponding barcode in the original plasmid pool to give the fold change in representation during the experiment. A z-score was calculated using the median and MAD for the fold change in counts across the entire shRNA library. The deep coverage shRNA libraries used in this work enable high confidence hit calling at the gene level, rather than analysis of individual shRNAs in the data set. For gene based hit calling, two statistical measures were used, (1) Redundant siRNA Activity or RSA, and (2) Q1 Z-score. To identify statistically significant correlations between shRNA sensitivity and genetic features of the cell lines, we first performed a k-means clustering for the RSA value for a particular gene across all the cell lines screened to identify groups of ‘sensitive’ and ‘in-sensitive’ cell lines. This partition was then used to calculate the statistical significance of the co-occurrence of all genetic features in the Cancer Cell Line Encyclopedia (CCLE) data set. Genotyping/copy number analysis was performed using Affymetrix Genome-Wide Human SNP Array 6.0 and expression analysis using the GeneChip Human Genome U133 Plus 2.0 Array. RSA is the Redundant siRNA Activity algorithm, which calculates gene-centric P-values. The RSA value provides a measure for each gene's statistical ranking of effects and is calculated for each cell line, which can then be compared across all cell lines screened. A more negative RSA value (<−3) is indicative of the gene being required for cell viability. The minimum RSA reflects the RSA value of the most sensitive cancer cell line, whereas median RSA represents the RSA value of the 90th (median) most sensitive cell line. Genes with broad anti-proliferative activity will display both a low minimum and median RSA value, as exemplified by controls targeting the proteasome (PSMA3) and mitotic machinery (PLK1). The epigenetic regulators BRD4, CHD4 and PHF5A showed ratios of minimum vs median RSA that were similar to PLK1, suggesting that inhibition of these targets results in relatively broad anti-proliferative effects.

Results

A pooled shRNA screen was carried out with a library of 55,000 shRNAs against 2702 genes, a depth of approximately 20 shRNAs per gene. Cells that had been transduced with the shRNA library were cultured for 14 days, and then the prevalence of shRNAs at the beginning and end of the experiment was counted by Illumina short-read DNA sequencing. The purpose of this screen was to find genes whose knockdown by shRNA was selectively lethal to specific cancer cells. It was expected that shRNAs that were selectively lethal would disappear from the population over time in sensitive cell lines. Over 270 cell lines of diverse cancer types from the Novartis/Broad Cancer Cell Line Encyclopedia (CCLE) were screened in this fashion, with the intent to discover selectively lethal genes in various subsets of cancer.

The pooled shRNA screening was able to recover known selective lethal genes, such as KRAS and BRAF as strongly depleting from the cell lines that were already known or suspected to be sensitive to their depletion.

RSA values were determined for depletion of KRAS across 228 canines.

The performance of known positive controls gave confidence that the screen was working as designed. In addition to these positive controls, the Protein Arginine Methyltransferase 5 gene (gene symbol PRMT5) showed a very strong depletion in a subset of cancer cell lines, while having no significant growth effect in the majority of lines screened.

RSA scores were determined for depletion of PRMT5 across 278 cell lines.

The top correlating feature in cell lines that were sensitive to depletion of PRMT5, versus those that were not, revealed methylthioadenosine phosphorylase (MTAP) copy number and expression to be the main stratifier between these two populations. Specifically, an overwhelming majority of cell lines sensitive to PRMT5 knockdown lacked MTAP and only two weakly sensitive lines (rkol and meljuso) were observed outside of this stratification. This pattern of specific lethality is highly suggestive of an important role for the PRMT5 gene in maintaining the proliferation and/or survival of these cells.

Expression of MTAP was the top distinguishing feature that correlated with PRMT5 knockdown sensitivity.

The top correlating expression and genetic features that associate with PRMT5 dependency include several genes located on the 9p21 locus and describes the extent of variability in size of deletion events. These events can be, but are not limited to, the region on chromosome 9 between chr9:20658308-22824212 encompassing the genomic region containing all genes between and including FOCAD to LINC01239 as assessed in the UCSC Genome Browser on Human February 2009 (GRCh37/hg19) assembly version. Under these circumstances the genes identified (as shown in the tables below) can also be used for stratification purposes in addition to MTAP and CDKN2A.

Top correlating expression features to PRMT5 dependence as assessed by RNASeq and microarray:

| FEATURE_NAME | FEATURE_TYPE | FOLD_CHANGE | NORMINAL . . . [symbol] | ADJUSTED_P . . . | CYTOBAND |
|---|---|---|---|---|---|
| MTAP | Expr | −2.70E+000 | 4.44E−016 | 8.27E−012 | 9p21 |
| MTAP | RNASeq_Expr | −4.65E+000 | 1.75E−015 | 4.15E−011 | 9p21 |
| CDKN2A | RNASeq_Expr | −5.38E+000 | 1.75E−010 | 2.08E−006 | 9p21 |
| CDKN2B | Expr | −8.30E−001 | 1.39E−009 | 9.22E−006 | 9p21 |
| CDKN2A | Expr | −1.89E+000 | 1.49E−009 | 9.22E−006 | 9p21 |
| CDKN2B | RNASeq_Expr | −2.7 [symbol] E+000 | 5.92E−009 | 4.62E−005 | 9p21 |
| CDKN2B-A51 | RNASeq_Expr | −1.43E+000 | 2.55E−007 | 1.35E−003 | 9p21.3 |
| [symbol] R31HG | RNASeq_Expr | −1.3 [symbol] E+000 | 2.8 [symbol] E−007 | 1.35E−003 | 9p21.3 |
| FOCAD | Expr | − [symbol] .71E−001 | [symbol] .73E−007 | 4.06E−003 | 9p21 |
| IFNE | Expr | −2. [symbol] 8E−001 | 4.15E−006 | 1.5 [symbol] E−002 | 9p21.3 |
| IFNE | RNASeq_Expr | −6.32E−001 | 5.23E−006 | 2.04E−002 | 9p21.3 |
| RECQL | Expr | 5. [symbol] 3E−001 | 1.07E−005 | 3.32E−002 | 12p12 |
| M [symbol] 31HG | Expr | −6.26E−001 | 1.89E−005 | 5.02E−002 | 9p21.3 |
| KLHL9 | Expr | −5.95E−001 | 2.22E−005 | 5.17E−002 | 9p22 |
| FOCAD | RNASeq_Expr | −7.53E−001 | 3.23E−005 | 1.08E−001 | 9p21 |

[symbol]

indicates data missing or illegible when filed

Top correlating copy number features to PRMT5 dependence as assessed by RNASeq and microarray:

| FEATURE_NAME | FEATURE_TYPE | FOLD_CHANGE | NORMINAL . . . [symbol] | ADJUSTED_P . . . | CYTOBAND |
|---|---|---|---|---|---|
| MTAP | CN | −1.50E+000 | 2.22E−015 | 4.87E−012 | 9p21 |
| CDKN2A | CN | −1.43E+000 | 8.53E−014 | 9.34E−01 [symbol] | 9p21 |
| C9orf53 | CN | −1.45E+000 | 2.90E−013 | 2.12E−009 | 9p21.3 |
| CDKN2B | CN | −1.41E+000 | 1.40E−012 | 7. [symbol] 5E−01 [symbol] | 9p21 |
| CDKN2B-AS1 | CN | −1.40E+000 | 2.75E−012 | 1.20E−005 | 9p21.3 |
| IFNE | CN | −1.49E+000 | 5.9 [symbol] E−01 [symbol] | 2.18E−005 | 9p21.3 |
| [symbol] R31 | CN | −1.51E+000 | 8.01E−010 | 2.51E−005 | 9p21.3 |
| [symbol] R31HG | CN | −1.42E+000 | 1.15E−009 | 3.15E−005 | 9p21.3 |
| IFNA1 | CN | −1.43E+000 | 1.10E−008 | 2. [symbol] E−005 | 9p22 |
| LINC01239 | CN | −1.39E+000 | 3.08E−009 | 6.76E−005 | 9p21.3 |
| DMRTA1 | CN | −1.44E+000 | 7.88E−009 | 1.57E−005 | 9p21.3 |
| IFNAB | CN | −6.15E−001 | 1.07E−007 | 1.95E−004 | 9p22 |
| PTPLAD2 | CN | −5.61E−001 | 2.30E−007 | 3.88E−004 | 9p21.3 |
| IFNB1 | CN | −5.60E−001 | 2. [symbol] E−007 | 4.09E−004 | 9p21 |
| IFNW1 | CN | −5.65E−001 | 2. [symbol] E−007 | 4.09E−004 | 9p22 |
| IFNA2 | CN | −5.64E−001 | 3.73E−007 | 4.09E−004 | 9p22 |
| IFNA4 | CN | −5.65E−001 | 3.79E−007 | 4.09E−004 | 9p22 |
| IFNA21 | CN | −5.65E−001 | 3.92E−007 | 4.09E−004 | 9p22 |
| IFNA7 | CN | −5.55E−001 | 3.92E−007 | 4.09E−004 | 9p22 |
| IFNA10 | CN | −5.55E−001 | 3.92E−007 | 4.09E−004 | 9p22 |
| IFNA16 | CN | −5.55E−001 | 3.92E−007 | 4.09E−004 | 9p22 |
| IFNA14 | CN | −5.55E−001 | 5.45E−007 | 5.07E−004 | 9p22 |
| IFNA17 | CN | −5.55E−001 | 5.45E−007 | 5.07E−004 | 9p22 |
| IFNA6 | CN | −5.55E−001 | 6.01E−007 | 5.07E−004 | 9p22 |
| IFNA13 | CN | −5.55E−001 | 6.01E−007 | 5.07E−004 | 9p22 |
| KLHL9 | CN | −5.55E−001 | 6.01E−007 | 5.07E−004 | 9p22 |
| IFNA5 | CN | −5.55E−001 | 6. [symbol] E−007 | 5.44E−004 | 9p22 |
| IFNA22P | CN | −5.55E−001 | 6. [symbol] E−007 | 5.44E−004 | 9p22 |
| FOCAD | CN | −5.05E−001 | 1.45E−005 | 1.12E−002 | 9p21 |

[symbol]

indicates data missing or illegible when filed

RSA values for PRMT5 were graphed for each cell line's MTAP expression status determined by microarray.

MTAP and CDKN2A expression levels in cell lines were screened in DRIVE. PRMT5 knockdown sensitive lines (ATARiS Q1 value <−1) are determined. ATARiS is an algorithim applied to the screen data to aggregate shRNA data for each individual gene in which only shRNAs with similar activity are aggregated together. Shao et al. 2013 Genome Res. 23: 665-78. A negative score indicates a decrease in proliferation; a positive score indicates proliferation.

MTAP is a gene located ˜100 kb telomeric to CDKN2A on chromosome 9p21 and as a result is a commonly co-deleted passenger event in cancer in the context of this tumor suppressor. MTAP functions in the methionine salvage pathway and is an enzyme required for the first step of this pathway after S-methyl-5′-thioadenosine (MTA) has been generated from S-adenosylmethionine (SAM), the methyl donor molecule required for the methyltransferase enzymatic reaction. It plays a major role in polyamine metabolism and its role is important for the salvage of both adenine and methionine within the cell. On top of this it is required for the proper recycling of SAM by catalyzing the reversible phosphorylation of MTA to adenine and 5-methylthioribose-1-phosphate.

The stratification with MTAP loss, as described, is specific to PRMT5 and was not observed with any of the other PRMTs tested in this work (PRMT1, CARM1, PRMT2, PRMT3, PRMT6, PRMT7, PRMT8 and PRMT10). This again highly suggests a specific role for PRMT5 biology in the cell cycle progression and survival of these cells. Additionally, accumulation of MTA has been shown to inhibit PRMT activity, and by blocking residual PRMT5 activity further this may result in a tipping point where cells go into crisis and exit from the cell cycle and/or death.

Frequency of MTAP homozygous deletions as published in the cBIO portal is determined. MTAP deletions occur in cancers with high unmet medical need eg. GBM, pancreatic, and melanoma.

RSA values are determined for PRMT1 and PRMT7 graphed for each cell line's MTAP expression status. RSA is used to show activity of PRMT5 across the cell lines.

Knockdown of the gene PRMT5 appears to very specifically inhibit the growth of cell lines exhibiting MTAP loss.

MTAP has close proximity to the frequently deleted tumor suppressor CDKN2A on chromosome 9; cell lines expressing MTAP expression are not affected. While this disclosure is not bound by any particular theory, it is noted that that the SAM salvage pathway is playing a role in the sensitivity seen. Lack of MTAP via a passenger deletion event presents a biological context where these cells are now sensitive to PRMT5 loss (an example of collateral lethality). MTAP loss is able to predict sensitivity to PRMT5 knockdown and can serve as a biomarker for patients that will likely benefit from PRMT5 inhibitors.

PRMT5 inhibition represents an attractive therapeutic target with the potential to impact a large patient population in cancers with high unmet medical need. MTAP deletions occur at a high frequency in several of these cancer types including glioblastoma (49.4%), bladder (46.2%), pancreatic (21.4%), melanoma (19%), lung (squamous −18.6%; adenocarcinoma −14.3%), DLBCL (14.3%) and head and neck (12.6%); TCGA provisional data sets as reported from Memorial Sloan-Kettering Cancer Center cBIO portal as of May 2014). Although loss of PRMT5 has been shown to be embryonic lethal in mice our data show synthetic lethality in the context of cancer lines with low MTAP expression and loss of CDKN2A.

Many methods of inhibiting PRMT5 are possible, including but not limited to: small molecules, siRNA therapeutics, cyclic peptides, aptamers, and CRISPRs. In addition this should not be limited to direct PRMT5 inhibition as given the correlation to synthetic lethality in DRIVE regulation of PRMT5 activity through its core complex member WDR77, or other binding partners that regulate substrate specificity eg. RIOK1, pICIn, target overlapping cell line models with statistical significance.

Top correlating shRNA synthetic lethal profiles to PRMT5 by Wilcoxon signed rank test in DRIVE with an FDR p-value <=0.25. Correlations were determined using each of the three metrics tested including RSA, ATARiS Quantile, and ATARiS Zmad.

Table 2 shows phenotypes of PRMT5 inhibition in 278 CCLE lines. RSA values below −2 indicate statistical significance of growth inhibition by PRMT5 knockdown.

In Table 2, “CN” indicates copy number. “Exp” indicates expression level as determined using a microarray and calculated according to Barretina et al. 2012 Nature 483: 603-607. A score of approximately 4.5 or less indicates deficiency.

Thus, Table 2 shows a strong correlation between MTAP deficiency (Exp less than about 4.5) and sensitivity to PRMT5 inhibition (RSA score of less than about −2).

TABLE 2
Phenotypes of PRMT5 inhibition in 278 CCLE lines. RSA values below −2
indicate statistical significance of growth inhibition by PRMT5 knockdown.

| Cell line name | Primary Site | RSA value | Expr.CDKN2A | CN.CDKN2A | Expr.MTAP | CN.MTAP |
|---|---|---|---|---|---|---|
| bftc909 | kidney | −16.22 | 4.163 | 0.57125231 | 3.39 | 0.57125231 |
| hec50b | endometrium | −15.975 | 4.427 | 0.667805555 | 3.834 | 0.807201075 |
| hel9217 | haematopoietic and | −15.87 | 4.678 | 0.469208157 | 3.891 | 0.469208157 |
| | lymphoid tissue | | | | | |
| a172 | central nervous system | −15.59 | 4.486 | 0.492603915 | 3.656 | 0.492603915 |
| ncih1437 | lung | −13.98 | 4.687 | 0.537821748 | 3.659 | 0.537821748 |
| sf268 | central nervous system | −12.88 | 4.386 | 0.677503357 | 3.758 | 0.735348476 |
| hcc1359 | lung | −12.17 | 4.678 | N/A | 3.711 | N/A |
| te14 | oesophagus | −12.16 | 4.436 | 0.648824409 | 3.626 | 0.648824409 |
| su8686 | pancreas | −11.24 | 4.353 | 0.469338267 | 3.462 | 0.469338267 |
| skhep1 | liver | −10.16 | 4.76 | 0.527484278 | 3.746 | 0.527484278 |
| miapaca2 | pancreas | −9.99 | 4.363 | 0.559922865 | 3.777 | 0.559922865 |
| rt112 | urinary tract | −9.95 | 4.386 | 0.532369564 | 3.624 | 0.532369564 |
| 1321n1 | central nervous system | −8.25 | 4.099 | 0.532185091 | 3.5 | 0.532185091 |
| a101d | skin | −8.02 | 4.436 | 0.674551267 | 3.559 | 0.674551267 |
| cmk115 | haematopoietic and | −7.94 | 4.839 | 0.645236519 | 3.757 | 0.645236519 |
| | lymphoid tissue | | | | | |
| heya8 | ovary | −7.86 | 4.743 | 0.585929909 | 3.727 | 0.585929909 |
| kp1n | pancreas | −7.54 | 4.807 | 0.63327323 | 3.79 | 0.846334569 |
| meljuso | skin | −7.462 | 4.191 | 0.719267339 | 3.685 | 1.265054904 |
| mdamb231 | breast | −7.31 | 4.838 | 0.555669901 | 3.747 | 0.555669901 |
| reh | haematopoietic and | −7.23 | 4.455 | 0.489778042 | 3.545 | 0.489778042 |
| | lymphoid tissue | | | | | |
| ks1 | central nervous system | −6.96 | 4.136 | 0.47371787 | 3.819 | 0.47371787 |
| a549 | lung | −6.53 | 4.537 | 0.618223328 | 3.822 | 0.89260884 |
| rko | large intestine | −6.35 | 4.751 | 2.290559438 | 7.963 | 2.290559438 |
| cas1 | central nervous system | −6.15 | 4.494 | 0.425284447 | 4.142 | 1.229694548 |
| snu1105 | central nervous system | −6.08 | 4.365 | 0.605878325 | 3.663 | 0.605878325 |
| sh4 | skin | −5.99 | 4.1 | 0.645862965 | 3.414 | 0.645862965 |
| kp4 | pancreas | −5.94 | 4.333 | 0.597661286 | 3.815 | 0.597661286 |
| dang | pancreas | −5.81 | 4.305 | 0.568014632 | 3.573 | 0.568014632 |
| ncih2126 | lung | −5.77 | 4.422 | 0.538418542 | 3.688 | 0.538418542 |
| daoy | central nervous system | −5.74 | 4.683 | 0.619252632 | 3.625 | 0.619252632 |
| hcc15 | lung | −5.47 | 4.814 | 0.833699861 | 4.057 | 1.113344444 |
| hs294t | skin | −5.44 | 4.485 | 1.082224645 | 3.978 | 1.082224645 |
| ncih838 | lung | −5.28 | 4.6 | 0.622221435 | 3.737 | 0.622221435 |
| abc1 | lung | −4.97 | 6.843 | 1.186078913 | 6.139 | 1.186078913 |
| achn | kidney | −4.82 | 4.227 | 0.565226104 | 3.621 | 0.565226104 |
| igr1 | skin | −4.48 | 4.466 | 0.487745342 | 3.727 | 0.487745342 |
| a2058 | skin | −4.37 | 7.749 | 1.986184991 | 6.947 | 1.986184991 |
| ncih2052 | pleura | −4.37 | 4.591 | 0.530490934 | 3.779 | 0.530490934 |
| mkn45 | stomach | −4.3 | 4.394 | 0.578023479 | 3.599 | 0.578023479 |
| cal851 | breast | −4.14 | 8.768 | 2.699140357 | 7.074 | 2.699140357 |
| sw1088 | central nervous system | −4.14 | 4.459 | 0.535329848 | 3.646 | 0.535329848 |
| 769p | kidney | −4.13 | 4.637 | 1.265142594 | 5.975 | 1.265142594 |
| ncih460 | lung | −3.94 | 4.037 | 0.589514825 | 7.282 | 1.64524222 |
| bxpc3 | pancreas | −3.859 | 4.716 | 0.590168981 | 3.52 | 0.590168981 |
| te6 | oesophagus | −3.83 | 4.231 | 0.621273318 | 3.548 | 0.621273318 |
| loximvi | skin | −3.73 | 4.504 | 0.604242682 | 4.32 | 2.445111067 |
| mcf7 | breast | −3.69 | 4.734 | 0.785999337 | 3.672 | 1.117054829 |
| ncih2122 | lung | −3.63 | 4.187 | 0.749343963 | 7.242 | 1.782373885 |
| hepg2 | liver | −3.57 | 5.207 | 1.994186031 | 6.591 | 1.994186031 |
| kasumi1 | haematopoietic and | −3.46 | 5.15 | 1.265493415 | 5.534 | 1.265493415 |
| | lymphoid tissue | | | | | |
| ku1919 | urinary tract | −3.42 | 4.378 | 0.522498934 | 3.763 | 0.522498934 |
| a2780 | ovary | −3.4 | 5.484 | 1.993080526 | 7.018 | 1.993080526 |
| sw403 | large intestine | −3.364 | 4.69 | 1.982196517 | 6.65 | 1.982196517 |
| huh6 | liver | −3.36 | 6.099 | 1.805501977 | 6.422 | 1.805501977 |
| hec265 | endometrium | −3.28 | 5.222 | 2.225608755 | 6.804 | 2.225608755 |
| ncih2009 | lung | −3.2 | 8.304 | 1.809134915 | 7.104 | 1.809134915 |
| hle | liver | −3.14 | 7.333 | 1.599809362 | 6.259 | 1.599809362 |
| skmel3 | skin | −3.1 | 5.916 | 1.275090812 | 6.618 | 1.275090812 |
| ln18 | central nervous system | −3.01 | 4.367 | 0.624424911 | 3.774 | 0.624424911 |
| sw1990 | pancreas | −2.99 | 4.436 | 0.656970274 | 6.435 | 1.598479232 |
| hs944t | skin | −2.88 | 7.852 | 1.621804589 | 6.454 | 1.621804589 |
| u87mg | central nervous system | −2.846 | 3.975 | 0.586376829 | 3.686 | 0.586376829 |
| chagok1 | lung | −2.81 | 6.928 | 1.214110726 | 5.552 | 1.214110726 |
| hec6 | endometrium | −2.774 | 4.294 | 2.325272828 | 6.838 | 2.325272828 |
| calu6 | lung | −2.75 | 7.9 | 1.583810796 | 6.303 | 1.583810796 |
| hmc18 | breast | −2.74 | 4.447 | 0.484074163 | 3.98 | 0.484074163 |
| snuc4 | large intestine | −2.73 | 3.991 | 1.279872414 | 6.871 | 1.973012368 |
| mdamb468 | breast | −2.66 | 7.536 | 1.611830457 | 6.172 | 1.611830457 |
| ncih1299 | lung | −2.64 | 7.511 | 1.745088255 | 6.492 | 1.745088255 |
| kyse70 | oesophagus | −2.62 | 4.542 | 0.637589599 | 6.38 | 2.469638707 |
| hct116 | large intestine | −2.61 | 6.851 | 2.328821397 | 8.349 | 2.328821397 |
| snb19 | central nervous system | −2.58 | 4.085 | 0.564521331 | 7.006 | 2.179655301 |
| colo829 | skin | −2.51 | 9.798 | 1.456191406 | 6.97 | 1.456191406 |
| uacc62 | skin | −2.48 | 4.688 | 0.69193096 | 7.644 | 1.795020101 |
| u118mg | central nervous system | −2.48 | 4.678 | 0.577703043 | 3.67 | 0.577703043 |
| ishikawaheraklio02er | endometrium | −2.45 | 7.764 | 2.02300516 | 6.371 | 2.02300516 |
| sem | haematopoietic and | −2.44 | 8.568 | 1.902504677 | 6.936 | 1.902504677 |
| | lymphoid tissue | | | | | |
| snu685 | endometrium | −2.42 | 7.883 | 1.725602295 | 6.099 | 1.725602295 |
| jimt1 | breast | −2.41 | 7.536 | 1.737002704 | 6.265 | 1.737002704 |
| g402 | soft tissue | −2.39 | 5.641 | 1.992251799 | 6.144 | 1.992251799 |
| kmrc20 | kidney | −2.39 | 4.611 | 0.817845368 | 6.689 | 2.097251677 |
| tccpan2 | pancreas | −2.39 | 4.626 | 0.608403349 | 3.618 | 0.686865725 |
| hcc38 | breast | −2.38 | 4.409 | 0.482666967 | 4.103 | 1.364715286 |
| hec1a | endometrium | −2.33 | 6.865 | 1.341363267 | 6.559 | 1.341363267 |
| patu8902 | pancreas | −2.33 | 7.61 | 2.179202102 | 8.098 | 2.179202102 |
| rerflcms | lung | −2.32 | 8.429 | 2.022724732 | 6.378 | 2.022724732 |
| wm793 | skin | −2.31 | 6.492 | 1.985909666 | 6.326 | 1.985909666 |
| jhh7 | liver | −2.29 | 6.779 | 1.498584048 | 6.512 | 1.498584048 |
| patu8988t | pancreas | −2.25 | 7.123 | 1.528906308 | 6.809 | 1.528906308 |
| sknsh | autonomic ganglia | −2.24 | 5.396 | 2.059080167 | 6.02 | 2.059080167 |
| pecapj41cloned2 | upper aerodigestive tract | −2.23 | 5.698 | 1.237990291 | 5.531 | 1.237990291 |
| lk2 | lung | −2.21 | 8.555 | 2.227615135 | 7.117 | 2.227615135 |
| cl34 | large intestine | −2.2 | 6.31 | 2.129625867 | 7.04 | 2.129625867 |
| a253 | salivary gland | −2.2 | 6.955 | 1.545956794 | 6.071 | 1.545956794 |
| ln229 | central nervous system | −2.181 | 4.435 | 0.767160317 | 6.92 | 2.410947106 |
| ncih1944 | lung | −2.18 | 4.307 | 0.604745486 | 6.1 | 1.364336959 |
| ncih1792 | lung | −2.17 | 7.902 | 1.161830815 | 6.252 | 1.161830815 |
| igr37 | skin | −2.14 | 4.254 | 1.357450885 | 6.108 | 1.357074572 |
| bc3c | urinary tract | −2.11 | 4.216 | 0.630426463 | 5.813 | 1.070066161 |
| km12 | large intestine | −2.1 | 7.75 | 1.999861375 | 6.598 | 1.999861375 |
| 786o | kidney | −2.06 | 4.725 | 0.699404993 | 6.795 | 1.797385664 |
| ncih2286 | lung | −2.05 | 7.171 | 1.259018952 | 6.353 | 1.259018952 |
| hcc827 | lung | −2.01 | 8.577 | 1.994739013 | 7.543 | 1.994739013 |
| skmel28 | skin | −2 | 7.256 | 1.767242476 | 6.853 | 1.767242476 |
| sudhl6 | haematopoietic and | −1.988 | 7.912 | 2.866718014 | 7.28 | 2.866718014 |
| | lymphoid tissue | | | | | |
| gb1 | central nervous system | −1.98 | 4.519 | 0.518530228 | 3.766 | 0.518530228 |
| molm16 | haematopoietic and | −1.96 | 9.061 | 2.199079865 | 6.767 | 2.199079865 |
| | lymphoid tissue | | | | | |
| wsudlcl2 | haematopoietic and | −1.96 | 7.539 | 1.950846141 | 6.624 | 1.950846141 |
| | lymphoid tissue | | | | | |
| mewo | skin | −1.95 | 5.874 | 1.158453391 | 7.07 | 1.158453391 |
| bt16 | central nervous system | −1.95 | 6.454 | N/A | 6.502 | N/A |
| cal51 | breast | −1.942 | 6.029 | 1.984533616 | 6.303 | 1.984533616 |
| cl11 | large intestine | −1.94 | 7.503 | 1.975064817 | 7.256 | 1.975064817 |
| 921 | eye | −1.93 | 6.147 | 1.619894661 | 7.097 | 1.619894661 |
| thp1 | haematopoietic and | −1.93 | 4.122 | 0.55953489 | 3.979 | 0.55953489 |
| | lymphoid tissue | | | | | |
| ht115 | large intestine | −1.92 | 6.986 | 2.281843741 | 6.958 | 2.281843741 |
| ls180 | large intestine | −1.91 | 4.37 | 2.014748836 | 6.7 | 2.014748836 |
| ncih2110 | lung | −1.89 | 7.53 | 1.77546879 | 6.469 | 1.77546879 |
| rcc4 | kidney | −1.87 | N/A | N/A | N/A | N/A |
| te11 | oesophagus | −1.87 | 4.57 | 0.607518396 | 7.292 | 2.370514142 |
| kyse510 | oesophagus | −1.86 | 4.294 | 0.55118174 | 6.422 | 1.75284691 |
| te1 | oesophagus | −1.85 | 7.471 | 1.542745408 | 6.432 | 1.624842634 |
| snu349 | kidney | −1.83 | 7.161 | 1.438235574 | 4.698 | 1.438235574 |
| kns81 | central nervous system | −1.82 | 4.398 | 0.746027129 | 6.343 | 1.985909666 |
| tyknu | ovary | −1.78 | 4.724 | 0.785563607 | 7.054 | 1.963054192 |
| kyse180 | oesophagus | −1.75 | 4.347 | 0.667342828 | 6.78 | 1.650496409 |
| vmrcrcw | kidney | −1.74 | 8.657 | 1.968640937 | 7.221 | 1.968640937 |
| huh7 | liver | −1.73 | 7.076 | 1.748357241 | 6.615 | 1.748357241 |
| sw948 | large intestine | −1.71 | 6.539 | 1.553583733 | 6.163 | 1.553583733 |
| colo320 | large intestine | −1.7 | 6.525 | 1.229950283 | 5.878 | 1.229950283 |
| kns62 | lung | −1.7 | 7.21 | 1.873193582 | 6.671 | 1.873193582 |
| sq1 | lung | −1.69 | 6.461 | 1.018114627 | 6.933 | 1.018114627 |
| corl105 | lung | −1.69 | 5.837 | 1.261202557 | 6.899 | 1.261202557 |
| ncih28 | pleura | −1.68 | 4.727 | 0.709906043 | 6.637 | 1.198973689 |
| ipc298 | skin | −1.65 | 8.296 | 1.402110449 | 6.837 | 1.402110449 |
| lmsu | stomach | −1.64 | 4.54 | 0.635119513 | 3.512 | 0.635119513 |
| ags | stomach | −1.63 | 4.228 | 1.992251799 | 7.065 | 1.992251799 |
| molm13 | haematopoietic and | −1.62 | 4.275 | 0.600318486 | 6.256 | 1.263214818 |
| | lymphoid tissue | | | | | |
| fadu | upper aerodigestive tract | −1.62 | 6.767 | 1.500454946 | 6.483 | 1.500454946 |
| hs852t | skin | −1.6 | 5.987 | 1.218832606 | 5.188 | 1.218832606 |
| ncih1568 | lung | −1.57 | 5.315 | 1.73868912 | 6.438 | 1.73868912 |
| skes1 | bone | −1.568 | 7.027 | 1.22603486 | 6.065 | 1.22603486 |
| sw1353 | bone | −1.56 | 6.174 | 1.962782073 | 6.047 | 1.962782073 |
| gi1 | central nervous system | −1.54 | 9.192 | 1.973012368 | 6.795 | 1.973012368 |
| me180 | cervix | −1.54 | N/A | N/A | N/A | N/A |
| colo205 | large intestine | −1.53 | 6.944 | 2.091589887 | 6.585 | 2.091589887 |
| ht55 | large intestine | −1.53 | 5.095 | 1.58952973 | 5.319 | 1.58952973 |
| ncih1048 | lung | −1.53 | 8.397 | 2.151436113 | 6.204 | 2.151436113 |
| sknmc | bone | −1.52 | 7.471 | 2.100161102 | 6.711 | 2.100161102 |
| kyse450 | oesophagus | −1.51 | 4.371 | 0.591274515 | 7.981 | 3.106952101 |
| monomac1 | haematopoietic and | −1.51 | 8.657 | 2.108182847 | 6.874 | 2.108182847 |
| | lymphoid tissue | | | | | |
| sw620 | large intestine | −1.5 | 7.493 | 2.053806124 | 7.082 | 2.053806124 |
| snu81 | large intestine | −1.5 | 7.264 | 2.031295318 | 5.902 | 2.031295318 |
| ncih747 | large intestine | −1.49 | 7.758 | 1.663820733 | 6.973 | 1.663820733 |
| ncih2228 | lung | −1.47 | 4.243 | 0.493663539 | 3.643 | 0.493663539 |
| kns42 | central nervous system | −1.47 | 7.114 | 1.864902234 | 5.721 | 1.864902234 |
| syo1 | soft tissue | −1.46 | N/A | N/A | N/A | N/A |
| lclc103h | lung | −1.45 | 4.188 | 0.554592496 | 7.046 | 2.180561983 |
| rchacv | haematopoietic and | −1.44 | 5.446 | 1.991837565 | 7.21 | 1.991837565 |
| | lymphoid tissue | | | | | |
| 697 | haematopoietic and | −1.43 | 4.711 | 2.014469552 | 6.852 | 2.014469552 |
| | lymphoid tissue | | | | | |
| colo679 | skin | −1.41 | 4.455 | 0.819036698 | 6.964 | 1.25989194 |
| sw480 | large intestine | −1.4 | 6.78 | 1.717845156 | 6.478 | 1.717845156 |
| wm2664 | skin | −1.4 | 4.559 | 0.480297432 | 5.994 | 1.272442091 |
| ls411n | large intestine | −1.39 | 5.041 | 2.059793913 | 6.869 | 2.059793913 |
| mfe296 | endometrium | −1.38 | 6.461 | 1.997367774 | 5.961 | 1.997367774 |
| panc0203 | pancreas | −1.38 | 7.336 | 1.520767915 | 6.428 | 1.520767915 |
| u251mg | central nervous system | −1.38 | 4.34 | 0.609078464 | 6.781 | 2.272373526 |
| te10 | oesophagus | −1.37 | 4.776 | 0.531300509 | 3.801 | 0.531300509 |
| a673 | bone | −1.37 | 4.201 | 0.547943862 | 5.586 | 1.268655181 |
| li7 | liver | −1.36 | 4.411 | 0.590619134 | 3.455 | 0.590619134 |
| ten | endometrium | −1.35 | 8.158 | 1.377354572 | 7.197 | 1.377354572 |
| huh1 | liver | −1.35 | 7.277 | 1.02023394 | 6.066 | 1.02023394 |
| gamg | central nervous system | −1.34 | 4.265 | 0.627157629 | 6.742 | 1.582493967 |
| hcc1806 | breast | −1.33 | 4.47 | 0.743549142 | 5.123 | 1.20163605 |
| ludlu1 | lung | −1.32 | 4.208 | 0.604955111 | 3.694 | 0.604955111 |
| pfeiffer | haematopoietic and | −1.31 | 6.522 | 1.941134386 | 6.893 | 1.941134386 |
| | lymphoid tissue | | | | | |
| detroit562 | upper aerodigestive tract | −1.3 | 5.24 | 1.517819253 | 6.888 | 1.517819253 |
| hlf | liver | −1.3 | 6.997 | 1.522138884 | 6.98 | 1.522138884 |
| hs695t | skin | −1.24 | 4.788 | 1.692786468 | 4.54 | 1.671333918 |
| rvh421 | skin | −1.24 | 4.415 | 1.010521488 | 6.678 | 1.479387509 |
| an3ca | endometrium | −1.21 | 7.444 | 1.945714453 | 5.972 | 1.945714453 |
| sbc5 | lung | −1.19 | 8.256 | 1.912951106 | 6.588 | 1.912951106 |
| a375 | skin | −1.19 | 4.943 | 0.738566638 | 8.41 | 2.633533844 |
| shp77 | lung | −1.19 | 6.662 | 1.337371242 | 5.956 | 1.337371242 |
| ncih1703 | lung | −1.19 | 7.681 | 1.275090812 | 6.8 | 1.275090812 |
| caki1 | kidney | −1.19 | 4.741 | 0.667065346 | 6.076 | 1.715227569 |
| pecapj34clonec12 | upper aerodigestive tract | −1.18 | 4.362 | 0.562802249 | 7.49 | 1.966186264 |
| skmel5 | skin | −1.18 | 4.174 | 0.579226695 | 3.703 | 0.579226695 |
| a204 | soft tissue | −1.18 | 5.964 | 1.966186264 | 6.77 | 1.966186264 |
| wm1799 | skin | −1.17 | 4.672 | 1.075867193 | 6.234 | 1.675277396 |
| tc71 | bone | −1.16 | 4.595 | 1.069695369 | 5.426 | 1.069695369 |
| snu1079 | biliary tract | −1.16 | 4.786 | 0.677691227 | 6.028 | 1.557249382 |
| kyse410 | oesophagus | −1.15 | 7.737 | 1.576363227 | 6.909 | 1.576363227 |
| ncih1355 | lung | −1.15 | 8.019 | 1.77620734 | 6.866 | 1.77620734 |
| l33 | pancreas | −1.15 | 7.795 | 1.906861419 | 6.648 | 1.906861419 |
| lovo | large intestine | −1.15 | 5.336 | 2.126086066 | 7.127 | 2.126086066 |
| bicr6 | upper aerodigestive tract | −1.14 | 4.922 | 0.842121323 | 5.725 | 1.547672266 |
| sw48 | large intestine | −1.14 | 4.399 | 2.004580008 | 7.628 | 2.004580008 |
| jl1 | pleura | −1.13 | 4.143 | 0.742210337 | 6.034 | 1.34835464 |
| sf295 | central nervous system | −1.13 | 4.6 | 0.724671971 | 6.27 | 1.217819231 |
| dms273 | lung | −1.1 | 8.025 | 1.813654941 | 6.82 | 1.813654941 |
| monomac6 | haematopoietic and | −1.1 | 8.517 | 2.058794738 | 7.144 | 2.058794738 |
| | lymphoid tissue | | | | | |
| bt549 | breast | −1.09 | 9.339 | 2.839624172 | 6.844 | 2.839624172 |
| sw1417 | large intestine | −1.08 | 5.325 | 1.972602135 | 6.232 | 1.972602135 |
| rpmi7951 | skin | −1.08 | 7.883 | 1.815541616 | 6.825 | 1.815541616 |
| skmel30 | skin | −1.06 | 6.968 | 1.377736509 | 6.679 | 1.377736509 |
| colo741 | skin | −1.06 | 4.23 | 0.557483109 | 3.446 | 0.557483109 |
| ovcar8 | ovary | −1.04 | 7.31 | 1.68868472 | 6.71 | 1.68868472 |
| panc0403 | pancreas | −1.04 | 7.418 | 1.683308962 | 6.678 | 1.683308962 |
| ncih196 | lung | −0.996 | 8.408 | 1.881521793 | 6.611 | 1.881521793 |
| mdamb436 | breast | −0.99 | 6.904 | 1.492261115 | 6.302 | 1.492261115 |
| wm115 | skin | −0.99 | 4.643 | 1.26006661 | 6.125 | 1.26006661 |
| mfe319 | endometrium | −0.98 | 7.173 | 2.022304162 | 5.482 | 2.022304162 |
| ncih1975 | lung | −0.97 | 6.332 | 1.334315654 | 6.319 | 1.334315654 |
| hcc1954 | breast | −0.94 | 7.469 | 1.686228443 | 6.85 | 1.686228443 |
| kyse150 | oesophagus | −0.94 | 7.772 | 1.482672538 | 6.341 | 1.482672538 |
| cal120 | breast | −0.94 | 8.026 | 1.511729484 | 6.492 | 1.511729484 |
| corl23 | lung | −0.94 | 7.65 | 2.081754583 | 7.103 | 1.274648976 |
| hrt18 | large intestine | −0.93 | N/A | 2.034254243 | N/A | 2.034254243 |
| snu61 | large intestine | −0.93 | 6.754 | 1.618099136 | 6.575 | 1.618099136 |
| ncih1793 | lung | −0.93 | 4.51 | 1.933748269 | 6.173 | 1.933748269 |
| hs939t | skin | −0.92 | 5.731 | 1.880869821 | 6.754 | 1.880869821 |
| skmel2 | skin | −0.92 | 6.369 | 1.249629139 | 6.986 | 1.954094178 |
| pk1 | pancreas | −0.91 | 4.52 | 0.631169764 | 7.053 | 1.408149002 |
| ncih1838 | lung | −0.89 | 4.389 | 0.555939579 | 5.226 | 1.120699895 |
| mfe280 | endometrium | −0.87 | 8.203 | 1.776453592 | 6.075 | 1.776453592 |
| snuc2a | large intestine | −0.87 | 6.962 | 1.242374394 | 5.582 | 1.242374394 |
| skco1 | large intestine | −0.85 | 5.251 | 3.170257443 | 7.878 | 3.170257443 |
| mdamb453 | breast | −0.85 | 5.979 | 1.235161766 | 5.712 | 1.235161766 |
| ncih23 | lung | −0.84 | 6.274 | 1.220523438 | 5.297 | 1.220523438 |
| hcc1500 | breast | −0.83 | 4.271 | 0.471719125 | 3.512 | 0.471719125 |
| mdst8 | large intestine | −0.82 | 6.037 | 1.663820733 | 6.336 | 1.663820733 |
| snu423 | liver | −0.8 | 7.379 | 1.987011194 | 7.077 | 1.987011194 |
| rh41 | soft tissue | −0.79 | 7.226 | 1.449042591 | 6.106 | 1.449042591 |
| hcc1833 | lung | −0.79 | 5.695 | 1.228076132 | 4.703 | 1.228076132 |
| hcc44 | lung | −0.79 | 8.862 | 1.758566632 | 6.928 | 1.758566632 |
| c32 | skin | −0.79 | 4.498 | 0.955282936 | 5.554 | 1.490297131 |
| a498 | kidney | −0.77 | 4.109 | 0.719815962 | 6.937 | 2.407940928 |
| ncih2066 | lung | −0.77 | 8.316 | 1.908183613 | 6.351 | 1.908183613 |
| ymb1 | breast | −0.77 | 6.055 | 1.913216316 | 6.17 | 1.913216316 |
| ncih441 | lung | −0.75 | 6.214 | 1.045070305 | 5.556 | 1.045070305 |
| igr39 | skin | −0.74 | 4.787 | 2.162949527 | 6.021 | 1.83961007 |
| igrov1 | ovary | −0.724 | 5.644 | 2.029325093 | 6.509 | 2.029325093 |
| mdamb157 | breast | −0.72 | 7.961 | 2.564362115 | 5.773 | 2.564362115 |
| sum52pe | breast | −0.72 | N/A | N/A | N/A | N/A |
| ncih661 | lung | −0.71 | 6.885 | 1.790546518 | 5.561 | 1.790546518 |
| sw1271 | lung | −0.7 | 4.53 | 0.527813443 | 5.278 | 1.025054062 |
| ncih716 | large intestine | −0.7 | 6.228 | 0.995849753 | 5.954 | 0.995849753 |
| mel285 | eye | −0.7 | 6.477 | 2.481478578 | 7.751 | 2.481478578 |
| ncih2030 | lung | −0.67 | 6.744 | 1.335796278 | 5.773 | 1.335796278 |
| panc0504 | pancreas | −0.66 | 4.313 | 0.806865439 | 6.342 | 1.691848048 |
| kasumi2 | haematopoietic and | −0.65 | 5.669 | 2.008752751 | 6.681 | 2.008752751 |
| | lymphoid tissue | | | | | |
| efm192a | breast | −0.65 | 5.84 | 1.308124631 | 5.898 | 1.308124631 |
| te9 | oesophagus | −0.64 | 4.556 | 0.61501777 | 6.387 | 1.41705917 |
| hcc95 | lung | −0.64 | 4.848 | 0.570579571 | 5.852 | 1.449042591 |
| mdamb415 | breast | −0.62 | 7.32 | 1.875272172 | 5.927 | 1.875272172 |
| colo201 | large intestine | −0.59 | 7.43 | N/A | 6.834 | N/A |
| cfpac1 | pancreas | −0.58 | 8.095 | 1.4063932 | 6.344 | 1.4063932 |
| hs578t | breast | −0.57 | 4.345 | 1.694195074 | 4.856 | 1.694195074 |
| uacc257 | skin | −0.57 | 7.453 | 1.446433503 | 6.515 | 1.446433503 |
| ncih1693 | lung | −0.55 | 7.956 | 1.578768894 | 6.476 | 1.578768894 |
| dld1 | large intestine | −0.55 | 4.515 | 2.023986967 | 6.825 | 2.023986967 |
| ncih2172 | lung | −0.55 | 7.636 | 1.802625869 | 6.264 | 1.802625869 |
| snu886 | liver | −0.54 | 7.917 | 1.121710203 | 5.751 | 1.121710203 |
| snu407 | large intestine | −0.53 | 5.151 | 2.023425876 | 6.18 | 2.023425876 |
| sudhl4 | haematopoietic and | −0.519 | 7.189 | 1.937639257 | 6.074 | 1.937639257 |
| | lymphoid tissue | | | | | |
| sw1783 | central nervous system | −0.51 | 9.082 | 2.133763084 | 7.039 | 2.133763084 |
| sngm | endometrium | −0.5 | 6.366 | 1.9970909 | 5.951 | 1.9970909 |
| ht1376 | urinary tract | −0.46 | 7.689 | 1.836806942 | 6.641 | 1.836806942 |
| t47d | breast | −0.44 | 6.022 | 1.494331263 | 5.774 | 1.494331263 |
| ncih2291 | lung | −0.44 | 7.832 | 1.541783293 | 6.832 | 1.541783293 |
| cama1 | breast | −0.42 | 6.586 | 2.095072254 | 5.845 | 2.095072254 |
| colo678 | large intestine | −0.4 | 4.257 | 0.546123824 | 3.591 | 0.546123824 |
| ncih1435 | lung | −0.36 | 6.298 | 1.386646456 | 6.068 | 1.386646456 |
| ncih1573 | lung | −0.359 | 7.405 | 1.333391098 | 5.532 | 1.333391098 |
| cw2 | large intestine | −0.31 | 5.385 | 2.001386775 | 6.684 | 2.001386775 |
| sjrh30 | soft tissue | −0.27 | 7.349 | 1.786084084 | 5.916 | 1.786084084 |
| ncih522 | lung | −0.09 | 8.283 | 1.897763218 | 7.963 | 1.897763218 |

### Example 2: Identification of shRNA Against PRMT5

shRNA sequences were designed by Cellecta Inc.

The sequences of the target sequences of the oligonucleotides used are set forth in Table 3, from 5′ to 3′.

Table 3. The sequences of the target sequences of the PRMT5 shRNA. The shRNAs are divided into two groups, Group 1 and Group 2, wherein Group 1 shRNAs are generally superior.

The two groups are broken down to reflect in which ATARIS solution they contributed to. The solution group shRNAs are behaving in the same way; the phenotype is the same. In this way we can account for off-target effects. Most of the shRNAs in group 1 track with being synthetic lethal in MTAP-null lines, whereas in group 2 very few of them do. Generally speaking, if the shRNAs are not having an obvious phenotype they also get grouped into 1 solution, which in this case would be group 2.

Alternative names (from Cellecta) for some of the shRNAs are also presented; for example, PRMT5-1243 is also designated sh4736. This molecule has been validated by rescue experiments with HA-PRMT5 (PRMT5 with a HA tag).

TABLE 3
PRMT5 shRNAs

| | | | |
|---|---|---|---|
| shRNA | ALTERNATIVE | | SEQ ID |
| NAME | shRNA NAME | TARGET SEQUENCE | NO: |

GROUP 1

| | | | |
|---|---|---|---|
| PRMT5-1832 | sh1700 | CCCATCCTCTTCCCTATTAAG | 1 |
| PRMT5-963 | sh4734 | GTCCTCCACCTAATGCCTATG | 2 |
| PRMT5-598 | | GAATGCACCAACTACACACAC | 3 |
| PRMT5-235 | | GCGTTTCAAGAGGGAGTTCAT | 4 |
| PRMT5-2178 | | GGCTCAAGCCACCAATCTATG | 5 |
| PRMT5-1290 | | CGCTAGAGAACTGGCAGTTTG | 6 |
| PRMT5-1952 | | GTCTGTTCTGCTATTCATAAC | 7 |
| PRMT5-1656 | sh4738 | GCCATCCCAACAGAGATCCTA | 8 |
| PRMT5-645 | | CGTGGATGTGGTGGCACAACT | 9 |
| PRMT5-1139 | | CCAGAAGAGGAGAAGGATACC | 10 |
| PRMT5-1243 | sh4736 | GCGGATAAAGCTGTATGCTGT | 11 |
| PRMT5-722 | sh4732 | GACCTCCCATCTAATCATGTC | 12 |
| PRMT5-1142 | | GAAGAGGAGAAGGATACCAAT | 13 |
| PRMT5-569 | | CCAGAGGACCTGAGAGATGAT | 14 |
| PRMT5-1323 | sh4737 | GCCAAGTGACCGTAGTCTCAT | 15 |
| PRMT5-317 | sh1699 | AGGGACTGGAATACGCTAATT | 16 |
| PRMT5-940 | | CCTGGAATACTTAAGCCAGAA | 17 |
| PRMT5-1801 | | GACTCACTCTCCTGGGATGTT | 18 |

GROUP 2

| | | | |
|---|---|---|---|
| PRMT5-893 | | GGCACCAACCACCACTCAGAG | 19 |
| PRMT5-1604 | | CGGCTGCACAACTTCCACCAG | 20 |
| PRMT5-1570 | | CCCTGAGGCCCAGTTTGAGAT | 21 |
| PRMT5-2246 | | CGCACTCAGCCTCAAGAACTC | 22 |
| PRMT5-522 | sh4728 | CTGGCCATCACTCTTCCATGT | 23 |
| PRMT5-1106 | sh4735 | CAGGCCATCTATAAATGTCTG | 24 |
| PRMT5-161 | sh4729 | CCCGAAATAGCTGACACACTA | 25 |
| PRMT5-1855 | | GCCCATAACGGTACGTGAAGG | 26 |
| PRMT5-234 | sh4731 | CGCGTTTCAAGAGGGAGTTCA | 27 |
| PRMT5-1240 | | CCGGCGGATAAAGCTGTATGC | 28 |
| PRMT5-2114 | | GGAGCATTTCAATCTGCTTTC | 29 |
| PRMT5-2255 | sh1166 | CCTCAAGAACTCCCTGGAATA | 30 |
| PRMT5-720 | sh4730 | CTGACCTCCCATCTAATCATG | 31 |
| PRMT5-1668 | | GAGATCCTATGATTGACAACA | 32 |
| PRMT5-1577 | sh1167 | GCCCAGTTTGAGATGCCTTAT | 33 |
| PRMT5-922 | sh4733 | CTGCTCCTACCTCCAATACCT | 34 |
| PRMT5-520 | sh4727 | CACTGGCCATCACTCTTCCAT | 35 |

Of these, sh1699, sh4736, and sh4737 were most effective. sh4732, sh4738, and sh4733 were also effective. The target sequences of these molecules, particularly those of Group I, can be used to generate additional shRNAs and siRNAs and other molecules capable of mediating RNA interference against PRMT5.

For example, RNAi agents comprising these sequences or its complement or a portion of the sequence or its complement (e.g., 15 or more contiguous nt thereof) can be prepared; these can readily be modified in accordance with knowledge of modification and preparation of RNAi agents, as known in the art.

PRMT7 was not effected by the effective anti-PRMT5 shRNAs, showing their specificity to PRMT5.

### Example 3: Method for Patient Stratification

Patients suitable to treatment with PRMT5 depletion, can be identified using a number of methods including but not limited to, testing for MTAP deficiency.

The methods will be briefly described below.

Testing for MTAP Deficiency

MTAP deficiency can be tested using any method known in the art. These assays are sensitive for the detection of MTAP deficiency and should identify patients who could benefit from PRMT5 inhibition. For example, MTAP deficiency can be detected using a reagent or technique involving immunohistochemistry utilizing an antibody to MTAP, and/or genomic sequencing, nucleic acid hybridization or amplification utilizing at least one probe or primer comprising a sequence of at least 12 contiguous nucleotides (nt) of the sequence of MTAP provided in SEQ ID NO: 98.

Screening for mutation and silencing of MTAP gene

Sequencing and expression studies can be performed to determine deficiency of MTAP gene or its protein product.

Patients with a cancer which is MTAP-deficient and/or MTA-accumulating can be treated with a PRMT5 inhibitor, as described herein.

### Example 4: Predicted HLA Presented PRMT5 Peptides

We predicted the PRMT5 peptide sequences that are likely to be presented by HLA, using the method described in Stabilized Matrix Method, Tenzer S et al, 2005. PMID 15868101, which takes a regularized regression approach to modeling these processes. Further, it allows for higher order, non-additive contributions from some residues. After model training, the input to the method is a file of protein sequences (such as a fasta formatted file). For a defined peptide length (e.g. 9 amino acid) it scans through the protein and reports a score for each peptide related to how well the method predicts the peptide to be processed by the proteasome, carried by the transporter proteins, and bound to a particular MHC allele, as well as an overall score representing the entire process. High scoring peptide sequences can then be chosen for downstream analyses. For instance, the PRMT5 wildtype protein sequence contains a number of peptides predicted to be well-processed and high-affinity MHC binders:

| C-terminal | | Total | Proteasome | TAP | MHC | SEQ |
|---|---|---|---|---|---|---|
| position | 9-mer-sequence | score | score | score | score | ID NO: |
| 98 | MLQELNFGA | 4.19 | 1.12 | -0.15 | 3.22 | 101 |
| 566 | GMFSWFPIL | 4.01 | 1.1 | 0.38 | 2.53 | 102 |
| 177 | WMWWHNFRT | 3.81 | 0.89 | -0.19 | 3.11 | 103 |
| 489 | FEMPYVVRL | 3.78 | 1.19 | 0.32 | 2.26 | 104 |
| 600 | KKVWYEWAV | 3.59 | 0.93 | 0.26 | 2.4 | 105 |
| 109 | GLPAFLLPL | 3.5 | 0.99 | 0.36 | 2.14 | 106 |
| 380 | YAVEKNPNA | 3.39 | 0.96 | -0.16 | 2.59 | 107 |
| 107 | YLGLPAFLL | 3.34 | 1.19 | 0.41 | 1.74 | 108 |
| 298 | YLQSPLQPL | 3.31 | 1.19 | 0.34 | 1.77 | 109 |
| 447 | FLKDDGVSI | 3.26 | 1.03 | 0.19 | 2.04 | 110 |
| 140 | SMFWMRVPL | 3.23 | 0.94 | 0.56 | 1.72 | 111 |
| 220 | AILPTSIFL | 3.22 | 1.14 | 0.61 | 1.46 | 112 |
| 604 | YEWAVTAPV | 3.19 | 0.77 | 0.12 | 2.31 | 113 |
| 487 | AQFEMPYVV | 3.14 | 1.28 | 0.21 | 1.65 | 114 |
| 270 | SYLQYLEYL | 3.12 | 1.08 | 0.56 | 1.48 | 115 |
| 569 | SWFPILFPI | 3.11 | 0.83 | 0.36 | 1.92 | 116 |
| 567 | MFSWFPILF | 3.09 | 1.02 | 1.18 | 0.9 | 117 |
| 141 | MFWMRVPLV | 3 | 0.98 | 0.33 | 1.7 | 118 |
| 309 | NLESQTYEV | 2.95 | 0.99 | 0.04 | 1.92 | 119 |
| 495 | VRLHNFHQL | 2.83 | 1.15 | 0.56 | 1.12 | 120 |
| 440 | CLDGAQHFL | 2.82 | 1.23 | 0.26 | 1.32 | 121 |
| 185 | TLCDYSKRI | 2.81 | 1.12 | 0.29 | 1.39 | 122 |
| 178 | MWWHNFRTL | 2.8 | 1.35 | 0.59 | 0.85 | 123 |
| 541 | GFAGYFETV | 2.8 | 1.02 | 0.13 | 1.65 | 124 |
| 455 | IPGEYTSFL | 2.78 | 1.15 | 0.25 | 1.39 | 125 |
| 527 | CTLEFPVEV | 2.76 | 1.08 | 0.09 | 1.58 | 126 |
| 538 | VLHGFAGYF | 2.76 | 0.87 | 1.15 | 0.74 | 127 |
| 105 | GAYLGLPAF | 2.74 | 1 | 1.08 | 0.66 | 128 |
| 248 | LLKLEVQFI | 2.71 | 0.93 | 0.3 | 1.49 | 129 |
| 239 | KMHQRLIFR | 2.54 | 1.02 | 0.78 | 0.73 | 130 |
| 176 | TWMWWHNFR | 2.52 | 1.04 | 0.81 | 0.66 | 131 |
| 249 | LKLEVQFII | 2.52 | 0.89 | 0.28 | 1.34 | 132 |
| 550 | LYQDITLSI | 2.51 | 1 | 0.35 | 1.15 | 133 |
| 106 | AYLGLPAFL | 2.5 | 1.04 | 0.59 | 0.87 | 134 |
| 470 | KLYNEVRAC | 2.49 | 0.91 | 0.17 | 1.41 | 135 |
| 175 | KTWMWWHNF | 2.46 | 1.03 | 1.14 | 0.29 | 136 |
| 537 | TVLHGFAGY | 2.46 | 1.02 | 1.39 | 0.05 | 137 |
| 100 | QELNFGAYL | 2.45 | 1.11 | 0.39 | 0.95 | 138 |
| 602 | VWYEWAVTA | 2.33 | 1.13 | 0.03 | 1.17 | 139 |
| 33 | CMPVFHPRF | 2.32 | 0.96 | 1.15 | 0.2 | 140 |
| 247 | RLLKLEVQF | 2.28 | 1 | 1.18 | 0.1 | 141 |
| 573 | ILFPIKQPI | 2.28 | 0.77 | 0.24 | 1.28 | 142 |
| 608 | VTAPVCSAI | 2.25 | 0.84 | 0.32 | 1.09 | 143 |
| 29 | FDFLCMPVF | 2.24 | 1.01 | 0.96 | 0.26 | 144 |
| 525 | RYCTLEFPV | 2.23 | 0.83 | 0.42 | 0.97 | 145 |
| 96 | AAMLQELNF | 2.23 | 0.94 | 1.14 | 0.15 | 146 |
| 221 | ILPTSIFLT | 2.22 | 0.72 | -0.23 | 1.72 | 147 |
| 462 | FLAPISSSK | 2.18 | 0.68 | 0.2 | 1.3 | 148 |
| 195 | VALEIGADL | 2.15 | 0.95 | 0.54 | 0.66 | 149 |
| 201 | ADLPSNHVI | 2.14 | 1.01 | 0.16 | 0.98 | 150 |
| 240 | MHQRLIFRL | 2.14 | 0.98 | 0.47 | 0.69 | 151 |
| 31 | FLCMPVFHP | 2.13 | 0.88 | -0.04 | 1.3 | 152 |
| 384 | KNPNAVVTL | 2.11 | 1.17 | 0.32 | 0.62 | 153 |
| 236 | VLSKMHQRL | 2.1 | 1.08 | 0.39 | 0.63 | 154 |
| 543 | AGYFETVLY | 2.09 | 1.12 | 1.25 | -0.28 | 155 |
| 542 | FAGYFETVL | 2.06 | 1.25 | 0.31 | 0.5 | 156 |
| 66 | GRDWNTLIV | 2.05 | 0.88 | 0.09 | 1.08 | 157 |
| 63 | LLSGRDWNT | 2 | 0.85 | -0.28 | 1.43 | 158 |

Unless defined otherwise, the technical and scientific terms used herein have the same meaning as that usually understood by a specialist familiar with the field to which the disclosure belongs.

### Example 5: MTA Accumulation and Sensitivity to PRMT5 Inhibition

MTA accumulation in cancer cells correlates with sensitivity to PRMT5 inhibition.

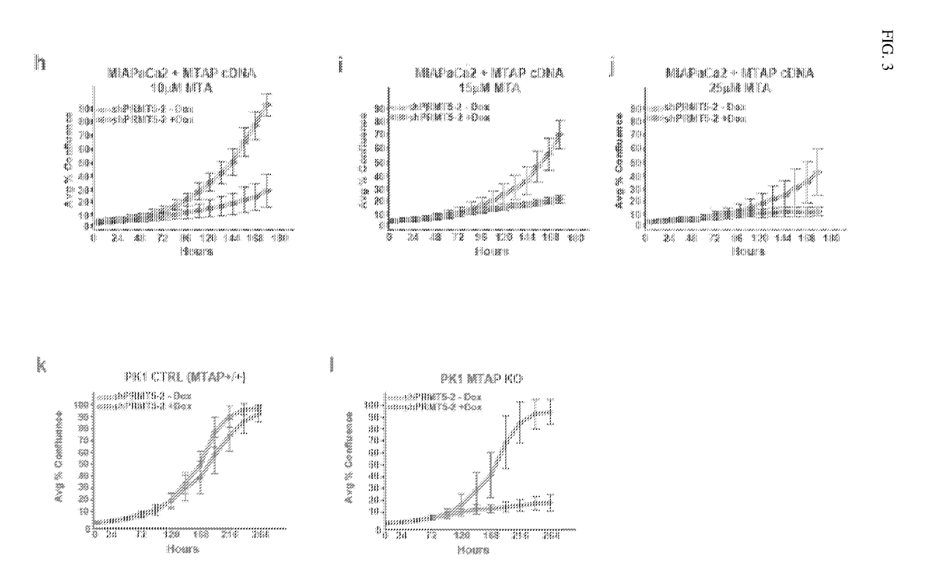

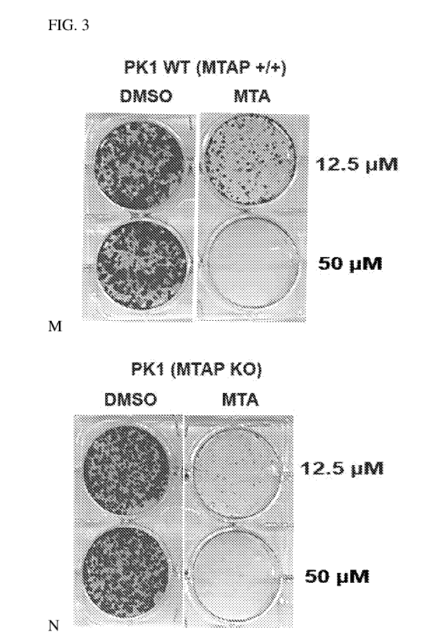

FIG. 3 shows that MTA accumulation sensitizes MTAP expressing cells to partial loss of PRMT5. FIGS. 3 A and B show complete inactivation of PRMT5 also inhibits growth in MTAP proficient cells. Top: Schematic representation of multi-allele dual-selection assay with P2A or STOP insertion cassettes and crystal violet staining of cells. FIG. 3 A shows that MTAP proficient HCT116 cells containing P2A insertion events, which allows for translation of the PRMT5 coding region due to exon skipping, were recovered with both single Puromycin (+Puro) or Blasticidin (+Blast) and double antibiotic selection (+Puro, +Blast). FIG. 3 B shows that STOP insertion events were only recovered after single antibiotic selection with Puro or Blast but not with double antibiotic selection, indicating that bi-allelic PRMT5 inactivation is not compatible with cell growth in this cell line. FIG. 3 C shows that MTA selectively inhibits PRMT5 but not other histone methyltransferases. Profiling of the inhibitory activity (IC50) of adenosine and MTA across a panel of histone methyltransferases (HMTs) in an LC/MS based biochemical assay. MTA and adenosine display single digit μM activity against the PRMT5/MEP50 complex (red) but have no activity (IC50>100 μM) across all other HMTs tested. FIG. 3 D shows an immunoblot of PK1 MTAP isogenic cell lines generated by CRISPR/Cas9 using a sgRNA non-targeting control (MTAP+/+) or an sgRNA targeting MTAP (MTAP KO) and probed with antibodies as indicated. FIG. 3 E shows an immunoblot of MIAPaCa2 cell lines stably expressing shPRMT5-2 and either MTAP or empty vector control (Empty) and blotted for PRMT5, MTAP, symmetric dimethylation of H4R3me2 (H4R3me2s) and loading control (Vinculin). FIGS. 3 F and G show the growth inhibitory effect of PRMT5 knockdown in MTAP deficient cells is abrogated by ectopic MTAP re-expression. MIAPaCa2 cells stably expressing inducible shPRMT5-2 were transduced with pRetro empty vector control or with MTAP cDNA and proliferation assessed by confluence measurements using Incucyte. FIG. 3 F shows PRMT5 shRNA mediated effects on proliferation in MIAPaCa2 shPRMT5-2 stable cells and empty vector (pRetro) control (compare shPRMT5-2-Dox (blue) vs shPRMT5-2+Dox (red); shown are mean and SD, n=6 p<0.001). FIG. 3 G shows an in vitro proliferation assay showing PRMT5 knockdown mediated defects are rescued by expression of shRNA-resistant MTAP (shown are mean and SD, n=6, NS p=0.7979). FIG. 3 H-J show that exogenous addition of MTA restores sensitivity of MTAP rescued cells to PRMT5 knockdown. In vitro proliferation assay of MIAPaCa2 cells stably expressing MTAP and inducible shPRMT5-2+/−Dox (n=6, shown are mean and SD) and treated with FIG. 3 H, 10 uM MTA (+/−Dox p=<0.001); FIG. 3 I, 15 uM MTA (+/−Dox p=<0.001); FIG. 3 J shows 25 uM MTA (+/−Dox p=<0.001). FIGS. 3 K and L show in vitro proliferation assay of PK1 MTAP isogenic cell lines generated from a non-targeting control sgRNA FIG. 3 K, PK1 CTRL; or with a sgRNA targeting MTAP 1, PK1 MTAP KO and stably expressing Dox-inducible PRMT5 (shPRMT5-2) with and without treatment with doxycycline (+/−Dox) over time (hours) as assessed by incucyte (n=24 per treatment condition). Additional experiments used the matched PK1 cell lines, parental (WT) vs MTAP KO (knockout), as shown in FIGS. 3, M and N. Treatment of the MTAP KO line shows greater sensitivity to MTA treatment (compare 12.5 uM dose), presumably due to the partial inhibition of PRMT5 imposed by higher levels of endogenous MTA as a result of MTAP loss. FIGS. 3 M and N show focus formation of PK1 (FIG. 3M), wild type (WT) and (FIG. 3N), MTAP knockout (MTAP KO) cells treated with DMSO or MTA at concentrations as indicated.

Thus, we show here that MTA itself or MTAP deficiency create sensitization to loss of PRMT5 activity. PRMT5 is essential, but when PRMT5 inhibitor MTA is aberrantly raised in some cells (e.g., accumulates), surviving cells will have a reduced but non-zero amount of PRMT5 activity. When a second PRMT5 inhibitor (or additional MTA) is systemically introduced (e.g., introduced into the entire body), it will lower the PRMT5 activity in all cells receiving the inhibitor (or additional MTA). The normal cells, with a normal level of PRMT5 activity, will be able to survive a decrease in PRMT5. But aberrant cells, wherein PRMT5 activity is already reduced (such as disease cells such as cancer cells), will have PRMT5 further reduced, such that these cells cannot survive and/or proliferate. The therapeutic window of administration of a PRMT5 inhibitor, therefore, would be the dosage of PRMT5 inhibitor which does not kill normal cells (with a normal level of PRMT5 activity), but which kills cells (e.g., cancer cells), which already have a reduced PRMT5 level (e.g., cells with MTAP deficiency or MTA accumulation).

### EMBODIMENTS

1. A method for inhibiting the proliferation of MTAP-deficient and/or MTA-accumulating cells in a subject in need thereof, the method comprising the step of administering to the subject a PRMT5 inhibitor in an amount that is effective to inhibit the proliferation of the MTAP-deficient and/or MTA-accumulating cells.

2. The method of claim 1, wherein the MTAP-deficient and/or MTA-accumulating cells are also deficient in CDKN2A.

3. The method according to any of the preceding claims, wherein the MTAP-deficient and/or MTA-accumulating cells are cancer cells.

4. The method according to claim 3, wherein the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

5. The method according to any of the preceding claims, wherein the PRMT5 inhibitor is selected from the group consisting of: a RNAi agent, a CRISPR, a TALEN, a zinc finger nuclease, an mRNA, an antibody or derivative thereof, an antibody-drug conjugate, a chimeric antigen receptor T cell (CART) or a low molecular weight compound.

6. The method according to claim 5, wherein the PRMT5 inhibitor is a low molecular weight compound.

7. The method according to claim 5, wherein the PRMT5 inhibitor is a RNAi agent.

8. The method according to claim 5, wherein the PRMT5 inhibitor is an antibody or derivative thereof.

9. The method of claim 8, wherein the antibody or a derivative thereof binds to a HLA-peptide complex comprising a peptide having the sequence of any of SEQ ID NOs: 101-158.

10. The method according to any of the preceding claims, wherein the method further comprises the step of administering to a subject a second therapeutic agent.

11. The method according to claim 10, wherein the second therapeutic agent is an anti-cancer agent, anti-allergic agent, anti-nausea agent or anti-emetic agent, pain reliever, cytoprotective agent.

12. The method according to claim 10, wherein the second therapeutic agent is an anti-cancer agent selected from the list consisting of: HDAC inhibitor, fluorouracil (5-FU) irinotecan, a HDM2 inhibitor, a purine analogue, 6-thioguanine, 6-mercaptopurine, a CDK4 inhibitor, and LEE011 and inhibitors of HDM2i, PI3K/mTOR-I, MAPKi, RTKi (EGFRi, FGFRi, METi, IGFiRi, JAKi, and WNTi.

13. A method of determining if a subject afflicted with a cancer will respond to therapeutic treatment with a PRMT5 inhibitor, comprising the steps of:

a) evaluating a test sample obtained from said subject for MTAP level and/or MTA level, and b) evaluating a reference sample from a non-cancerous or normal control subject for MTAP level and/or MTA level, wherein steps a) and b) can be performed in any order; and c) comparing the levels, wherein MTAP deficiency and/or MTA accumulation in the test sample relative to the reference sample indicates that the subject will respond to therapeutic treatment with a PRMT5 inhibitor;

wherein the method comprises the following optional steps:

d) determining the level of PRMT5 in the subject, wherein steps a) and b) can be performed in any order;

e) administering a therapeutically effective amount of a PRMT5 inhibitor to the subject; and

f) determining the level of PRMT5 in the subject following step e), wherein a decrease in the level of PRMT5 is correlated with the inhibition of the proliferation of the cancer, and wherein steps d), e) and f) are performed after steps a) and b).

14. The method of claim 13, wherein the MTAP-deficient and/or MTA-accumulating cells are also deficient in CDKN2A.

15. The method according to any of claims 13-14, wherein the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

16. The method according to any of claims 13-15, wherein the PRMT5 inhibitor is selected from the group consisting of: a RNAi agent, a CRISPR, a TALEN, a zinc finger nuclease, an mRNA, an antibody or derivative thereof, an antibody-drug conjugate, a chimeric antigen receptor T cell (CART) or a low molecular weight compound.

17. The method according to claim 16, wherein the PRMT5 inhibitor is a low molecular weight compound.

18. The method according to claim 16, wherein the PRMT5 inhibitor is a RNAi agent.

19. The method according to claim 16, wherein the PRMT5 inhibitor is an antibody or derivative thereof.

20. The method according to claim 19, wherein the antibody or a derivative thereof binds to a HLA-peptide complex comprising a peptide having the sequence of any of SEQ ID NOs: 101-158.

21. The method according to any of claims 13-21, wherein the method further comprises the step of administering to a subject a second therapeutic agent.

22. The method according to claim 21, wherein the second therapeutic agent is an anti-cancer agent, anti-allergic agent, anti-nausea agent or anti-emetic agent, pain reliever, cytoprotective agent.

23. The method according to claim 21, wherein the second therapeutic agent is an anti-cancer agent selected from the list consisting of: HDAC inhibitor, fluorouracil (5-FU) irinotecan, a HDM2 inhibitor, a purine analogue, 6-thioguanine, 6-mercaptopurine, a CDK4 inhibitor, and LEE011, and inhibitors of HDM2i, PI3K/mTOR-I, MAPKi, RTKi (EGFRi, FGFRi, METi, IGFiRi, JAKi, and WNTi.

24. A composition comprising a PRMT5 inhibitor for use in treatment of cancer in a selected patient population, wherein the patient population is selected on the basis of being afflicted with a MTAP-deficient and/or MTA-accumulating cancer.

25. The composition of claim 24, wherein the MTAP-deficient and/or MTA-accumulating cancer is also CDKN2A-deficient.

26. The composition according to any of claims 24-25, wherein the cancer is selected from a group consisting of glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura and large intestine.

27. The composition according to any of claims 24-26, wherein the PRMT5 inhibitor is selected from the group consisting of: a RNAi agent, a CRISPR, a TALEN, a zinc finger nuclease, an mRNA, an antibody or derivative thereof, an antibody-drug conjugate, a chimeric antigen receptor T cell (CART) or a low molecular weight compound.

28. The composition according to claim 27, wherein the PRMT5 inhibitor is a low molecular weight compound.

29. The composition according to claim 27, wherein the PRMT5 inhibitor is a RNAi agent.

30. The composition according to claim 27, wherein the PRMT5 inhibitor is an antibody or derivative thereof.

31. The composition according to claim 30, wherein the antibody or a derivative thereof binds to a HLA-peptide complex comprising a peptide having the sequence of any of SEQ ID NOs: 101-158.

Unless indicated otherwise, all methods, steps, techniques and manipulations that are not specifically described in detail can be performed and/or have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of various materials and methods is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A method for inhibiting the proliferation of MTAP-deficient or MTA-accumulating cells in a subject in need thereof, the method comprising administering to the subject a PRMT5 inhibitor in an amount that is effective to inhibit the proliferation of the MTAP-deficient or MTA-accumulating cells.

2. The method of claim 1, wherein the MTAP-deficient or MTA-accumulating cells are also deficient in CDKN2A.

3. A method for inhibiting the proliferation of MTAP-deficient or MTA-accumulating cells in a subject in need thereof, the method comprising administering to the subject a PRMT5 inhibitor in an amount that is effective to inhibit the proliferation of the MTAP-deficient or MTA-accumulating cells.

4. The method of claim 1, wherein the MTAP-deficient or MTA-accumulating cells are also deficient in CDKN2A.

5. The method of claim 1, wherein the MTAP-deficient or MTA-accumulating cells are cancer cells.

6. The method according to claim 3, wherein the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

7. A method for inhibiting the proliferation of MTAP-deficient or MTA-accumulating cells in a subject in need thereof, the method comprising administering to the subject a PRMT5 inhibitor in an amount that is effective to inhibit the proliferation of the MTAP-deficient or MTA-accumulating cells.

8. A method for inhibiting the proliferation of MTAP-deficient or MTA-accumulating cells in a subject in need thereof, the method comprising ef-administering to the subject a PRMT5 inhibitor in an amount that is effective to inhibit the proliferation of the MTAP-deficient or MTA-accumulating cells.

9. The method of claim 1, wherein the MTAP-deficient or MTA-accumulating cells are also deficient in CDKN2A.

10. The method of claim 1, wherein the MTAP-deficient and/or or MTA-accumulating cells are cancer cells.

11. The method according to claim 3, wherein the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

12. The method of claim 1, wherein the PRMT5 inhibitor is selected from the group consisting of: a RNAi agent, a CRISPR, a TALEN, a zinc finger nuclease, an mRNA, an antibody or derivative thereof, an antibody-drug conjugate, a chimeric antigen receptor T cell (CART) or a low molecular weight compound.

13. The method according to claim 5, wherein the PRMT5 inhibitor is a low molecular weight compound.

14. The method according to claim 5, wherein the PRMT5 inhibitor is a RNAi agent.

15. The method according to claim 5, wherein the PRMT5 inhibitor is an antibody or derivative thereof.

16. The method of claim 8, wherein the antibody or a derivative thereof binds to a HLA-peptide complex comprising a peptide having the sequence of any of SEQ ID NOs: 101-158.

17. The method of claim 1, wherein the method further comprises administering to the subject a second therapeutic agent.

18. The method according to claim 10, wherein the second therapeutic agent is an anti-cancer agent, anti-allergic agent, anti-nausea agent or anti-emetic agent, pain reliever, or cytoprotective agent.

19. The method according to claim 10, wherein the second therapeutic agent is an anti-cancer agent selected from: HDAC inhibitor, fluorouracil (5-FU) irinotecan, a HDM2 inhibitor, a purine analogue, 6-thioguanine, 6-mercaptopurine, a CDK4 inhibitor, and LEE011 and inhibitors of HDM2i, PI3K/mTOR-I, MAPKi, RTKi (EGFRi, FGFRi, METi, IGFiRi, JAKi, or WNTi.

20. A method of selecting a subject afflicted with cancer for treatment with a PRMT5 inhibitor, comprising the steps of:
a) evaluating the MTAP level or MTA level in a test sample obtained from a subject who has cancer; b) comparing the MTAP level or MTA level in the test sample to the MTAP level or MTA level in a reference sample from a non-cancerous or normal control subject; c) selecting the subject for treatment with a PRMT5 inhibitor when the MTAP level in the test sample is lower than the MTAP level in the reference sample or when the MTA level in the test sample is higher than the MTA level in the reference sample; and optionally administering a therapeutically effective amount of a PRMT5 inhibitor to the subject.

21. The method of claim 13, wherein the MTAP-deficient or MTA-accumulating cells are also deficient in CDKN2A.

22. The method of claim 13, wherein the cancer is glioblastoma, bladder cancer, pancreatic cancer, mesothelioma, melanoma, lung squamous, lung adenocarcinoma, diffuse large B-cell lymphoma (DLBCL), leukemia, or head and neck cancer, or cancer of the kidney, breast, endometrium, urinary tract, liver, soft tissue, pleura or large intestine.

23. The method of claim 13, wherein the PRMT5 inhibitor is selected from the group consisting of: a RNAi agent, a CRISPR, a TALEN, a zinc finger nuclease, an mRNA, an antibody or derivative thereof, an antibody-drug conjugate, a chimeric antigen receptor T cell (CART) or a low molecular weight compound.

24. The method according to claim 16, wherein the PRMT5 inhibitor is a low molecular weight compound.

25. The method according to claim 16, wherein the PRMT5 inhibitor is a RNAi agent.

26. The method according to claim 16, wherein the PRMT5 inhibitor is an antibody or derivative thereof.

27. The method according to claim 19, wherein the antibody or a derivative thereof binds to a HLA-peptide complex comprising a peptide having the sequence of any of SEQ ID NOs: 101-158.

28. The method of claim 13, wherein the method comprises administering to the subject a PRMT5 inhibitor.

29. The method according to claim 21, further comprising administering to the subject a second therapeutic agent, wherein the second therapeutic agent is an anti-cancer agent, anti-allergic agent, anti-nausea agent or anti-emetic agent, pain reliever, or cytoprotective agent.

30. The method according to claim 22, wherein the second therapeutic agent is an anti-cancer agent selected from: HDAC inhibitor, fluorouracil (5-FU) irinotecan, a HDM2 inhibitor, a purine analogue, 6-thioguanine, 6-mercaptopurine, a CDK4 inhibitor, and LEE011, and inhibitors of HDM2i, PI3K/mTOR-I, MAPKi, RTKi (EGFRi, FGFRi, METi, IGFiRi, JAKi, or WNTi.

24.-31. (canceled)
